(19) United States
(12) Patent Application Publication
(10) Pub. No.: US 2024/0188315 A1
(43) Pub. Date: Jun. 6, 2024

(54) LIGHT-EMITTING DEVICE INCLUDING HETEROCYCLIC COMPOUND, ELECTRONIC DEVICE INCLUDING THE LIGHT-EMITTING DEVICE, AND THE HETEROCYCLIC COMPOUND

(71) Applicant: Samsung Display Co., Ltd., Yongin-si (KR)

(72) Inventors: Kyoung Sunwoo, Yongin-si (KR); Chanseok Oh, Yongin-si (KR); Sunyoung Pak, Yongin-si (KR); Munki Sim, Yongin-si (KR)

(73) Assignee: Samsung Display Co., Ltd., Yongin-si (KR)

(21) Appl. No.: 18/323,647

(22) Filed: May 25, 2023

(30) Foreign Application Priority Data
Nov. 2, 2022 (KR) 10-2022-0144617

(51) Int. Cl. H10K 50/11; C07D 487/04; C07F 5/02; C07F 9/6561; C09K 11/06; H10K 50/15; H10K 50/16; H10K 50/18; H10K 50/81; H10K 50/82; H10K 85/60

(52) U.S. Cl. CPC H10K 50/11; C07D 487/04; C07F 5/027; C07F 9/6561; C09K 11/06; H10K 50/156; H10K 50/16; H10K 50/181; H10K 50/81; H10K 50/82; H10K 85/6572; H10K 85/658; C09K 2211/1018

(57) ABSTRACT

Embodiments provide a light-emitting device including a heterocyclic compound, and an electronic device including the light-emitting device. The light-emitting device includes a first electrode, a second electrode facing the first electrode, an interlayer between the first electrode and the second electrode and including an interlayer, and a heterocyclic compound. The heterocyclic compound is represented by Formula 1, which is explained in the specification:

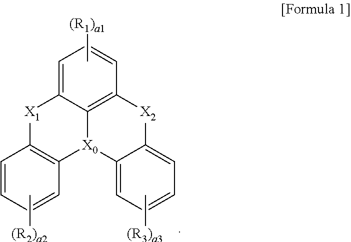

### CROSS-REFERENCE TO RELATED APPLICATION(S)

This application claims priority to and benefits of Korean Patent Application No. 10-2022-0144617 under 35 U.S.C. § 119, filed on Nov. 2, 2022, in the Korean Intellectual Property Office, the entire contents of which are incorporated herein by reference.

### BACKGROUND

### 1. Technical Field

Embodiments relate to a light-emitting device including a heterocyclic compound, an electronic device including the light-emitting device, and the heterocyclic compound.

### 2. Description of the Related Art

From among light-emitting devices, self-emissive devices have wide viewing angles, high contrast ratios, short response times, and excellent characteristics in terms of luminance, driving voltage, and response speed.

In a light-emitting device, a first electrode is located on a substrate, and a hole transport region, an emission layer, an electron transport region, and a second electrode are arranged on the first electrode. Holes provided from the first electrode move toward the emission layer through the hole transport region, and electrons provided from the second electrode move toward the emission layer through the electron transport region. Carriers, such as holes and electrons, recombine in the emission layer to produce excitons. These excitons transition from an excited state to a ground state to thereby generate light.

It is to be understood that this background of the technology section is, in part, intended to provide useful background for understanding the technology. However, this background of the technology section may also include ideas, concepts, or recognitions that were not part of what was known or appreciated by those skilled in the pertinent art prior to a corresponding effective filing date of the subject matter disclosed herein.

### SUMMARY

Embodiments include a light-emitting device including a heterocyclic compound, an electronic device including the light-emitting device, and the heterocyclic compound.

Additional aspects will be set forth in part in the description which follows and, in part, will be apparent from the description, or may be learned by practice of the embodiments of the disclosure.

According to embodiments, a light-emitting device may include a first electrode, a second electrode facing the first electrode, an interlayer between the first electrode and the second electrode and including an emission layer, and a heterocyclic compound represented by Formula 1:

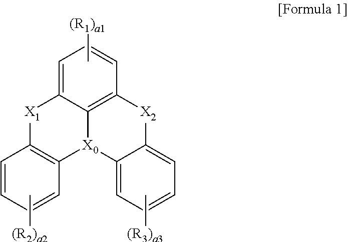

In Formula 1,
X₀ may be B, P(═O), or P(═S),
X₁ may be N(R₄), O, or S,
X₂ may be N(R₅), O, or S,
at least one of X₁ and X₂ may each independently be a group represented by one of Formulae 2-1 to 2-4,

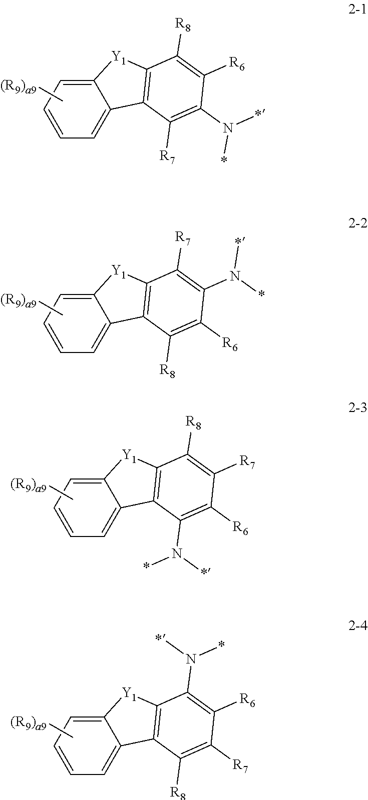

In Formulae 1 and 2-1 to 2-4,
Y₁ may be N(R₁₀), O, or S,
a1 to a3 and a9 may each independently be an integer from 1 to 4,
* and *′ may each independently indicate a binding site to a neighboring carbon atom,
R₁ to R₁₀ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
in Formulae 2-1 and 2-2, at least one of R₆ and R₇ may be a C₃-C₆₀ carbocyclic group unsubstituted or substituted with at least one R₁₀ₐ or a C₁-C₆₀ heterocyclic group unsubstituted or substituted with at least one R₁₀ₐ,
in Formulae 2-3 and 2-4, R₆ may be a C₃-C₆₀ carbocyclic group unsubstituted or substituted with at least one R₁₀ₐ or a C₁-C₆₀ heterocyclic group unsubstituted or substituted with at least one R₁₀ₐ,
R₁₀ₐ may be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to 023, and Q₃₁ to Q₃₃ may each independently be: hydrogen; deuterium; —F; —Cl; —Br; —I; a hydroxyl group; a cyano group; a nitro group; or a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof.

In an embodiment, the first electrode may be an anode; the second electrode may be a cathode;
the interlayer may further include a hole transport region between the first electrode and the emission layer, and an electron transport region between the emission layer and the second electrode;
the hole transport region may include a hole injection layer, a hole transport layer, an emission auxiliary layer, an electron blocking layer, or any combination thereof; and
the electron transport region may include a hole blocking layer, an electron transport layer, an electron injection layer, an electron control layer, or any combination thereof.

In an embodiment, the interlayer may include the heterocyclic compound.

In an embodiment, the emission layer may include the heterocyclic compound.

In an embodiment, the emission layer may further include at least one of a first host represented by Formula 5, a second host that includes a group represented by Formula 7, and a sensitizer, wherein Formulae 5 and 7 are described in the specification.

Embodiments provide an electronic device which may include the light-emitting device.

In an embodiment, the electronic device may further include a color filter, a color conversion layer, a touch screen layer, a polarizing layer, or any combination thereof.

Embodiments provide an electronic apparatus which may include the light-emitting device.

In an embodiment, the electronic apparatus may be a flat panel display, a curved display, a computer monitor, a medical monitor, a television, a billboard, an indoor light, an outdoor light, a signal light, a head-up display, a fully transparent display, a partially transparent display, a flexible display, a rollable display, a foldable display, a stretchable display, a laser printer, a telephone, a portable phone, a tablet personal computer, a phablet, a personal digital assistant (FDA), a wearable device, a laptop computer, a digital camera, a camcorder, a viewfinder, a micro display, a three-dimensional (3D) display, a virtual reality display, an augmented reality display, a vehicle, a video wall with multiple displays tiled together, a theater screen, a stadium screen, a phototherapy device, or a signboard.

Embodiments provide a heterocyclic compound which may be represented by Formula 1, which is described herein.

In an embodiment, X₁ and X₂ may be identical to each other.

In an embodiment, X₁ and X₂ may be different from each other.

In an embodiment, a1 to a3 may each be 1.

In an embodiment, R₁ to R₅, R₉, and R₁₀ may each independently be:
hydrogen, deuterium, —F, —Cl, —Br, —I, a cyano group, or a C₁-C₂₀ alkyl group;
a C₁-C₂₀ alkyl group substituted with at least one of deuterium, —F, —Cl, —Br, —I, —CD₃, —CD₂H,-CDH₂, —CF₃, —CF₂H, —CFH₂, a cyano group, a nitro group, a C₁-C₁₀ alkyl group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a naphthyl group, a pyridinyl group, and a pyrimidinyl group;
a phenyl group, a pyridine group, a dibenzofuranyl group, a dibenzothiophenyl group, a carbazolyl group, an azadibenzofuranyl group, an azadibenzothiophenyl group, or an azacarbazolyl group, each unsubstituted or substituted with at least one of deuterium, —F, —Cl, —Br, —I, —CD₃, —CD₂H,-CDH₂, —CF₃, —CF₂H, —CFH₂, a cyano group, a C₁-C₂₀ alkyl group, a phenyl group, a biphenyl group, a C₁-C₁₀ alkylphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzoimidazolyl group, a benzofuranyl group, a benzothiophenyl group, a benzoisothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, —Si(Q₃₁)(Q₃₂)(Q₃₃), —N(Q₃₁)(Q₃₂), or —B(Q₃₁)(Q₃₂); or
—Si(Q₁)(Q₂)(Q₃), —N(Q₁)(Q₂), or —B(Q₁)(Q₂), and
Q₁ to Q₃ and Q₃₁ to Q₃₃ may each independently be:
—H₃, —CD₃, —CD₂H,-CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, or —CD₂CDH₂; or
an n-propyl group, an isopropyl group, an n-butyl group, an isobutyl group, a sec-butyl group, a tert-butyl group, an n-pentyl group, an isopentyl group, a sec-pentyl group, a tert-pentyl group, a phenyl group, a naphthyl group, a pyridinyl group, a pyrimidinyl group, a pyridazinyl group, a pyrazinyl group, or a triazinyl group, each unsubstituted or substituted with at least one of deuterium, a C₁-C₁₀ alkyl group, a phenyl group, a biphenyl group, a pyridinyl group, a pyrimidinyl group, a pyridazinyl group, a pyrazinyl group, and a triazinyl group.

In an embodiment, R₁ to R₅, R₉, and R₁₀ may each independently be:
a methyl group, an ethyl group, an n-propyl group, an iso-propyl group, an n-butyl group, an isobutyl group, a sec-butyl group, a tert-butyl group, an n-pentyl group, an isopentyl group, a sec-pentyl group, a tert-pentyl group, a 2-methylbutyl group, a 2-2dimethylpropyl group, a 1-ethylpropyl group, or a 1,2-dimethylpropyl group, each unsubstituted or substituted with deuterium, —F, —Cl, —Br, —I, a hydroxyl group, a cyano group, or a nitro group; or
a benzene group, a pyridine group, a dibenzofuran group, a dibenzothiophene group, a carbazole group, an azadibenzofuran group, an azadibenzothiophene group, or an azacarbazole group, each unsubstituted or substituted with a methyl group, an ethyl group, an n-propyl group, an iso-propyl group, an n-butyl group, an isobutyl group, a sec-butyl group, a tert-butyl group, an n-pentyl group, an isopentyl group, a sec-pentyl group, a tert-pentyl group, a 2-methylbutyl group, a 2-2 dimethylpropyl group, 1-ethylpropyl, 1,2-dimethylpropyl, or a phenyl group, each unsubstituted or substituted with deuterium, —F, —Cl, —Br, —I, a hydroxyl group, a cyano group, or a nitro group.

In an embodiment, the heterocyclic compound may be represented by Formula 1-1, which is described in the specification.

In an embodiment, at least one of R₆ to R₈ may each independently be a C₆-C₆₀ aryl group unsubstituted or substituted with at least one R₁₀ₐ.

In an embodiment, at least one of R₆ to R₈ may each independently be a phenyl group, a pentalenyl group, a naphthyl group, an azulenyl group, an indacenyl group, an acenaphthyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a heptalenyl group, a naphthacenyl group, a picenyl group, a hexacenyl group, a pentacenyl group, a rubicenyl group, a coronenyl group, or an ovalenyl group.

In an embodiment, in Formula 1, at least one of X₁ and X₂ may each independently be a group represented by one of Formulae 3-1 to 3-10, which are described in the specification.

In an embodiment, the heterocyclic compound represented by Formula 1 may be any one of Compounds 1 to 192, which are described in the specification.

It is to be understood that the embodiments above are described in a generic and explanatory sense only and not for the purpose of limitation, and the disclosure is not limited to the embodiments described above.

### BRIEF DESCRIPTION OF THE DRAWINGS

The above and other aspects and features of the disclosure will be more apparent by describing in detail embodiments thereof with reference to the accompanying drawings, in which:

FIG. 1 is a schematic cross-sectional view of a light-emitting device according to an embodiment;

FIG. 2 is a schematic cross-sectional view of an electronic device according to an embodiment;

FIG. 3 is a schematic cross-sectional view of an electronic device according to another embodiment;

FIG. 4 is a schematic perspective view of an electronic apparatus according to an embodiment;

FIG. 5 is a schematic perspective view of an exterior of a vehicle as an electronic device including a light emitting device, according to an embodiment; and

FIGS. 6A to 6C are each a schematic diagram of the exterior of a vehicle as an electronic apparatus according to an embodiment.

### DETAILED DESCRIPTION OF THE EMBODIMENTS

The disclosure will now be described more fully hereinafter with reference to the accompanying drawings, in which embodiments are shown. This disclosure may, however, be embodied in different forms and should not be construed as limited to the embodiments set forth herein. Rather, these embodiments are provided so that this disclosure will be thorough and complete, and will fully convey the scope of the disclosure to those skilled in the art.

In the drawings, the sizes, thicknesses, ratios, and dimensions of the elements may be exaggerated for ease of description and for clarity. Like reference numbers and/or like reference characters refer to like elements throughout.

In the description, it will be understood that when an element (or region, layer, part, etc.) is referred to as being “on”, “connected to”, or “coupled to” another element, it can be directly on, connected to, or coupled to the other element, or one or more intervening elements may be present therebetween. In a similar sense, when an element (or region, layer, part, etc.) is described as “covering” another element, it can directly cover the other element, or one or more intervening elements may be present therebetween.

In the description, when an element is “directly on,” “directly connected to,” or “directly coupled to” another element, there are no intervening elements present. For example, “directly on” may mean that two layers or two elements are disposed without an additional element such as an adhesion element therebetween.

It will be understood that the terms “connected to” or “coupled to” may refer to a physical, electrical and/or fluid connection or coupling, with or without intervening elements.

As used herein, the expressions used in the singular such as “a,” “an,” and “the,” are intended to include the plural forms as well, unless the context clearly indicates otherwise.

As used herein, the term “and/or” includes any and all combinations of one or more of the associated listed items. For example, “A and/or B” may be understood to mean “A, B, or A and B.” The terms “and” and “or” may be used in the conjunctive or disjunctive sense and may be understood to be equivalent to “and/or”.

In the specification and the claims, the term “at least one of” is intended to include the meaning of “at least one selected from the group of” for the purpose of its meaning and interpretation. For example, “at least one of A and B” may be understood to mean “A, B, or A and B.” When preceding a list of elements, the term, “at least one of,” modifies the entire list of elements and does not modify the individual elements of the list.

It will be understood that, although the terms first, second, etc. may be used herein to describe various elements, these elements should not be limited by these terms. These terms are only used to distinguish one element from another element. Thus, a first element could be termed a second element without departing from the teachings of the disclosure. Similarly, a second element could be termed a first element, without departing from the scope of the disclosure.

The spatially relative terms “below”, “beneath”, “lower”, “above”, “upper”, or the like, may be used herein for ease of description to describe the relations between one element or component and another element or component as illustrated in the drawings. It will be understood that the spatially relative terms are intended to encompass different orientations of the device in use or operation, in addition to the orientation depicted in the drawings. For example, in the case where a device illustrated in the drawing is turned over, the device positioned “below” or “beneath” another device may be placed “above” another device. Accordingly, the illustrative term “below” may include both the lower and upper positions. The device may also be oriented in other directions and thus the spatially relative terms may be interpreted differently depending on the orientations.

The terms “about” or “approximately” as used herein is inclusive of the stated value and means within an acceptable range of deviation for the recited value as determined by one of ordinary skill in the art, considering the measurement in question and the error associated with measurement of the recited quantity (i.e., the limitations of the measurement system). For example, “about” may mean within one or more standard deviations, or within ±20%, ±10%, or ±5% of the stated value.

It should be understood that the terms “comprises,” “comprising,” “includes,” “including,” “have,” “having,” “contains,” “containing,” and the like are intended to specify the presence of stated features, integers, steps, operations, elements, components, or combinations thereof in the disclosure, but do not preclude the presence or addition of one or more other features, integers, steps, operations, elements, components, or combinations thereof.

Unless otherwise defined or implied herein, all terms (including technical and scientific terms) used have the same meaning as commonly understood by those skilled in the art to which this disclosure pertains. It will be further understood that terms, such as those defined in commonly used dictionaries, should be interpreted as having a meaning that is consistent with their meaning in the context of the relevant art and should not be interpreted in an ideal or excessively formal sense unless clearly defined in the specification.

Embodiments provide a light-emitting device which may include:
a first electrode; a second electrode facing the first electrode; an interlayer between the first electrode and the second electrode and including an emission layer; and a heterocyclic compound represented by Formula 1:

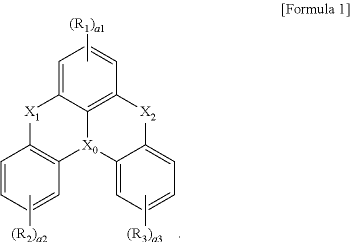

In Formula 1, X₀ may be B, P(═O), or P(═S).

In an embodiment, X₀ may be B.

In Formula 1, X₁ may be N(R₄), O, or S; and X₂ may be N(R₅), O, or S.

In Formula 1, at least one of X₁ and X₂ may each independently be a group represented by one of Formulae 2-1 to 2-4:

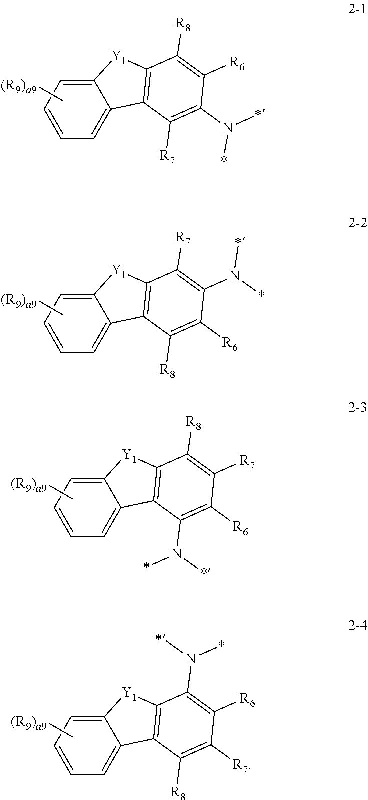

In an embodiment, at least one of X₁ and X₂ may each independently be a group represented by one of Formulae 2-1 to 2-2.

In Formulae 1 and 2-1 to 2-4, Y₁ may be N(R₁₀), O, or S.

In Formulae 1 and 2-1 to 2-4, a1 to a3 and a9 may each independently be an integer from 1 to 4.

In an embodiment, at least one of a1 to a3 may each be 1. In an embodiment, a1 to a3 may each be 1.

In Formulae 2-1 to 2-4* and *′ may each independently indicate a binding site to a neighboring carbon atom.

In Formulae 1 and 2-1 to 2-4, R₁ to R₁₀ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂).

In Formulae 2-1 and 2-2, at least one of R₆ and R₇ may be a C₃-C₆₀ carbocyclic group unsubstituted or substituted with at least one R₁₀ₐ or a C₁-C₆₀ heterocyclic group unsubstituted or substituted with at least one R₁₀ₐ.

In Formulae 2-3 and 2-4, R₆ may be a C₃-C₆₀ carbocyclic group unsubstituted or substituted with at least one R₁₀ₐ or a C₁-C₆₀ heterocyclic group unsubstituted or substituted with at least one R₁₀ₐ.

In Formulae 1 and 2-1 to 2-4, R₁₀ₐ may be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ may each independently be: hydrogen; deuterium; —F; —Cl; —Br; —I; a hydroxyl group; a cyano group; a nitro group; or a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof.

In an embodiment, R₁ to R₅, R₉, and R₁₀ may each independently be:
hydrogen, deuterium, —F, a cyano group, a C₁-C₂₀ alkyl group unsubstituted or substituted with at least one R₁₀ₐ, a C₂-C₆ monocyclic group unsubstituted or substituted with at least one R₁₀ₐ, or a C₄-C₁₃ polycyclic group unsubstituted or substituted with at least one R₁₀ₐ.

In an embodiment, R₁ to R₅, R₉, and R₁₀ may each independently be:
hydrogen, deuterium, —F, or a cyano group;
a C₁-C₂₀ alkyl group unsubstituted or substituted with hydrogen, deuterium, —F, a cyano group, or any combination thereof; or
a benzene group, a pyridine group, a dibenzofuran group, a dibenzothiophene group, a carbazole group, an azadibenzofuran group, an azadibenzothiophene group, or an azacarbazole group, each unsubstituted or substituted with hydrogen, deuterium, —F, a cyano group, a C₁-C₂₀ alkyl group, a deuterated C₁-C₂₀ alkyl group, a fluorinated C₁-C₂₀ alkyl group, a phenyl group, a deuterated phenyl group, a fluorinated phenyl group, a (C₁-C₂₀ alkyl)phenyl group, a biphenyl group, a dibenzofuranyl group, a dibenzothiophenyl group, a carbazolyl group, a fluorenyl group, a dibenzosilolyl group, or any combination thereof.

In an embodiment, R₁ to R₅, R₉, and R₁₀ may each independently be:
hydrogen, deuterium, —F, —Cl, —Br, —I, a cyano group, or a C₁-C₂₀ alkyl group;
a C₁-C₂₀ alkyl group substituted with at least one of deuterium, —F, —Cl, —Br, —I, —CDs, —CD₂H,-CDH₂, —CF₃, —CF₂H, —CFH₂, a cyano group, a nitro group, a C₁-C₁₀ alkyl group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a naphthyl group, a pyridinyl group, and a pyrimidinyl group;
a phenyl group, a pyridine group, a dibenzofuranyl group, a dibenzothiophenyl group, a carbazolyl group, an azadibenzofuranyl group, an azadibenzothiophenyl group, or an azacarbazolyl group, each unsubstituted or substituted with at least one of deuterium, —F, —Cl, —Br, —I, —CDs, —CD₂H,-CDH₂, —CF₃, —CF₂H, —CFH₂, a cyano group, a C₁-C₂₀ alkyl group, a phenyl group, a biphenyl group, a C₁-C₁₀ alkylphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzoimidazolyl group, a benzofuranyl group, a benzothiophenyl group, a benzoisothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, —Si(Q₃₁)(Q₃₂)(Q₃₃), —N(Q₃₁)(Q₃₂), or —B(Q₃₁)(Q₃₂); or
—Si(Q₁)(Q₂)(Q₃), —N(Q₁)(Q₂), or —B(Q₁)(Q₂), and
Q₁ to Q₃ and Q₃₁ to Q₃₃ may each independently be:
—CH₃, —CD₃, —CD₂H,-CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, or —CD₂CDH₂; or
an n-propyl group, an iso-propyl group, an n-butyl group, an isobutyl group, a sec-butyl group, a tert-butyl group, an n-pentyl group, an isopentyl group, a sec-pentyl group, a tert-pentyl group, a phenyl group, a naphthyl group, a pyridinyl group, a pyrimidinyl group, a pyridazinyl group, a pyrazinyl group, or a triazinyl group, each unsubstituted or substituted with at least one of deuterium, a C₁-C₁₀ alkyl group, a phenyl group, a biphenyl group, a pyridinyl group, a pyrimidinyl group, a pyridazinyl group, a pyrazinyl group, and a triazinyl group.

In an embodiment, R₁ to R₅, R₉, and R₁₀ may each independently be:
a methyl group, an ethyl group, an n-propyl group, an iso-propyl group, an n-butyl group, an isobutyl group, a sec-butyl group, a tert-butyl group, an n-pentyl group, an isopentyl group, a sec-pentyl group, a tert-pentyl group, a 2-methylbutyl group, a 2-2dimethylpropyl group, a 1-ethylpropyl group, or a 1,2-dimethylpropyl group, each unsubstituted or substituted with deuterium, —F, —Cl, —Br, —I, a hydroxyl group, a cyano group, or a nitro group; or
a benzene group, a pyridine group, a dibenzofuran group, a dibenzothiophene group, a carbazole group, an azadibenzofuran group, an azadibenzothiophene group, or an azacarbazole group, each unsubstituted or substituted with a methyl group, an ethyl group, an n-propyl group, an iso-propyl group, an n-butyl group, an isobutyl group, a sec-butyl group, a tert-butyl group, an n-pentyl group, an isopentyl group, a sec-pentyl group, a tert-pentyl group, a 2-methylbutyl group, a 2-2 dimethylpropyl group, 1-ethylpropyl, 1,2-dimethylpropyl, a phenyl group, each unsubstituted or substituted with deuterium, —F, —Cl, —Br, —I, a hydroxyl group, a cyano group, or a nitro group.

In an embodiment, the heterocyclic compound may be represented by Formula 1-1:

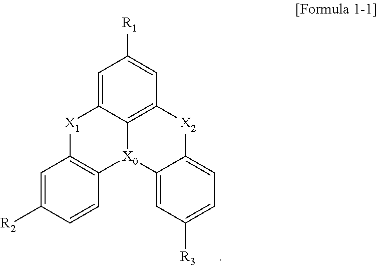

In Formula 1-1, X₀ to X₂ and R₁ to R₃ may be the same as described herein; and at least one of R₁ to R₃ may each not be hydrogen.

In an embodiment, in Formula 1-1, R₁ and R₂ may each not be hydrogen, R₂ and R₃ may each not be hydrogen, or R₁ and R₃ may each not be hydrogen.

In an embodiment, at least one of R₆ to R₈ may each independently be a C₆-C₆₀ aryl group unsubstituted or substituted with at least one R₁₀ₐ.

In an embodiment, at least one of R₆ to R₈ may each independently be a phenyl group, a pentalenyl group, a naphthyl group, an azulenyl group, an indacenyl group, an acenaphthyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a heptalenyl group, a naphthacenyl group, a picenyl group, a hexacenyl group, a pentacenyl group, a rubicenyl group, a coronenyl group, an ovalenyl group, a pyridinyl group, a pyrimidinyl group, a pyrazinyl group, a pyridazinyl group, a triazinyl group, a quinolinyl group, a benzoquinolinyl group, an isoquinolinyl group, a benzoisoquinolinyl group, a quinoxalinyl group, a benzoquinoxalinyl group, a quinazolinyl group, a benzoquinazolinyl group, a cinnolinyl group, a phenanthrolinyl group, a phthalazinyl group, or a naphthyridinyl group.

In an embodiment, at least one of R₆ to R₈ may each independently be a phenyl group, a pentalenyl group, a naphthyl group, an azulenyl group, an indacenyl group, an acenaphthyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a heptalenyl group, a naphthacenyl group, a picenyl group, a hexacenyl group, a pentacenyl group, a rubicenyl group, a coronenyl group, or an ovalenyl group.

In an embodiment, in Formula 1, at least one of X₁ and X₂ may each independently be a group represented by one of Formulae 3-1 to 3-10:

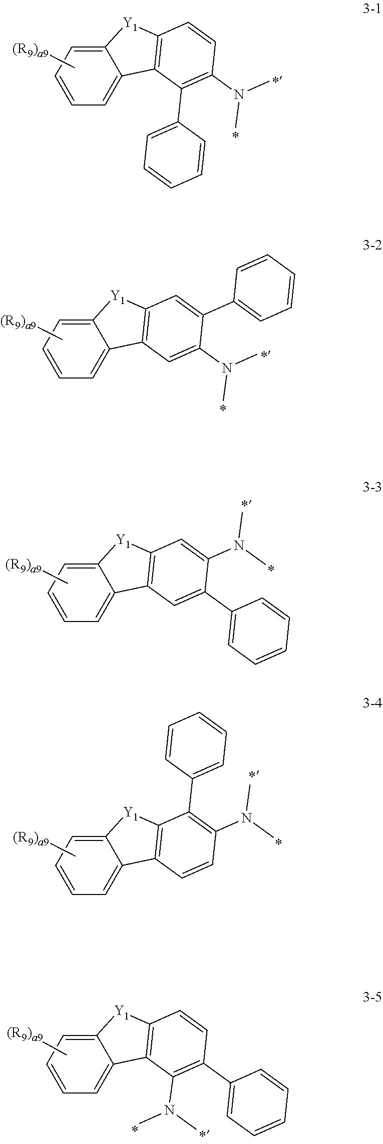

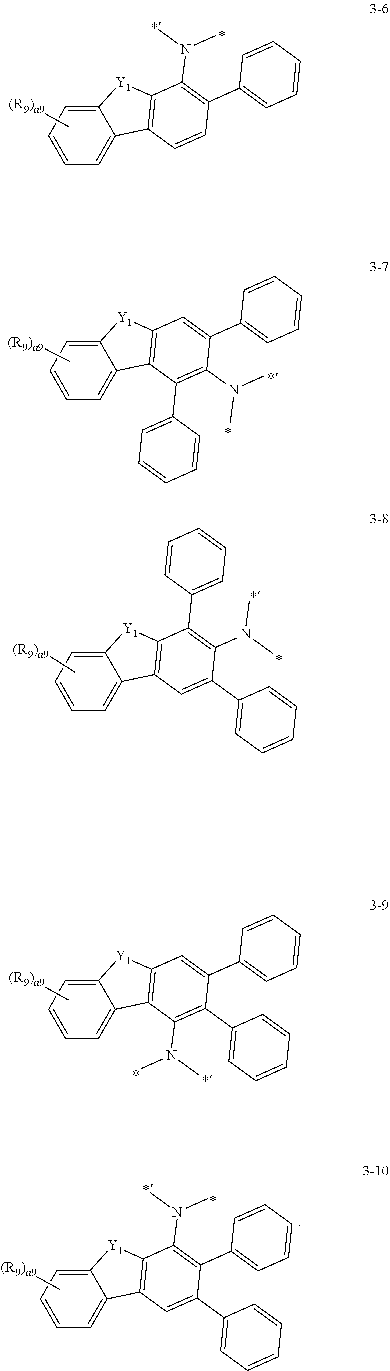

In Formulae 3-1 to 3-10, Y₁, a9, R₉, * and *′ may be the same as described herein.

In an embodiment, X₁ and X₂ may be identical to each other.

In an embodiment, X₁ and X₂ may be different from each other.

In an embodiment, at least one of R₁ to R₃ may each independently be a carbazolyl group unsubstituted or substituted with at least one R₁₀ₐ, and the carbazolyl group unsubstituted or substituted with at least one R₁₀ₐ may be represented by Formula 4:

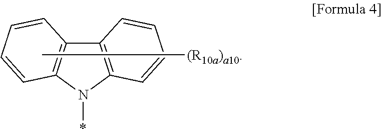

In Formula 4, R₁₀ₐ may be the same as described herein; a10 may be an integer from 1 to 8; and * may indicate a binding site to a neighboring carbon atom.

In an embodiment, the heterocyclic compound represented by Formula 1 may be any one of Compounds 1 to 192:

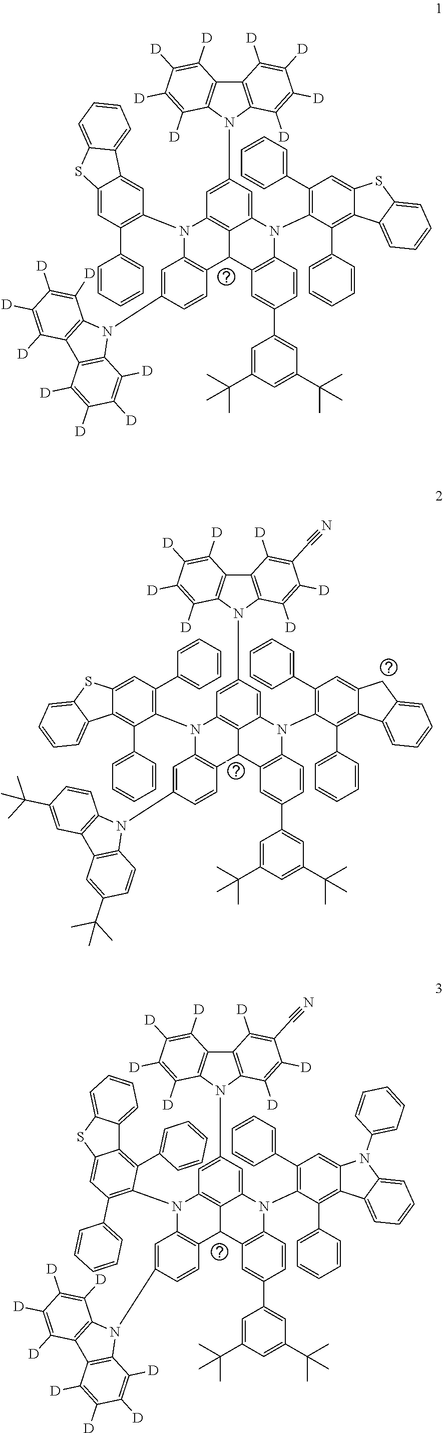

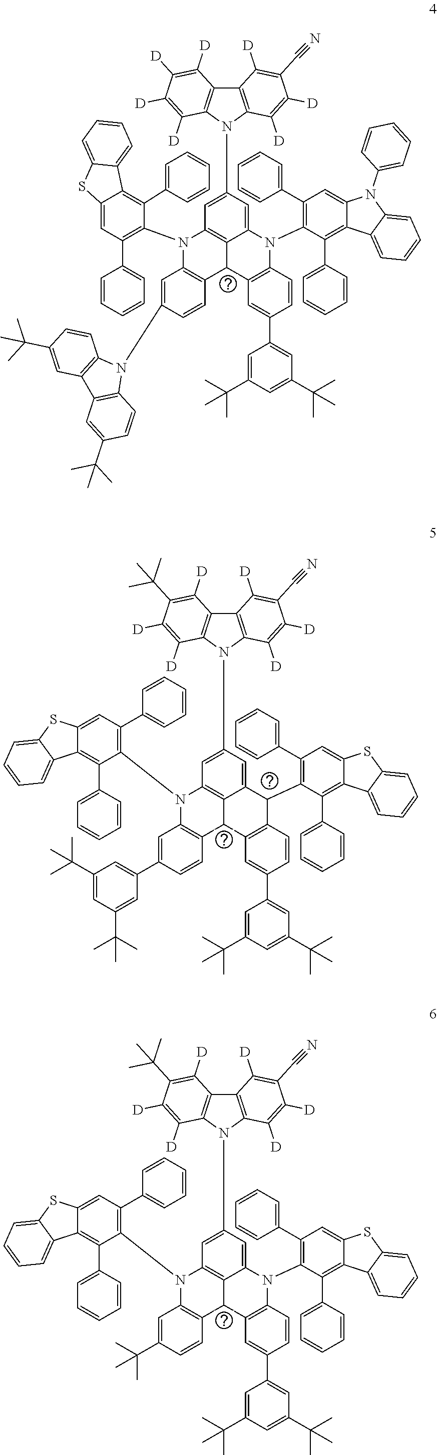

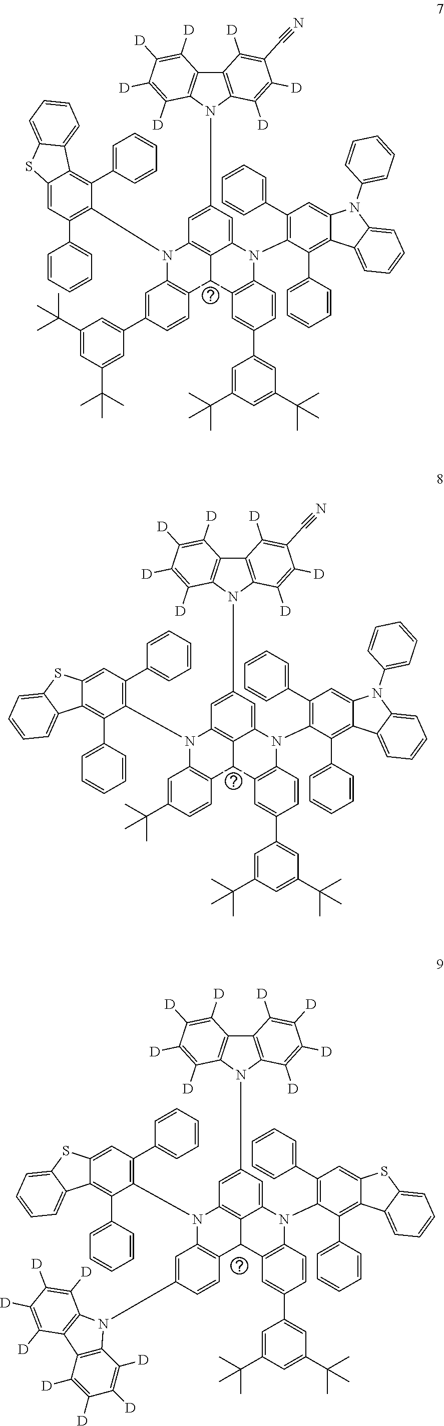

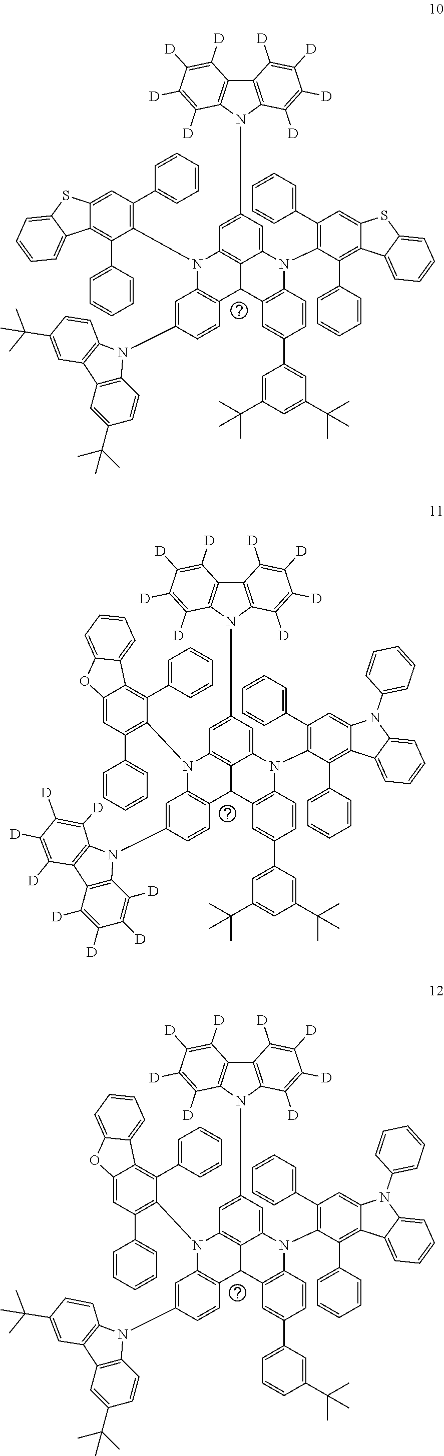

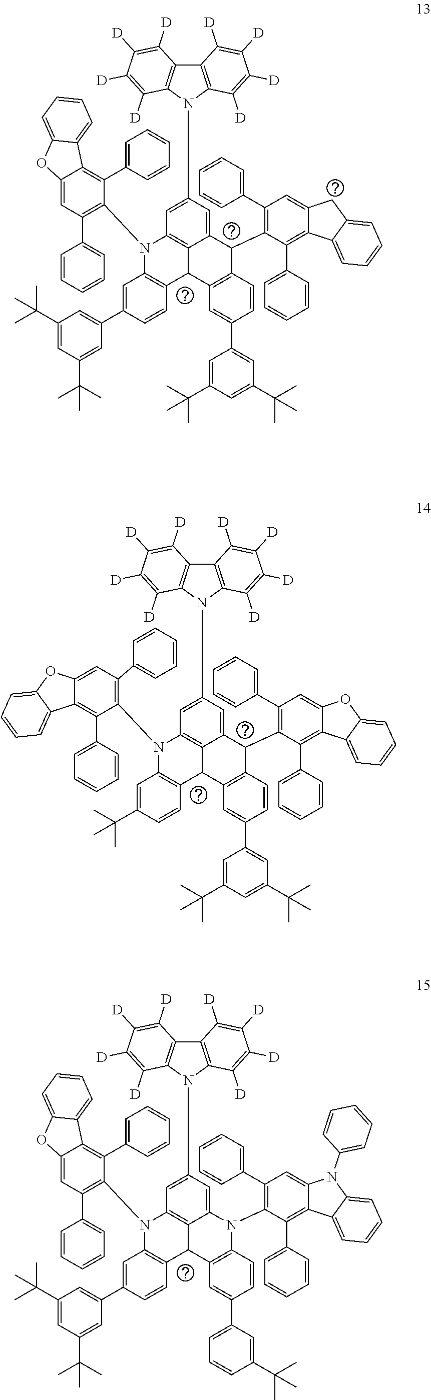

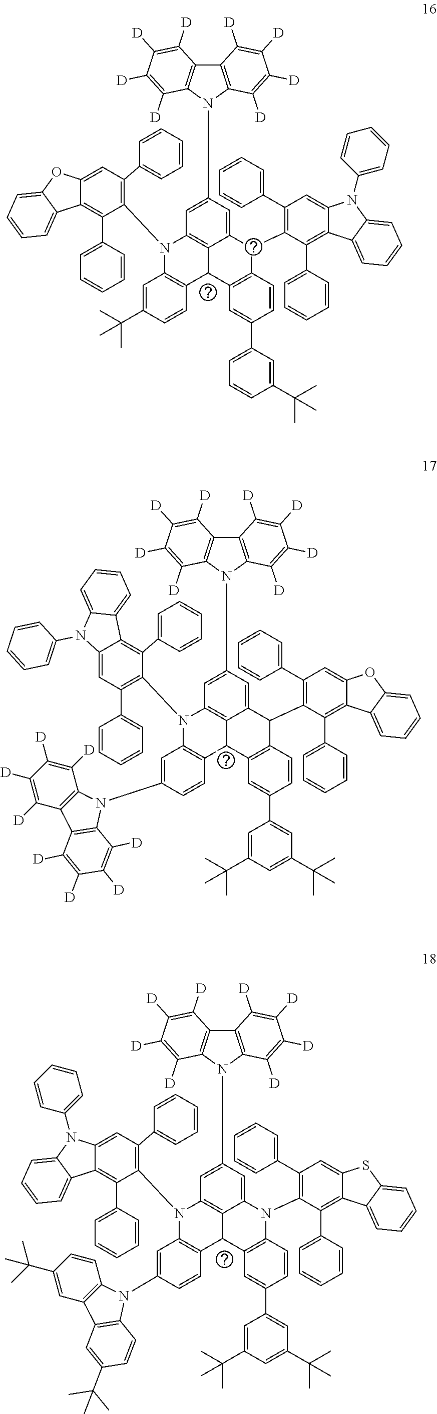

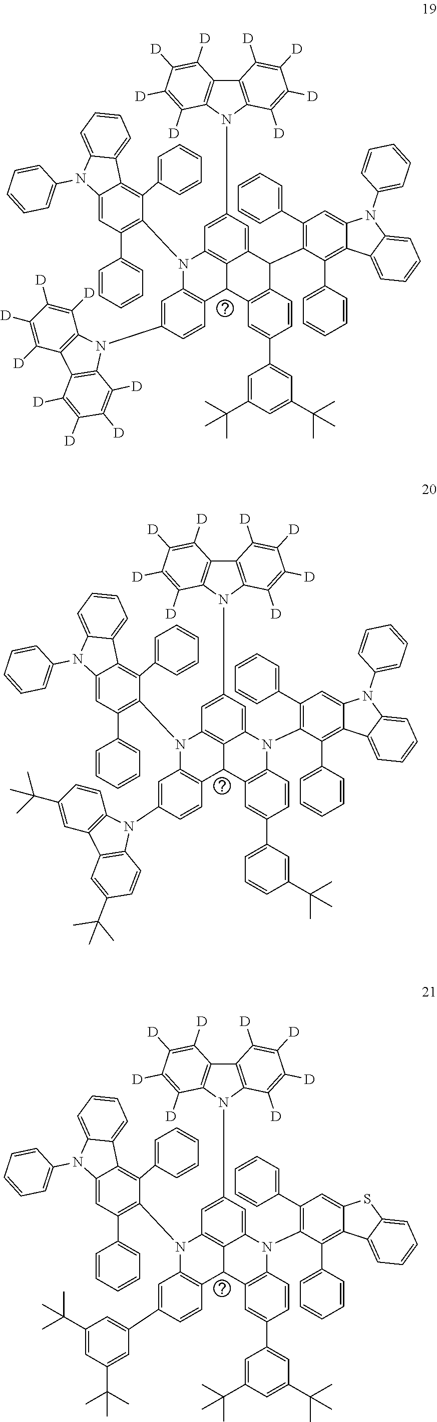

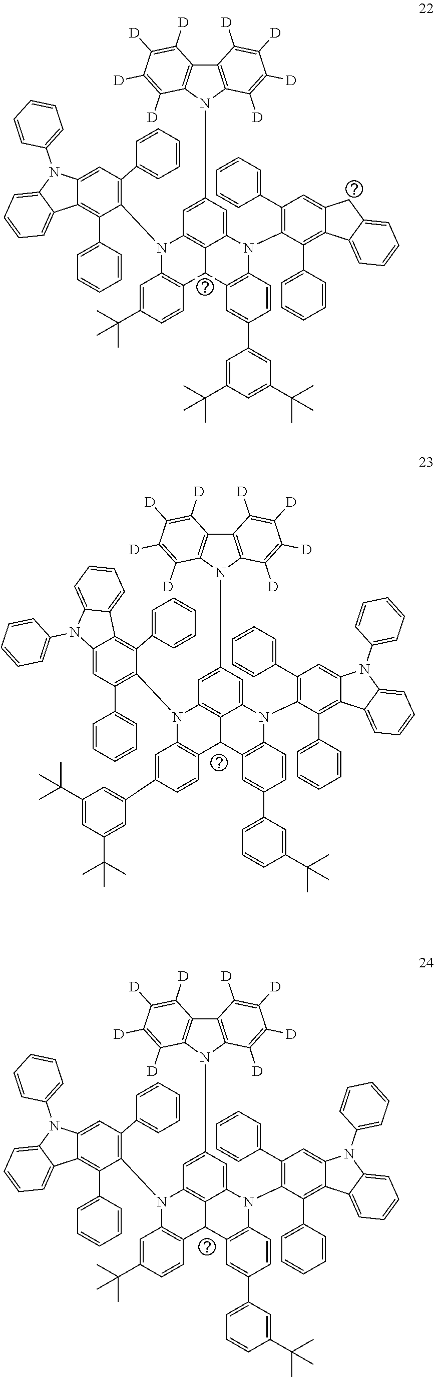

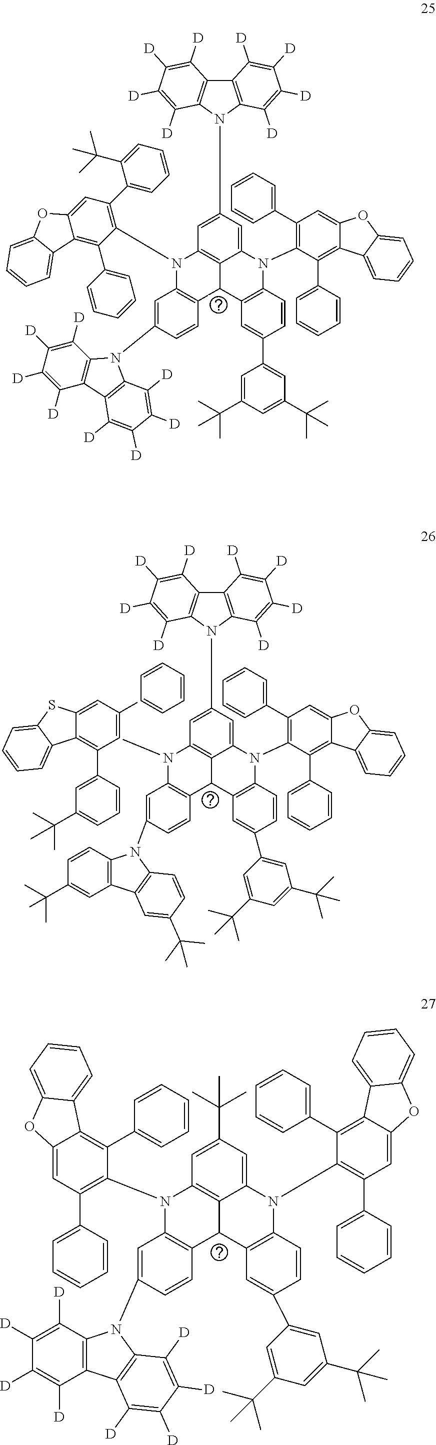

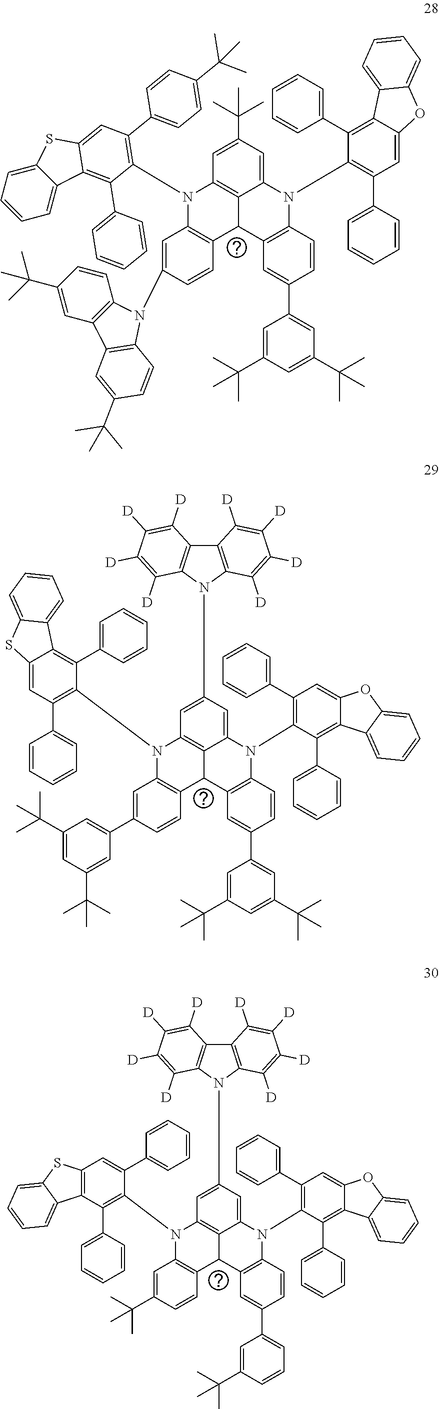

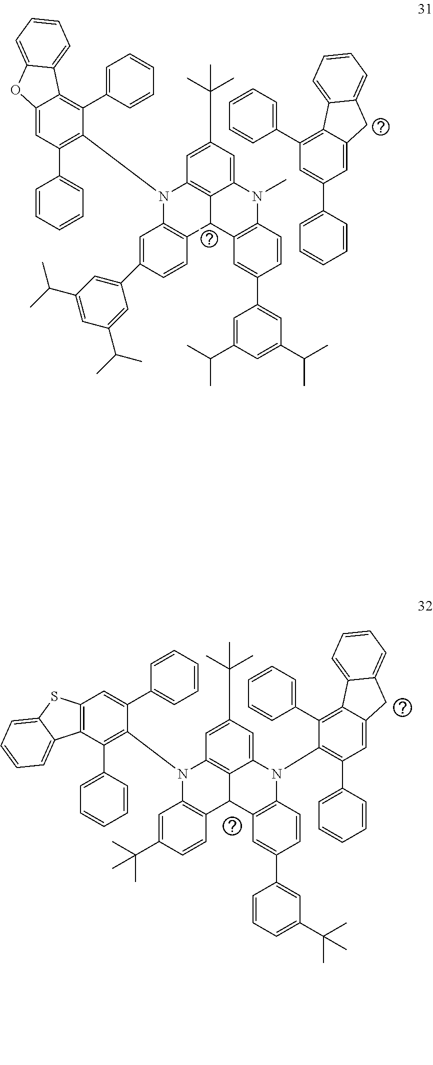

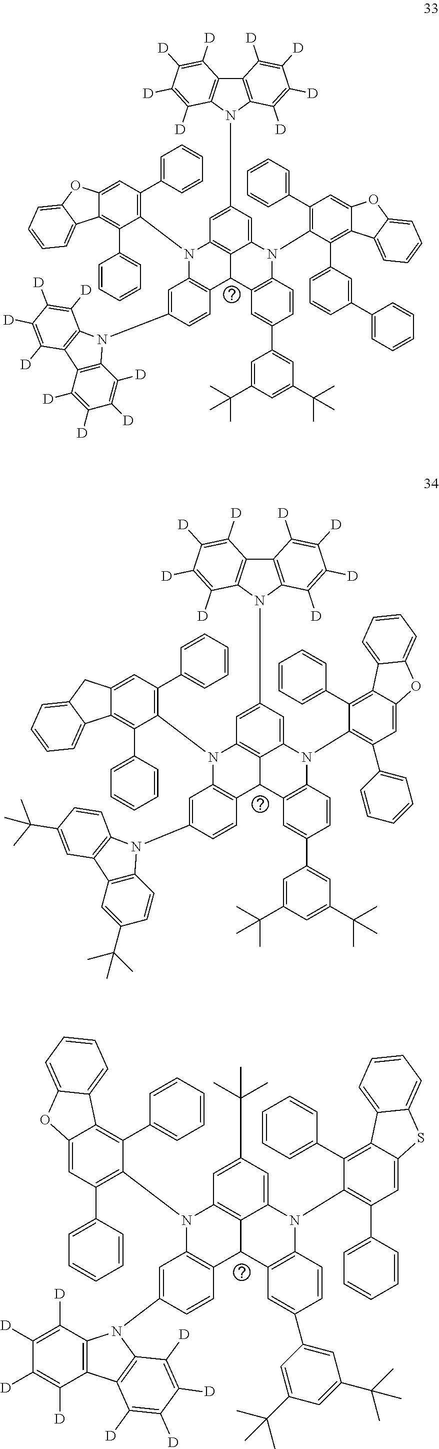

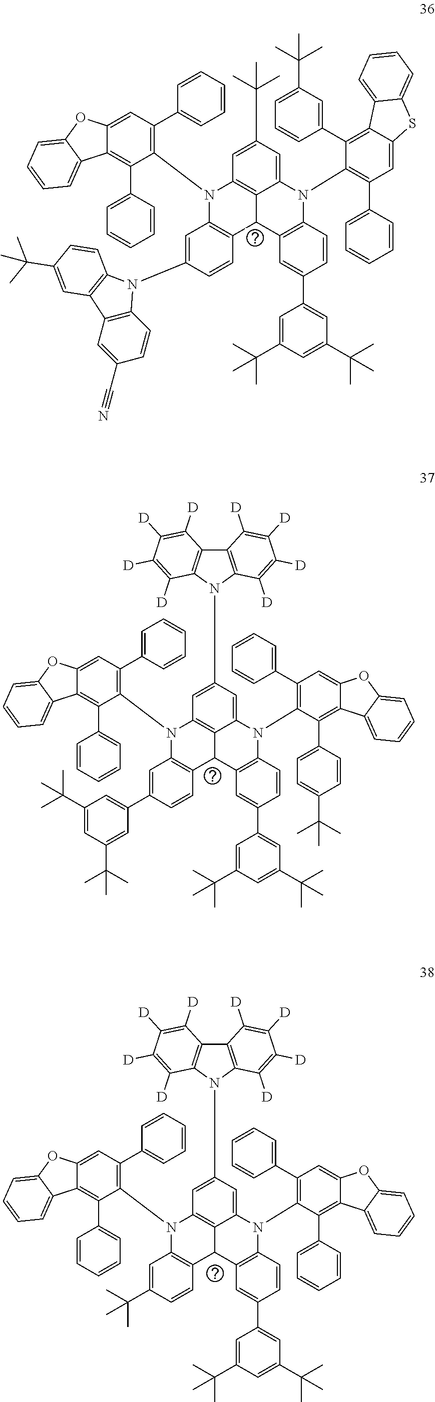

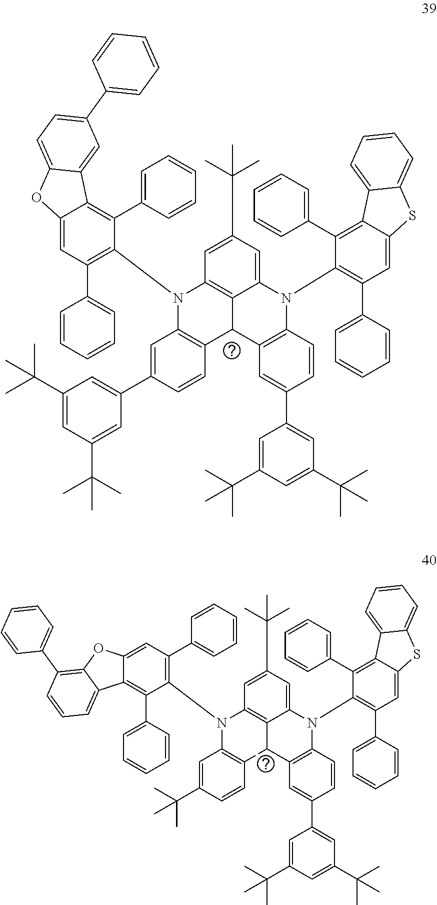 [SMILES: CC(C)(C)c1cc(-c2ccc3c(c2)C2c4ccc(-c5cc(C(C)(C)C)cc(C(C)(C)C)c5)cc4N(c4c(-c5ccccc5)cc5oc6ccc(-c7ccccc7)cc6c5c4-c4ccccc4)c4cc(C(C)(C)C)cc(c42)N3c2c(-c3ccccc3)cc3sc4ccccc4c3c2-c2ccccc2)cc(C(C)(C)C)c1 ; CC(C)(C)c1cc(-c2ccc3c(c2)C2c4ccc(C(C)(C)C)cc4N(c4c(-c5ccccc5)cc5oc6c(-c7ccccc7)cccc6c5c4-c4ccccc4)c4cc(C(C)(C)C)cc(c42)N3c2c(-c3ccccc3)cc3sc4ccccc4c3c2-c2ccccc2)cc(C(C)(C)C)c1]

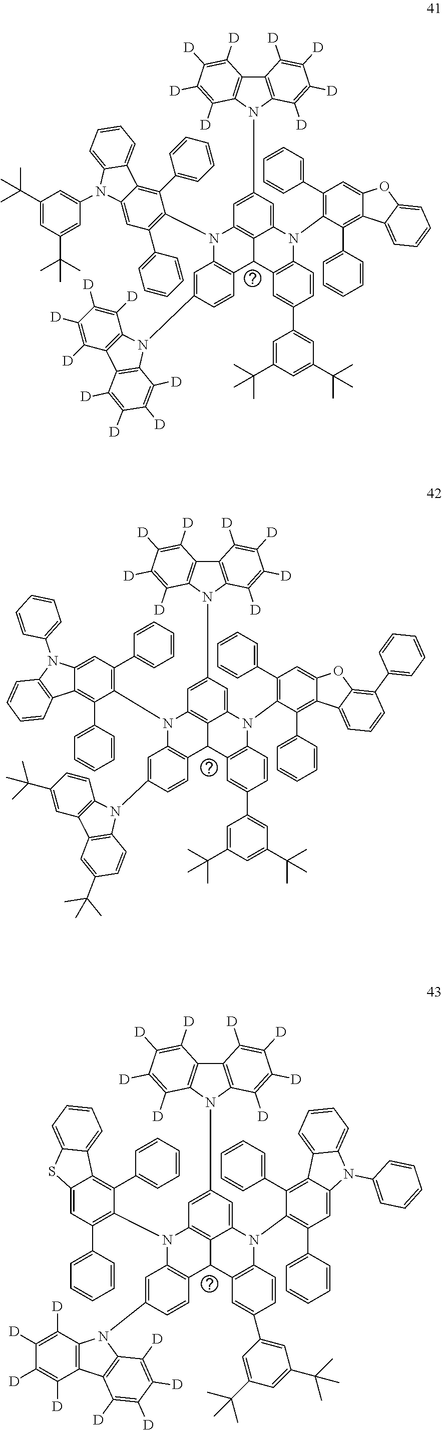

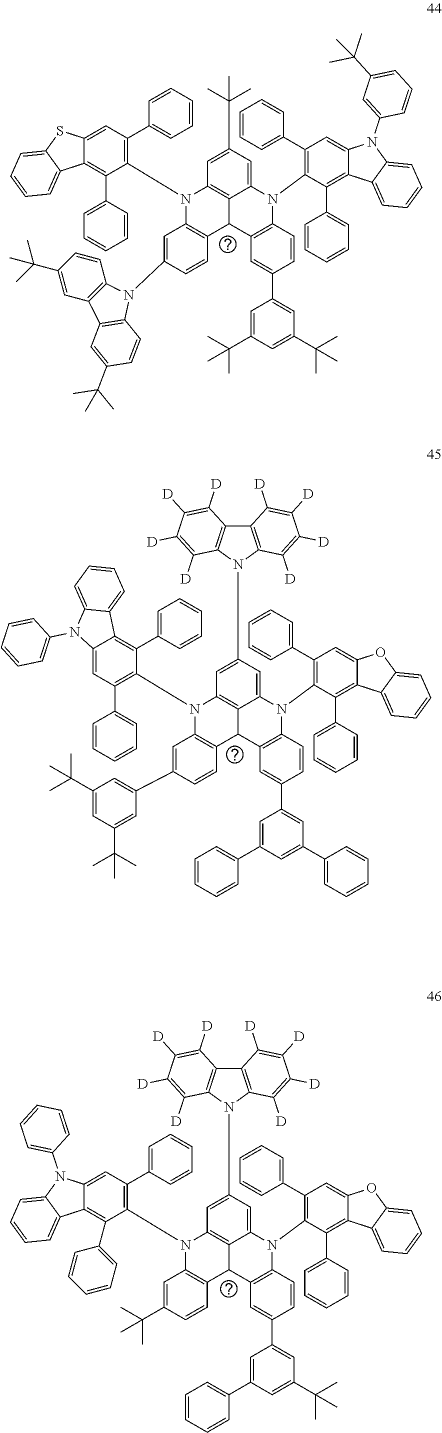

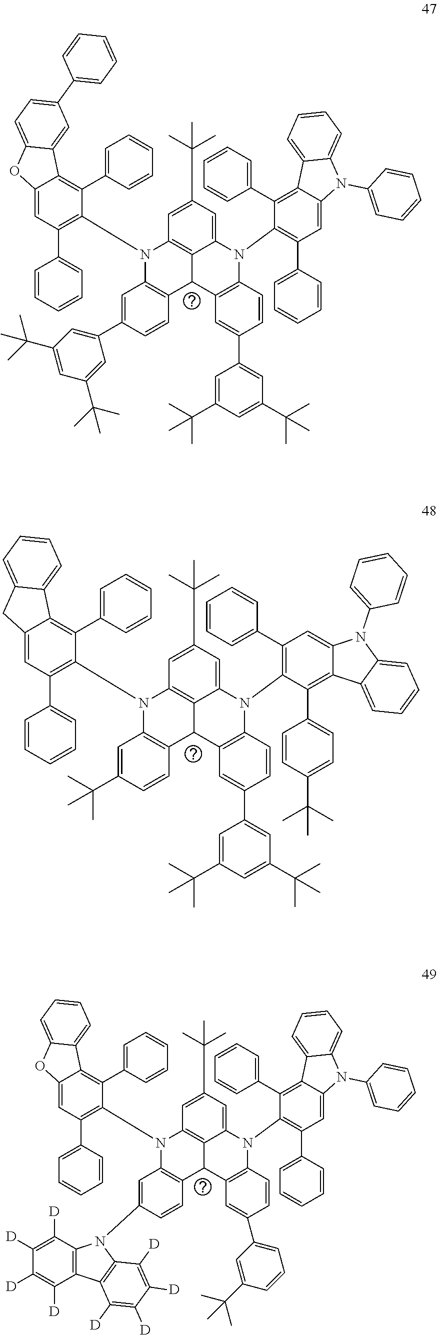

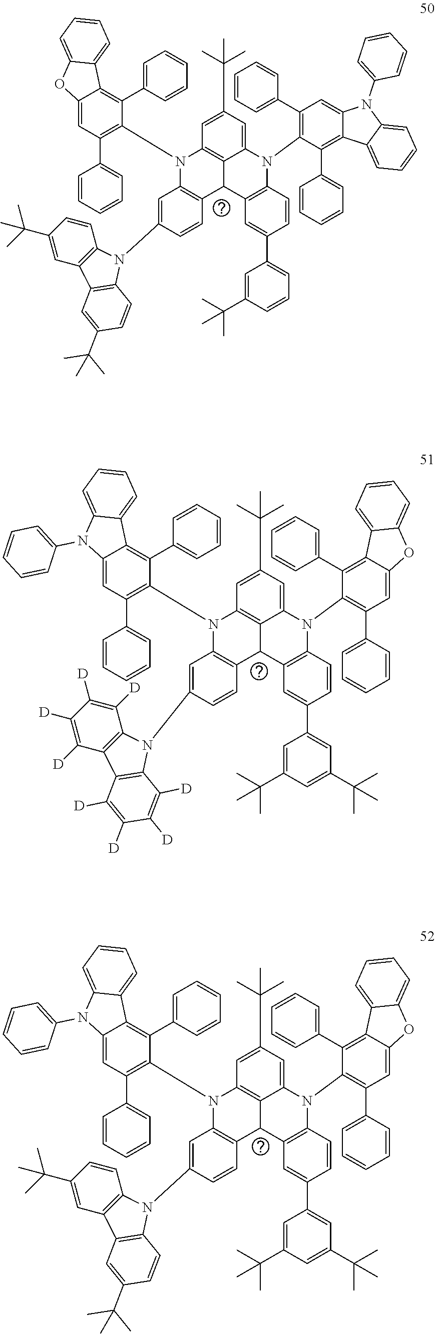

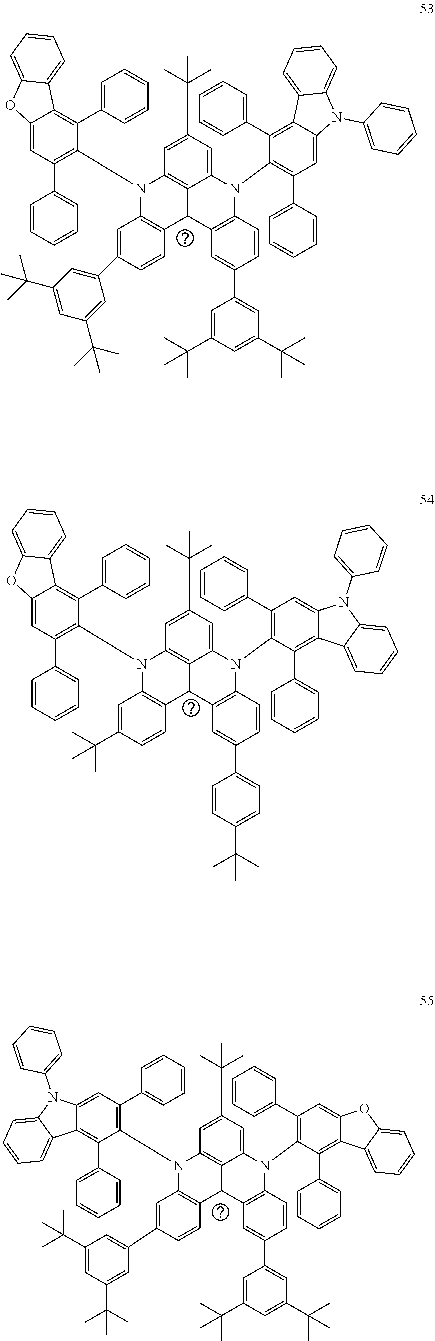

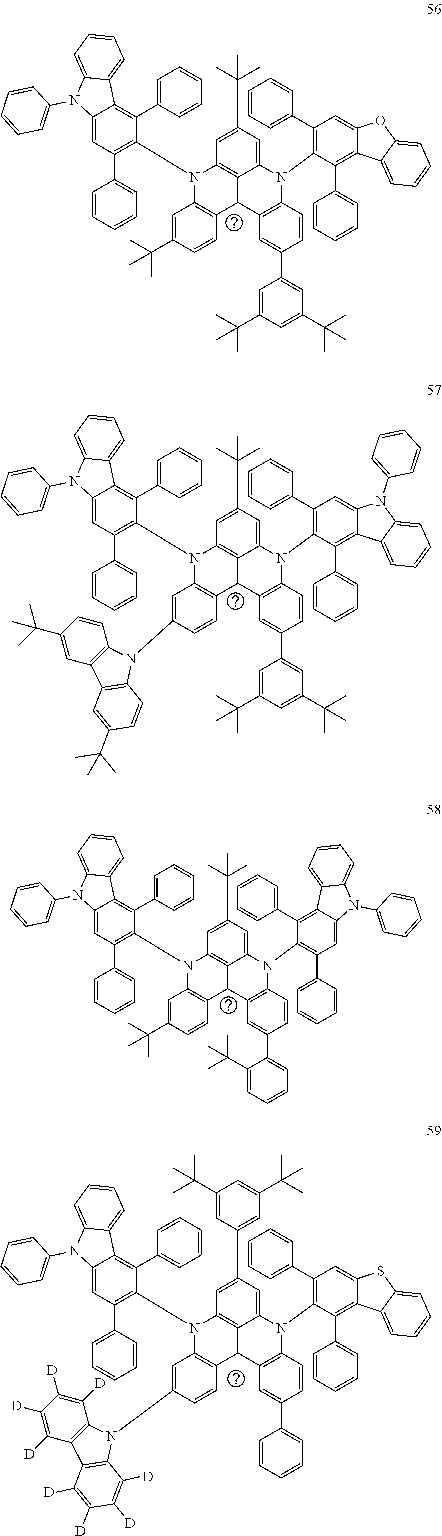

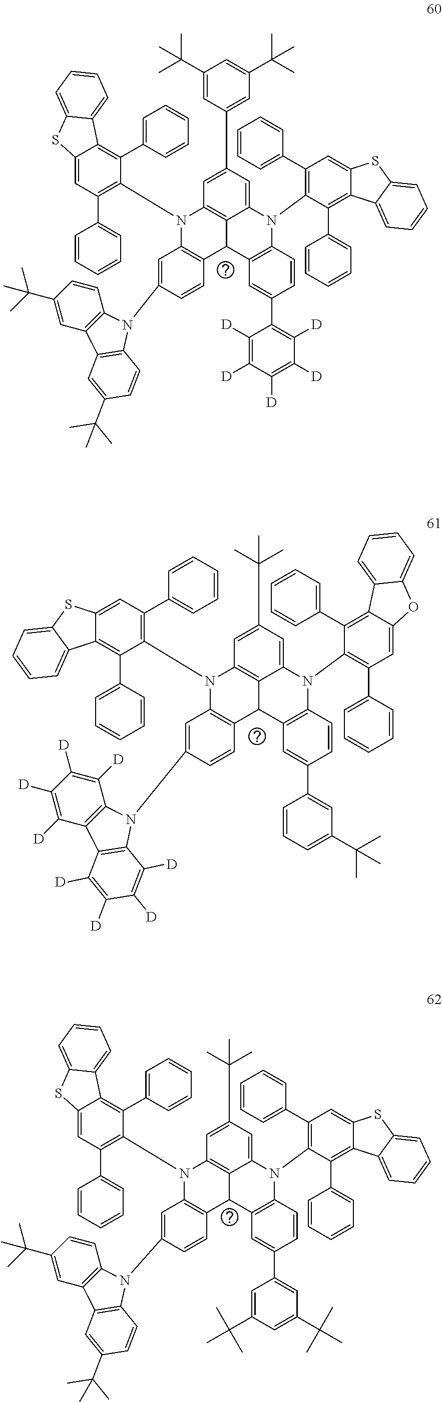

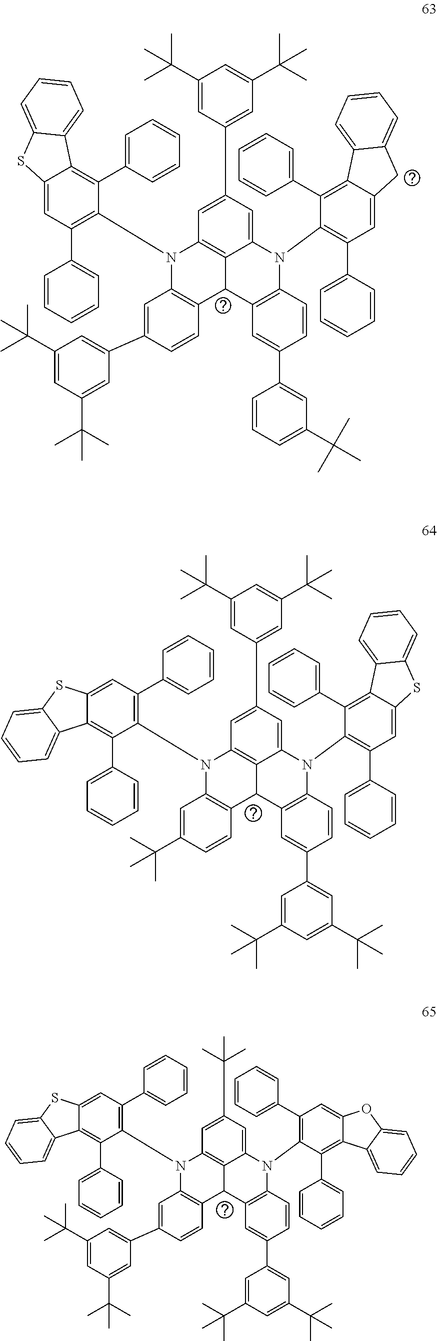

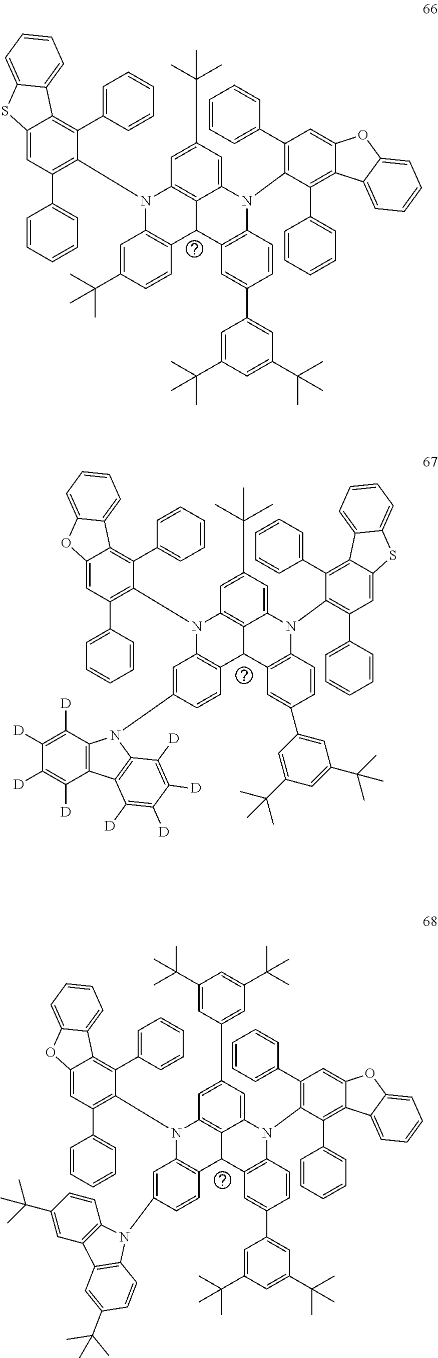

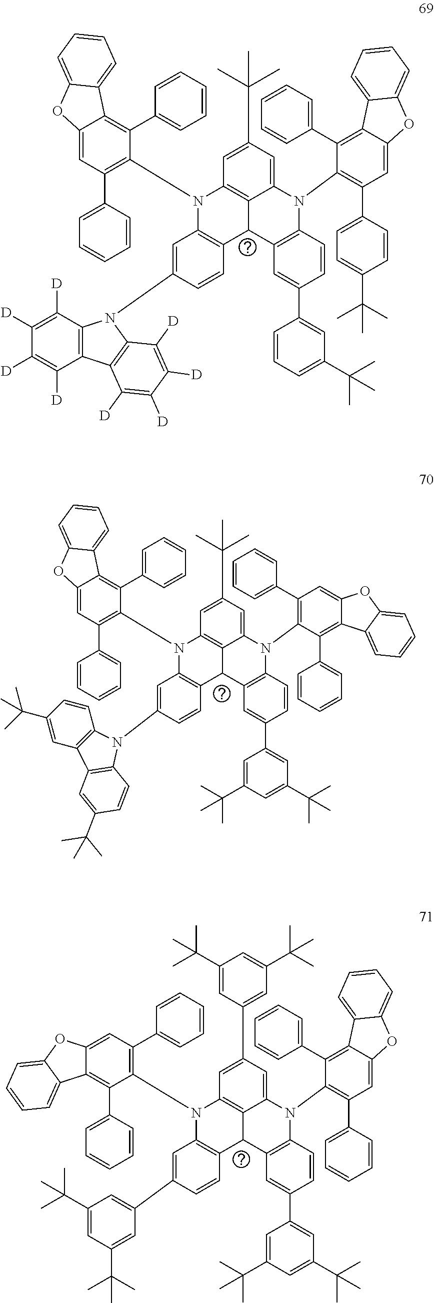

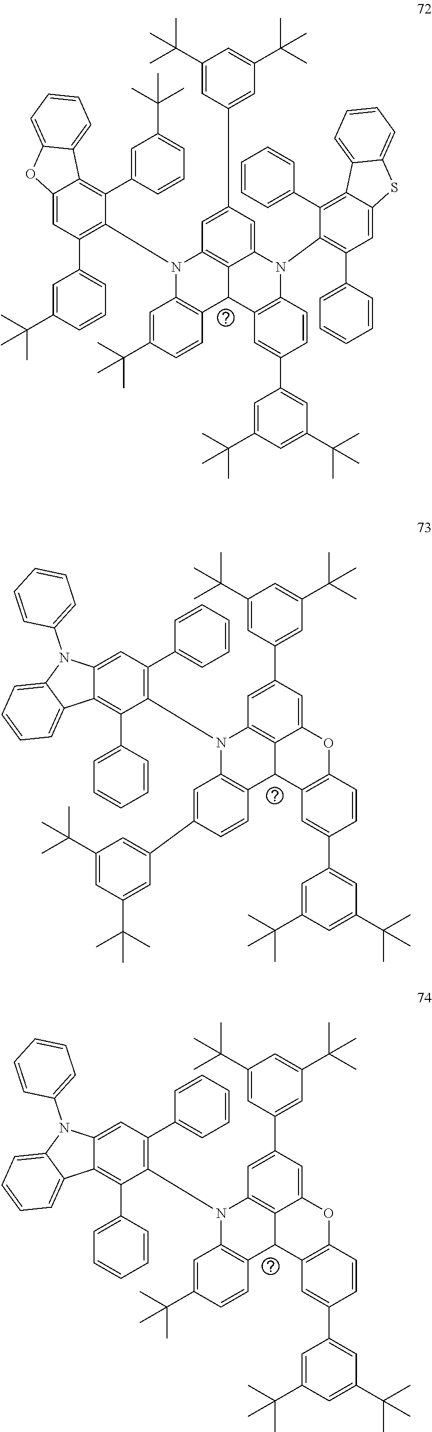

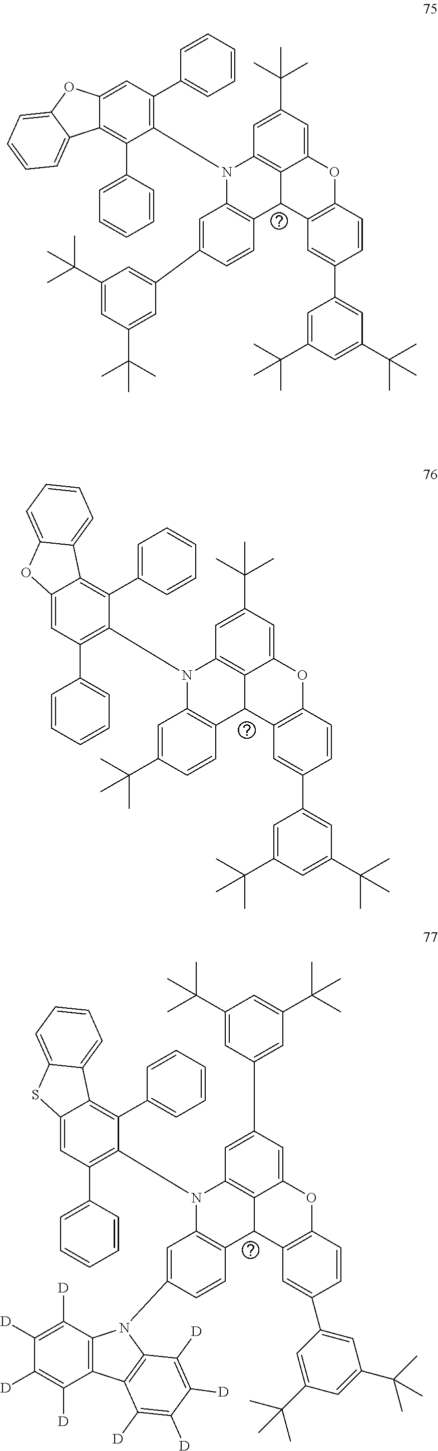 [SMILES: CC(C)(C)c1cc(-c2ccc3c(c2)C2c4ccc(-c5cc(C(C)(C)C)cc(C(C)(C)C)c5)cc4N(c4c(-c5ccccc5)cc5oc6ccccc6c5c4-c4ccccc4)c4cc(C(C)(C)C)cc(c42)O3)cc(C(C)(C)C)c1 ; CC(C)(C)c1cc(-c2ccc3c(c2)C2c4ccc(C(C)(C)C)cc4N(c4c(-c5ccccc5)cc5oc6ccccc6c5c4-c4ccccc4)c4cc(C(C)(C)C)cc(c42)O3)cc(C(C)(C)C)c1 ; [2H]c1c([2H])c([2H])c2c(c1[2H])c1c([2H])c([2H])c([2H])c([2H])c1n2-c1ccc2c(c1)N(c1c(-c3ccccc3)cc3sc4ccccc4c3c1-c1ccccc1)c1cc(-c3cc(C(C)(C)C)cc(C(C)(C)C)c3)cc3c1C2c1cc(-c2cc(C(C)(C)C)cc(C(C)(C)C)c2)ccc1O3]

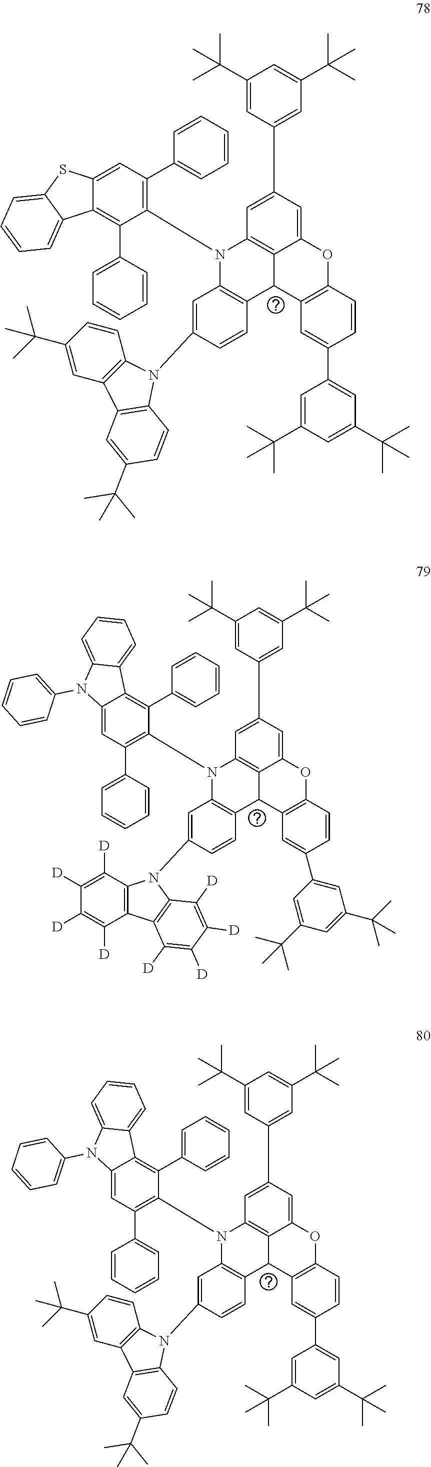

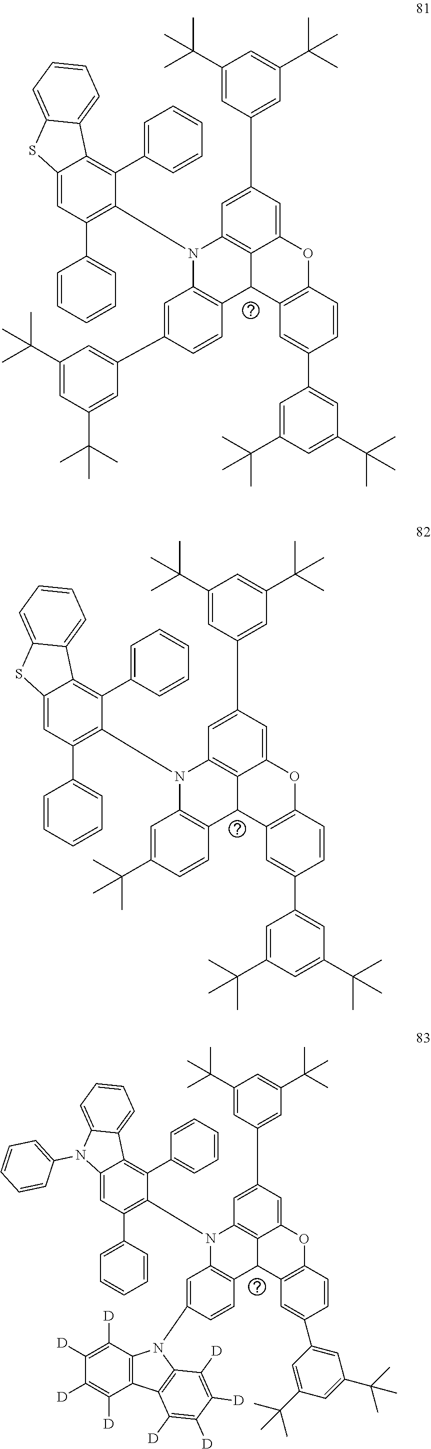

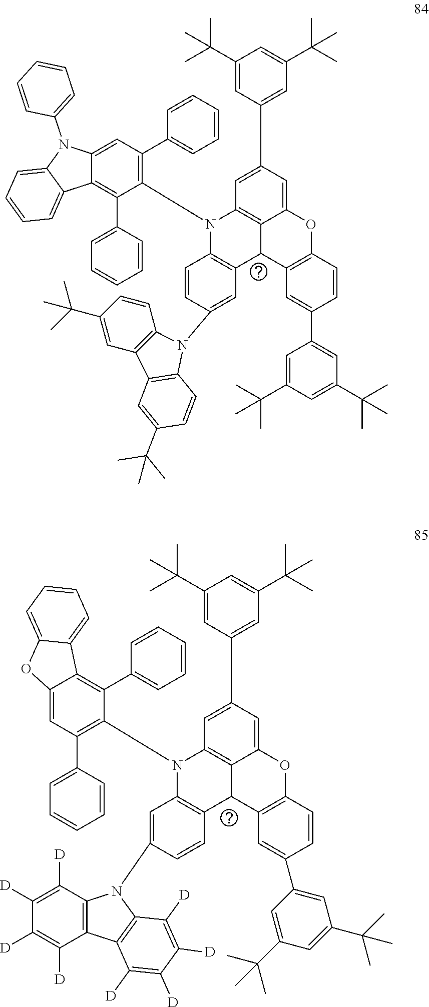 [SMILES: CC(C)(C)c1cc(-c2ccc3c(c2)C2c4cc(-n5c6ccc(C(C)(C)C)cc6c6cc(C(C)(C)C)ccc65)ccc4N(c4c(-c5ccccc5)cc5c(c4-c4ccccc4)c4ccccc4n5-c4ccccc4)c4cc(-c5cc(C(C)(C)C)cc(C(C)(C)C)c5)cc(c42)O3)cc(C(C)(C)C)c1 ; [2H]c1c([2H])c([2H])c2c(c1[2H])c1c([2H])c([2H])c([2H])c([2H])c1n2-c1ccc2c(c1)N(c1c(-c3ccccc3)cc3oc4ccccc4c3c1-c1ccccc1)c1cc(-c3cc(C(C)(C)C)cc(C(C)(C)C)c3)cc3c1C2c1cc(-c2cc(C(C)(C)C)cc(C(C)(C)C)c2)ccc1O3]

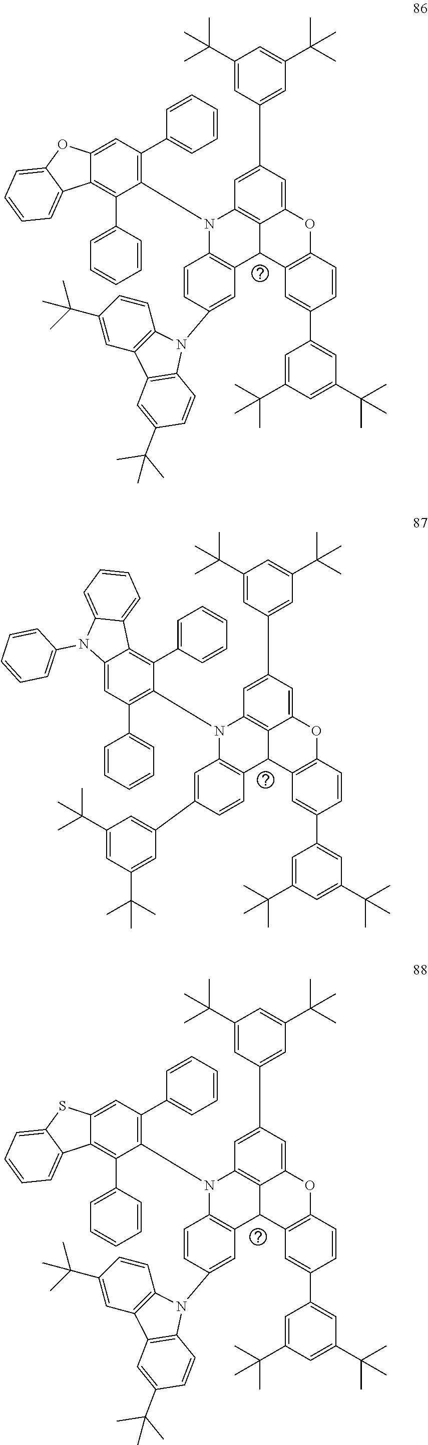

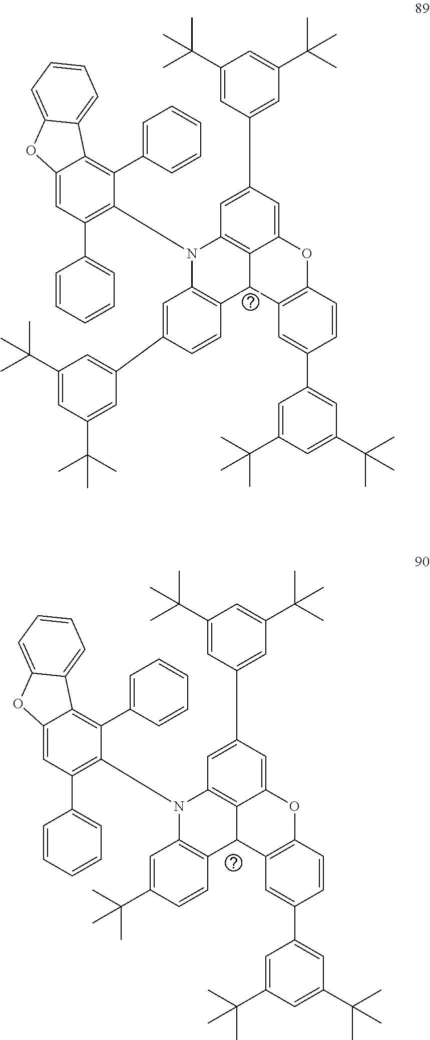 [SMILES: CC(C)(C)c1cc(-c2ccc3c(c2)C2c4ccc(-c5cc(C(C)(C)C)cc(C(C)(C)C)c5)cc4N(c4c(-c5ccccc5)cc5oc6ccccc6c5c4-c4ccccc4)c4cc(-c5cc(C(C)(C)C)cc(C(C)(C)C)c5)cc(c42)O3)cc(C(C)(C)C)c1 ; CC(C)(C)c1cc(-c2ccc3c(c2)C2c4ccc(C(C)(C)C)cc4N(c4c(-c5ccccc5)cc5oc6ccccc6c5c4-c4ccccc4)c4cc(-c5cc(C(C)(C)C)cc(C(C)(C)C)c5)cc(c42)O3)cc(C(C)(C)C)c1]

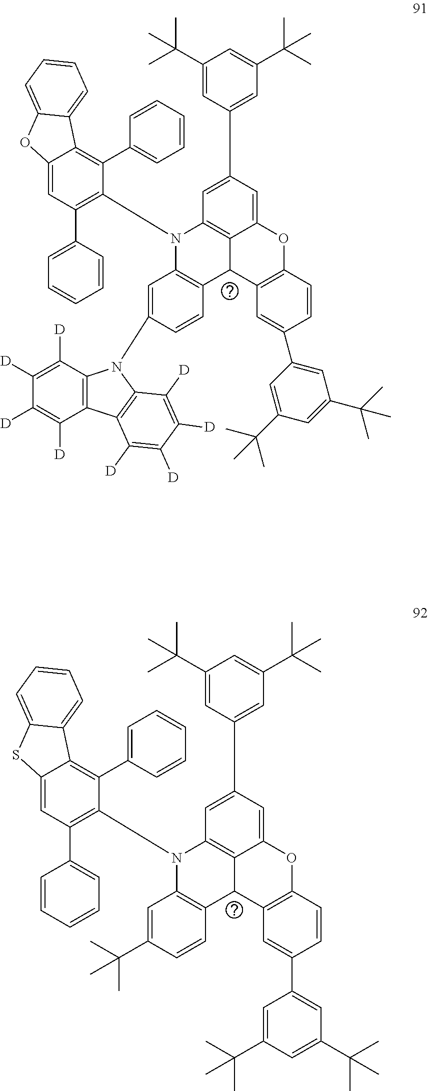 [SMILES: [2H]c1c([2H])c([2H])c2c(c1[2H])c1c([2H])c([2H])c([2H])c([2H])c1n2-c1ccc2c(c1)N(c1c(-c3ccccc3)cc3oc4ccccc4c3c1-c1ccccc1)c1cc(-c3cc(C(C)(C)C)cc(C(C)(C)C)c3)cc3c1C2c1cc(-c2cc(C(C)(C)C)cc(C(C)(C)C)c2)ccc1O3 ; CC(C)(C)c1cc(-c2ccc3c(c2)C2c4ccc(C(C)(C)C)cc4N(c4c(-c5ccccc5)cc5sc6ccccc6c5c4-c4ccccc4)c4cc(-c5cc(C(C)(C)C)cc(C(C)(C)C)c5)cc(c42)O3)cc(C(C)(C)C)c1]

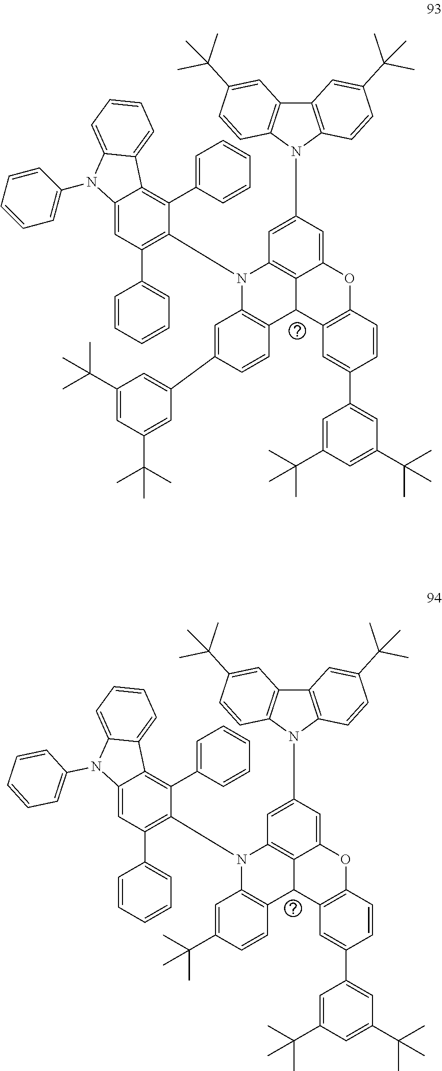 [SMILES: CC(C)(C)c1cc(-c2ccc3c(c2)C2c4ccc(-c5cc(C(C)(C)C)cc(C(C)(C)C)c5)cc4N(c4c(-c5ccccc5)cc5c(c4-c4ccccc4)c4ccccc4n5-c4ccccc4)c4cc(-n5c6ccc(C(C)(C)C)cc6c6cc(C(C)(C)C)ccc65)cc(c42)O3)cc(C(C)(C)C)c1 ; CC(C)(C)c1cc(-c2ccc3c(c2)C2c4ccc(C(C)(C)C)cc4N(c4c(-c5ccccc5)cc5c(c4-c4ccccc4)c4ccccc4n5-c4ccccc4)c4cc(-n5c6ccc(C(C)(C)C)cc6c6cc(C(C)(C)C)ccc65)cc(c42)O3)cc(C(C)(C)C)c1]

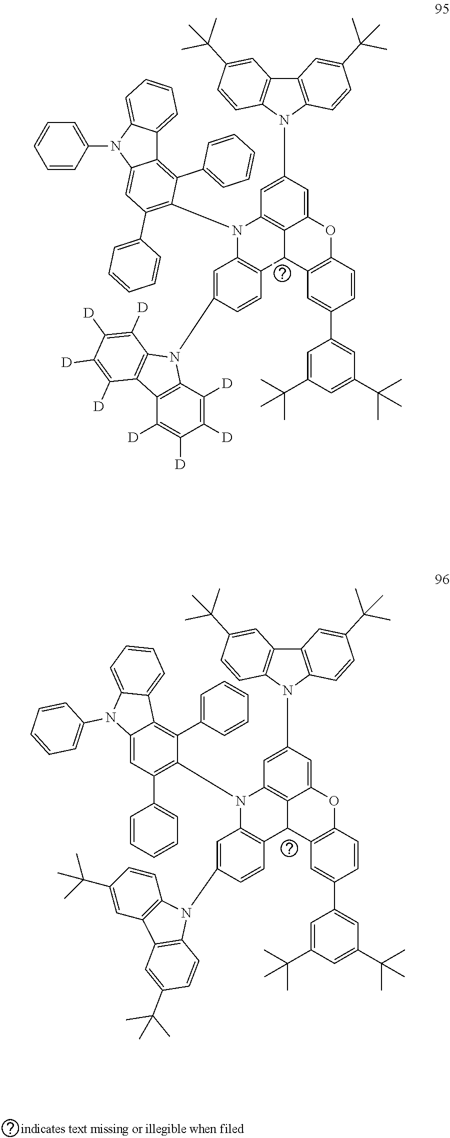 [SMILES: [2H]c1c([2H])c([2H])c2c(c1[2H])c1c([2H])c([2H])c([2H])c([2H])c1n2-c1ccc2c(c1)N(c1c(-c3ccccc3)cc3c(c1-c1ccccc1)c1ccccc1n3-c1ccccc1)c1cc(-n3c4ccc(C(C)(C)C)cc4c4cc(C(C)(C)C)ccc43)cc3c1C2c1cc(-c2cc(C(C)(C)C)cc(C(C)(C)C)c2)ccc1O3 ; CC(C)(C)c1cc(-c2ccc3c(c2)C2c4ccc(-n5c6ccc(C(C)(C)C)cc6c6cc(C(C)(C)C)ccc65)cc4N(c4c(-c5ccccc5)cc5c(c4-c4ccccc4)c4ccccc4n5-c4ccccc4)c4cc(-n5c6ccc(C(C)(C)C)cc6c6cc(C(C)(C)C)ccc65)cc(c42)O3)cc(C(C)(C)C)c1]

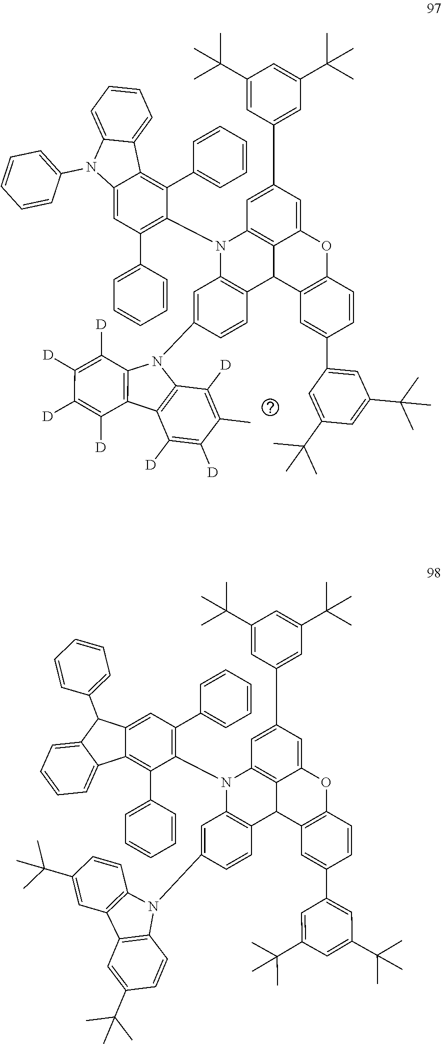

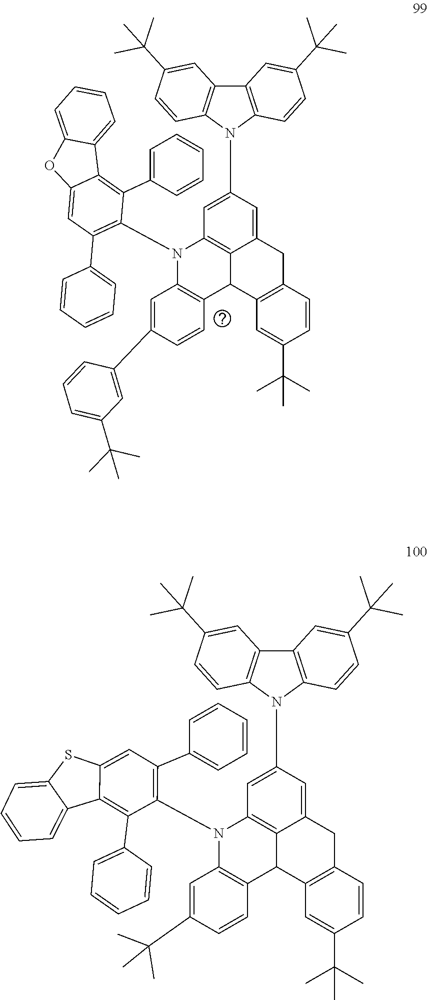 [SMILES: CC(C)(C)c1cccc(-c2ccc3c(c2)N(c2c(-c4ccccc4)cc4oc5ccccc5c4c2-c2ccccc2)c2cc(-n4c5ccc(C(C)(C)C)cc5c5cc(C(C)(C)C)ccc54)cc4c2C3c2cc(C(C)(C)C)ccc2C4)c1 ; CC(C)(C)c1ccc2c(c1)C1c3ccc(C(C)(C)C)cc3N(c3c(-c4ccccc4)cc4sc5ccccc5c4c3-c3ccccc3)c3cc(-n4c5ccc(C(C)(C)C)cc5c5cc(C(C)(C)C)ccc54)cc(c31)C2]

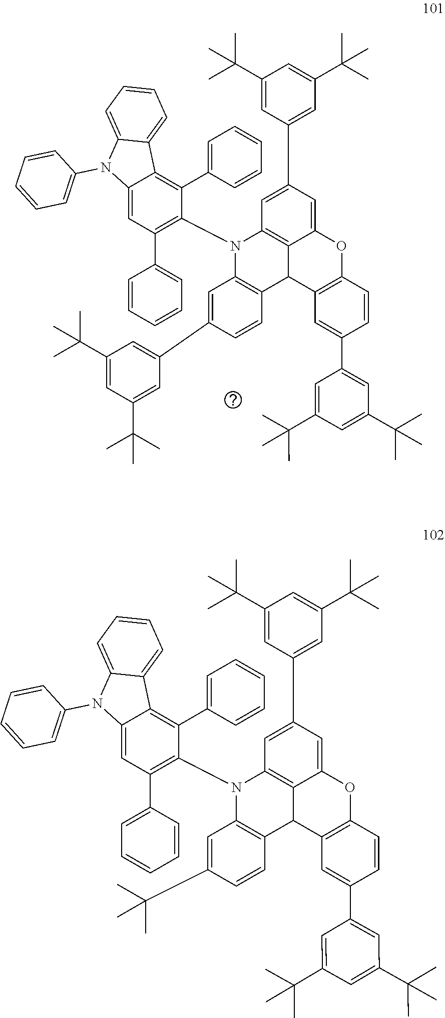 [SMILES: CC(C)(C)c1cc(-c2ccc3c(c2)C2c4ccc(-c5cc(C(C)(C)C)cc(C(C)(C)C)c5)cc4N(c4c(-c5ccccc5)cc5c(c4-c4ccccc4)c4ccccc4n5-c4ccccc4)c4cc(-c5cc(C(C)(C)C)cc(C(C)(C)C)c5)cc(c42)O3)cc(C(C)(C)C)c1 ; CC(C)(C)c1cc(-c2ccc3c(c2)C2c4ccc(C(C)(C)C)cc4N(c4c(-c5ccccc5)cc5c(c4-c4ccccc4)c4ccccc4n5-c4ccccc4)c4cc(-c5cc(C(C)(C)C)cc(C(C)(C)C)c5)cc(c42)O3)cc(C(C)(C)C)c1]

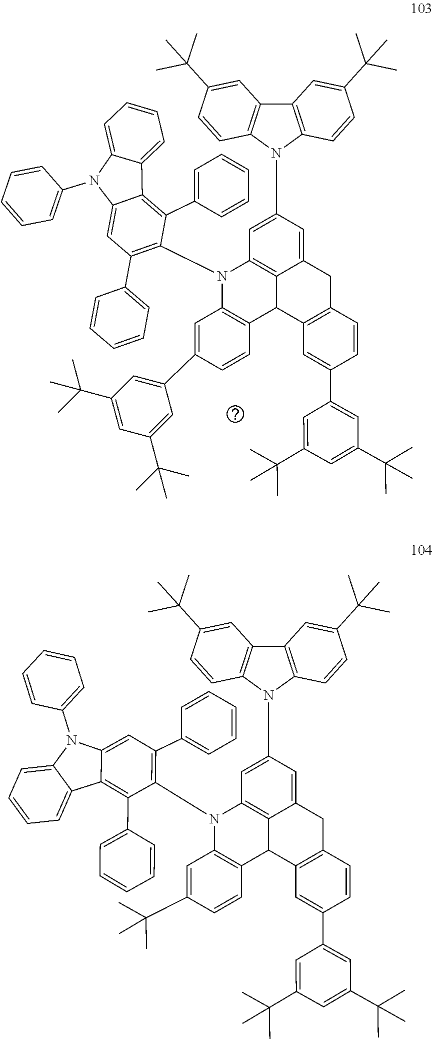 [SMILES: CC(C)(C)c1cc(-c2ccc3c(c2)C2c4ccc(-c5cc(C(C)(C)C)cc(C(C)(C)C)c5)cc4N(c4c(-c5ccccc5)cc5c(c4-c4ccccc4)c4ccccc4n5-c4ccccc4)c4cc(-n5c6ccc(C(C)(C)C)cc6c6cc(C(C)(C)C)ccc65)cc(c42)C3)cc(C(C)(C)C)c1 ; CC(C)(C)c1cc(-c2ccc3c(c2)C2c4ccc(C(C)(C)C)cc4N(c4c(-c5ccccc5)cc5c(c4-c4ccccc4)c4ccccc4n5-c4ccccc4)c4cc(-n5c6ccc(C(C)(C)C)cc6c6cc(C(C)(C)C)ccc65)cc(c42)C3)cc(C(C)(C)C)c1]

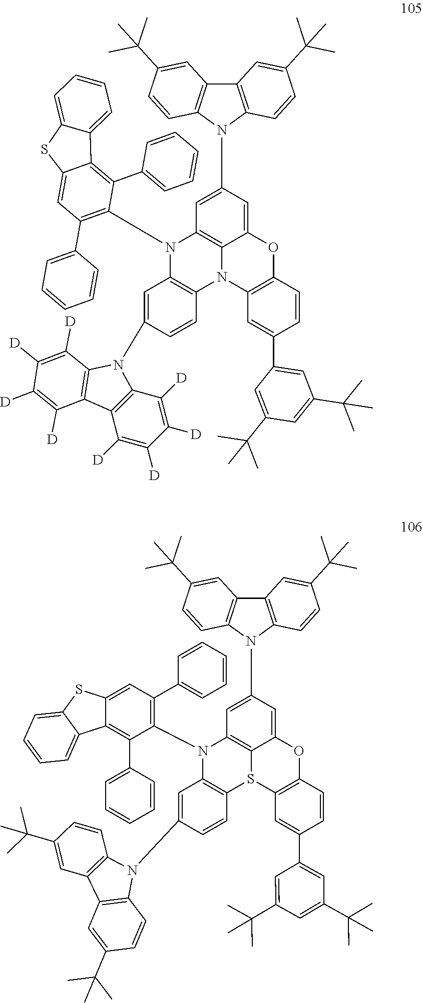

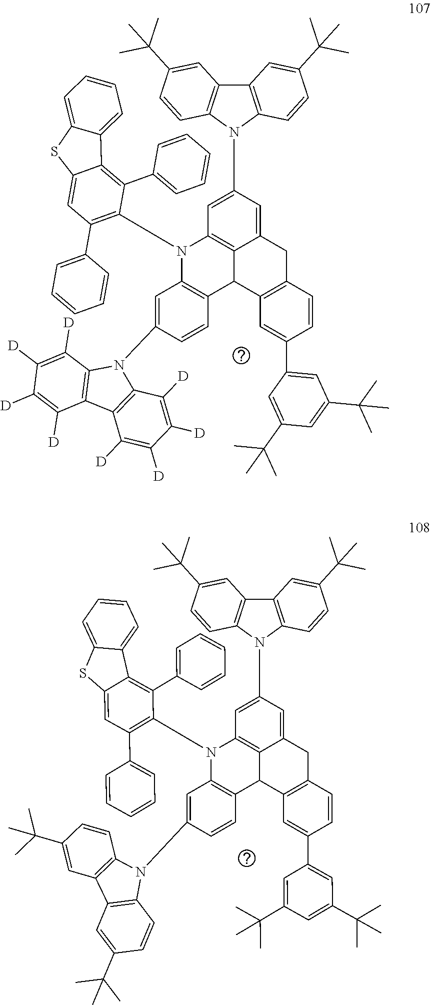

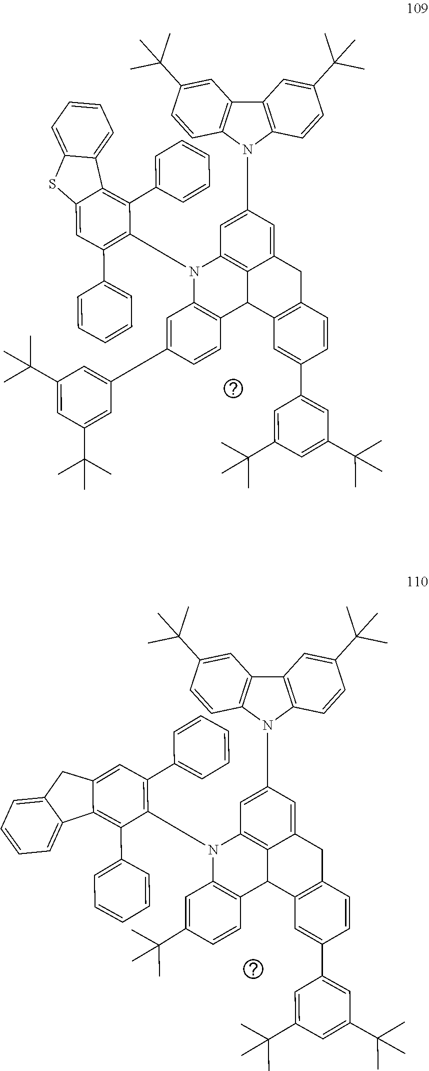 [SMILES: CC(C)(C)c1cc(-c2ccc3c(c2)C2c4ccc(-c5cc(C(C)(C)C)cc(C(C)(C)C)c5)cc4N(c4c(-c5ccccc5)cc5sc6ccccc6c5c4-c4ccccc4)c4cc(-n5c6ccc(C(C)(C)C)cc6c6cc(C(C)(C)C)ccc65)cc(c42)C3)cc(C(C)(C)C)c1 ; CC(C)(C)c1cc(-c2ccc3c(c2)C2c4ccc(C(C)(C)C)cc4N(c4c(-c5ccccc5)cc5c(c4-c4ccccc4)-c4ccccc4C5)c4cc(-n5c6ccc(C(C)(C)C)cc6c6cc(C(C)(C)C)ccc65)cc(c42)C3)cc(C(C)(C)C)c1]

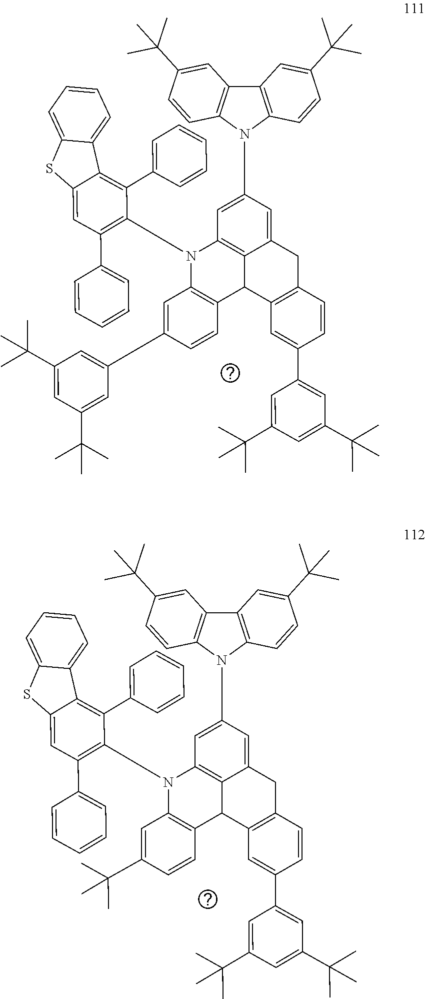 [SMILES: CC(C)(C)c1cc(-c2ccc3c(c2)C2c4ccc(-c5cc(C(C)(C)C)cc(C(C)(C)C)c5)cc4N(c4c(-c5ccccc5)cc5sc6ccccc6c5c4-c4ccccc4)c4cc(-n5c6ccc(C(C)(C)C)cc6c6cc(C(C)(C)C)ccc65)cc(c42)C3)cc(C(C)(C)C)c1 ; CC(C)(C)c1cc(-c2ccc3c(c2)C2c4ccc(C(C)(C)C)cc4N(c4c(-c5ccccc5)cc5sc6ccccc6c5c4-c4ccccc4)c4cc(-n5c6ccc(C(C)(C)C)cc6c6cc(C(C)(C)C)ccc65)cc(c42)C3)cc(C(C)(C)C)c1]

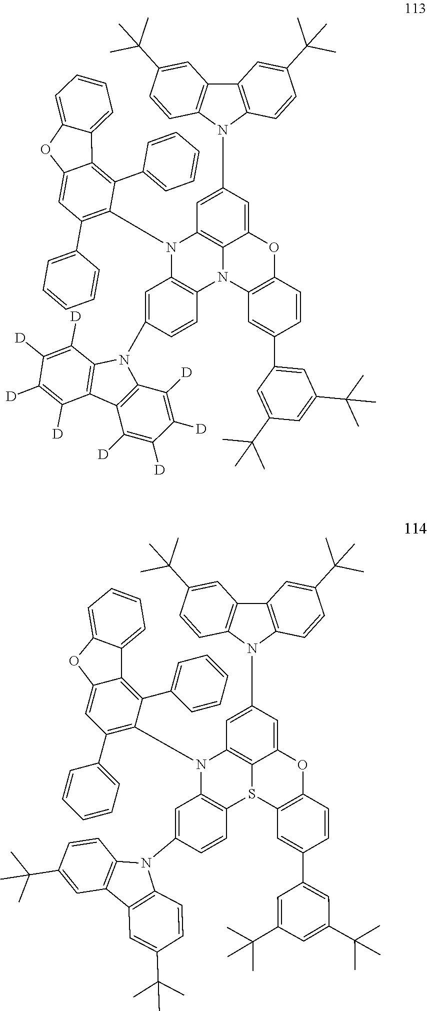

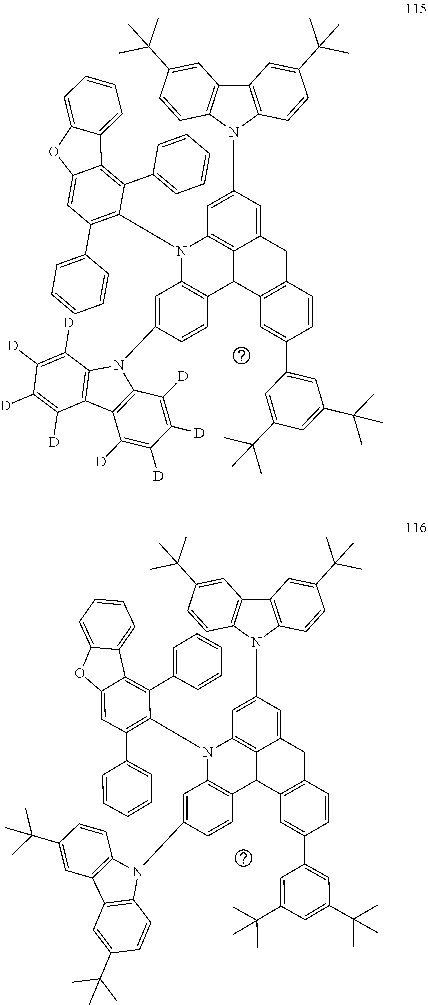

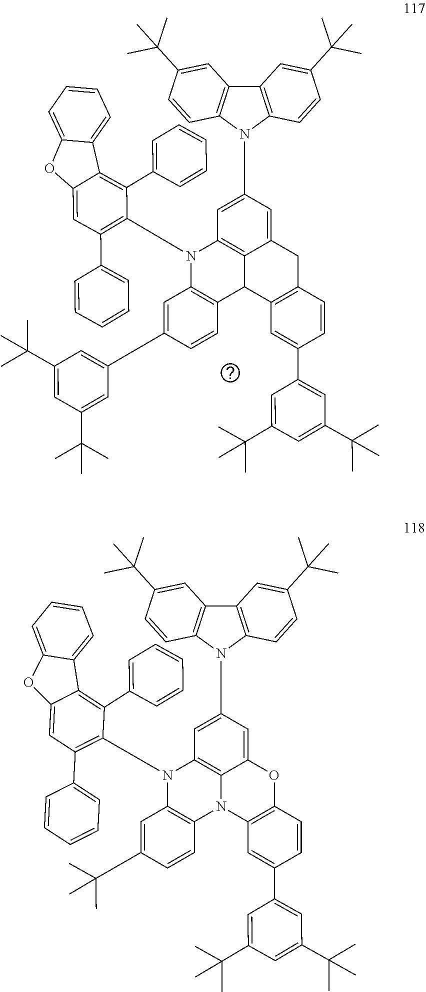 [SMILES: CC(C)(C)c1cc(-c2ccc3c(c2)C2c4ccc(-c5cc(C(C)(C)C)cc(C(C)(C)C)c5)cc4N(c4c(-c5ccccc5)cc5oc6ccccc6c5c4-c4ccccc4)c4cc(-n5c6ccc(C(C)(C)C)cc6c6cc(C(C)(C)C)ccc65)cc(c42)C3)cc(C(C)(C)C)c1 ; CC(C)(C)c1cc(-c2ccc3c(c2)N2c4ccc(C(C)(C)C)cc4N(c4c(-c5ccccc5)cc5oc6ccccc6c5c4-c4ccccc4)c4cc(-n5c6ccc(C(C)(C)C)cc6c6cc(C(C)(C)C)ccc65)cc(c42)O3)cc(C(C)(C)C)c1]

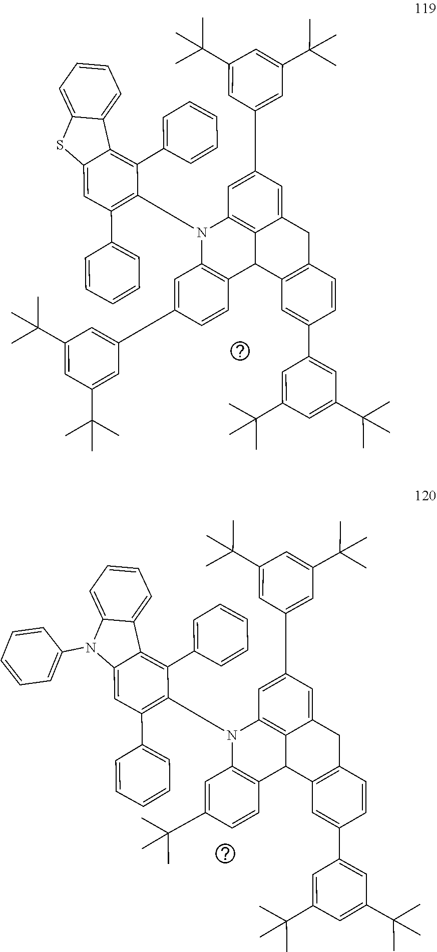 [SMILES: CC(C)(C)c1cc(-c2ccc3c(c2)C2c4ccc(-c5cc(C(C)(C)C)cc(C(C)(C)C)c5)cc4N(c4c(-c5ccccc5)cc5sc6ccccc6c5c4-c4ccccc4)c4cc(-c5cc(C(C)(C)C)cc(C(C)(C)C)c5)cc(c42)C3)cc(C(C)(C)C)c1 ; CC(C)(C)c1cc(-c2ccc3c(c2)C2c4ccc(C(C)(C)C)cc4N(c4c(-c5ccccc5)cc5c(c4-c4ccccc4)c4ccccc4n5-c4ccccc4)c4cc(-c5cc(C(C)(C)C)cc(C(C)(C)C)c5)cc(c42)C3)cc(C(C)(C)C)c1]

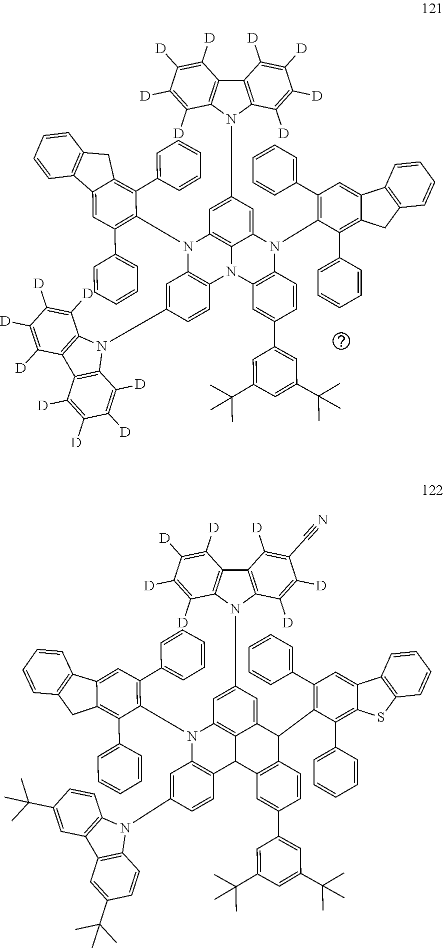

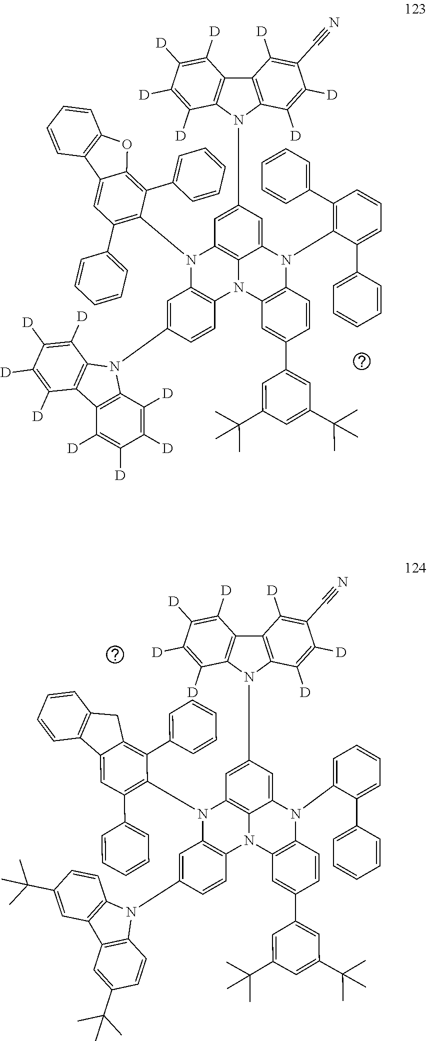

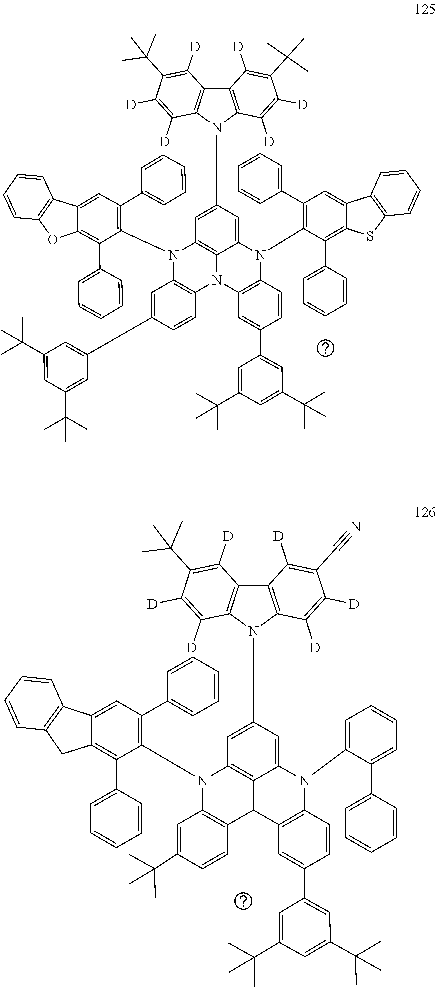 [SMILES: [2H]c1c(C(C)(C)C)c([2H])c2c3c([2H])c(C(C)(C)C)c([2H])c([2H])c3n(-c3cc4c5c(c3)N(c3c(-c6ccccc6)cc6c(sc7ccccc76)c3-c3ccccc3)c3ccc(-c6cc(C(C)(C)C)cc(C(C)(C)C)c6)cc3N5c3ccc(-c5cc(C(C)(C)C)cc(C(C)(C)C)c5)cc3N4c3c(-c4ccccc4)cc4c(oc5ccccc54)c3-c3ccccc3)c2c1[2H] ; [2H]c1c(C#N)c([2H])c2c3c([2H])c(C(C)(C)C)c([2H])c([2H])c3n(-c3cc4c5c(c3)N(c3c(-c6ccccc6)cc6c(c3-c3ccccc3)Cc3ccccc3-6)c3cc(C(C)(C)C)ccc3C5c3cc(-c5cc(C(C)(C)C)cc(C(C)(C)C)c5)ccc3N4c3ccccc3-c3ccccc3)c2c1[2H]]

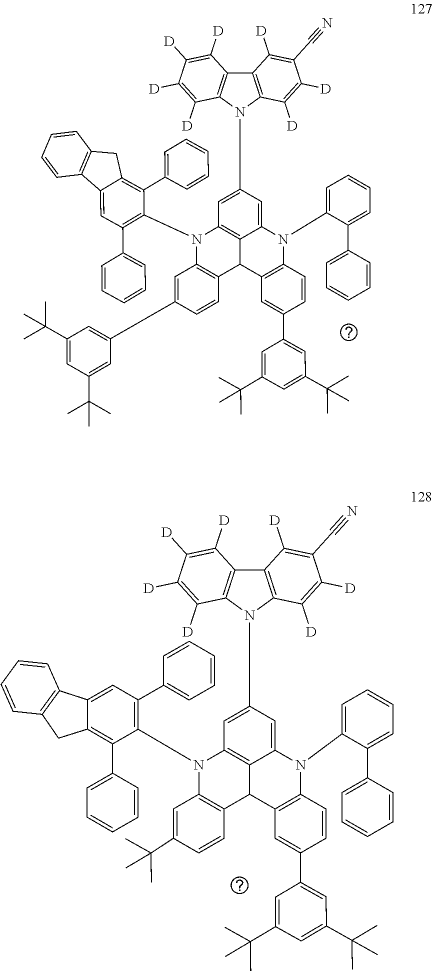 [SMILES: [2H]c1c([2H])c([2H])c2c(c1[2H])c1c([2H])c(C#N)c([2H])c([2H])c1n2-c1cc2c3c(c1)N(c1c(-c4ccccc4)cc4c(c1-c1ccccc1)Cc1ccccc1-4)c1cc(-c4cc(C(C)(C)C)cc(C(C)(C)C)c4)ccc1C3c1cc(-c3cc(C(C)(C)C)cc(C(C)(C)C)c3)ccc1N2c1ccccc1-c1ccccc1 ; [2H]c1c([2H])c([2H])c2c(c1[2H])c1c([2H])c(C#N)c([2H])c([2H])c1n2-c1cc2c3c(c1)N(c1c(-c4ccccc4)cc4c(c1-c1ccccc1)Cc1ccccc1-4)c1cc(C(C)(C)C)ccc1C3c1cc(-c3cc(C(C)(C)C)cc(C(C)(C)C)c3)ccc1N2c1ccccc1-c1ccccc1]

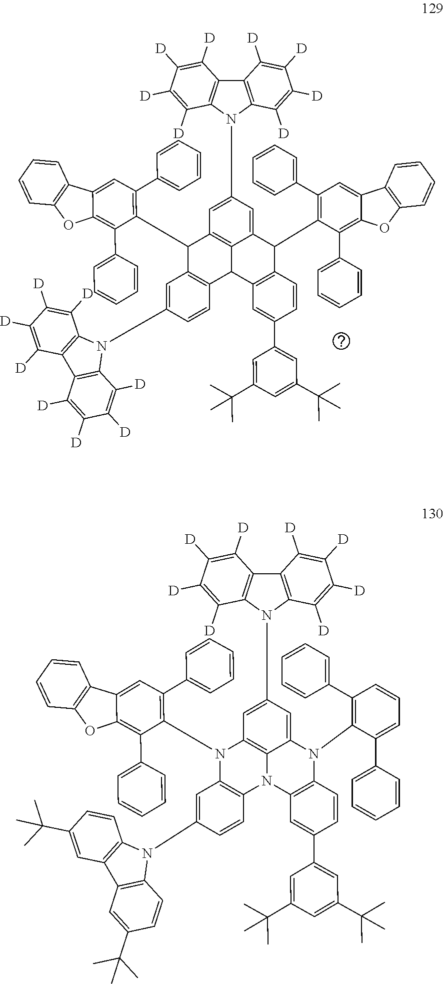

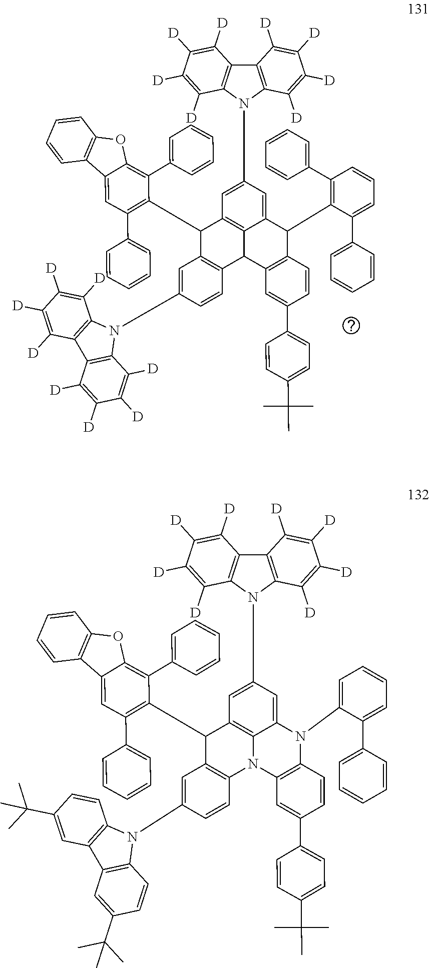 [SMILES: [2H]c1c([2H])c([2H])c2c(c1[2H])c1c([2H])c([2H])c([2H])c([2H])c1n2-c1ccc2c(c1)C(c1c(-c3ccccc3)cc3c(oc4ccccc43)c1-c1ccccc1)c1cc(-n3c4c([2H])c([2H])c([2H])c([2H])c4c4c([2H])c([2H])c([2H])c([2H])c43)cc3c1C2c1cc(-c2ccc(C(C)(C)C)cc2)ccc1C3c1c(-c2ccccc2)cccc1-c1ccccc1 ; [2H]c1c([2H])c([2H])c2c(c1[2H])c1c([2H])c([2H])c([2H])c([2H])c1n2-c1cc2c3c(c1)N(c1ccccc1-c1ccccc1)c1ccc(-c4ccc(C(C)(C)C)cc4)cc1N3c1ccc(-n3c4ccc(C(C)(C)C)cc4c4cc(C(C)(C)C)ccc43)cc1C2c1c(-c2ccccc2)cc2c(oc3ccccc32)c1-c1ccccc1]

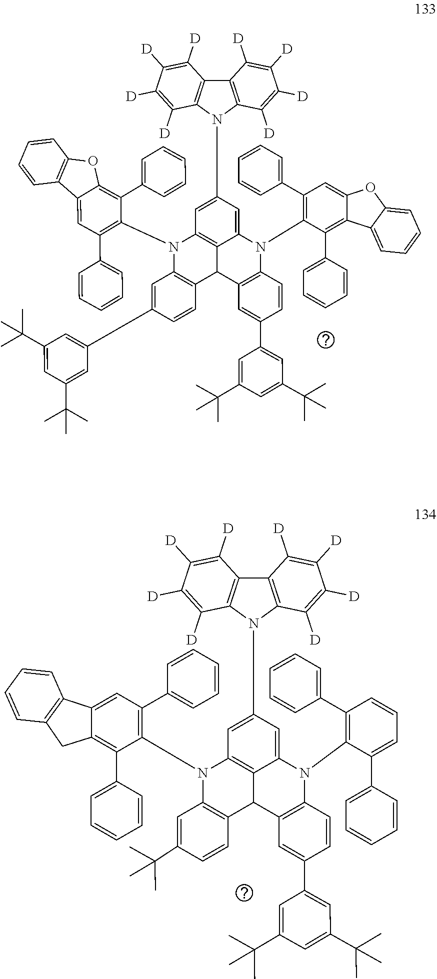 [SMILES: [2H]c1c([2H])c([2H])c2c(c1[2H])c1c([2H])c([2H])c([2H])c([2H])c1n2-c1cc2c3c(c1)N(c1c(-c4ccccc4)cc4oc5ccccc5c4c1-c1ccccc1)c1ccc(-c4cc(C(C)(C)C)cc(C(C)(C)C)c4)cc1C3c1ccc(-c3cc(C(C)(C)C)cc(C(C)(C)C)c3)cc1N2c1c(-c2ccccc2)cc2c(oc3ccccc32)c1-c1ccccc1 ; [2H]c1c([2H])c([2H])c2c(c1[2H])c1c([2H])c([2H])c([2H])c([2H])c1n2-c1cc2c3c(c1)N(c1c(-c4ccccc4)cc4c(c1-c1ccccc1)Cc1ccccc1-4)c1cc(C(C)(C)C)ccc1C3c1cc(-c3cc(C(C)(C)C)cc(C(C)(C)C)c3)ccc1N2c1c(-c2ccccc2)cccc1-c1ccccc1]

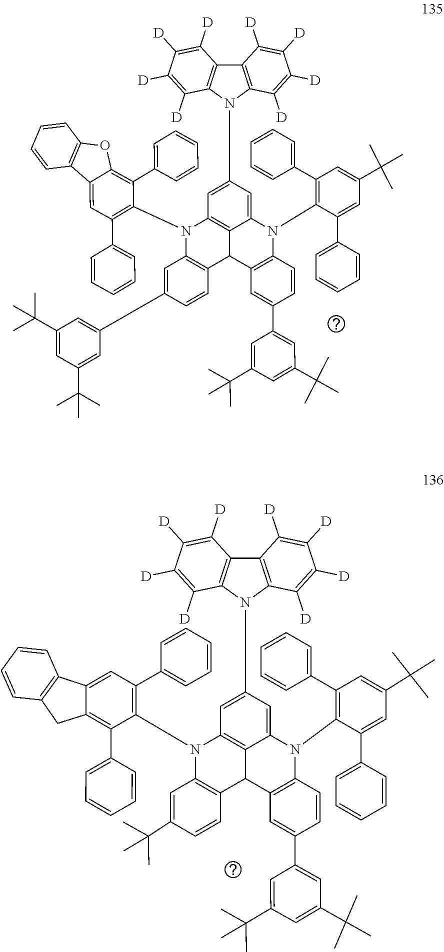 [SMILES: [2H]c1c([2H])c([2H])c2c(c1[2H])c1c([2H])c([2H])c([2H])c([2H])c1n2-c1cc2c3c(c1)N(c1c(-c4ccccc4)cc4c(oc5ccccc54)c1-c1ccccc1)c1cc(-c4cc(C(C)(C)C)cc(C(C)(C)C)c4)ccc1C3c1cc(-c3cc(C(C)(C)C)cc(C(C)(C)C)c3)ccc1N2c1c(-c2ccccc2)cc(C(C)(C)C)cc1-c1ccccc1 ; [2H]c1c([2H])c([2H])c2c(c1[2H])c1c([2H])c([2H])c([2H])c([2H])c1n2-c1cc2c3c(c1)N(c1c(-c4ccccc4)cc4c(c1-c1ccccc1)Cc1ccccc1-4)c1cc(C(C)(C)C)ccc1C3c1cc(-c3cc(C(C)(C)C)cc(C(C)(C)C)c3)ccc1N2c1c(-c2ccccc2)cc(C(C)(C)C)cc1-c1ccccc1]

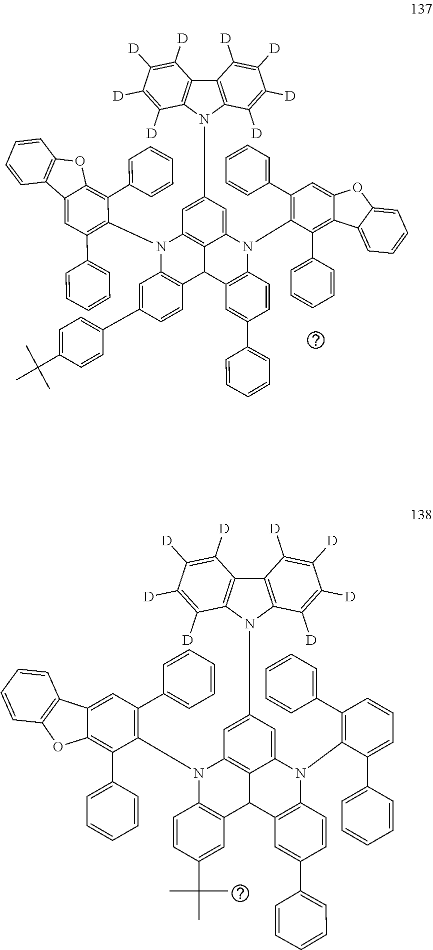 [SMILES: [2H]c1c([2H])c([2H])c2c(c1[2H])c1c([2H])c([2H])c([2H])c([2H])c1n2-c1cc2c3c(c1)N(c1c(-c4ccccc4)cc4oc5ccccc5c4c1-c1ccccc1)c1ccc(-c4ccccc4)cc1C3c1ccc(-c3ccc(C(C)(C)C)cc3)cc1N2c1c(-c2ccccc2)cc2c(oc3ccccc32)c1-c1ccccc1 ; [2H]c1c([2H])c([2H])c2c(c1[2H])c1c([2H])c([2H])c([2H])c([2H])c1n2-c1cc2c3c(c1)N(c1c(-c4ccccc4)cc4c(oc5ccccc54)c1-c1ccccc1)c1ccc(C(C)(C)C)cc1C3c1cc(-c3ccccc3)ccc1N2c1c(-c2ccccc2)cccc1-c1ccccc1]

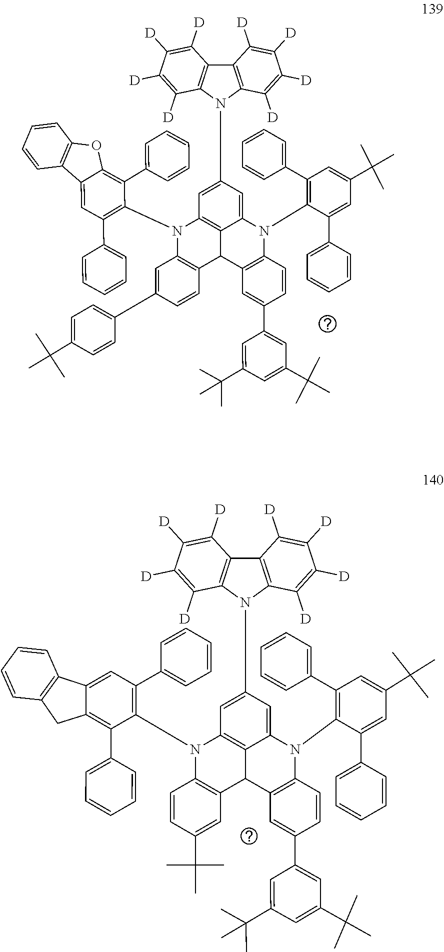 [SMILES: [2H]c1c([2H])c([2H])c2c(c1[2H])c1c([2H])c([2H])c([2H])c([2H])c1n2-c1cc2c3c(c1)N(c1c(-c4ccccc4)cc4c(oc5ccccc54)c1-c1ccccc1)c1cc(-c4ccc(C(C)(C)C)cc4)ccc1C3c1cc(-c3cc(C(C)(C)C)cc(C(C)(C)C)c3)ccc1N2c1c(-c2ccccc2)cc(C(C)(C)C)cc1-c1ccccc1 ; [2H]c1c([2H])c([2H])c2c(c1[2H])c1c([2H])c([2H])c([2H])c([2H])c1n2-c1cc2c3c(c1)N(c1c(-c4ccccc4)cc4c(c1-c1ccccc1)Cc1ccccc1-4)c1ccc(C(C)(C)C)cc1C3c1cc(-c3cc(C(C)(C)C)cc(C(C)(C)C)c3)ccc1N2c1c(-c2ccccc2)cc(C(C)(C)C)cc1-c1ccccc1]

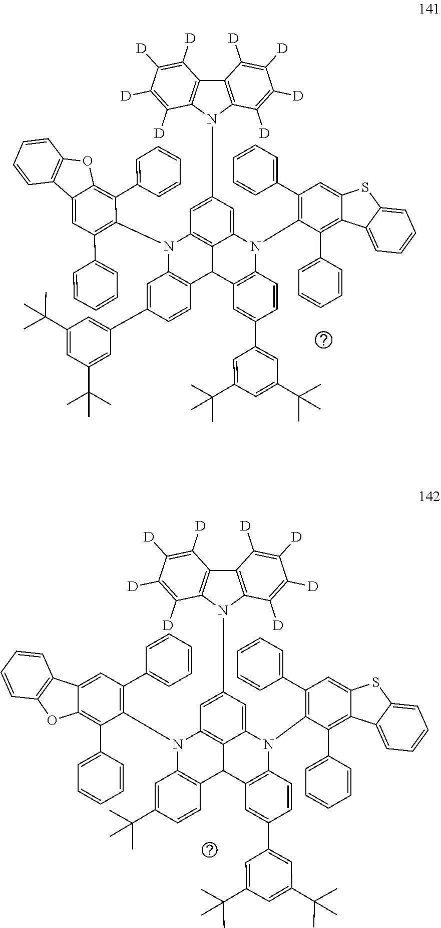 [SMILES: [2H]c1c([2H])c([2H])c2c(c1[2H])c1c([2H])c([2H])c([2H])c([2H])c1n2-c1cc2c3c(c1)N(c1c(-c4ccccc4)cc4sc5ccccc5c4c1-c1ccccc1)c1ccc(-c4cc(C(C)(C)C)cc(C(C)(C)C)c4)cc1C3c1ccc(-c3cc(C(C)(C)C)cc(C(C)(C)C)c3)cc1N2c1c(-c2ccccc2)cc2c(oc3ccccc32)c1-c1ccccc1 ; [2H]c1c([2H])c([2H])c2c(c1[2H])c1c([2H])c([2H])c([2H])c([2H])c1n2-c1cc2c3c(c1)N(c1c(-c4ccccc4)cc4sc5ccccc5c4c1-c1ccccc1)c1ccc(-c4cc(C(C)(C)C)cc(C(C)(C)C)c4)cc1C3c1ccc(C(C)(C)C)cc1N2c1c(-c2ccccc2)cc2c(oc3ccccc32)c1-c1ccccc1]

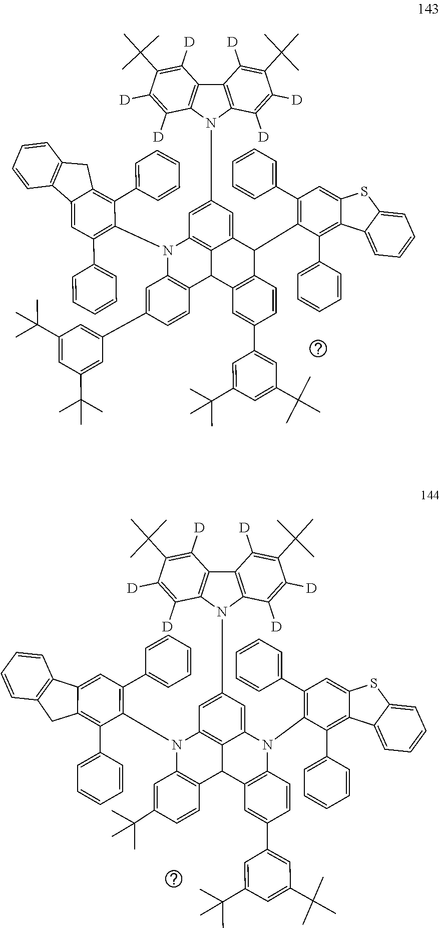 [SMILES: [2H]c1c(C(C)(C)C)c([2H])c2c3c([2H])c(C(C)(C)C)c([2H])c([2H])c3n(-c3cc4c5c(c3)N(c3c(-c6ccccc6)cc6c(c3-c3ccccc3)Cc3ccccc3-6)c3cc(-c6cc(C(C)(C)C)cc(C(C)(C)C)c6)ccc3C5c3cc(-c5cc(C(C)(C)C)cc(C(C)(C)C)c5)ccc3C4c3c(-c4ccccc4)cc4sc5ccccc5c4c3-c3ccccc3)c2c1[2H] ; [2H]c1c(C(C)(C)C)c([2H])c2c3c([2H])c(C(C)(C)C)c([2H])c([2H])c3n(-c3cc4c5c(c3)N(c3c(-c6ccccc6)cc6sc7ccccc7c6c3-c3ccccc3)c3ccc(-c6cc(C(C)(C)C)cc(C(C)(C)C)c6)cc3C5c3ccc(C(C)(C)C)cc3N4c3c(-c4ccccc4)cc4c(c3-c3ccccc3)Cc3ccccc3-4)c2c1[2H]]

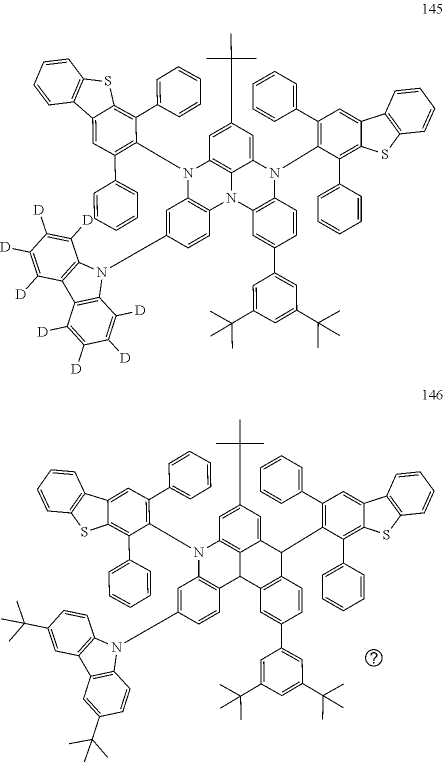 [SMILES: [2H]c1c([2H])c([2H])c2c(c1[2H])c1c([2H])c([2H])c([2H])c([2H])c1n2-c1ccc2c(c1)N(c1c(-c3ccccc3)cc3c(sc4ccccc43)c1-c1ccccc1)c1cc(C(C)(C)C)cc3c1N2c1cc(-c2cc(C(C)(C)C)cc(C(C)(C)C)c2)ccc1N3c1c(-c2ccccc2)cc2c(sc3ccccc32)c1-c1ccccc1 ; CC(C)(C)c1cc(-c2ccc3c(c2)C2c4ccc(-n5c6ccc(C(C)(C)C)cc6c6cc(C(C)(C)C)ccc65)cc4N(c4c(-c5ccccc5)cc5c(sc6ccccc65)c4-c4ccccc4)c4cc(C(C)(C)C)cc(c42)C3c2c(-c3ccccc3)cc3c(sc4ccccc43)c2-c2ccccc2)cc(C(C)(C)C)c1]

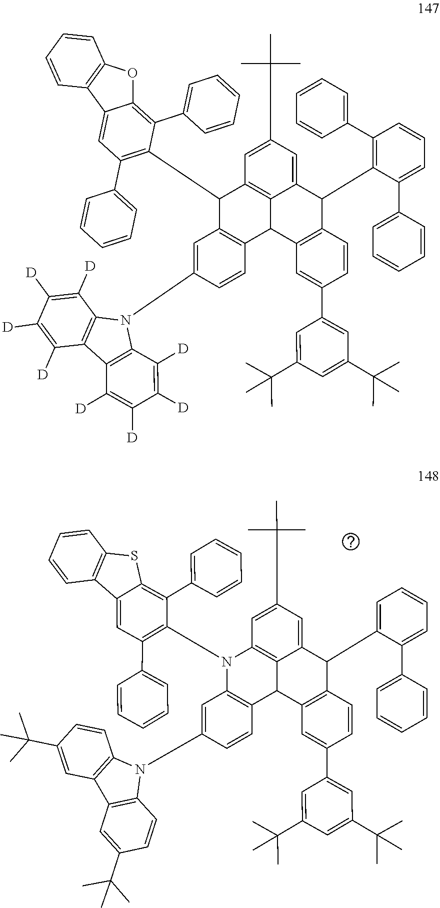 [SMILES: [2H]c1c([2H])c([2H])c2c(c1[2H])c1c([2H])c([2H])c([2H])c([2H])c1n2-c1ccc2c(c1)C(c1c(-c3ccccc3)cc3c(oc4ccccc43)c1-c1ccccc1)c1cc(C(C)(C)C)cc3c1C2c1cc(-c2cc(C(C)(C)C)cc(C(C)(C)C)c2)ccc1C3c1c(-c2ccccc2)cccc1-c1ccccc1 ; CC(C)(C)c1cc(-c2ccc3c(c2)C2c4ccc(-n5c6ccc(C(C)(C)C)cc6c6cc(C(C)(C)C)ccc65)cc4N(c4c(-c5ccccc5)cc5c(sc6ccccc65)c4-c4ccccc4)c4cc(C(C)(C)C)cc(c42)C3c2ccccc2-c2ccccc2)cc(C(C)(C)C)c1]

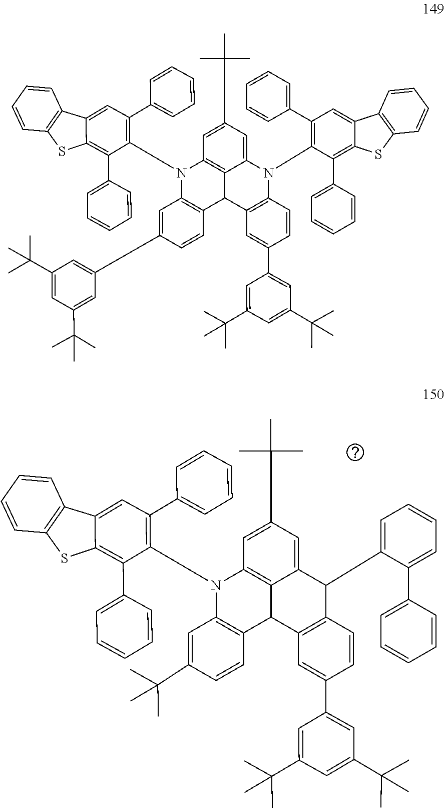 [SMILES: CC(C)(C)c1cc(-c2ccc3c(c2)C2c4ccc(-c5cc(C(C)(C)C)cc(C(C)(C)C)c5)cc4N(c4c(-c5ccccc5)cc5c(sc6ccccc65)c4-c4ccccc4)c4cc(C(C)(C)C)cc(c42)N3c2c(-c3ccccc3)cc3c(sc4ccccc43)c2-c2ccccc2)cc(C(C)(C)C)c1 ; CC(C)(C)c1cc(-c2ccc3c(c2)C2c4ccc(C(C)(C)C)cc4N(c4c(-c5ccccc5)cc5c(sc6ccccc65)c4-c4ccccc4)c4cc(C(C)(C)C)cc(c42)C3c2ccccc2-c2ccccc2)cc(C(C)(C)C)c1]

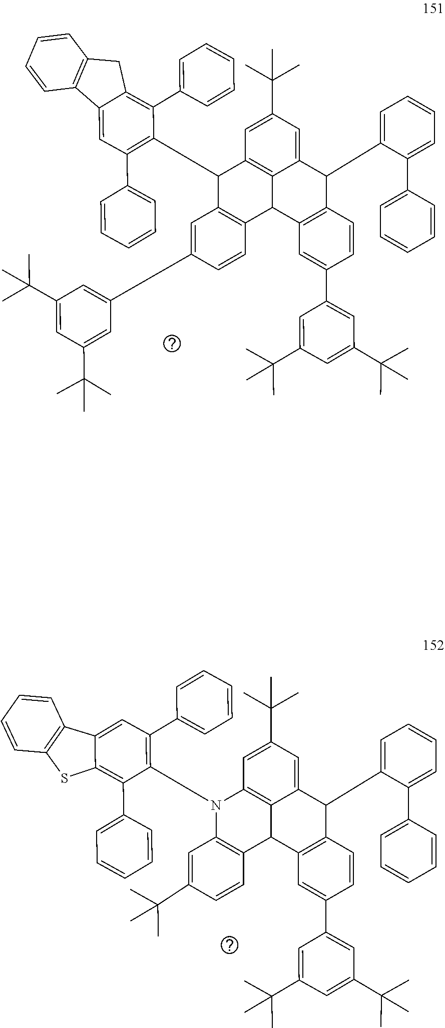

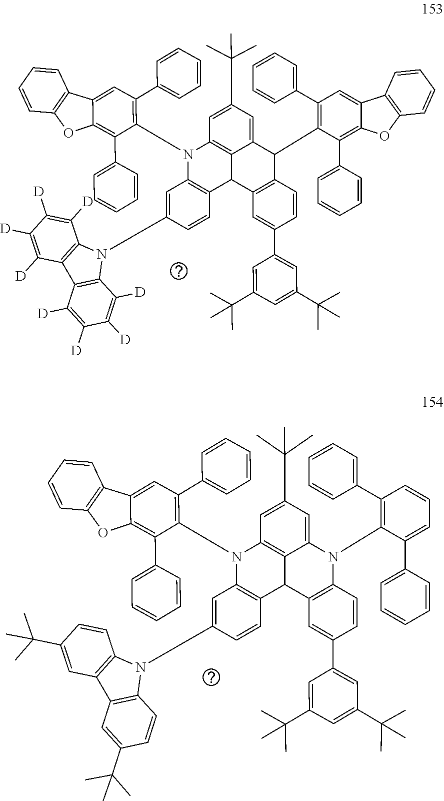 [SMILES: [2H]c1c([2H])c([2H])c2c(c1[2H])c1c([2H])c([2H])c([2H])c([2H])c1n2-c1ccc2c(c1)N(c1c(-c3ccccc3)cc3c(oc4ccccc43)c1-c1ccccc1)c1cc(C(C)(C)C)cc3c1C2c1cc(-c2cc(C(C)(C)C)cc(C(C)(C)C)c2)ccc1C3c1c(-c2ccccc2)cc2c(oc3ccccc32)c1-c1ccccc1 ; CC(C)(C)c1cc(-c2ccc3c(c2)C2c4ccc(-n5c6ccc(C(C)(C)C)cc6c6cc(C(C)(C)C)ccc65)cc4N(c4c(-c5ccccc5)cc5c(oc6ccccc65)c4-c4ccccc4)c4cc(C(C)(C)C)cc(c42)N3c2c(-c3ccccc3)cccc2-c2ccccc2)cc(C(C)(C)C)c1]

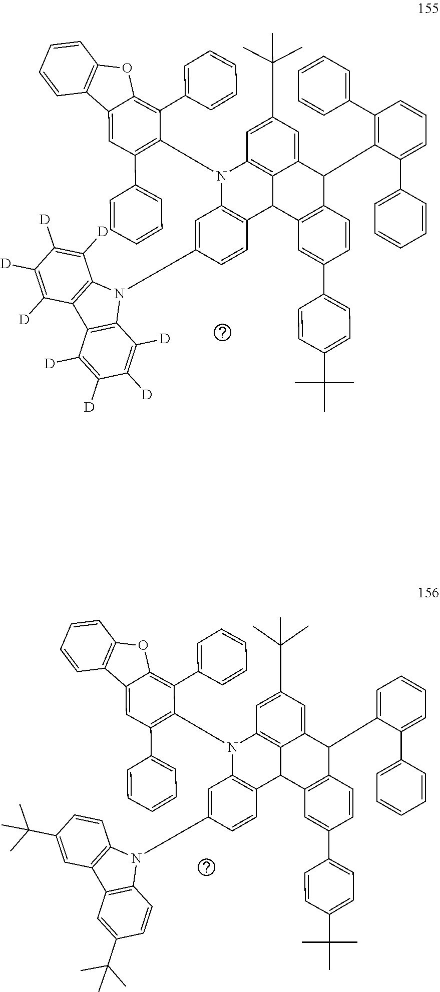 [SMILES: [2H]c1c([2H])c([2H])c2c(c1[2H])c1c([2H])c([2H])c([2H])c([2H])c1n2-c1ccc2c(c1)N(c1c(-c3ccccc3)cc3c(oc4ccccc43)c1-c1ccccc1)c1cc(C(C)(C)C)cc3c1C2c1cc(-c2ccc(C(C)(C)C)cc2)ccc1C3c1c(-c2ccccc2)cccc1-c1ccccc1 ; CC(C)(C)c1ccc(-c2ccc3c(c2)C2c4ccc(-n5c6ccc(C(C)(C)C)cc6c6cc(C(C)(C)C)ccc65)cc4N(c4c(-c5ccccc5)cc5c(oc6ccccc65)c4-c4ccccc4)c4cc(C(C)(C)C)cc(c42)C3c2ccccc2-c2ccccc2)cc1]

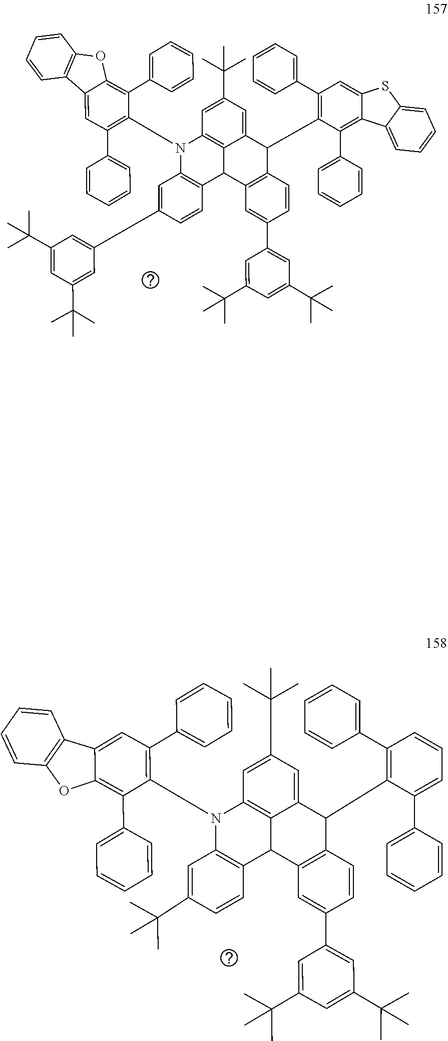 [SMILES: CC(C)(C)c1cc(-c2ccc3c(c2)C2c4ccc(-c5cc(C(C)(C)C)cc(C(C)(C)C)c5)cc4N(c4c(-c5ccccc5)cc5c(oc6ccccc65)c4-c4ccccc4)c4cc(C(C)(C)C)cc(c42)C3c2c(-c3ccccc3)cc3sc4ccccc4c3c2-c2ccccc2)cc(C(C)(C)C)c1 ; CC(C)(C)c1cc(-c2ccc3c(c2)C2c4ccc(C(C)(C)C)cc4N(c4c(-c5ccccc5)cc5c(oc6ccccc65)c4-c4ccccc4)c4cc(C(C)(C)C)cc(c42)C3c2c(-c3ccccc3)cccc2-c2ccccc2)cc(C(C)(C)C)c1]

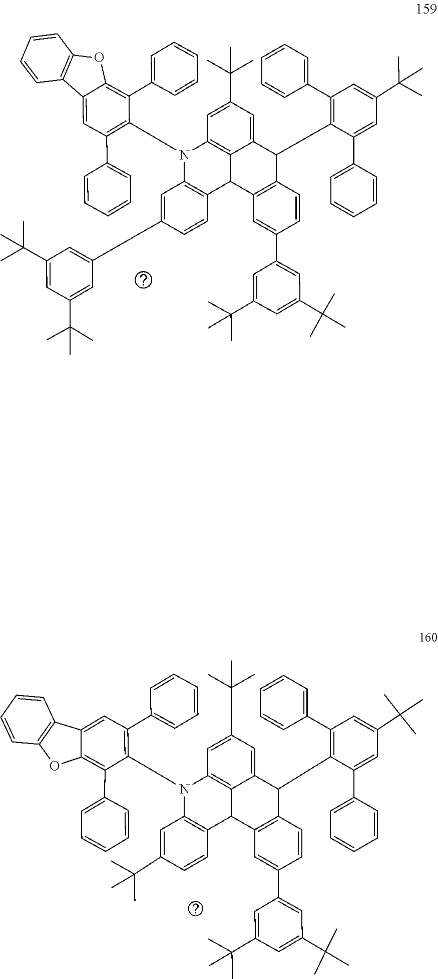 [SMILES: CC(C)(C)c1cc(-c2ccc3c(c2)C2c4ccc(-c5cc(C(C)(C)C)cc(C(C)(C)C)c5)cc4N(c4c(-c5ccccc5)cc5c(oc6ccccc65)c4-c4ccccc4)c4cc(C(C)(C)C)cc(c42)C3c2c(-c3ccccc3)cc(C(C)(C)C)cc2-c2ccccc2)cc(C(C)(C)C)c1 ; CC(C)(C)c1cc(-c2ccc3c(c2)C2c4ccc(C(C)(C)C)cc4N(c4c(-c5ccccc5)cc5c(oc6ccccc65)c4-c4ccccc4)c4cc(C(C)(C)C)cc(c42)C3c2c(-c3ccccc3)cc(C(C)(C)C)cc2-c2ccccc2)cc(C(C)(C)C)c1]

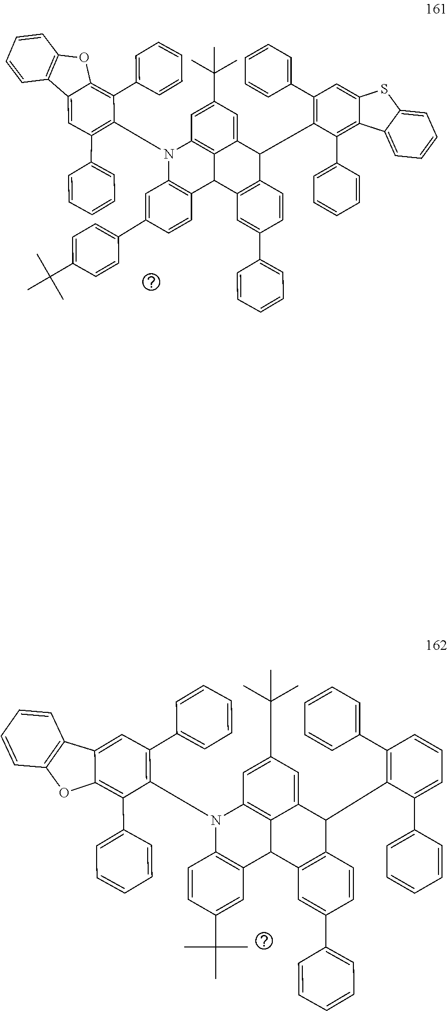 [SMILES: CC(C)(C)c1ccc(-c2ccc3c(c2)N(c2c(-c4ccccc4)cc4c(oc5ccccc54)c2-c2ccccc2)c2cc(C(C)(C)C)cc4c2C3c2cc(-c3ccccc3)ccc2C4c2c(-c3ccccc3)cc3sc4ccccc4c3c2-c2ccccc2)cc1 ; CC(C)(C)c1ccc2c(c1)C1c3cc(-c4ccccc4)ccc3C(c3c(-c4ccccc4)cccc3-c3ccccc3)c3cc(C(C)(C)C)cc(c31)N2c1c(-c2ccccc2)cc2c(oc3ccccc32)c1-c1ccccc1]

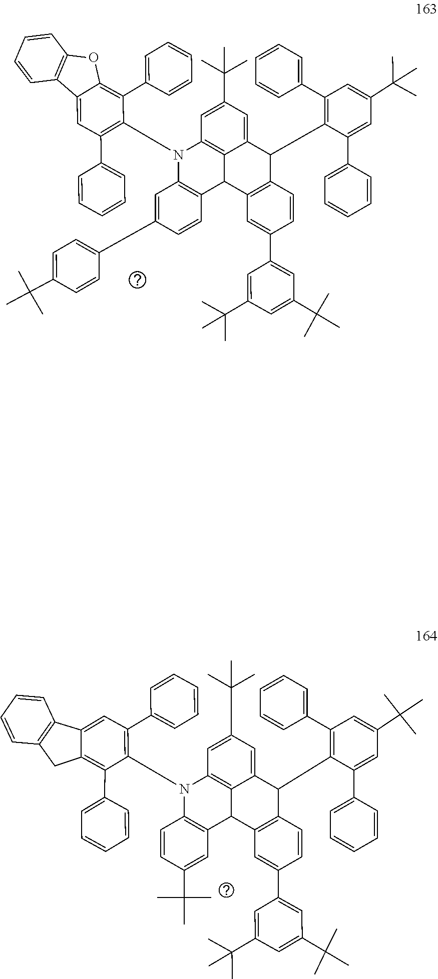 [SMILES: CC(C)(C)c1ccc(-c2ccc3c(c2)N(c2c(-c4ccccc4)cc4c(oc5ccccc54)c2-c2ccccc2)c2cc(C(C)(C)C)cc4c2C3c2cc(-c3cc(C(C)(C)C)cc(C(C)(C)C)c3)ccc2C4c2c(-c3ccccc3)cc(C(C)(C)C)cc2-c2ccccc2)cc1 ; CC(C)(C)c1cc(-c2ccc3c(c2)C2c4cc(C(C)(C)C)ccc4N(c4c(-c5ccccc5)cc5c(c4-c4ccccc4)Cc4ccccc4-5)c4cc(C(C)(C)C)cc(c42)C3c2c(-c3ccccc3)cc(C(C)(C)C)cc2-c2ccccc2)cc(C(C)(C)C)c1]

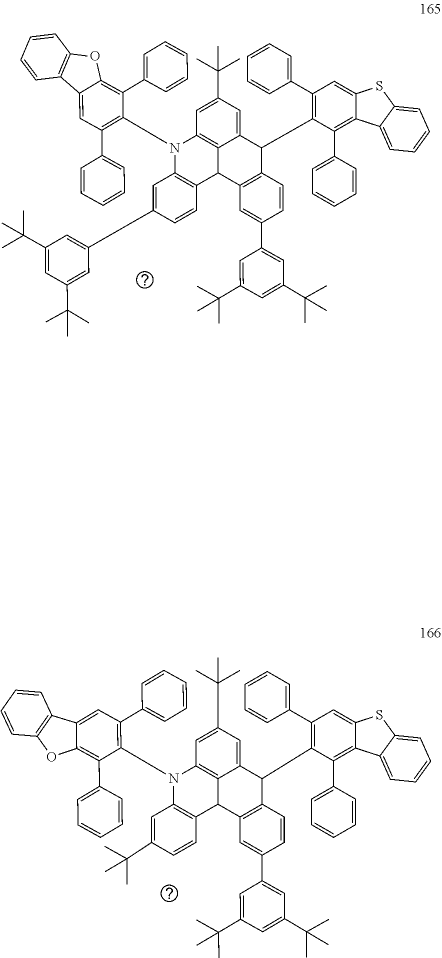 [SMILES: CC(C)(C)c1cc(-c2ccc3c(c2)C2c4ccc(-c5cc(C(C)(C)C)cc(C(C)(C)C)c5)cc4N(c4c(-c5ccccc5)cc5c(oc6ccccc65)c4-c4ccccc4)c4cc(C(C)(C)C)cc(c42)C3c2c(-c3ccccc3)cc3sc4ccccc4c3c2-c2ccccc2)cc(C(C)(C)C)c1 ; CC(C)(C)c1cc(-c2ccc3c(c2)C2c4ccc(C(C)(C)C)cc4N(c4c(-c5ccccc5)cc5c(oc6ccccc65)c4-c4ccccc4)c4cc(C(C)(C)C)cc(c42)C3c2c(-c3ccccc3)cc3sc4ccccc4c3c2-c2ccccc2)cc(C(C)(C)C)c1]

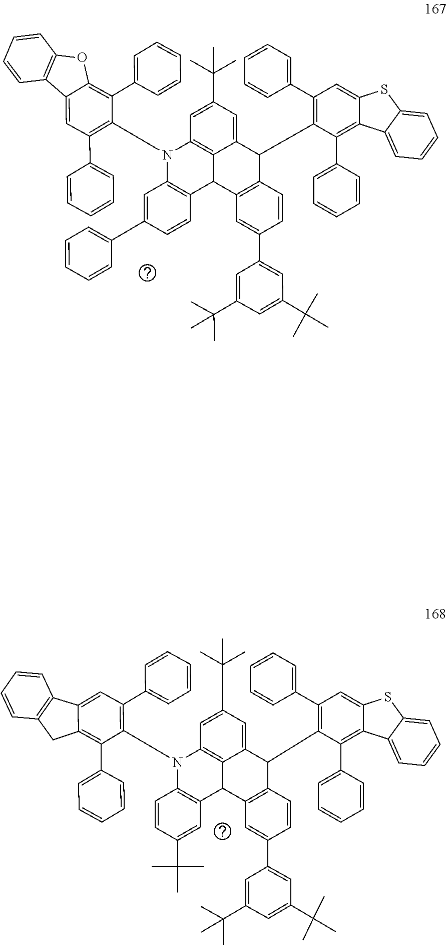 [SMILES: CC(C)(C)c1cc(-c2ccc3c(c2)C2c4ccc(-c5ccccc5)cc4N(c4c(-c5ccccc5)cc5c(oc6ccccc65)c4-c4ccccc4)c4cc(C(C)(C)C)cc(c42)C3c2c(-c3ccccc3)cc3sc4ccccc4c3c2-c2ccccc2)cc(C(C)(C)C)c1 ; CC(C)(C)c1cc(-c2ccc3c(c2)C2c4cc(C(C)(C)C)ccc4N(c4c(-c5ccccc5)cc5c(c4-c4ccccc4)Cc4ccccc4-5)c4cc(C(C)(C)C)cc(c42)C3c2c(-c3ccccc3)cc3sc4ccccc4c3c2-c2ccccc2)cc(C(C)(C)C)c1]

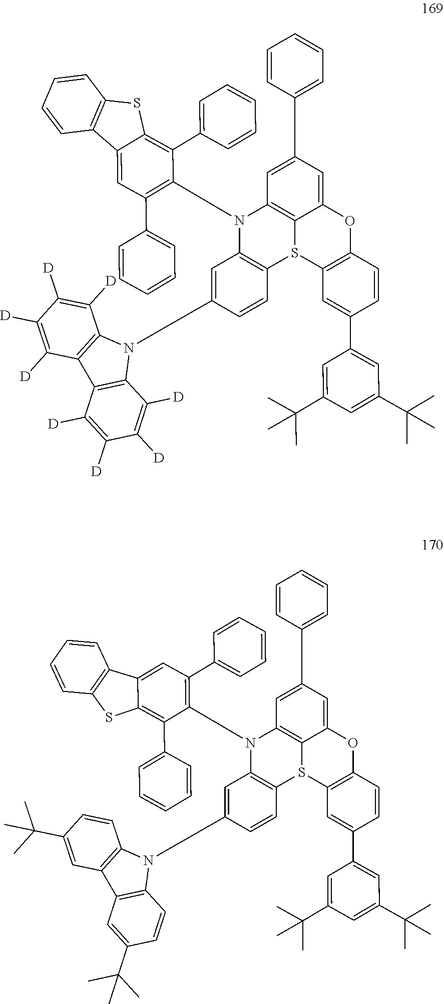

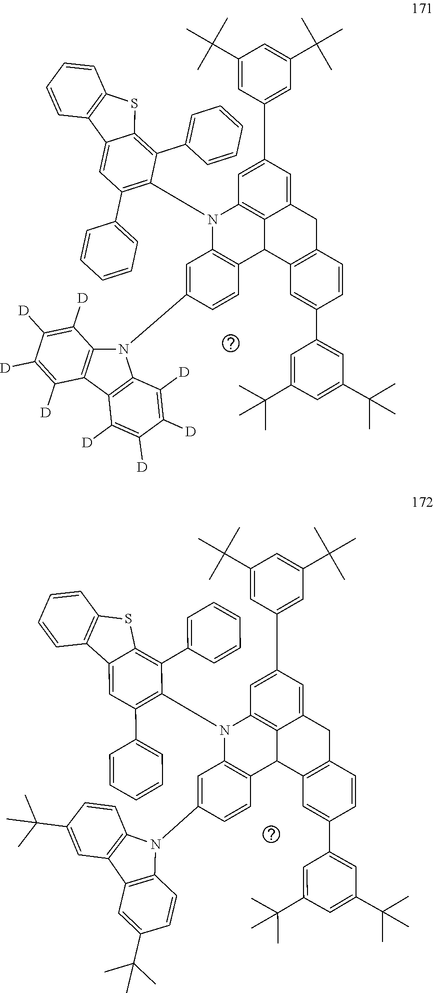

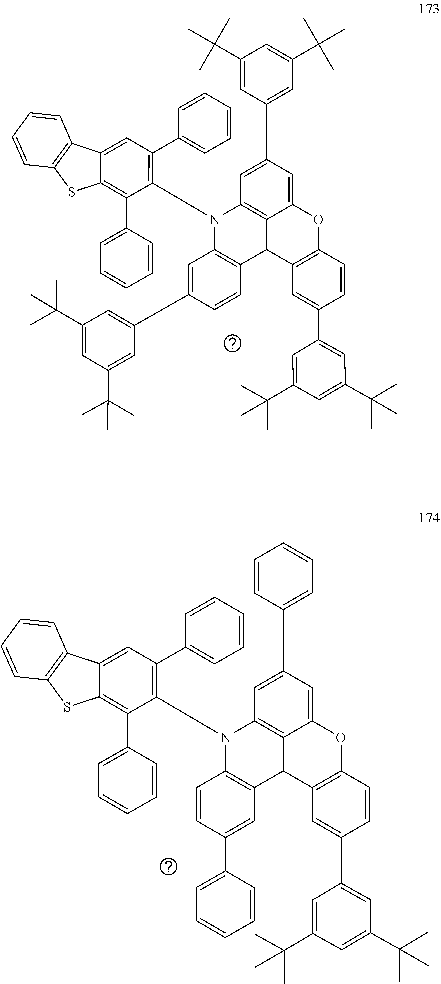 [SMILES: CC(C)(C)c1cc(-c2ccc3c(c2)C2c4ccc(-c5cc(C(C)(C)C)cc(C(C)(C)C)c5)cc4N(c4c(-c5ccccc5)cc5c(sc6ccccc65)c4-c4ccccc4)c4cc(-c5cc(C(C)(C)C)cc(C(C)(C)C)c5)cc(c42)O3)cc(C(C)(C)C)c1 ; CC(C)(C)c1cc(-c2ccc3c(c2)C2c4cc(-c5ccccc5)ccc4N(c4c(-c5ccccc5)cc5c(sc6ccccc65)c4-c4ccccc4)c4cc(-c5ccccc5)cc(c42)O3)cc(C(C)(C)C)c1]

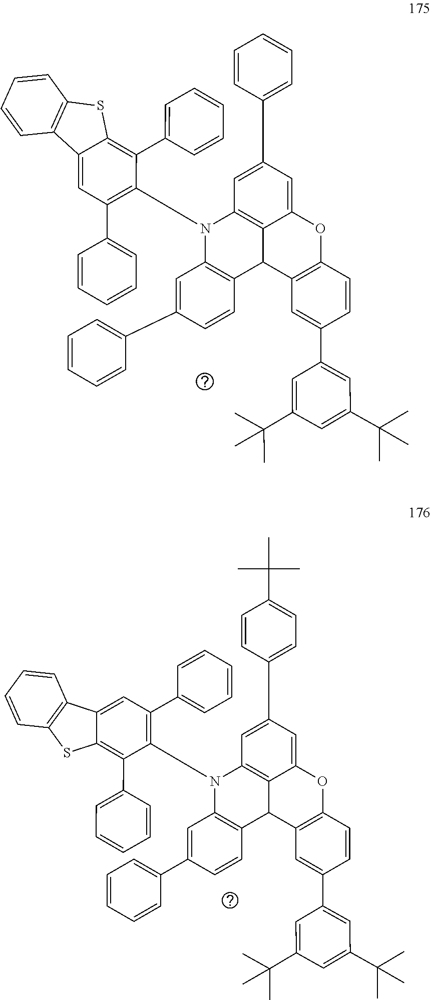 [SMILES: CC(C)(C)c1cc(-c2ccc3c(c2)C2c4ccc(-c5ccccc5)cc4N(c4c(-c5ccccc5)cc5c(sc6ccccc65)c4-c4ccccc4)c4cc(-c5ccccc5)cc(c42)O3)cc(C(C)(C)C)c1 ; CC(C)(C)c1ccc(-c2cc3c4c(c2)N(c2c(-c5ccccc5)cc5c(sc6ccccc65)c2-c2ccccc2)c2cc(-c5ccccc5)ccc2C4c2cc(-c4cc(C(C)(C)C)cc(C(C)(C)C)c4)ccc2O3)cc1]

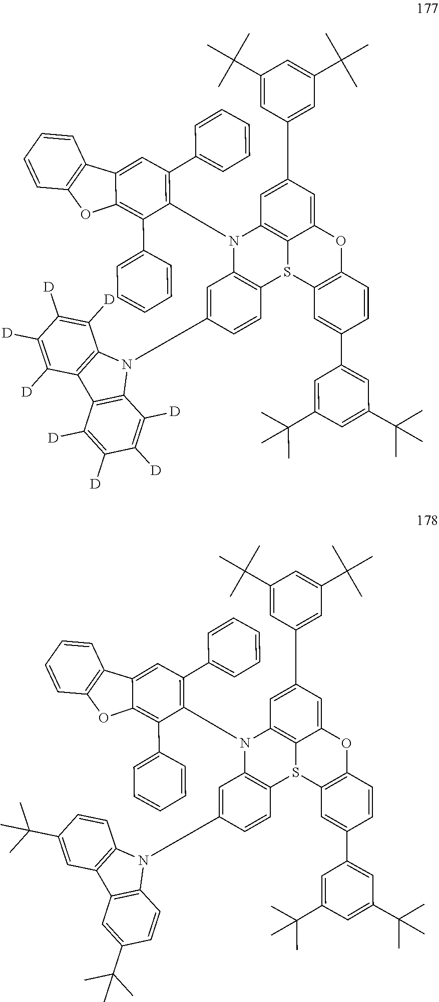

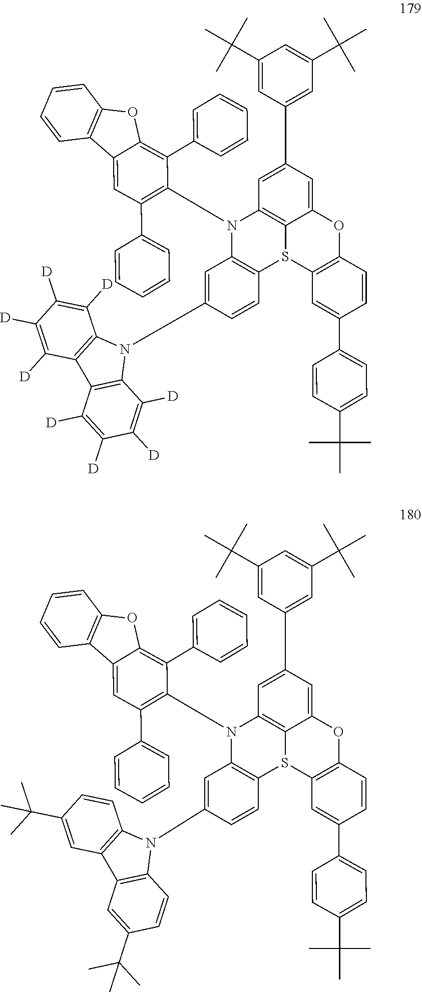

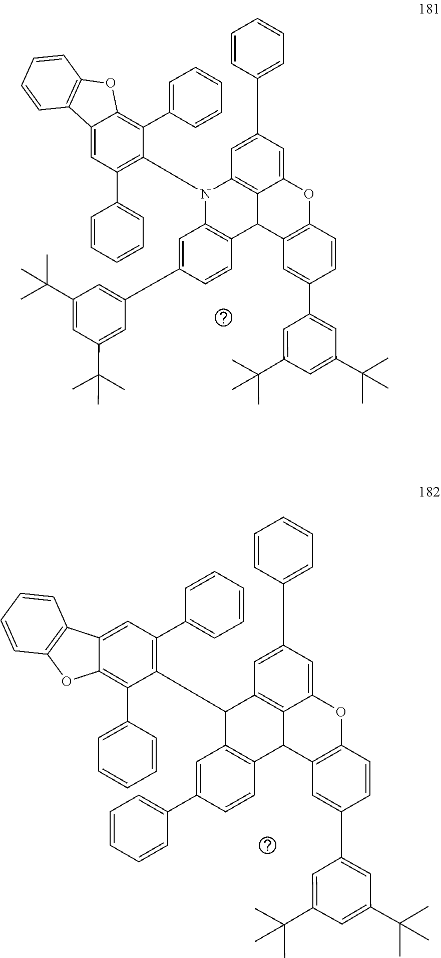 [SMILES: CC(C)(C)c1cc(-c2ccc3c(c2)C2c4ccc(-c5cc(C(C)(C)C)cc(C(C)(C)C)c5)cc4N(c4c(-c5ccccc5)cc5c(oc6ccccc65)c4-c4ccccc4)c4cc(-c5ccccc5)cc(c42)O3)cc(C(C)(C)C)c1 ; CC(C)(C)c1cc(-c2ccc3c(c2)C2c4ccc(-c5ccccc5)cc4C(c4c(-c5ccccc5)cc5c(oc6ccccc65)c4-c4ccccc4)c4cc(-c5ccccc5)cc(c42)O3)cc(C(C)(C)C)c1]

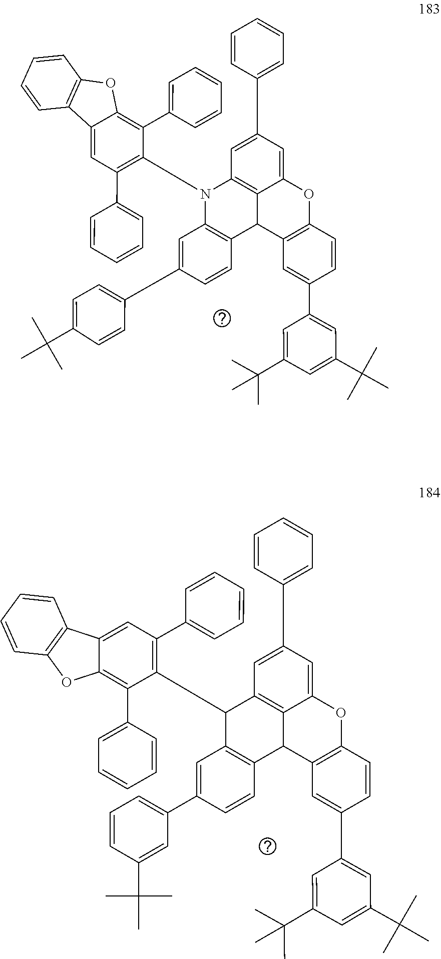 [SMILES: CC(C)(C)c1ccc(-c2ccc3c(c2)N(c2c(-c4ccccc4)cc4c(oc5ccccc54)c2-c2ccccc2)c2cc(-c4ccccc4)cc4c2C3c2cc(-c3cc(C(C)(C)C)cc(C(C)(C)C)c3)ccc2O4)cc1 ; CC(C)(C)c1cccc(-c2ccc3c(c2)C(c2c(-c4ccccc4)cc4c(oc5ccccc54)c2-c2ccccc2)c2cc(-c4ccccc4)cc4c2C3c2cc(-c3cc(C(C)(C)C)cc(C(C)(C)C)c3)ccc2O4)c1]

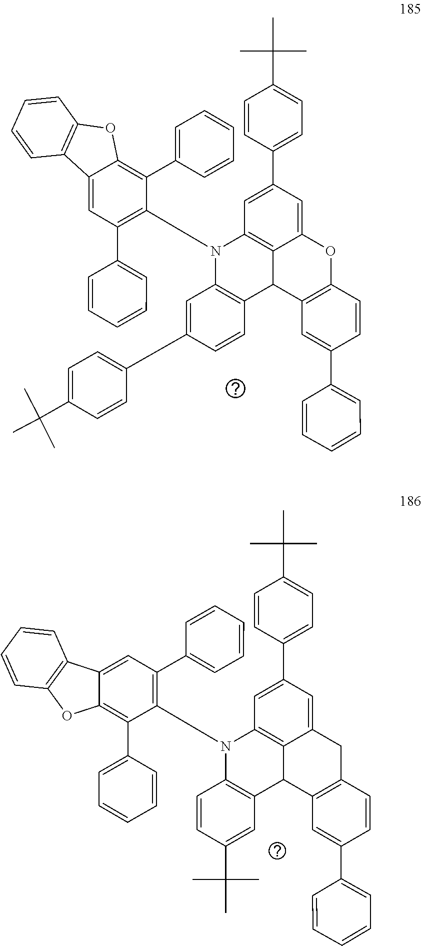 [SMILES: CC(C)(C)c1ccc(-c2ccc3c(c2)N(c2c(-c4ccccc4)cc4c(oc5ccccc54)c2-c2ccccc2)c2cc(-c4ccc(C(C)(C)C)cc4)cc4c2C3c2cc(-c3ccccc3)ccc2O4)cc1 ; CC(C)(C)c1ccc(-c2cc3c4c(c2)N(c2c(-c5ccccc5)cc5c(oc6ccccc65)c2-c2ccccc2)c2ccc(C(C)(C)C)cc2C4c2cc(-c4ccccc4)ccc2C3)cc1]

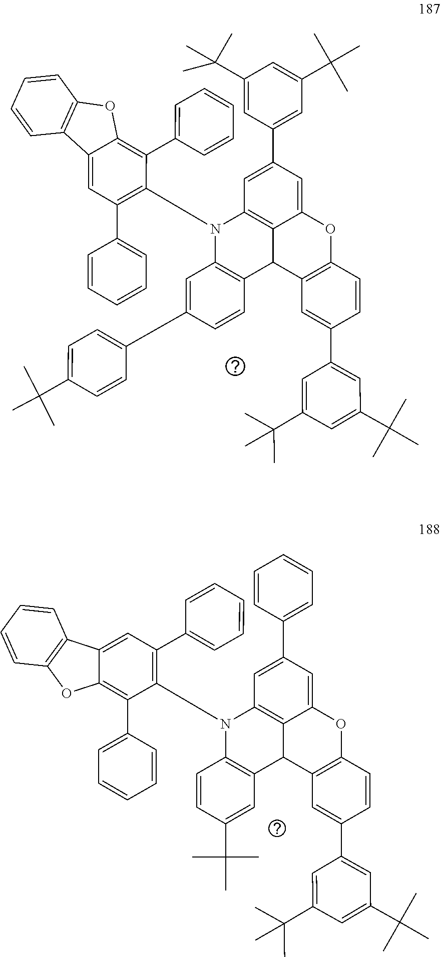 [SMILES: CC(C)(C)c1ccc(-c2ccc3c(c2)N(c2c(-c4ccccc4)cc4c(oc5ccccc54)c2-c2ccccc2)c2cc(-c4cc(C(C)(C)C)cc(C(C)(C)C)c4)cc4c2C3c2cc(-c3cc(C(C)(C)C)cc(C(C)(C)C)c3)ccc2O4)cc1 ; CC(C)(C)c1cc(-c2ccc3c(c2)C2c4cc(C(C)(C)C)ccc4N(c4c(-c5ccccc5)cc5c(oc6ccccc65)c4-c4ccccc4)c4cc(-c5ccccc5)cc(c42)O3)cc(C(C)(C)C)c1]

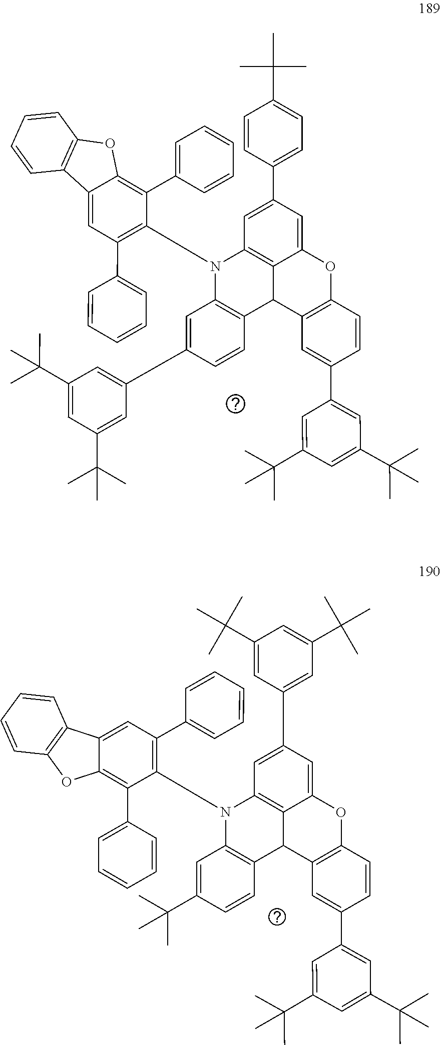 [SMILES: CC(C)(C)c1ccc(-c2cc3c4c(c2)N(c2c(-c5ccccc5)cc5c(oc6ccccc65)c2-c2ccccc2)c2cc(-c5cc(C(C)(C)C)cc(C(C)(C)C)c5)ccc2C4c2cc(-c4cc(C(C)(C)C)cc(C(C)(C)C)c4)ccc2O3)cc1 ; CC(C)(C)c1cc(-c2ccc3c(c2)C2c4ccc(C(C)(C)C)cc4N(c4c(-c5ccccc5)cc5c(oc6ccccc65)c4-c4ccccc4)c4cc(-c5cc(C(C)(C)C)cc(C(C)(C)C)c5)cc(c42)O3)cc(C(C)(C)C)c1]

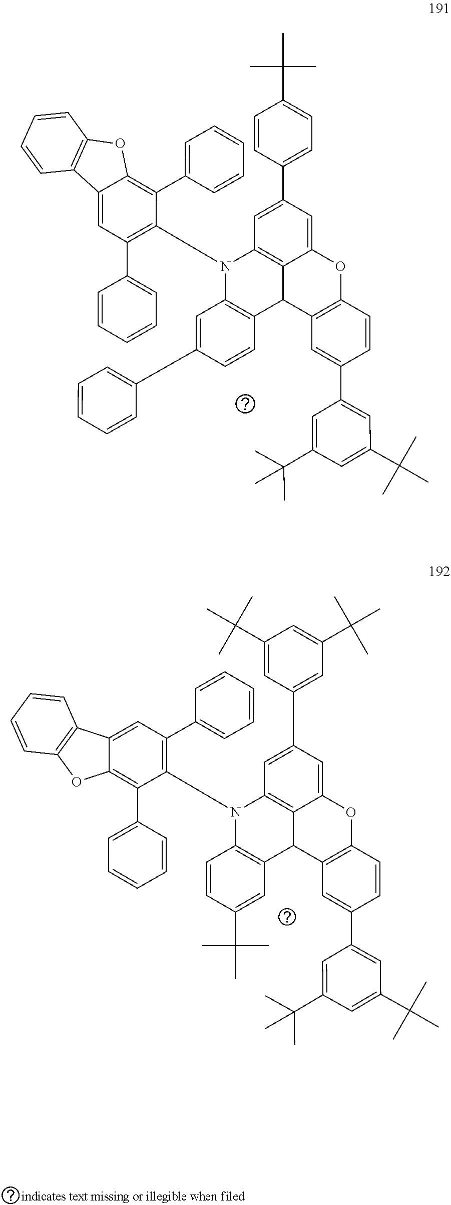 [SMILES: CC(C)(C)c1ccc(-c2cc3c4c(c2)N(c2c(-c5ccccc5)cc5c(oc6ccccc65)c2-c2ccccc2)c2cc(-c5ccccc5)ccc2C4c2cc(-c4cc(C(C)(C)C)cc(C(C)(C)C)c4)ccc2O3)cc1 ; CC(C)(C)c1cc(-c2ccc3c(c2)C2c4cc(C(C)(C)C)ccc4N(c4c(-c5ccccc5)cc5c(oc6ccccc65)c4-c4ccccc4)c4cc(-c5cc(C(C)(C)C)cc(C(C)(C)C)c5)cc(c42)O3)cc(C(C)(C)C)c1]

In a group represented by one of Formulae 2-1 to 2-4, Y₁ may be N(R₁₀), O, or S.

The heterocyclic compound represented by Formula 1 may include at least one group represented by one of Formulae 2-1 to 2-4, and the condensed cyclic group may include a C₃-C₆₀ carbocyclic group unsubstituted or substituted with at least one R₁₀ₐ or a C₁-C₆₀ heterocyclic group unsubstituted or substituted with at least one R₁₀ₐ.

The heterocyclic compound represented by Formula 1 may not only maximize the steric hindrance in a planar structure and thus increase a distance between molecules, but may also improve the chemical stability of the core element X₀ and ultimately, improve the color purity and driving voltage of a light-emitting device. Moreover, in the light-emitting device employing the heterocyclic compound, energy may be readily transferred from a host, which leads to improved lifespan characteristics.

Synthesis methods of the heterocyclic compound represented by Formula 1 may be recognizable by one of ordinary skill in the art by referring to the Synthesis Examples and/or the Examples provided below.

At least one heterocyclic compound represented by Formula 1 may be used in a light-emitting device (for example, an organic light-emitting device). Accordingly, provided is a light-emitting device which may include: a first electrode; a second electrode facing the first electrode; an interlayer between the first electrode and the second electrode and including an emission layer; and a heterocyclic compound represented by Formula 1 as described herein.

In embodiments, the first electrode of the light-emitting device may be an anode,
the second electrode of the light-emitting device may be a cathode,
the interlayer may further include a hole transport region between the first electrode and the emission layer, and an electron transport region between the emission layer and the second electrode,
the hole transport region may include a hole injection layer, a hole transport layer, an emission auxiliary layer, an electron blocking layer, or any combination thereof, and
the electron transport region may include a hole blocking layer, an electron transport layer, an electron injection layer, an electron control layer, or any combination thereof.

In embodiments, the interlayer may include the heterocyclic compound.

In embodiments, the emission layer may include the heterocyclic compound.

In embodiments, the emission layer may emit blue light.

In embodiments, the heterocyclic compound represented by Formula 1 may be a delayed fluorescence material.

In embodiments, the heterocyclic compound included in the emission layer may be an emitter.

In an embodiment, the emission layer may include a compound represented by Formula 6:

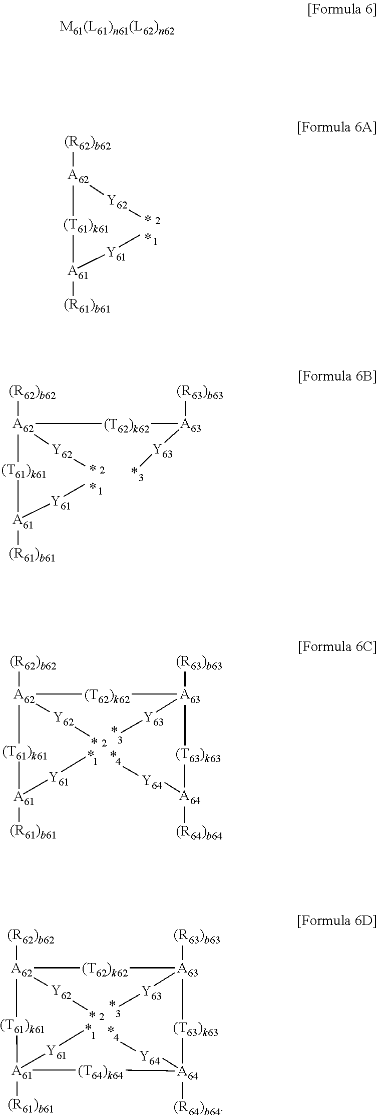

In Formula 6,
M₆₁ may be a first-row transition metal of the Periodic Table of Elements, a second-row transition metal of the Periodic Table of Elements, or a third-row transition metal of the Periodic Table of Elements,
L₆₁ may be a ligand represented by one of Formulae 6A to 6D,
L₆₂ may be a monodentate ligand, a bidentate ligand, or a tridentate ligand,
n61 may be 1 or 2,
n62 may be 0, 1, 2, 3, or 4,
A₆₁ to A₆₄ may each independently be a C₅-C₃₀ carbocyclic group or a C₁-C₃₀ heterocyclic group,
T₆₁ to T₆₄ may each independently be a single bond, a double bond, *—S—*′, *′C(═O)—*′, *′S(═O)—*′, *′C(R₆₅)(R₆₆)—*′, *′C(R₆₅)═C(R₆₆)—*′, *′C(R₆₅)=*′, *—Si(R₆₅)(R₆₆)—*′, *′B(R₆₅)—*′, *′N(R₆₅)—*′, or*′P(R₆₅)—*′,
k61 to k64 may each independently be 1, 2, or 3,
Y₆₁ to Y₆₄ may each independently be a single bond (e.g., a covalent bond or a coordinate bond), *—O—*′, *—S—*′, *—C(R₆₇)(R₆₈)—*′, *—Si(R₆₇)(R₆₈)—*′, *—B(R₆₇)—*′, *—N(R₆₇)—*′, or *—P(R₆₇)—*′,
*₁, *₂, *₃, and *₄ may each indicate a binding site to M₆₁,
R₆₁ to R₆₈ may each independently be hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —C(Q₁)(Q₂)(Q₃), —Si(Q₁)(Q₂)(Q₃), —B(Q₁)(Q₂), —N(Q₁)(Q₂), —P(Q₁)(Q₂), —C(═O)(Q₁), —S(═O)(Q₁), —S(═O)₂(Q₁), —P(═O)(Q₁)(Q₂), or —P(═S)(Q₁)(Q₂),
R₆₁ to R₆₈ may optionally be bonded to each other to form a substituted or unsubstituted C₅-C₆₀ carbocyclic group or a substituted or unsubstituted C₁-C₆₀ heterocyclic group, and
b61 to b64 may each independently be an integer from 0 to 10.

In an embodiment, in Formula 6, M₆₁ may be platinum (Pt), palladium (Pd), copper (Cu), silver (Ag), gold (Au), rhodium (Rh), iridium (Ir), ruthenium (Ru), osmium (Os), titanium (Ti), zirconium (Zr), hafnium (Hf), europium (Eu), terbium (Tb), or thulium (Tm).

In an embodiment, in Formula 6, M₆₁ may be Pt or Ir.

In an embodiment, in Formulae 6A to 6D, A₆₁ to A₆₄ may each independently be a first ring, a second ring, a condensed ring in which two or more first rings are condensed with each other, a condensed ring in which two or more second rings are condensed with each other, or a condensed ring in which one or more first rings and one or more second rings are condensed with each other,
wherein the first ring may be a cyclopentane group, a cyclopentene group, a cyclopentadiene group, a furan group, a thiophene group, a pyrrole group, a borole group, a phosphole group, a silole group, a germole group, a selenophene group, an oxazole group, a dihydroxazole group, an isoxazole group, a dihydroisoxazole group, an oxadiazole group, a dihydroxaddiazole group, an isoxadiazole group, a dihydroisoxadiazole group, an oxatriazole group, a dihydroxatriazole group, an isoxatriazole group, a dihydroisoxatriazole group, a thiazole group, a dihydrothiazole group, an isothiazole group, a dihydroisothiazole group, a thiadiazole group, a dihydrothiadiazole group, an isothiadiazole group, a dihydroisothiadiazole group, a thiatriazole group, a dihydrothiatriazole group, an isothiatriazole group, a dihydroisothiatriazole group, a pyrazole group, a dihydropyrazole group, an imidazole group, a dihydroimidazole group, a triazole group, a dihydrotriazole group, a tetrazole group, a dihydrotetrazole group, an azasilole group, a diazasilole group, or a triazasilole group, and
the second ring may be a cyclohexane group, a cyclohexene group, a cyclohexadiene group, an admantane group, a norbornane group, a norbornene group, a benzene group, a pyridine group, a dihydropyridine group, a tetrahydropyridine group, a pyrimidine group, a dihydropyrimidine group, a tetrahydropyrimidine group, a pyrazine group, a dihydropyrazine group, a tetrahydropyrazine group, a pyridazine group, a dihydropyridazine group, a tetrahydropyridazine group, or a triazine group.

In an embodiment, in Formulae 6A to 6D, A₆₁ to A₆₄ may each independently be a benzene group, a naphthalene group, an anthracene group, a phenanthrene group, a triphenylene group, a pyrene group, a chrysene group, a furan group, a thiophene group, a silole group, an indene group, a fluorene group, a benzofuran group, a dibenzofuran group, a benzothiophene group, a dibenzothiophene group, a benzosilole group, a dibenzosilole group, an indole group, a carbazole group, an indenopyridine group, an indolopyridine group, a benzofuropyridine group, a benzothienopyridine group, a benzosilolopyridine group, an indenopyrimidine group, an indolopyrimidine group, a benzofuropyrimidine group, a benzothienopyrimidine group, a benzosilolopyrimidine group, a pyridine group, a pyrimidine group, a pyrazine group, a pyridazine group, a triazine group, a quinoline group, an isoquinoline group, a quinoxaline group, a quinazoline group, a cinnoline group, a phthalazine group, a phenanthroline group, a pyrrole group, a pyrazole group, an imidazole group, a dihydroimidazole group, a triazole group, a dihydrotriazole group, an oxazole group, a dihydroxazole group, an isoxazole group, a thiazole group, a dihydrothiazole group, an isothiazole group, an oxadiazole group, a dihydroxaddiazole group, a thiadiazole group, a dihydrothiadiazole group, a benzopyrazole group, a benzimidazole group, a dihydrobenzimidazole group, an imidazopyridine group, an imidazopyrimidine group, an imidazopyrazine group, a benzoxazole group, a dihydrobenzoxazole group, a benzothiazole group, a dihydrobenzothiazole group, a benzoxadiazole group, a dihydrobenzoxadiazole group, a benzothiadiazole group, or a dihydrobenzothiadiazole group.

In an embodiment, in Formulae 6A to 6D T₆₁ to T₆₄ may each independently be a single bond, a double bond, *—O—*′, *—S—*′, *—C(R₆₅)(R₆₆)—*′, or *—N(R₆₅)—*′.

In an embodiment, in Formulae 6A to 6D, Y₆₁ to Y₆₄ may each independently be a single bond, *—O—*′, or *—S—*′.

In an embodiment, in Formulae 6A to 6D, R₆₁ to R₆₈ may each independently be:
hydrogen, deuterium, —F, —Cl, —Br, —I, a cyano group, a C₁-C₂₀ alkyl group, or a C₁-C₂₀ alkoxy group;
a phenyl group, a biphenyl group, a terphenyl group, a naphthyl group, a phenanthrenyl group, a triphenylenyl group, a chrysenyl group, a fluoranthenyl group, a fluorenyl group, a carbazolyl group, a dibenzofuranyl group, a dibenzothiophenyl group, a benzofluorenyl group, a benzocarbazolyl group, a benzonaphthofuranyl group, a benzonaphthothiophenyl group, a dibenzofluorenyl group, a dibenzocarbazolyl group, a dinaphthofuranyl group, a dinaphthothiophenyl group, a pyridinyl group, a pyrazinyl group, a pyridazinyl group, a pyrimidinyl group, a triazinyl group, a quinolinyl group, an isoquinolinyl group, a quinolinyl group, an isoquinolinyl group, a naphthyridinyl group, a quinoxalinyl group, a quinazolinyl group, an azafluorenyl group, an azacarbazolyl group, an azadibenzofuranyl group, an azadibenzothiophenyl group, a diazafluorenyl group, a diazacarbazolyl group, a diazadibenzofuranyl group, or a diazadibenzothiophenyl group; or
a phenyl group, a biphenyl group, a terphenyl group, a naphthyl group, a phenanthrenyl group, a triphenylenyl group, a chrysenyl group, a fluoranthenyl group, a fluorenyl group, a carbazolyl group, a dibenzofuranyl group, a dibenzothiophenyl group, a benzofluorenyl group, a benzocarbazolyl group, a benzonaphthofuranyl group, a benzonaphthothiophenyl group, a dibenzofluorenyl group, a dibenzocarbazolyl group, a dinaphthofuranyl group, a dinaphthothiophenyl group, a pyridinyl group, a pyrazinyl group, a pyridazinyl group, a pyrimidinyl group, a triazinyl group, a quinolinyl group, an isoquinolinyl group, a quinolinyl group, an isoquinolinyl group, a naphthyridinyl group, a quinoxalinyl group, a quinazolinyl group, an azafluorenyl group, an azacarbazolyl group, an azadibenzofuranyl group, an azadibenzothiophenyl group, a diazafluorenyl group, a diazacarbazolyl group, a diazadibenzofuranyl group, or a diazadibenzothiophenyl group, each substituted with at least one selected from deuterium, —F, —Cl, —Br, —I, a cyano group, a C₁-C₂₀ alkyl group, a C₁-C₂₀ alkoxy group, a phenyl group, a biphenyl group, a terphenyl group, a naphthyl group, a phenanthrenyl group, a triphenylenyl group, a chrysenyl group, a fluoranthenyl group, a fluorenyl group, a carbazolyl group, a dibenzofuranyl group, a dibenzothiophenyl group, a benzofluorenyl group, a benzocarbazolyl group, a benzonaphthofuranyl group, a benzonaphthothiophenyl group, a dibenzofluorenyl group, a dibenzocarbazolyl group, a dinaphthofuranyl group, a dinaphthothiophenyl group, a pyridinyl group, a pyrazinyl group, a pyridazinyl group, a pyrimidinyl group, a triazinyl group, a quinolinyl group, an isoquinolinyl group, a quinolinyl group, an isoquinolinyl group, a naphthyridinyl group, a quinoxalinyl group, a quinazolinyl group, an azafluorenyl group, an azacarbazolyl group, an azadibenzofuranyl group, an azadibenzothiophenyl group, a diazafluorenyl group, a diazacarbazolyl group, a diazadibenzofuranyl group, a diazadibenzothiophenyl group; or

—B(Q₆₁)(Q₆₂) or —N(Q₆₁)(Q₆₂), and
Q₆₁ and Q₆₂ may each independently be:
hydrogen, deuterium, or a C₁-C₂₀ alkyl group;
a phenyl group, a biphenyl group, a terphenyl group, a naphthyl group, a phenanthrenyl group, a triphenylenyl group, a chrysenyl group, a fluoranthenyl group, a fluorenyl group, a carbazolyl group, a dibenzofuranyl group, a dibenzothiophenyl group, a benzofluorenyl group, a benzocarbazolyl group, a benzonaphthofuranyl group, a benzonaphthothiophenyl group, a dibenzofluorenyl group, a dibenzocarbazolyl group, a dinaphthofuranyl group, a dinaphthothiophenyl group, a pyridinyl group, a pyrazinyl group, a pyridazinyl group, a pyrimidinyl group, a triazinyl group, a quinolinyl group, an isoquinolinyl group, a quinolinyl group, an isoquinolinyl group, a naphthyridinyl group, a quinoxalinyl group, a quinazolinyl group, an azafluorenyl group, an azacarbazolyl group, an azadibenzofuranyl group, an azadibenzothiophenyl group, a diazafluorenyl group, a diazacarbazolyl group, a diazadibenzofuranyl group, or a diazadibenzothiophenyl group; or
a phenyl group, a biphenyl group, a terphenyl group, a naphthyl group, a phenanthrenyl group, a triphenylenyl group, a chrysenyl group, a fluoranthenyl group, a fluorenyl group, a carbazolyl group, a dibenzofuranyl group, a dibenzothiophenyl group, a benzofluorenyl group, a benzocarbazolyl group, a benzonaphthofuranyl group, a benzonaphthothiophenyl group, a dibenzofluorenyl group, a dibenzocarbazolyl group, a dinaphthofuranyl group, a dinaphthothiophenyl group, a pyridinyl group, a pyrazinyl group, a pyridazinyl group, a pyrimidinyl group, a triazinyl group, a quinolinyl group, an isoquinolinyl group, a quinolinyl group, an isoquinolinyl group, a naphthyridinyl group, a quinoxalinyl group, a quinazolinyl group, an azafluorenyl group, an azacarbazolyl group, an azadibenzofuranyl group, an azadibenzothiophenyl group, a diazafluorenyl group, a diazacarbazolyl group, a diazadibenzofuranyl group, or a diazadibenzothiophenyl group, each substituted with at least one selected from deuterium, a C₁-C₂₀ alkyl group, a phenyl group, a biphenyl group, a terphenyl group, a naphthyl group, a phenanthrenyl group, a triphenylenyl group, a chrysenyl group, a fluoranthenyl group, a fluorenyl group, a carbazolyl group, a dibenzofuranyl group, a dibenzothiophenyl group, a benzofluorenyl group, a benzocarbazolyl group, a benzonaphthofuranyl group, a benzonaphthothiophenyl group, a dibenzofluorenyl group, a dibenzocarbazolyl group, a dinaphthofuranyl group, a dinaphthothiophenyl group, a pyridinyl group, a pyrazinyl group, a pyridazinyl group, a pyrimidinyl group, a triazinyl group, a quinolinyl group, an isoquinolinyl group, a quinolinyl group, an isoquinolinyl group, a naphthyridinyl group, a quinoxalinyl group, a quinazolinyl group, an azafluorenyl group, an azacarbazolyl group, an azadibenzofuranyl group, an azadibenzothiophenyl group, a diazafluorenyl group, a diazacarbazolyl group, a diazadibenzofuranyl group, and a diazadibenzothiophenyl group.

In an embodiment, in Formulae 6A to 6D, R₆₁ to R₆₈ may each independently be:
hydrogen, deuterium, —F, —Cl, —Br, —I, a cyano group, a methyl group, an ethyl group, an n-propyl group, an isopropyl group, an n-butyl group, an isobutyl group, a sec-butyl group, a tert-butyl group, a methoxy group, an ethoxy group, a propoxy group, or a butoxy group;
a phenyl group, a biphenyl group, a terphenyl group, a naphthyl group, a phenanthrenyl group, a triphenylenyl group, a chrysenyl group, a fluoranthenyl group, a fluorenyl group, a carbazolyl group, a dibenzofuranyl group, or a dibenzothiophenyl group;
a phenyl group, a biphenyl group, a terphenyl group, a naphthyl group, a phenanthrenyl group, a triphenylenyl group, a chrysenyl group, a fluoranthenyl group, a fluorenyl group, a carbazolyl group, a dibenzofuranyl group, or a dibenzothiophenyl group, each substituted with at least one selected from deuterium, —F, —Cl, —Br, —I, a cyano group, a methyl group, an ethyl group, an n-propyl group, an isopropyl group, an n-butyl group, an isobutyl group, a sec-butyl group, a tert-butyl group, a methoxy group, an ethoxy group, a propoxy group, a butoxy group, a phenyl group, a biphenyl group, a terphenyl group, a naphthyl group, a phenanthrenyl group, a triphenylenyl group, a chrysenyl group, a fluoranthenyl group, a fluorenyl group, a carbazolyl group, a dibenzofuranyl group, and a dibenzothiophenyl group; or
—B(Q₆₁)(Q₆₂) or —N(Q₆₁)(Q₆₂), and
Q₆₁ and Q₆₂ may each independently be:
hydrogen, deuterium, a methyl group, an ethyl group, an n-propyl group, an isopropyl group, an n-butyl group, an isobutyl group, a sec-butyl group, or a tert-butyl group;
a phenyl group, a biphenyl group, a terphenyl group, a naphthyl group, a phenanthrenyl group, a triphenylenyl group, a chrysenyl group, a fluoranthenyl group, a fluorenyl group, a carbazolyl group, a dibenzofuranyl group, or a dibenzothiophenyl group; or
a phenyl group, a biphenyl group, a terphenyl group, a naphthyl group, a phenanthrenyl group, a triphenylenyl group, a chrysenyl group, a fluoranthenyl group, a fluorenyl group, a carbazolyl group, a dibenzofuranyl group, or a dibenzothiophenyl group, each substituted with at least one selected from deuterium, a methyl group, an ethyl group, an n-propyl group, an isopropyl group, an n-butyl group, an isobutyl group, a sec-butyl group, a tert-butyl group, a phenyl group, a biphenyl group, a terphenyl group, a naphthyl group, a phenanthrenyl group, a triphenylenyl group, a chrysenyl group, a fluoranthenyl group, a fluorenyl group, a carbazolyl group, a dibenzofuranyl group, and a dibenzothiophenyl group.

In an embodiment, a compound represented by Formula 6 may be a carbene complex.

In embodiments, in Formula 6, L₆₁ may be a ligand represented by Formula 6C.

In embodiments, the compound represented by Formula 6 may be a carbene complex, and in Formula 6, L₆₁ may be a ligand represented by Formula 6C.

The term “carbene complex” as used herein may be a complex which includes a metal and a ligand bonded to the metal, wherein at least one bond between the metal and the ligand is a bond between the metal and a carbon atom of a carbene moiety. For example, in Compound PS-2 used in Example 1-1, as a bond between platinum and carbon of an imidazole ring is a bond between a metal and a carbon atom of a carbene moiety, PS-2 may be a carbene complex.

In embodiments, the compound represented by Formula 6 may be represented by any one of Formulae 6-1 and 6-2:

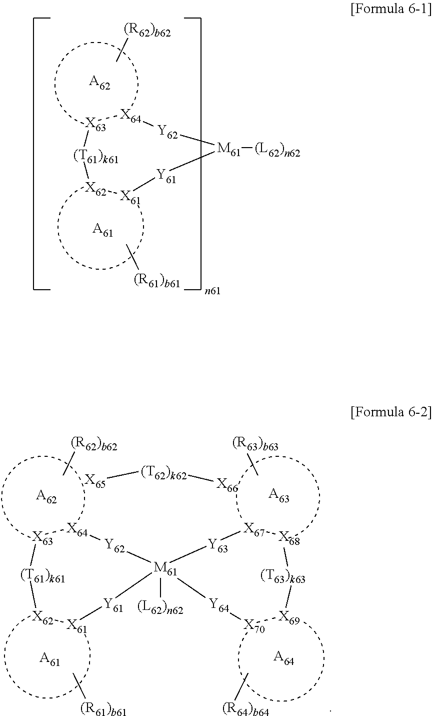

In Formulae 6-1 and 6-2,
X₆₁ to X₇₀ may each independently be N or C, and
the remaining components may each be understood by referring to the corresponding descriptions thereof provided herein.

In Formulae 6-1 and 6-2, X₆₁ and X₆₂ may be a ring member of A₆₁, and X₆₃ to X₇₀ may also be understood by referring to descriptions provided in connection with Formulae 6-1 and 6-2, X₆₁, and X₆₂.

In an embodiment, in Formula 6-2, X₆₁ may be C, and Y₆₁ may be a coordinate bond. For example, X₆₁ in Formula 6-2 may be a carbon atom of a carbene moiety.

In an embodiment, in Formula 6-2, X₇₀ may be C, and Y₆₄ may be a coordinate bond. For example, X₇₀ in Formula 6-2 may be a carbon atom of a carbene moiety.

In embodiments, the compound represented by Formula 6 may be a carbene complex or a compound represented by Formula 6-2. For example, M₆₁ in Formula 6-2 may be platinum.

In an embodiment, the emission layer may further include at least one of an auxiliary dopant and a sensitizer.

In embodiments, the auxiliary dopant and the sensitizer may each independently be an organometallic compound including platinum and a tetradentate ligand bonded to platinum, and the tetradentate ligand may include a carbene moiety chemically bonded to platinum. For example, the auxiliary dopant and/or the sensitizer may include the compound represented by Formula 6.

In an embodiment, the sensitizer may include the compound represented by Formula 6.

In embodiments, the emission layer of the light-emitting device may include a dopant and a host, and the dopant may include the heterocyclic compound represented by Formula 1. For example, the heterocyclic compound may also function as a dopant.

In an embodiment, the emission layer may further include a first host and a second host, wherein the first host may be an electron-transporting host and the second host may be a hole-transporting host.

In an embodiment, the first host may include at least one azine moiety, and the second host may include at least one carbazole moiety.

In an embodiment, the first host may include a compound represented by Formula 5:

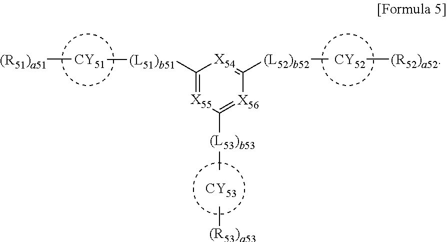

In Formula 5,

X₅₄ to X₅₆ may each independently be C(R₅₀), CH, or N; and at least one of X₅₄ to X₅₆ may each be N,
ring CY₅₁ to ring CY₅₃ may each independently be a C₅-C₆₀ carbocyclic group or a C₁-C₆₀ heterocyclic group,
L₅₁ to L₅₃ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
b51 to b53 may each independently be an integer from 0 to 3,
when b51 is 0, *-(L₅₁)_{b51}-*′ may be a single bond,
when b52 is 0, *-(L₅₂)_{b52}-*′ may be a single bond,
when b53 is 0, *-(L₅₃)_{ab3}-*′ may be a single bond,
R₅₀ to R₅₃ may each independently be the same as described herein in connection with R₁ to R₁₀, and
a51 to a53 may each independently be an integer from 0 to 10.

In an embodiment, in Formula 5, ring CY₅₁ to ring CY₅₃ may each independently be a first ring, a second ring, a condensed ring in which at least two first rings are condensed, a condensed ring in which at least two second rings are condensed, or a condensed ring in which at least one first ring is condensed with at least one second ring, wherein
the first ring may include a cyclopentadiene group, an adamantane group, a norbornane group, a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, or an indenoanthracene group, and
the second ring may include a pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, or an azadibenzofuran group.

In an embodiment, in Formula 5, ring CY₅₁ to ring CY₅₃ may each independently include a benzene group, a naphthalene group, an anthracene group, a phenanthrene group, a triphenylene group, a pyrene group, a chrysene group, a cyclopentadiene group, a 1,2,3,4-tetrahydronaphthalene group, a thiophene group, a furan group, an indole group, a benzoborole group, a benzophosphole group, an indene group, a benzosilole group, a benzogermole group, a benzothiophene group, a benzoselenophene group, a benzofuran group, a carbazole group, a dibenzoborole group, a dibenzophosphole group, a fluorene group, a dibenzosilole group, a dibenzogermole group, a dibenzothiophene group, a dibenzoselenophene group, a dibenzofuran group, a dibenzothiophene 5-oxide group, 9H-fluorene-9-one group, a dibenzothiophene 5,5-dioxide group, an azaindole group, an azabenzoborole group, an azabenzophosphole group, an azaindene group, an azabenzosilole group, an azabenzogermole group, an azabenzothiophene group, an azabenzoselenophene group, an azabenzofuran group, an azacarbazole group, an azadibenzoborole group, an azadibenzophosphole group, an azafluorene group, an azadibenzosilole group, an azadibenzogermole group, an azadibenzothiophene group, an azadibenzoselenophene group, an azadibenzofuran group, an azadibenzothiophene 5-oxide group, an aza-9H-fluorene-9-one group, an azadibenzothiophene 5,5-dioxide group, a pyridine group, a pyrimidine group, a pyrazine group, a pyridazine group, a triazine group, a quinoline group, an isoquinoline group, a quinoxaline group, a quinazoline group, a phenanthroline group, a pyrrole group, a pyrazole group, an imidazole group, a triazole group, an oxazole group, an iso-oxazole group, a thiazole group, an isothiazole group, an oxadiazole group, a thiadiazole group, a benzopyrazole group, a benzimidazole group, a benzoxazole group, a benzothiazole group, a benzoxadiazole group, a benzothiadiazole group, a 5,6,7,8-tetrahydroisoquinoline group, or a 5,6,7,8-tetrahydroquinoline group.

In an embodiment, in Formula 5, L₅₁ to L₅₃ may each independently include a benzene group, a naphthalene group, an anthracene group, a phenanthrene group, a triphenylene group, a pyrene group, a chrysene group, cyclopentadiene group, a furan group, a thiophene group, a silole group, an indene group, a fluorene group, an indole group, a carbazole group, a benzofuran group, a dibenzofuran group, a benzothiophene group, a dibenzothiophene group, a benzosilole group, a dibenzosilole group, an azafluorene group, an azacarbazole group, an azadibenzofuran group, an azadibenzothiophene group, an azadibenzosilole group, a pyridine group, a pyrimidine group, a pyrazine group, a pyridazine group, a triazine group, a quinoline group, an isoquinoline group, a quinoxaline group, a quinazoline group, a phenanthroline group, a pyrrole group, a pyrazole group, an imidazole group, a triazole group, an oxazole group, an isooxazole group, a thiazole group, an isothiazole group, an oxadiazole group, a thiadiazole group, a benzopyrazole group, a benzimidazole group, a benzoxazole group, a benzothiazole group, a benzoxadiazole group, or a benzothiadiazole group.

In an embodiment, the first host may include at least one compound selected from Compounds ETH1 to ETH32:

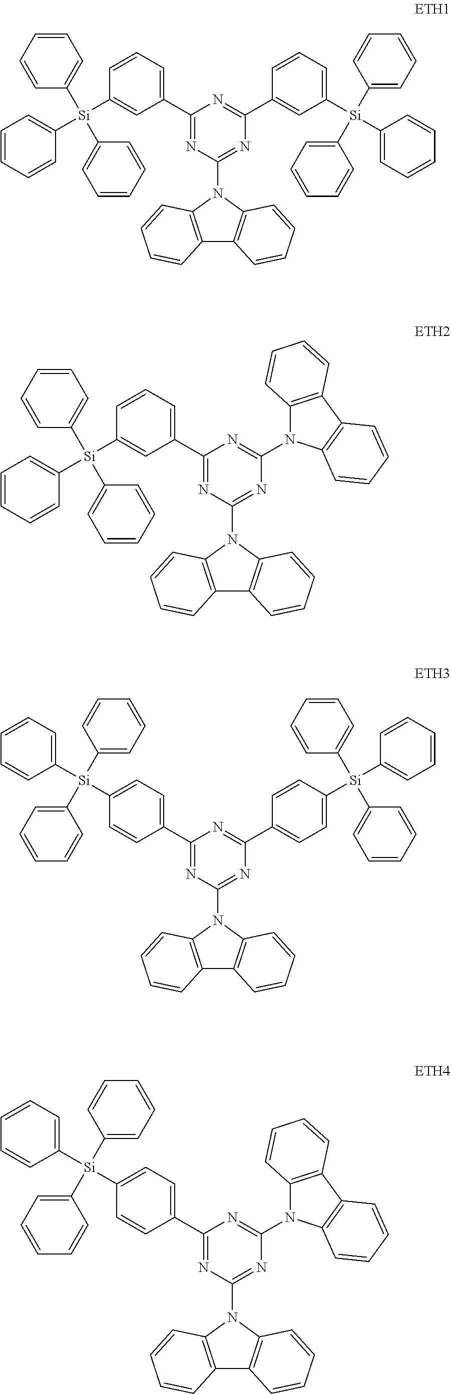 [SMILES: c1ccc([Si](c2ccccc2)(c2ccccc2)c2cccc(-c3nc(-c4cccc([Si](c5ccccc5)(c5ccccc5)c5ccccc5)c4)nc(-n4c5ccccc5c5ccccc54)n3)c2)cc1 ; c1ccc([Si](c2ccccc2)(c2ccccc2)c2cccc(-c3nc(-n4c5ccccc5c5ccccc54)nc(-n4c5ccccc5c5ccccc54)n3)c2)cc1 ; c1ccc([Si](c2ccccc2)(c2ccccc2)c2ccc(-c3nc(-c4ccc([Si](c5ccccc5)(c5ccccc5)c5ccccc5)cc4)nc(-n4c5ccccc5c5ccccc54)n3)cc2)cc1 ; c1ccc([Si](c2ccccc2)(c2ccccc2)c2ccc(-c3nc(-n4c5ccccc5c5ccccc54)nc(-n4c5ccccc5c5ccccc54)n3)cc2)cc1]

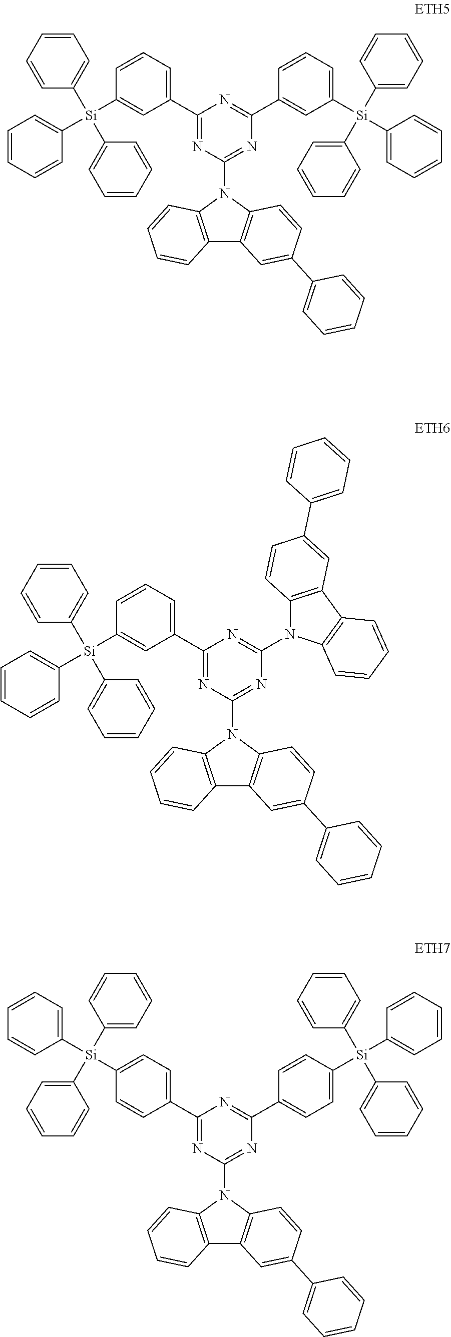 [SMILES: c1ccc(-c2ccc3c(c2)c2ccccc2n3-c2nc(-c3cccc([Si](c4ccccc4)(c4ccccc4)c4ccccc4)c3)nc(-c3cccc([Si](c4ccccc4)(c4ccccc4)c4ccccc4)c3)n2)cc1 ; c1ccc(-c2ccc3c(c2)c2ccccc2n3-c2nc(-c3cccc([Si](c4ccccc4)(c4ccccc4)c4ccccc4)c3)nc(-n3c4ccccc4c4cc(-c5ccccc5)ccc43)n2)cc1 ; c1ccc(-c2ccc3c(c2)c2ccccc2n3-c2nc(-c3ccc([Si](c4ccccc4)(c4ccccc4)c4ccccc4)cc3)nc(-c3ccc([Si](c4ccccc4)(c4ccccc4)c4ccccc4)cc3)n2)cc1]

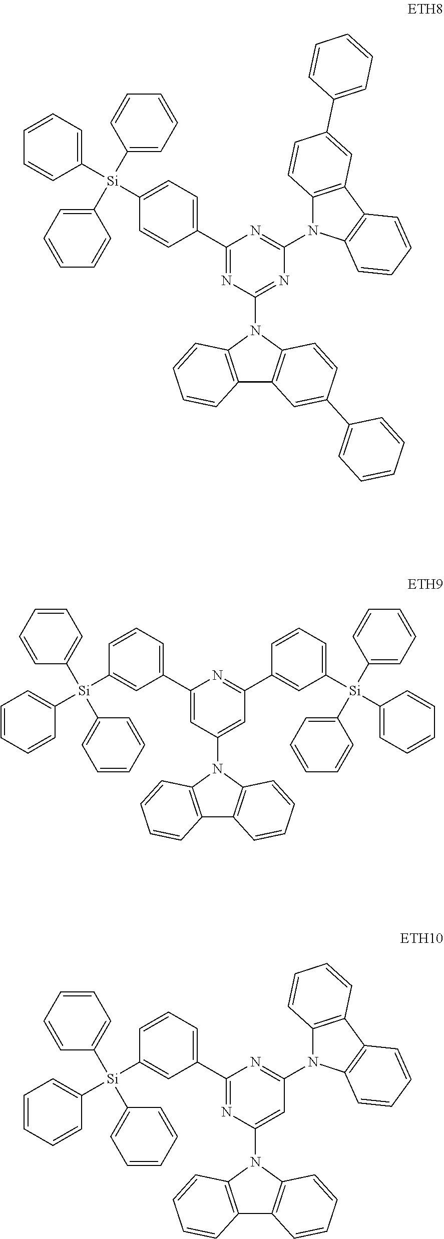

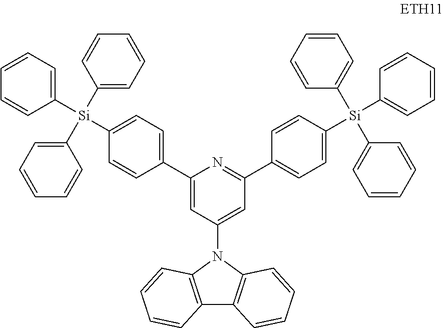 [SMILES: c1ccc([Si](c2ccccc2)(c2ccccc2)c2ccc(-c3cc(-n4c5ccccc5c5ccccc54)cc(-c4ccc([Si](c5ccccc5)(c5ccccc5)c5ccccc5)cc4)n3)cc2)cc1]

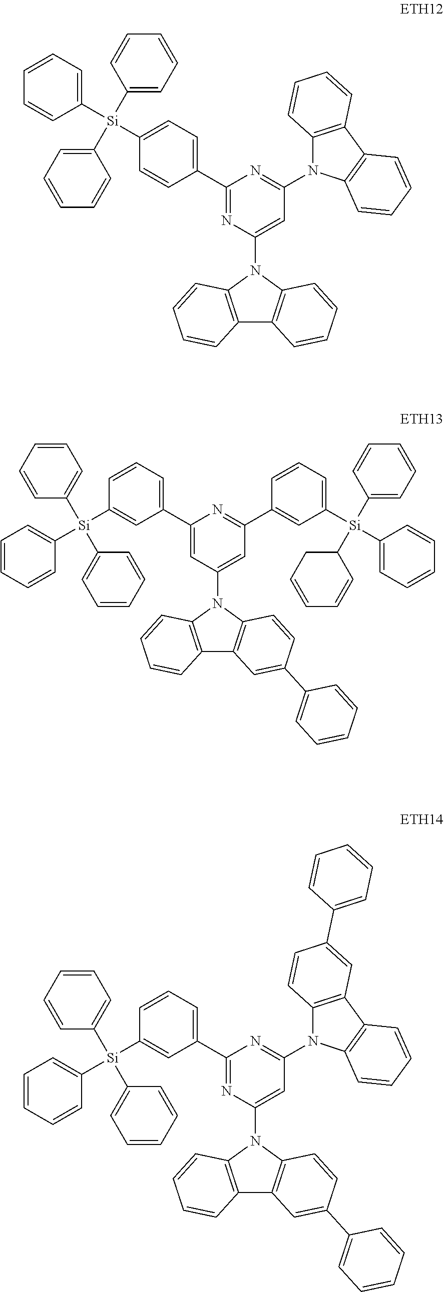 [SMILES: c1ccc([Si](c2ccccc2)(c2ccccc2)c2ccc(-c3nc(-n4c5ccccc5c5ccccc54)cc(-n4c5ccccc5c5ccccc54)n3)cc2)cc1 ; C1=CCC([Si](c2ccccc2)(c2ccccc2)c2cccc(-c3cc(-n4c5ccccc5c5cc(-c6ccccc6)ccc54)cc(-c4cccc([Si](c5ccccc5)(c5ccccc5)c5ccccc5)c4)n3)c2)C=C1 ; c1ccc(-c2ccc3c(c2)c2ccccc2n3-c2cc(-n3c4ccccc4c4cc(-c5ccccc5)ccc43)nc(-c3cccc([Si](c4ccccc4)(c4ccccc4)c4ccccc4)c3)n2)cc1]

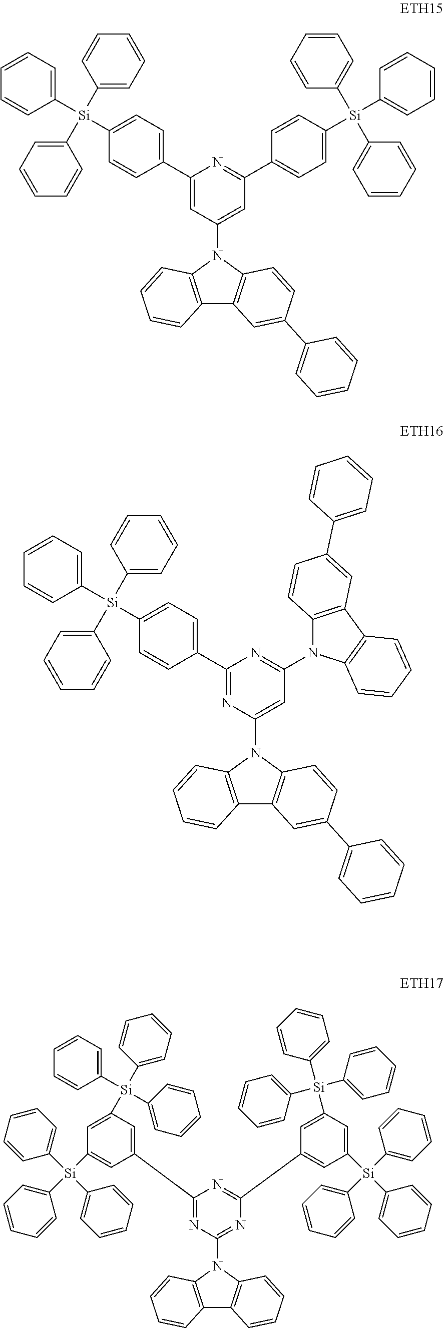 [SMILES: c1ccc(-c2ccc3c(c2)c2ccccc2n3-c2cc(-c3ccc([Si](c4ccccc4)(c4ccccc4)c4ccccc4)cc3)nc(-c3ccc([Si](c4ccccc4)(c4ccccc4)c4ccccc4)cc3)c2)cc1 ; c1ccc(-c2ccc3c(c2)c2ccccc2n3-c2cc(-n3c4ccccc4c4cc(-c5ccccc5)ccc43)nc(-c3ccc([Si](c4ccccc4)(c4ccccc4)c4ccccc4)cc3)n2)cc1 ; c1ccc([Si](c2ccccc2)(c2ccccc2)c2cc(-c3nc(-c4cc([Si](c5ccccc5)(c5ccccc5)c5ccccc5)cc([Si](c5ccccc5)(c5ccccc5)c5ccccc5)c4)nc(-n4c5ccccc5c5ccccc54)n3)cc([Si](c3ccccc3)(c3ccccc3)c3ccccc3)c2)cc1]

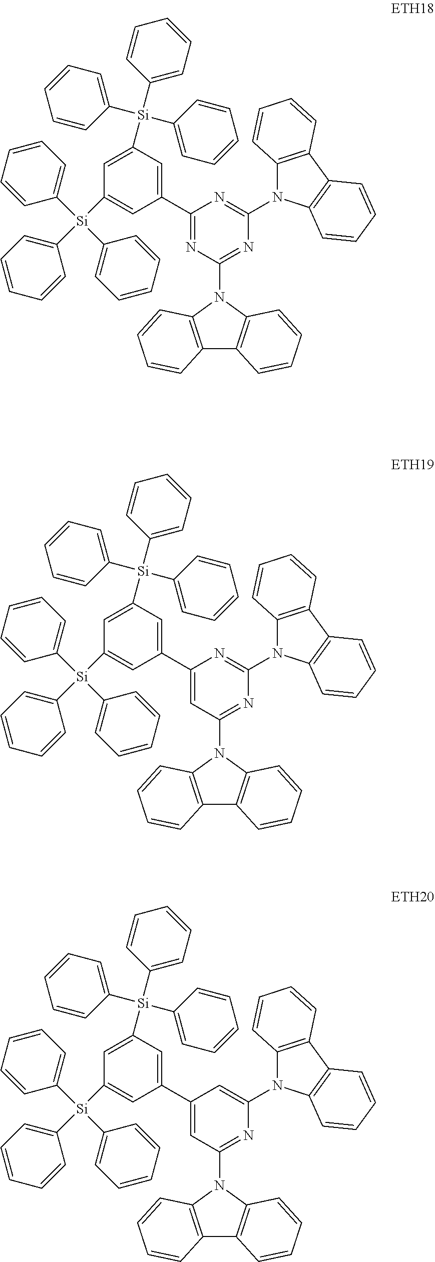 [SMILES: c1ccc([Si](c2ccccc2)(c2ccccc2)c2cc(-c3nc(-n4c5ccccc5c5ccccc54)nc(-n4c5ccccc5c5ccccc54)n3)cc([Si](c3ccccc3)(c3ccccc3)c3ccccc3)c2)cc1 ; c1ccc([Si](c2ccccc2)(c2ccccc2)c2cc(-c3cc(-n4c5ccccc5c5ccccc54)nc(-n4c5ccccc5c5ccccc54)n3)cc([Si](c3ccccc3)(c3ccccc3)c3ccccc3)c2)cc1 ; c1ccc([Si](c2ccccc2)(c2ccccc2)c2cc(-c3cc(-n4c5ccccc5c5ccccc54)nc(-n4c5ccccc5c5ccccc54)c3)cc([Si](c3ccccc3)(c3ccccc3)c3ccccc3)c2)cc1]

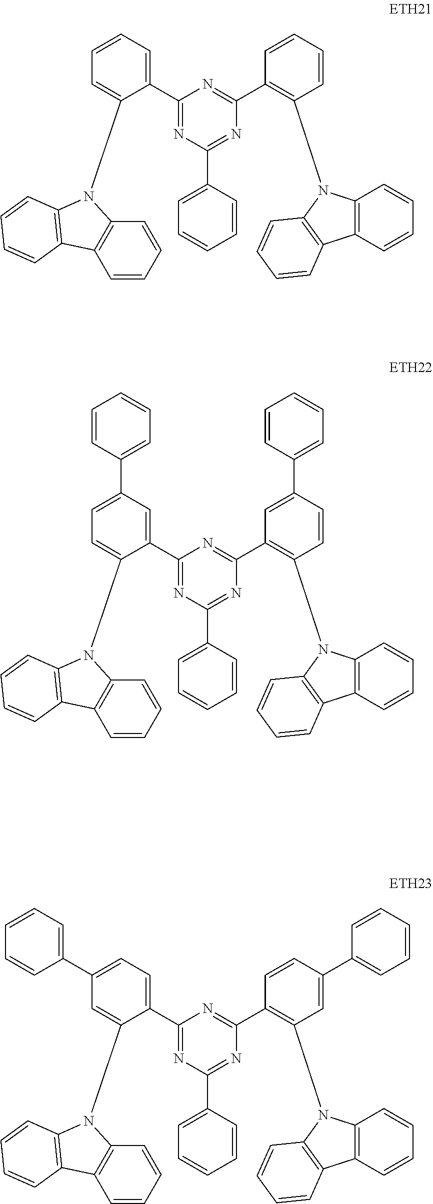 [SMILES: c1ccc(-c2nc(-c3ccccc3-n3c4ccccc4c4ccccc43)nc(-c3ccccc3-n3c4ccccc4c4ccccc43)n2)cc1 ; c1ccc(-c2ccc(-n3c4ccccc4c4ccccc43)c(-c3nc(-c4ccccc4)nc(-c4cc(-c5ccccc5)ccc4-n4c5ccccc5c5ccccc54)n3)c2)cc1 ; c1ccc(-c2ccc(-c3nc(-c4ccccc4)nc(-c4ccc(-c5ccccc5)cc4-n4c5ccccc5c5ccccc54)n3)c(-n3c4ccccc4c4ccccc43)c2)cc1]

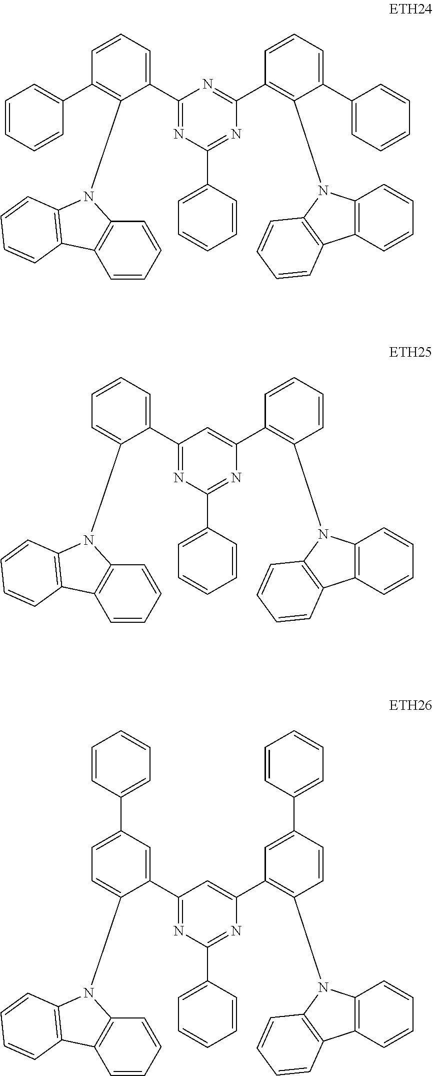 [SMILES: c1ccc(-c2nc(-c3cccc(-c4ccccc4)c3-n3c4ccccc4c4ccccc43)nc(-c3cccc(-c4ccccc4)c3-n3c4ccccc4c4ccccc43)n2)cc1 ; c1ccc(-c2nc(-c3ccccc3-n3c4ccccc4c4ccccc43)cc(-c3ccccc3-n3c4ccccc4c4ccccc43)n2)cc1 ; c1ccc(-c2ccc(-n3c4ccccc4c4ccccc43)c(-c3cc(-c4cc(-c5ccccc5)ccc4-n4c5ccccc5c5ccccc54)nc(-c4ccccc4)n3)c2)cc1]

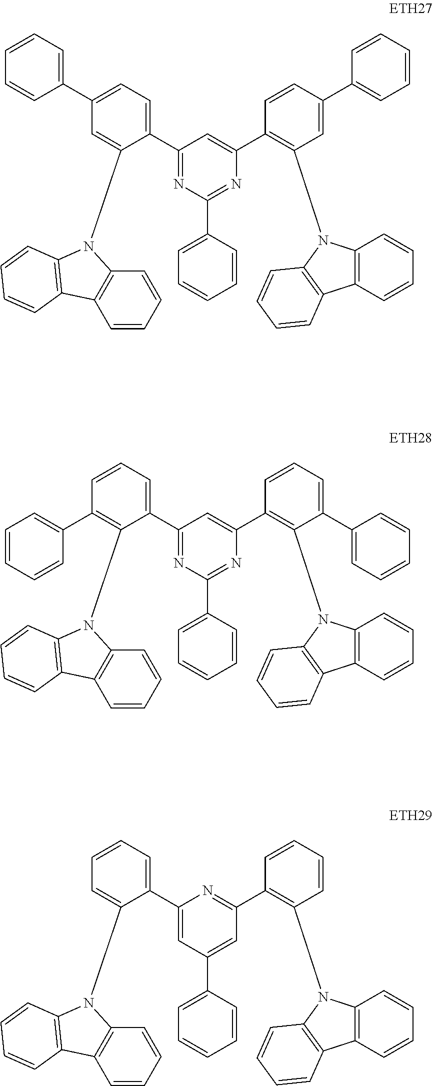 [SMILES: c1ccc(-c2ccc(-c3cc(-c4ccc(-c5ccccc5)cc4-n4c5ccccc5c5ccccc54)nc(-c4ccccc4)n3)c(-n3c4ccccc4c4ccccc43)c2)cc1 ; c1ccc(-c2nc(-c3cccc(-c4ccccc4)c3-n3c4ccccc4c4ccccc43)cc(-c3cccc(-c4ccccc4)c3-n3c4ccccc4c4ccccc43)n2)cc1 ; c1ccc(-c2cc(-c3ccccc3-n3c4ccccc4c4ccccc43)nc(-c3ccccc3-n3c4ccccc4c4ccccc43)c2)cc1]

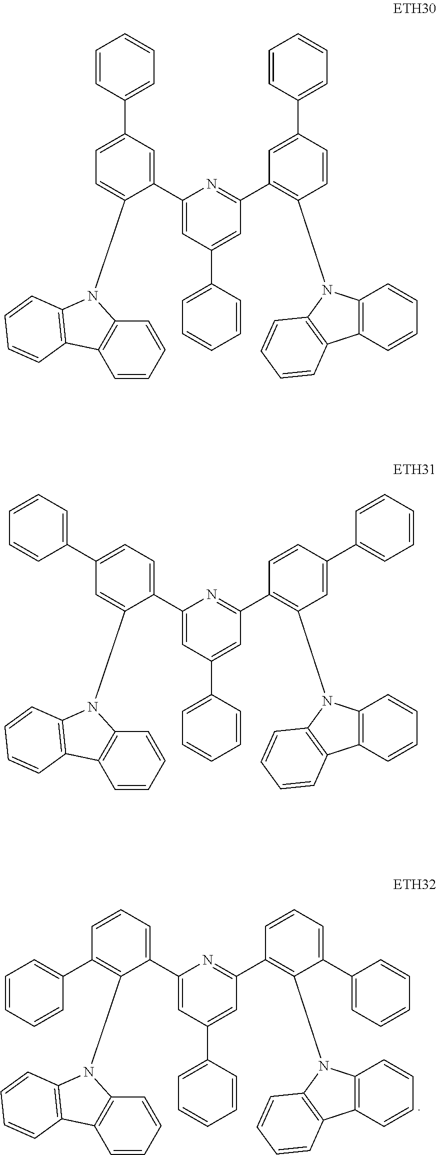 [SMILES: c1ccc(-c2cc(-c3cc(-c4ccccc4)ccc3-n3c4ccccc4c4ccccc43)nc(-c3cc(-c4ccccc4)ccc3-n3c4ccccc4c4ccccc43)c2)cc1 ; c1ccc(-c2cc(-c3ccc(-c4ccccc4)cc3-n3c4ccccc4c4ccccc43)nc(-c3ccc(-c4ccccc4)cc3-n3c4ccccc4c4ccccc43)c2)cc1 ; c1ccc(-c2cc(-c3cccc(-c4ccccc4)c3-n3c4ccccc4c4ccccc43)nc(-c3cccc(-c4ccccc4)c3-n3c4ccccc4c4ccccc43)c2)cc1]

In an embodiment, the second host may include a group represented by Formula 7:

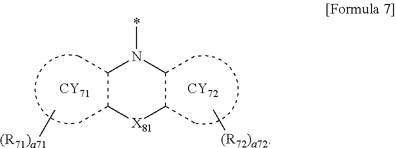

In Formula 7,
ring CY₇₁ and ring CY₇₂ may each independently include a C₅-C₆₀ carbocyclic group or a C₁-C₆₀ heterocyclic group,
X₈₁ may be a single bond, O, S, N(R₈₁), B(R₈₁), C(R₈₁ₐ)(R_{81b}), or Si(R₈₁ₐ)(R_{81b}),
R₇₁, R₇₂, R₈₁, R₈₁ₐ, and R_{81b} may each independently be the same as described herein in connection with R₁ to R₁₀,
a71 and a72 may each independently be an integer from 0 to 10, and
* may indicate a binding site to a neighboring atom.

In an embodiment, in Formula 7, ring CY₇₁ and ring CY₇₂ may each independently be a first ring, a second ring, a condensed ring in which at least two first rings are condensed, a condensed ring in which at least two second rings are condensed, or a condensed ring in which at least one first ring is condensed with at least one second ring, wherein
the first ring may include a cyclopentadiene group, an adamantane group, a norbornane group, a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, or an indenoanthracene group, and
the second ring may include a pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, or an azadibenzofuran group.

In an embodiment, in Formula 7, ring CY₇₁ and ring CY₇₂ may each independently include a benzene group, a naphthalene group, an anthracene group, a phenanthrene group, a triphenylene group, a pyrene group, a chrysene group, a cyclopentadiene group, a 1,2,3,4-tetrahydronaphthalene group, a thiophene group, a furan group, an indole group, a benzoborole group, a benzophosphole group, an indene group, a benzosilole group, a benzogermole group, a benzothiophene group, a benzoselenophene group, a benzofuran group, a carbazole group, a dibenzoborole group, a dibenzophosphole group, a fluorene group, a dibenzosilole group, a dibenzogermole group, a dibenzothiophene group, a dibenzoselenophene group, a dibenzofuran group, a dibenzothiophene 5-oxide group, 9H-fluorene-9-one group, a dibenzothiophene 5,5-dioxide group, an azaindole group, an azabenzoborole group, an azabenzophosphole group, an azaindene group, an azabenzosilole group, an azabenzogermole group, an azabenzothiophene group, an azabenzoselenophene group, an azabenzofuran group, an azacarbazole group, an azadibenzoborole group, an azadibenzophosphole group, an azafluorene group, an azadibenzosilole group, an azadibenzogermole group, an azadibenzothiophene group, an azadibenzoselenophene group, an azadibenzofuran group, an azadibenzothiophene 5-oxide group, an aza-9H-fluorene-9-one group, an azadibenzothiophene 5,5-dioxide group, a pyridine group, a pyrimidine group, a pyrazine group, a pyridazine group, a triazine group, a quinoline group, an isoquinoline group, a quinoxaline group, a quinazoline group, a phenanthroline group, a pyrrole group, a pyrazole group, an imidazole group, a triazole group, an oxazole group, an iso-oxazole group, a thiazole group, an isothiazole group, an oxadiazole group, a thiadiazole group, a benzopyrazole group, a benzimidazole group, a benzoxazole group, a benzothiazole group, a benzoxadiazole group, a benzothiadiazole group, a 5,6,7,8-tetrahydroisoquinoline group, or a 5,6,7,8-tetrahydroquinoline group.

In an embodiment, the second host may include a compound represented by one of Formulae 7-1 to 7-5:

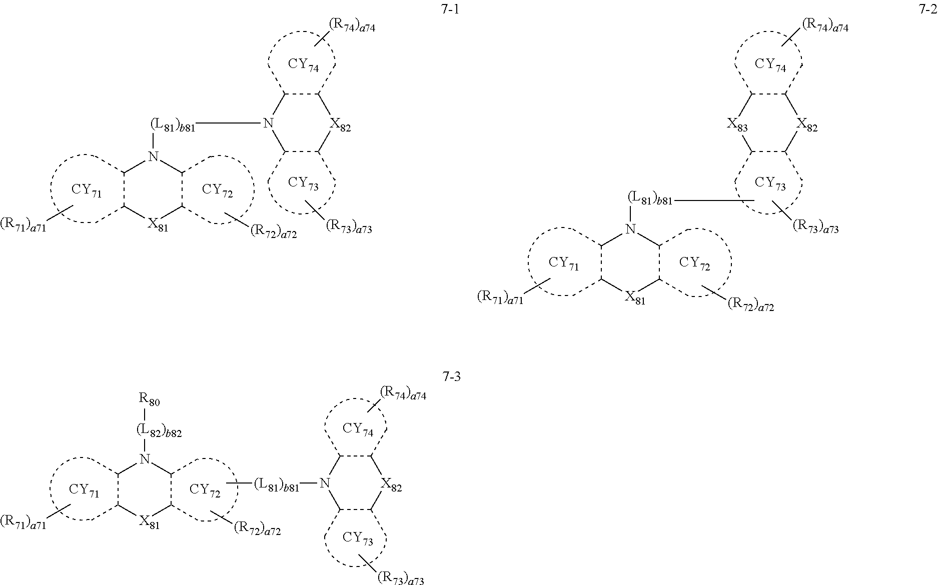

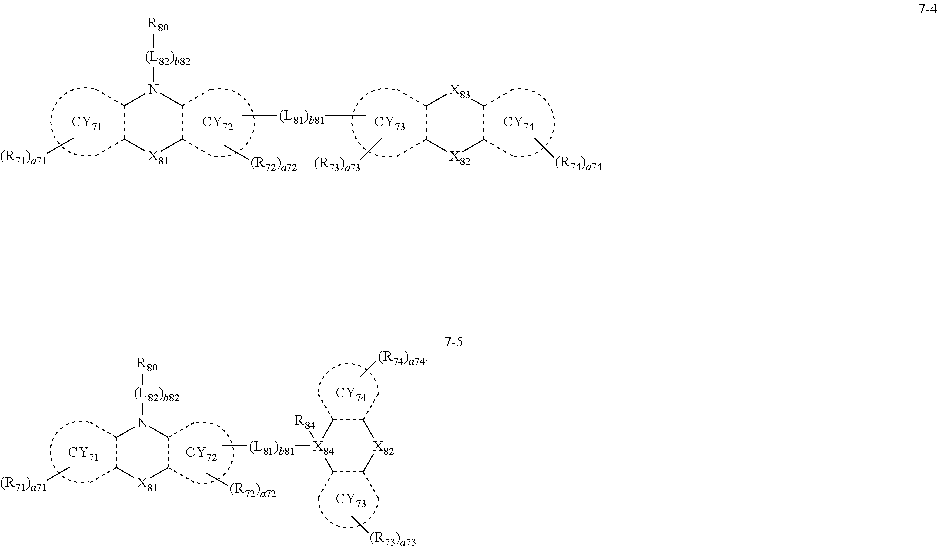

In Formulae 7-1 to 7-5,
ring CY₇₁, ring CY₇₂, X₈₁, R₇₁, R₇₂, a71, and a72 may be the same as described in the specification,
ring CY₇₃, ring CY₇₄, R₇₃, R₇₄, a73, and a74 may respectively be the same as described herein in connection with ring CY₇₁, ring CY₇₂, R₇₁, R₇₂, a71, and a72,
L₈₁ and L₈₂ may each independently be *—C(Q₄)(Q₅)-*′, *—Si(Q₄)(Q₅)-*′, a substituted or unsubstituted C₅-C₃₀ carbocyclic group, or a substituted or unsubstituted C₁-C₃₀ heterocyclic group, and 04 and 05 may each independently be the same as described in connection with Q₁,
b81 and b82 may each be an integer from 0 to 5, wherein
when b81 is 0, *-(L₈₁)_{b81}-*′ may be a single bond; when b81 is 2 or more, two or more of L₈₁(s) may be identical to or different from each other; when b82 is 0, *-(L₈₂)_{b82}—*′ may be a single bond; and when b82 is 2 or more, two or more of L₈₂(s) may be identical to or different from each other,
X₈₂ may be a single bond, O, S, N(R₈₂), B(R₈₂), C(R₈₂ₐ)(R_{82b}), or Si(R₈₂ₐ)(R_{82b}),
X₈₃ may be a single bond, O, S, N(R₈₃), B(R₈₃), C(R₈₃ₐ)(R_{83b}), or Si(R₈₃ₐ)(R_{83b}),

In Formulae 7-2 and 7-4, X₈₂ and X₈₃ may each not be a single bond,
X₈₄ may be C or Si,
R₈₀, R₈₂, R₈₃, R₈₂ₐ, R_{82b}, R₈₃ₐ, R_{83b}, and R₈₄ may each independently be the same as described in connection with R₈₁, and
* and *′ each indicate a binding site to a neighboring atom.

In embodiments,
a moiety represented by

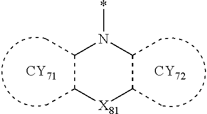

in Formulae 7-1 and 7-2 may be a moiety represented by one of Formulae CY71-1(1) to CY71-1(8),
a moiety represented by

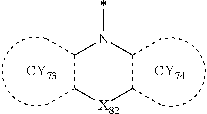

in Formulae 7-1 and 7-3 may be a moiety represented by one of Formulae CY71-2(1) to CY71-2(8),
a moiety represented by

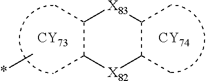

in Formulae 7-2 and 7-4 may be a moiety represented by one of Formulae CY71-3(1) to CY71-3(32),
a moiety represented by

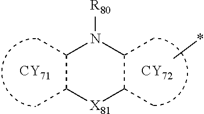

in Formulae 7-3 to 7-5 may be a moiety represented by one of Formulae CY71-4(1) to CY71-4(32), and
a moiety represented by

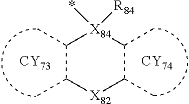

in Formula 7-5 may be a moiety represented by one of Formulae CY71-5(1) to CY71-5(8):

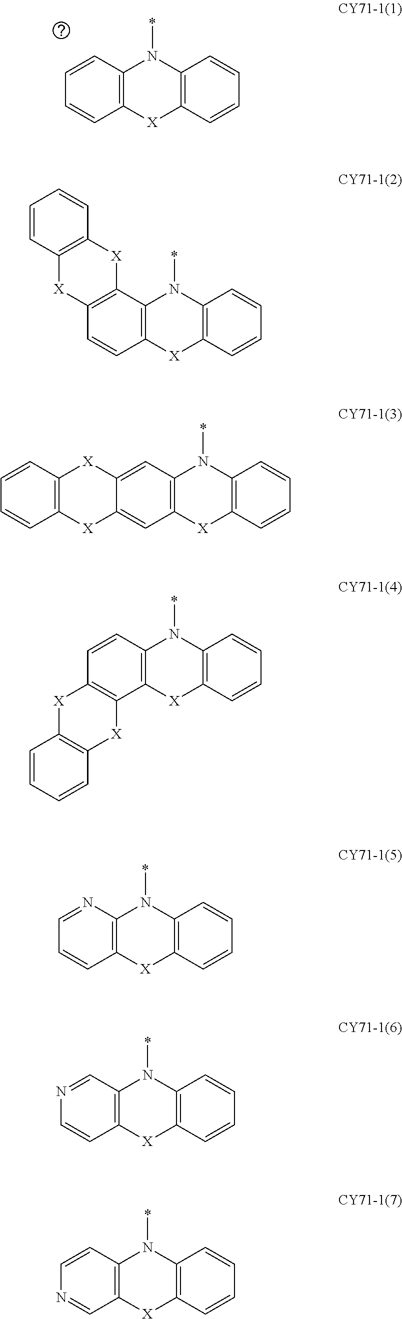

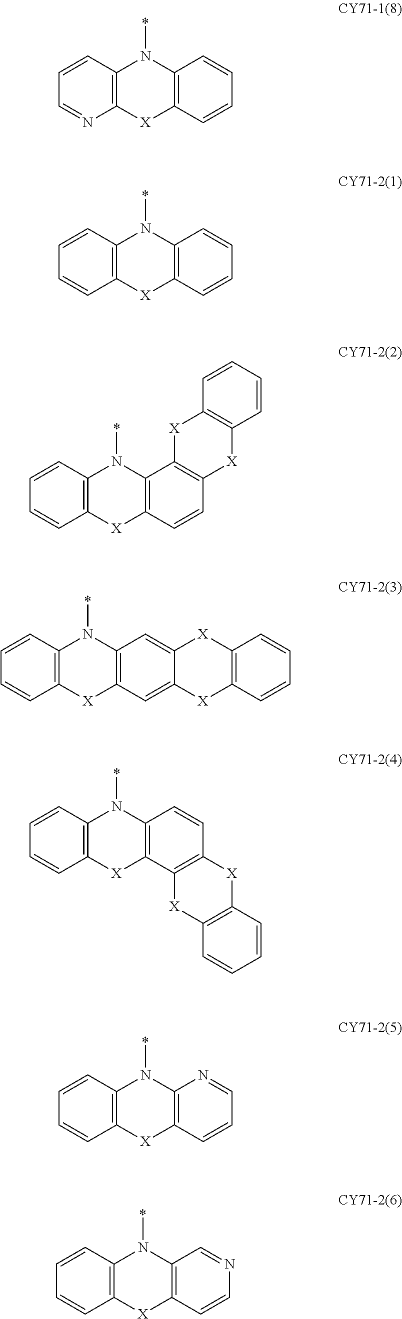

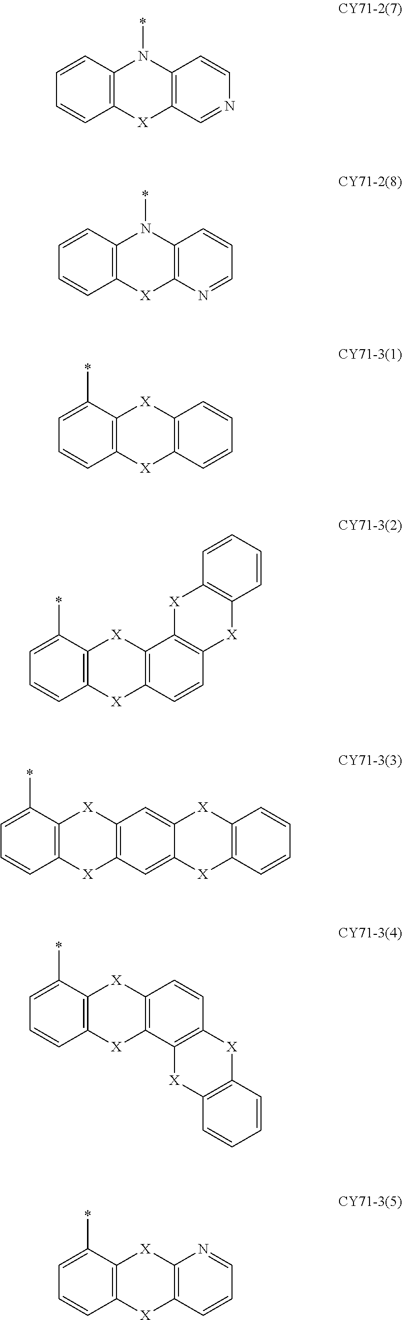

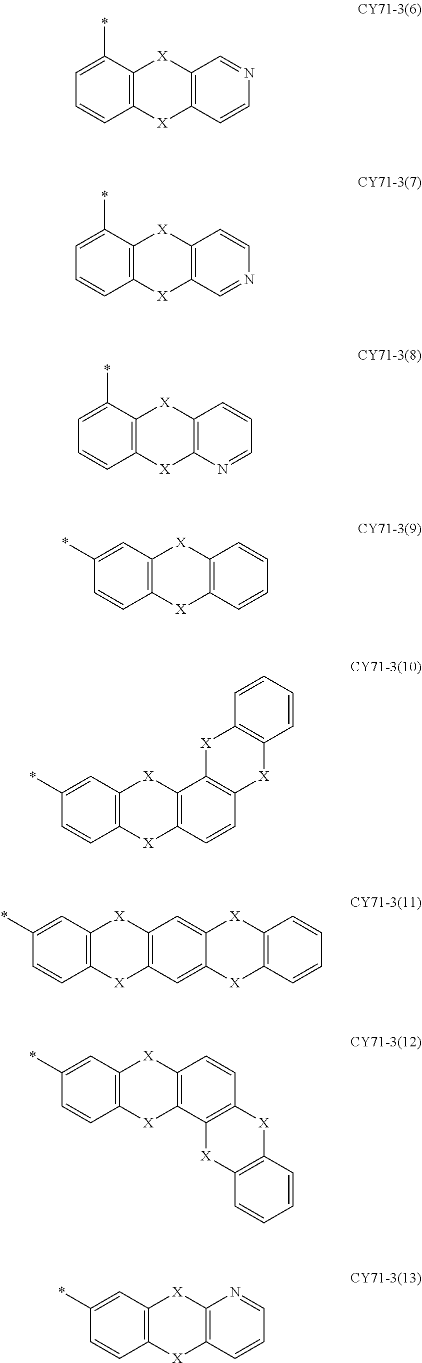

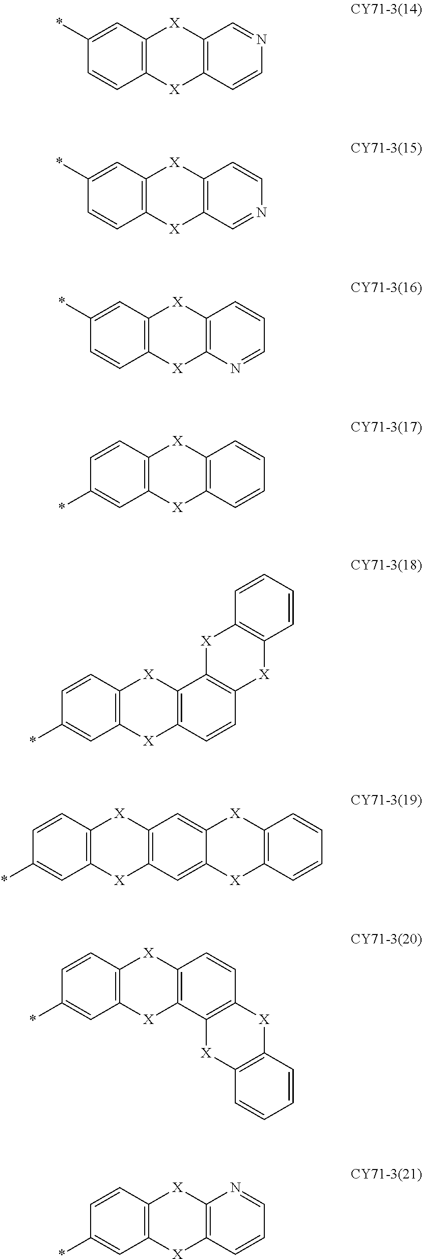

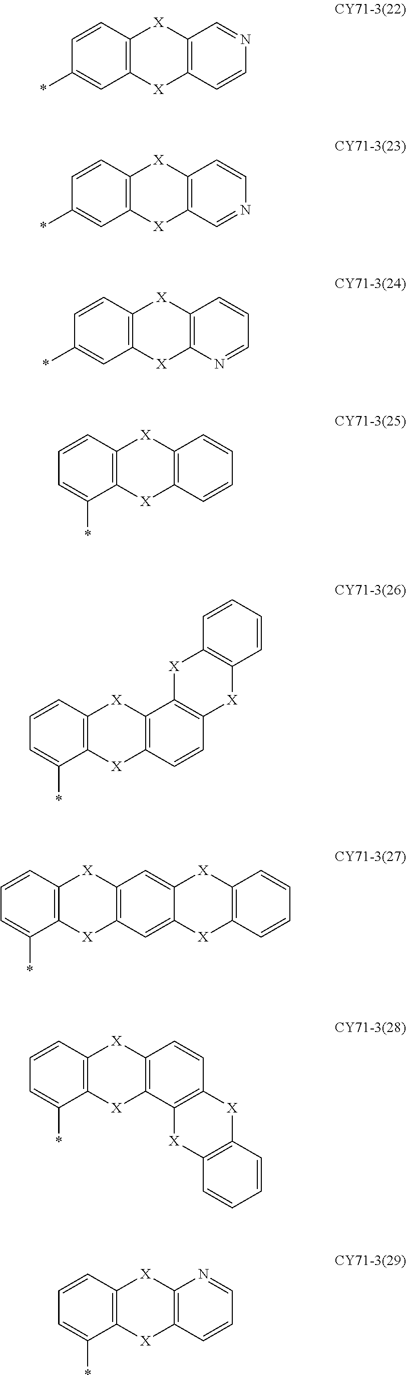

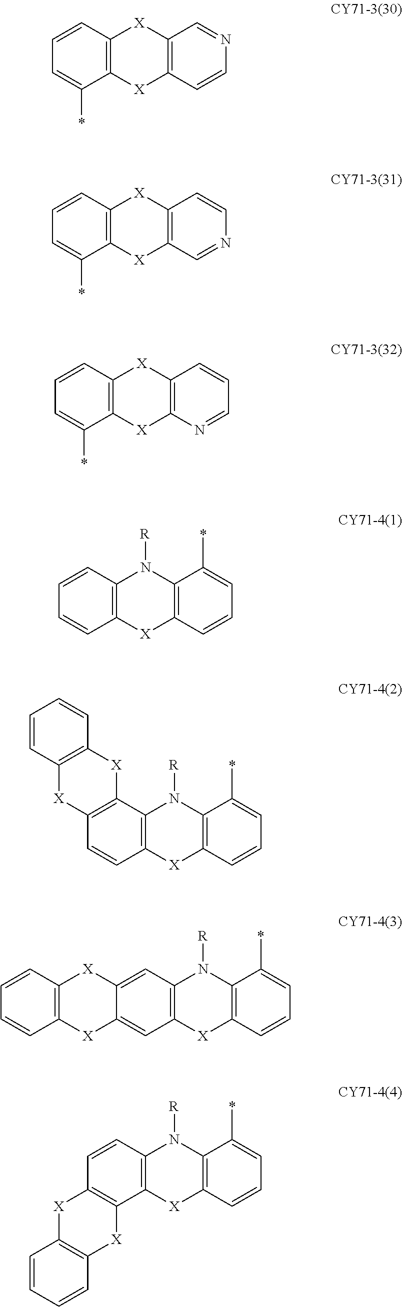

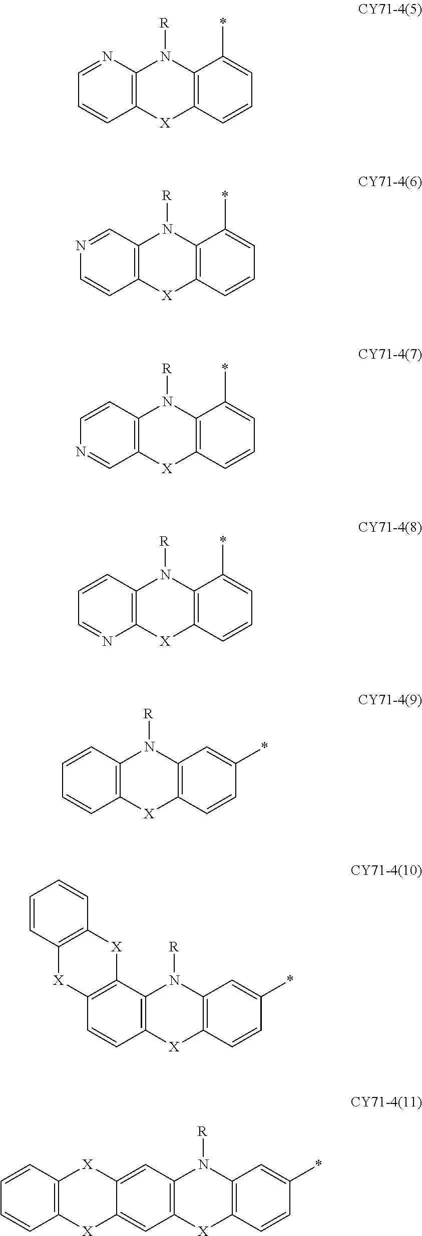

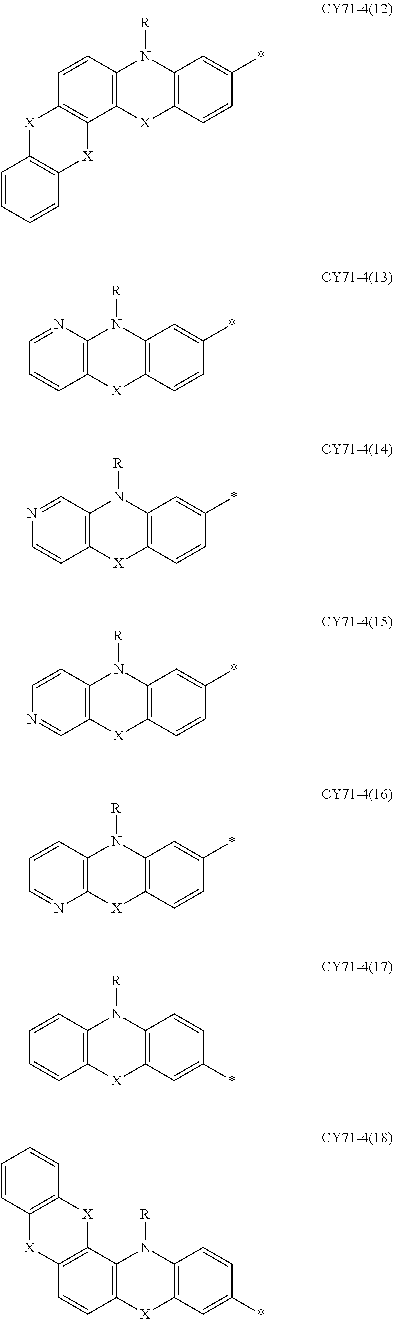

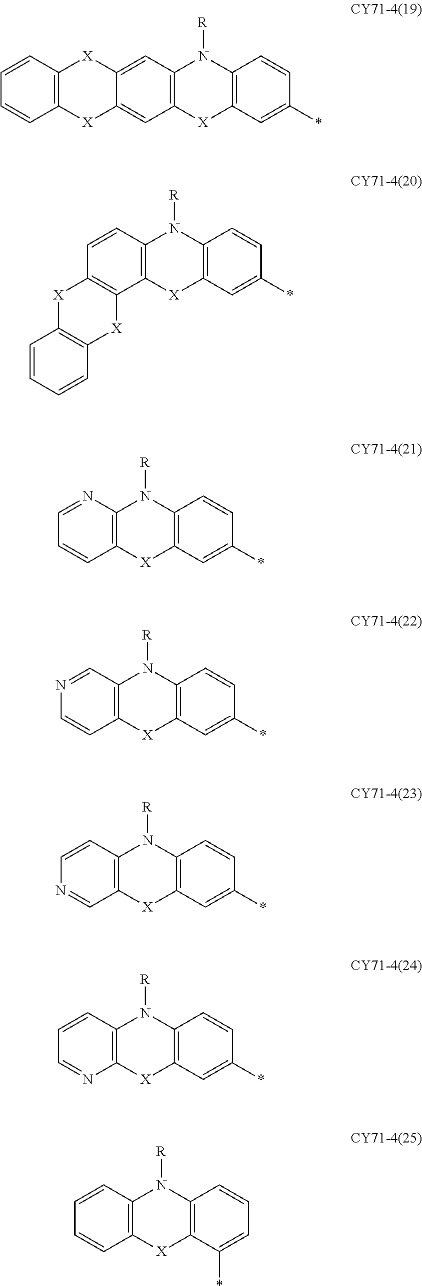

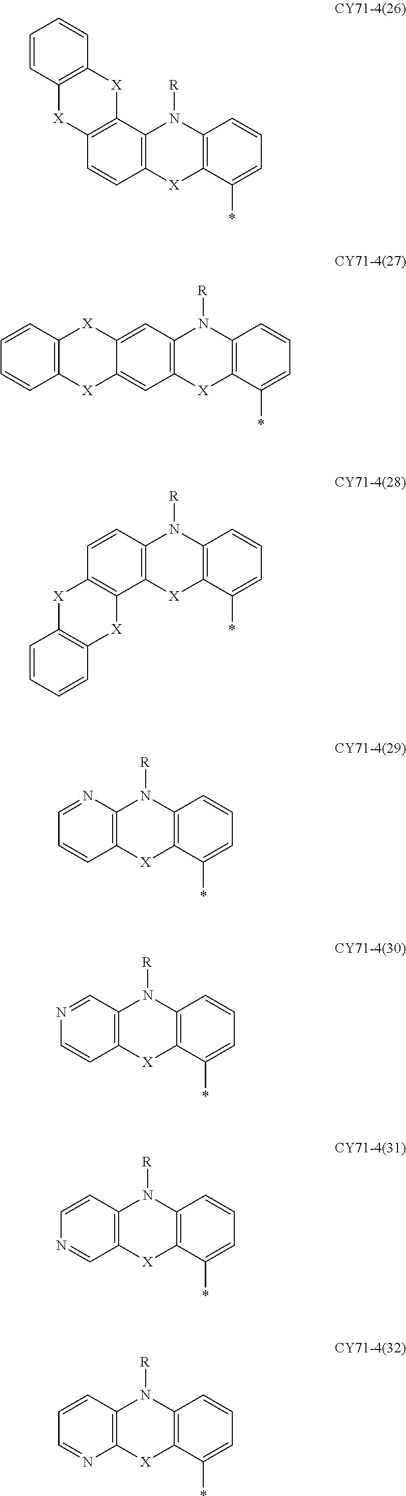

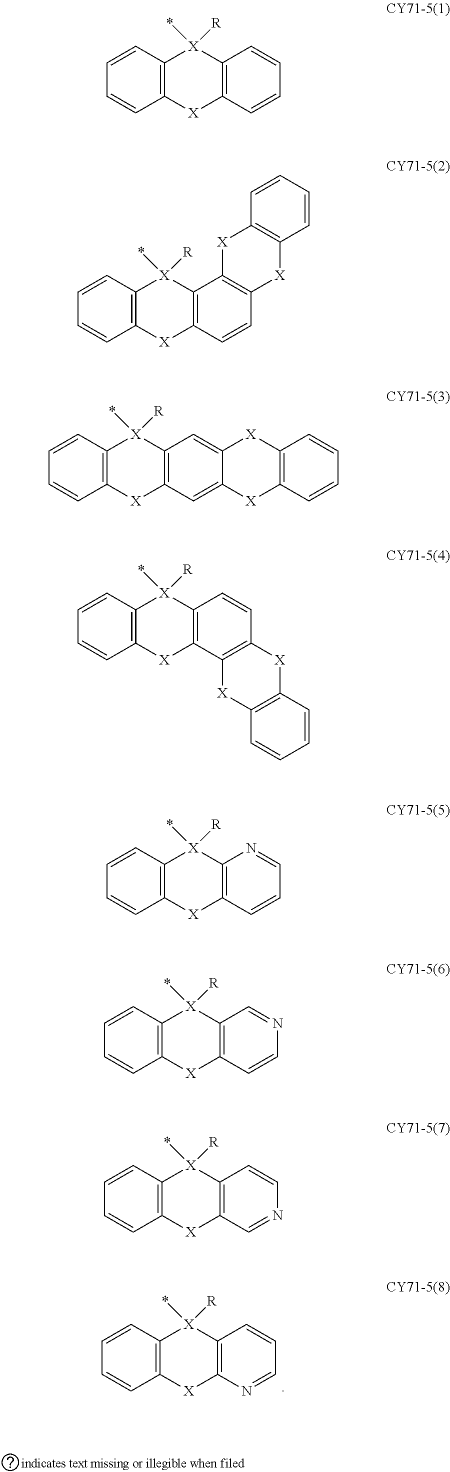

In Formulae CY71-1(1) to CY71-1(8), CY71-2(1) to CY71-2(8), CY71-3(1) to CY71-3(32), CY71-4(1) to CY71-4(32), and CY71-5(1) to CY71-5(8),
X₈₁ to X₈₄, R₈₀ and R₈₄ may be the same as described in the specification,
X₈₅ may be a single bond, O, S, N(R₈₅), B(R₈₅), C(R₈₅ₐ)(R_{85b}), or Si(R₈₅ₐ)(R_{85b}),
X₈₆ may be a single bond, O, S, N(R₈₆), B(R₈₆), C(R₈₆ₐ)(R_{86b}), or Si(R₈₆ₐ)(R_{86b}),
in Formulae CY71-1(1) to CY71-1(8) and CY71-4(1) to CY71-4(32), X₈₅ and X₈₆ may each not be a single bond,
X₈₇ may be a single bond, O, S, N(R₈₇), B(R₈₇), C(R₈₇ₐ)(R_{87b}), or Si(R₈₇ₐ)(R_{87b}),
X₈₈ may be a single bond, O, S, N(R₈₈), B(R₈₈), C(R₈₈ₐ)(R_{88b}), or Si(R₈₈ₐ)(R_{88b}),
in Formulae CY71-2(1) to CY71-2(8), CY71-3(1) to CY71-3(32) and CY71-5(1) to CY71-5(8), X₈₇ and X₈₈ may each not be a single bond, and
R₈₅ to R₈₈, R₈₅ₐ, R_{85b}, R₈₆ₐ, R_{86b}, R₈₇ₐ, R_{87b}, R₈₈ₐ and R_{88b} may each independently be the same as described in connection with R₈₁.

In an embodiment, the second host may include at least one compound selected from Compounts HTH1 to HTH40:

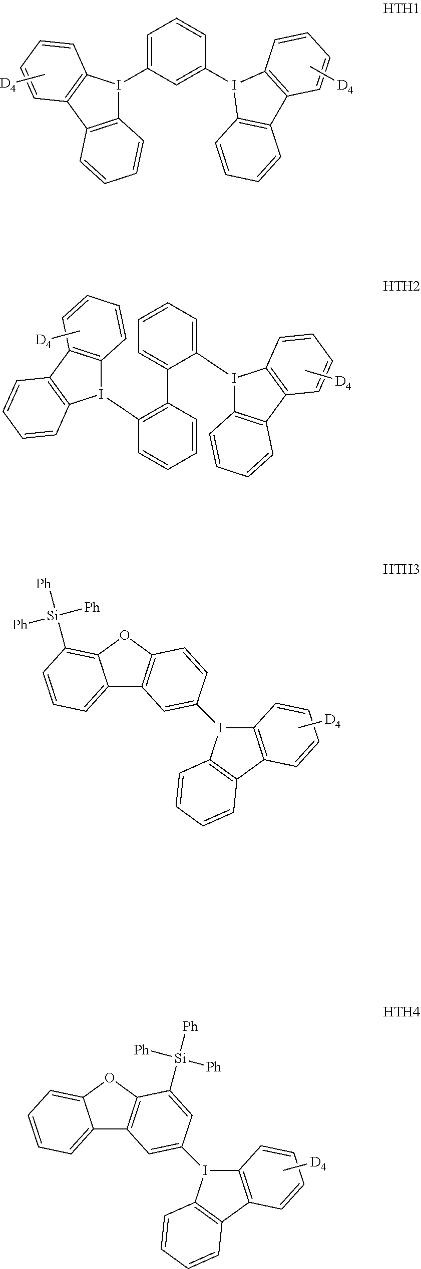

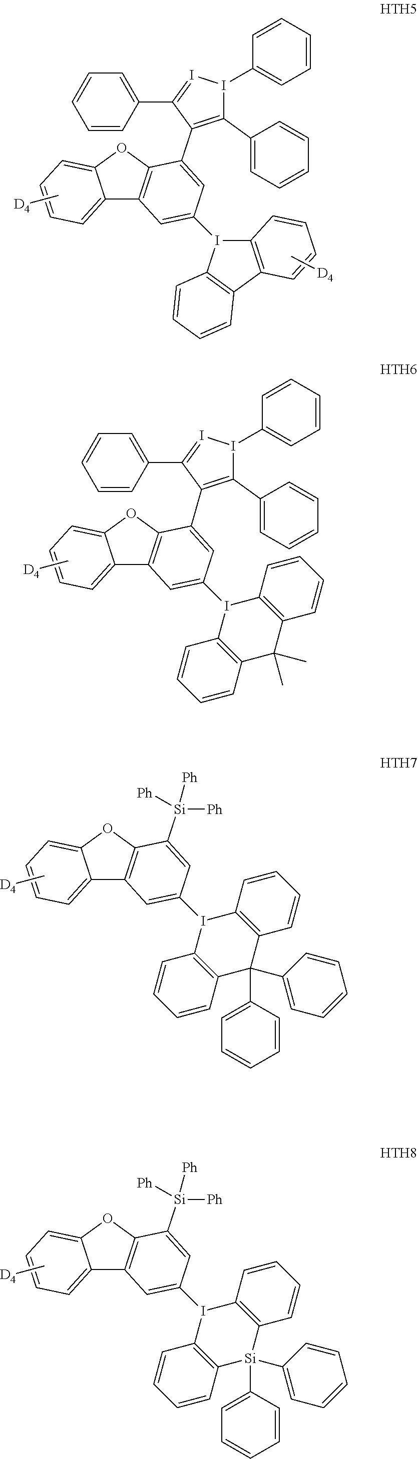

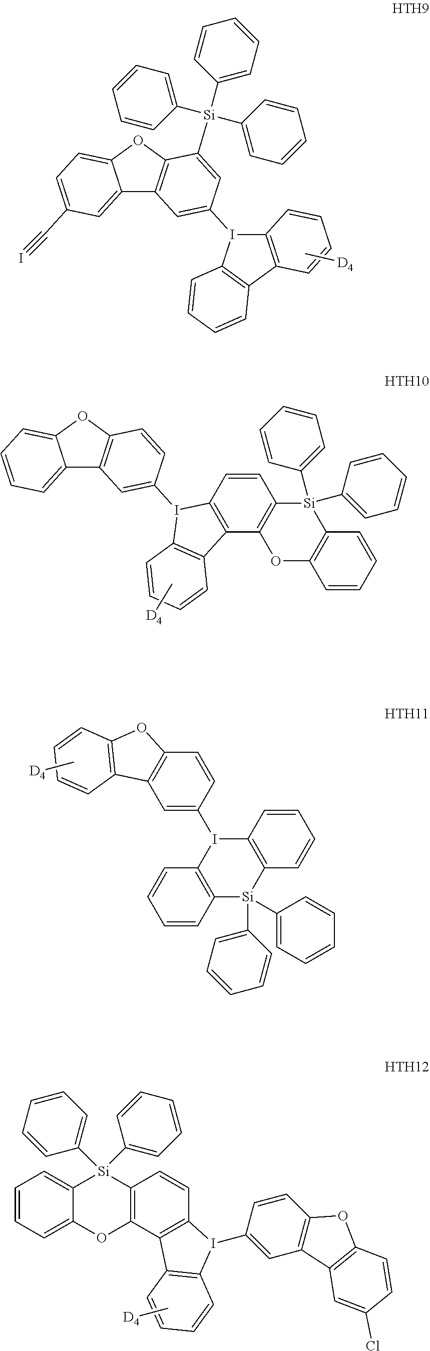

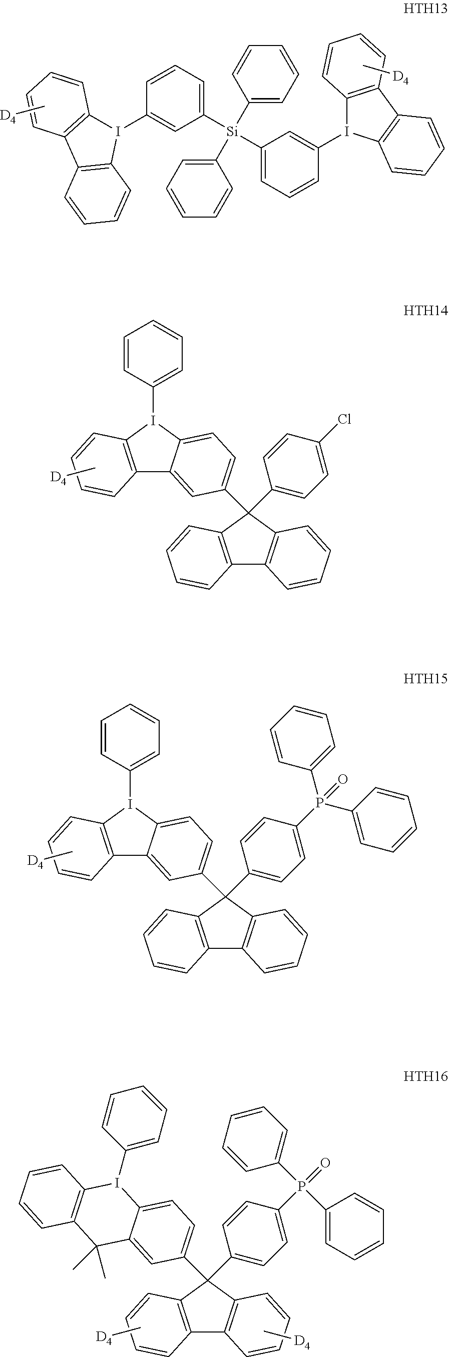

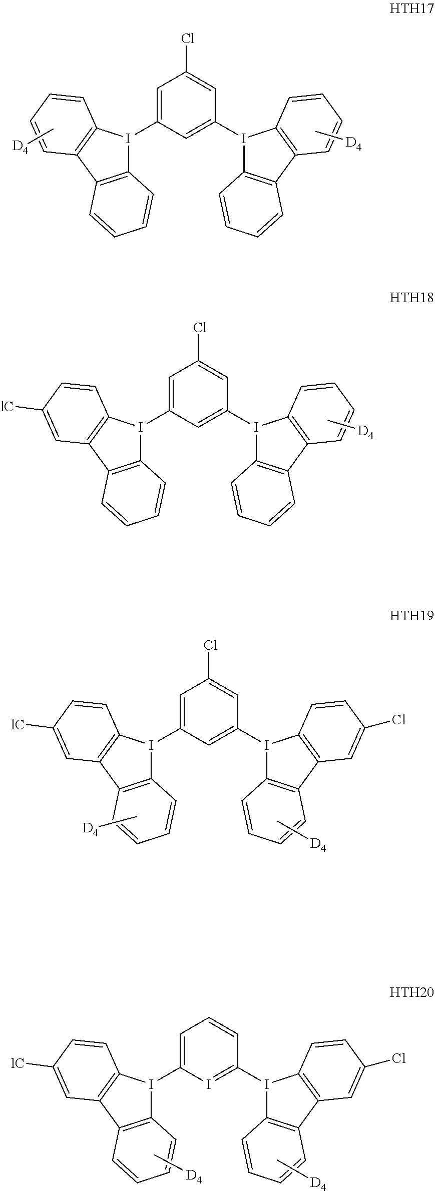

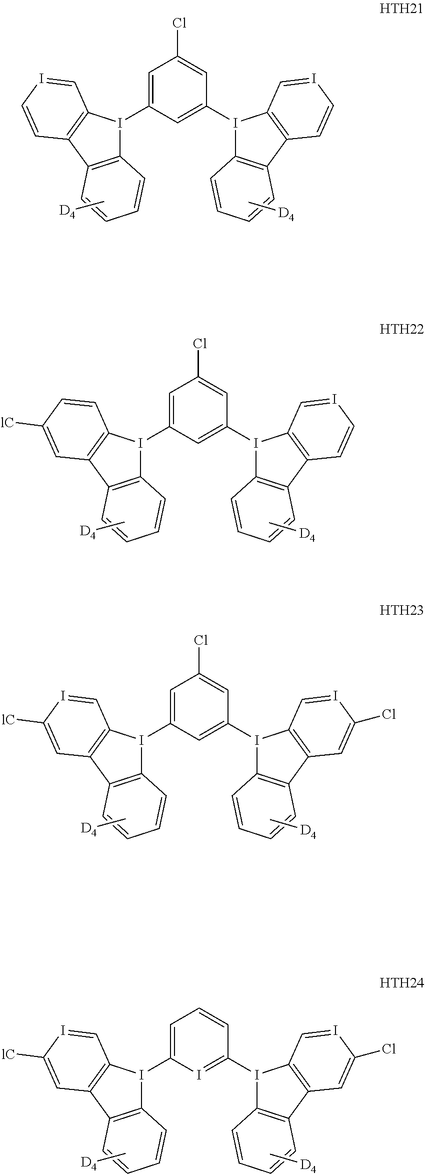

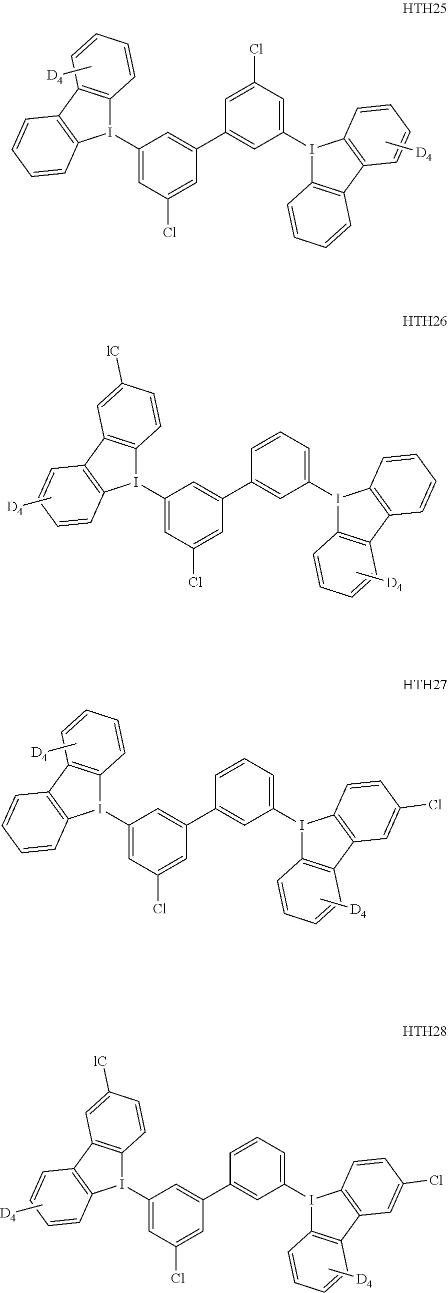

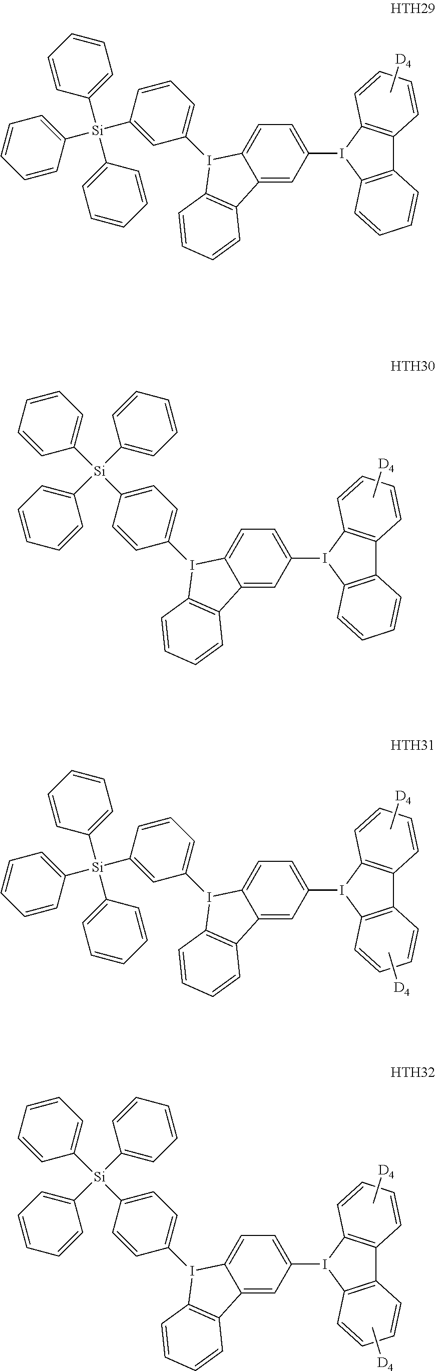

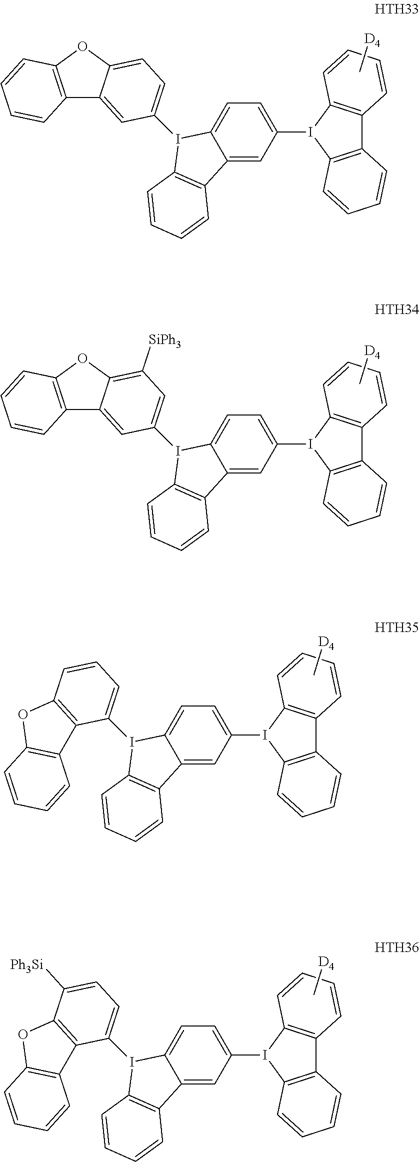

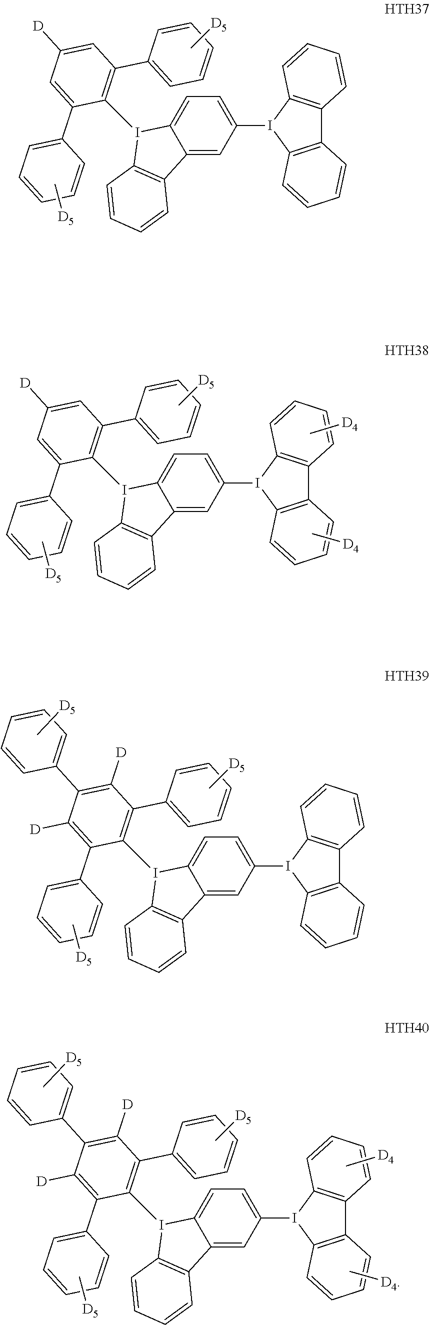

In a light-emitting device according to an embodiment, the first host and the second host may form an exciplex, and the organometallic compound and the first host or the organometallic compound and the second host may not form an exciplex.

In embodiments, the electron transport region of the light-emitting device may include a hole-blocking layer, and the hole-blocking layer may include a phosphine oxide-containing compound, a silicon-containing compound, or any combination thereof. In an embodiment, the hole blocking layer may contact (e.g., directly contact) the emission layer.

In an embodiment, the light-emitting device may include a capping layer located outside the first electrode or outside the second electrode.

In an embodiment, the light-emitting device may further include at least one of a first capping layer located outside the first electrode and a second capping layer located outside the second electrode, and at least one of the first capping layer and the second capping layer may include the heterocyclic compound represented by Formula 1. The first capping layer and/or the second capping layer may be the same as described in the specification.

In an embodiment, the light-emitting device may further include:

the first capping layer located outside the first electrode and including the heterocyclic compound represented by Formula 1;
the second capping layer located outside the second electrode and including the heterocyclic compound represented by Formula 1; or

the first capping layer and the second capping layer.

The expression that an “(interlayer and/or a capping layer) includes at least one heterocyclic compound” as used herein may be construed as meaning that the “(interlayer and/or the capping layer) may include one heterocyclic compound of Formula 1 or two or more different heterocyclic compounds of Formula 1”.

For example, the interlayer and/or capping layer may include Compound 1 only as the heterocyclic compound. In this regard, Compound 1 may be present in the emission layer of the light-emitting device. In embodiments, the interlayer may include, as the heterocyclic compound, Compound 1 and Compound 2. In this regard, Compound 1 and Compound 2 may be present in the same layer (for example, all of Compound 1 and Compound 2 may be present in the emission layer), or may be present in different layers (for example, Compound 1 may be present in the emission layer, and Compound 2 may be present in the electron transport region).

The term “interlayer” as used herein refers to a single layer and/or all layers between a first electrode and a second electrode of a light-emitting device.

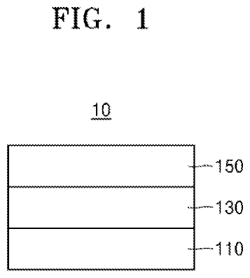

Embodiments provide an electronic device which may include the light-emitting device. The electronic device may further include a thin-film transistor. For example, the electronic device may further include a thin-film transistor including a source electrode and a drain electrode, wherein the first electrode of the light-emitting device may be electrically connected to the source electrode or the drain electrode. In an embodiment, the electronic device may further include a color filter, a color conversion layer, a touch screen layer, a polarizing layer, or any combination thereof. The electronic device may be the same as described herein.

[Description of FIG. 1]

FIG. 1 is a schematic cross-sectional view of a light-emitting device 10 according to an embodiment. The light-emitting device 10 includes a first electrode 110, an interlayer 130, and a second electrode 150.

Hereinafter, the structure of the light-emitting device 10 according to an embodiment and a method of manufacturing the light-emitting device 10 will be described with reference to FIG. 1.

[First Electrode 110]

In FIG. 1, a substrate may be further included under the first electrode 110 or on the second electrode 150. The substrate may be a glass substrate or a plastic substrate but is not limited thereto. In embodiments, the substrate may be a flexible substrate, and may include plastics with excellent heat resistance and durability, such as polyimide, polyethylene terephthalate (PET), polycarbonate, polyethylene naphthalate, polyarylate (PAR), polyetherimide, or any combination thereof.

The first electrode 110 may be formed by, for example, depositing or sputtering a material for forming the first electrode 110 on the substrate. When the first electrode 110 is an anode, a material for forming the first electrode 110 may be a high-work function material that facilitates injection of holes.

The first electrode 110 may be a reflective electrode, a semi-transmissive electrode, or a transmissive electrode. When the first electrode 110 is a transmissive electrode, a material for forming the first electrode 110 may include indium tin oxide (ITO), indium zinc oxide (IZO), tin oxide (SnO₂), zinc oxide (ZnO), or any combination thereof. In embodiments, when the first electrode 110 is a semi-transmissive electrode or a reflective electrode, a material for forming the first electrode 110 may include magnesium (Mg), silver (Ag), aluminum (Al), aluminum-lithium (Al—Li), calcium (Ca), magnesium-indium (Mg—In), magnesium-silver (Mg—Ag), or any combination thereof.

The first electrode 110 may have a structure consisting of a single layer or a structure including multiple layers. For example, the first electrode 110 may have a three-layered structure of ITO/Ag/ITO.

[Interlayer 130]

The interlayer 130 may be located on the first electrode 110. The interlayer 130 may include an emission layer.

The interlayer 130 may further include a hole transport region located between the first electrode 110 and the emission layer, and an electron transport region located between the emission layer and the second electrode 150.

The interlayer 130 may further include, in addition to various organic materials, a metal-containing compound such as an organometallic compound, an inorganic material such as quantum dots, or the like.

In embodiments, the interlayer 130 may include two or more emitting units stacked between the first electrode 110 and the second electrode 150, and at least one charge generation layer between the two or more emitting units. When the interlayer 130 includes the two or more emitting units and the at least one charge generation layer as described above, the light-emitting device 10 may be a tandem light-emitting device.

[Hole Transport Region in Interlayer 130]

The hole transport region may have a structure consisting of a layer consisting of a single material, a structure consisting of a layer including different materials, or a structure including multiple layers including different materials.

The hole transport region may include a hole injection layer, a hole transport layer, an emission auxiliary layer, an electron-blocking layer, or any combination thereof.

In an embodiment, the hole transport region may have a multi-layered structure including a hole injection layer/hole transport layer structure, a hole injection layer/hole transport layer/emission auxiliary layer structure, a hole injection layer/emission auxiliary layer structure, a hole transport layer/emission auxiliary layer structure, or a hole injection layer/hole transport layer/electron-blocking layer structure, wherein the layers of each structure may be stacked from the first electrode 110 in its respective stated order, but the structure of the hole transport region is not limited thereto.

The hole transport region may include a compound represented by Formula 201, a compound represented by Formula 202, or any combination thereof:

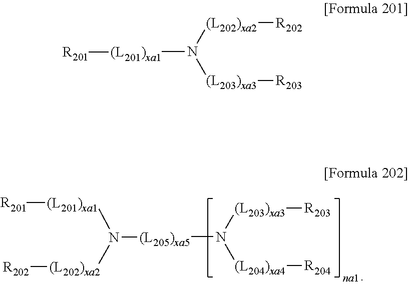

In Formulae 201 and 202,
L₂₀₁ to L₂₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
L₂₀₅ may be *—O—*′, *—S—*′, *—N(Q₂₀₁)-*′, a C₁-C₂₀ alkylene group unsubstituted or substituted with at least one R₁₀ₐ, a C₂-C₂₀ alkenylene group unsubstituted or substituted with at least one R₁₀ₐ, a C₃-C₆₀ carbocyclic group unsubstituted or substituted with at least one R₁₀ₐ, or a C₁-C₆₀ heterocyclic group unsubstituted or substituted with at least one R₁₀ₐ,
xa1 to xa4 may each independently be an integer from 0 to 5,
xa5 may be an integer from 1 to 10,
R₂₀₁ to R₂₀₄ and 0201 may each independently be a C₃-C₆₀ carbocyclic group unsubstituted or substituted with at least one R₁₀ₐ, or a C₁-C₆₀ heterocyclic group unsubstituted or substituted with at least one R₁₀ₐ,
R₂₀₁ and R₂₀₂ may optionally be linked to each other via a single bond, a C₁-C₅ alkylene group unsubstituted or substituted with at least one R₁₀ₐ, or a C₂-C₅ alkenylene group unsubstituted or substituted with at least one R₁₀ₐ, to form a C₈-C₆₀ polycyclic group (for example, a carbazole group or the like) unsubstituted or substituted with at least one R₁₀ₐ (for example, Compound HT16),
R₂₀₃ and R₂₀₄ may optionally be linked to each other via a single bond, a C₁-C₅ alkylene group unsubstituted or substituted with at least one R₁₀ₐ, or a C₂-C₅ alkenylene group unsubstituted or substituted with at least one R₁₀ₐ, to form a C₈-C₆₀ polycyclic group unsubstituted or substituted with at least one R₁₀ₐ, and
na1 may be an integer from 1 to 4.

For example, each of Formulae 201 and 202 may include at least one of groups represented by Formulae CY201 to CY217:

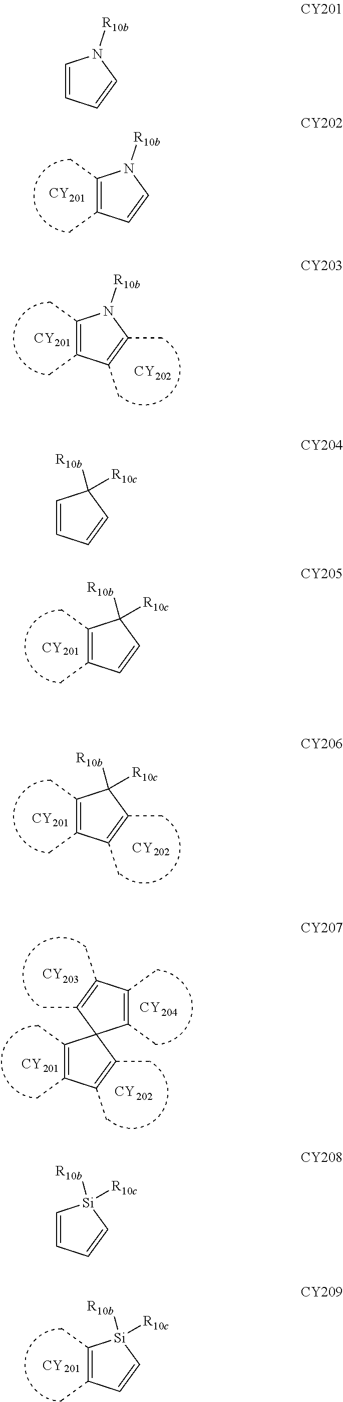

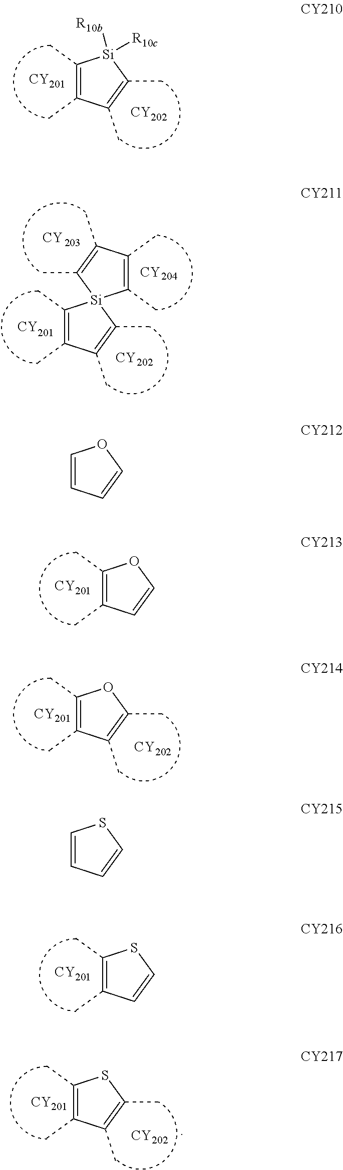

In Formulae CY201 to CY217, R_{10b} and R_{10c} may each independently be the same as described with respect to R₁₀ₐ, ring CY₂₀₁ to ring CY₂₀₄ may each independently be a C₃-C₂₀ carbocyclic group or a C₁-C₂₀ heterocyclic group, and at least one hydrogen in Formulae CY201 to CY217 may be unsubstituted or substituted with R₁₀ₐ as described herein.

In an embodiment, ring CY₂₀₁ to ring CY₂₀₄ in Formulae CY201 to CY217 may each independently be a benzene group, a naphthalene group, a phenanthrene group, or an anthracene group.

In embodiments, the compound represented by Formula 201 and the compound represented by Formula 202 may each independently include at least one of groups represented by Formulae CY201 to CY203.

In embodiments, the compound represented by Formula 201 may include at least one of the groups represented by Formulae CY201 to CY203 and at least one of the groups represented by Formulae CY204 to CY217.

In embodiments, in Formula 201, xa1 may be 1, R₂₀₁ may be a group represented by one of Formulae CY201 to CY203, xa2 may be 0, and R₂₀₂ may be a group represented by one of Formulae CY204 to CY207.

In embodiments, the compound represented by Formula 201 and the compound represented by Formula 202 may each not include a group represented by one of Formulae CY201 to CY203.

In embodiments, the compound represented by Formula 201 and the compound represented by Formula 202 may not include a group represented by one of Formulae CY201 to CY203, and may each independently include at least one of the groups represented by Formulae CY204 to CY217.

In embodiments, the compound represented by Formula 201 and the compound represented by Formula 202 may each not include a group represented by one of Formulae CY201 to CY217.

In an embodiment, the hole transport region may include one of Compounds HT1 to HT46, m-MTDATA, TDATA, 2-TNATA, NPB(NPD), β-NPB, TPD, Spiro-TPD, Spiro-NPB, methylated NPB, TAPC, HMTPD, 4,4′,4″-tris(N-carbazolyl)triphenylamine (TCTA), polyaniline/dodecylbenzenesulfonic acid (PANT/DBSA), poly(3,4-ethylenedioxythiophene)/poly(4-styrenesulfonate) (PEDOT/PSS), polyaniline/camphor sulfonic acid (PANI/CSA), polyaniline/poly(4-styrenesulfonate) (PANT/PSS), or any combination thereof:

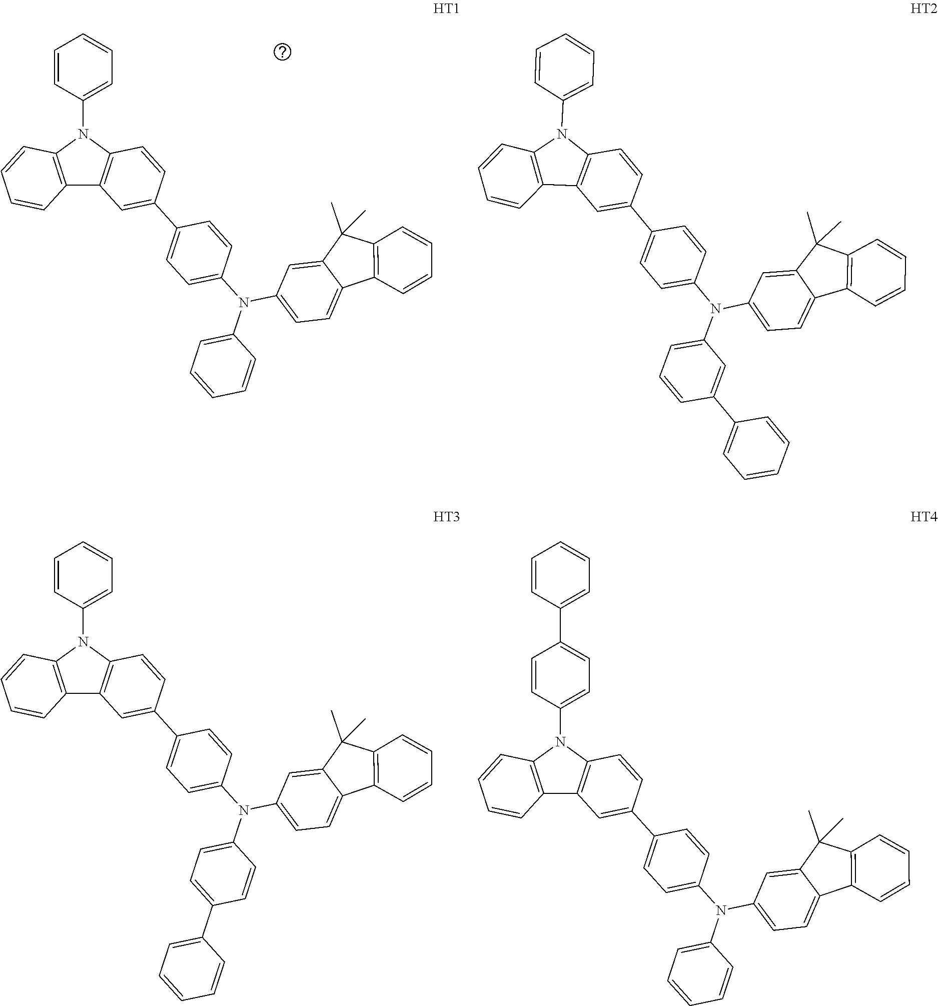 [SMILES: CC1(C)c2ccccc2-c2ccc(N(c3ccccc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4ccccc4)c3)cc21 ; CC1(C)c2ccccc2-c2ccc(N(c3ccc(-c4ccccc4)cc3)c3ccc(-c4ccc5c(c4)c4ccccc4n5-c4ccccc4)cc3)cc21 ; CC1(C)c2ccccc2-c2ccc(N(c3ccccc3)c3ccc(-c4ccc5c(c4)c4ccccc4n5-c4ccc(-c5ccccc5)cc4)cc3)cc21]

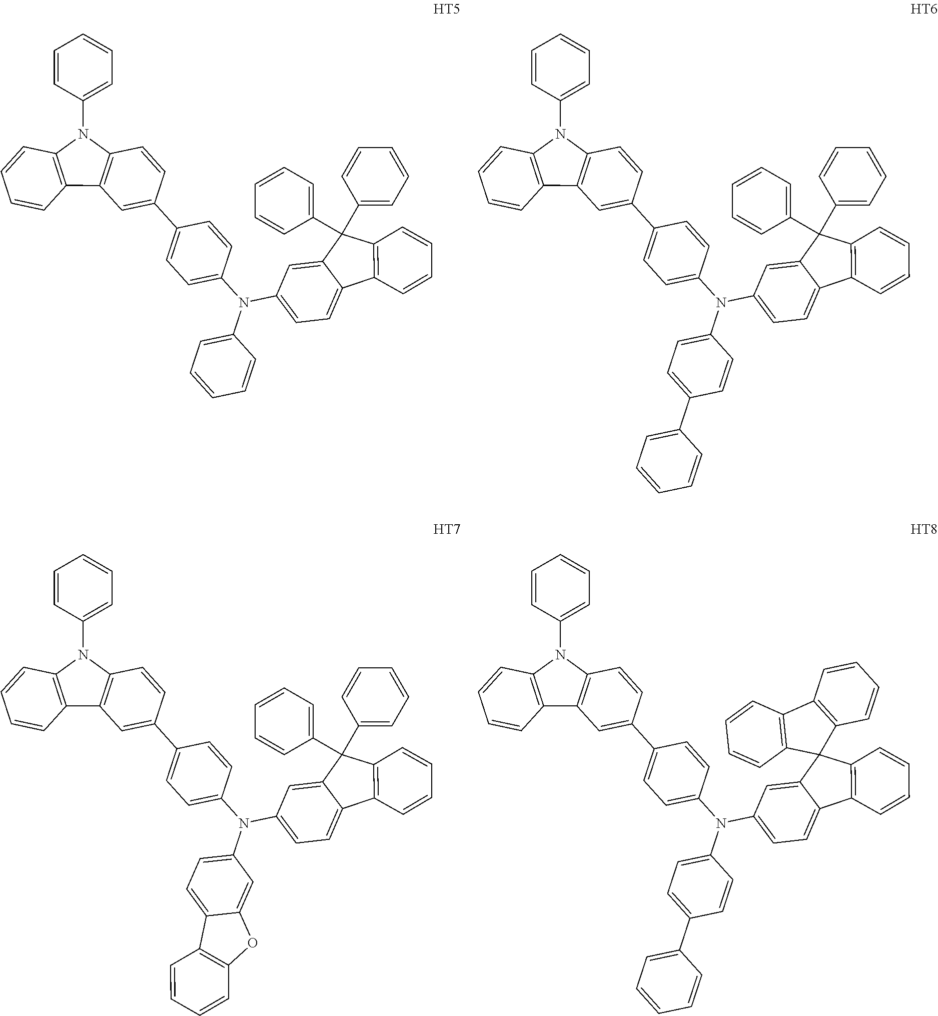 [SMILES: c1ccc(N(c2ccc(-c3ccc4c(c3)c3ccccc3n4-c3ccccc3)cc2)c2ccc3c(c2)C(c2ccccc2)(c2ccccc2)c2ccccc2-3)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1 ; c1ccc(-n2c3ccccc3c3cc(-c4ccc(N(c5ccc6c(c5)C(c5ccccc5)(c5ccccc5)c5ccccc5-6)c5ccc6c(c5)oc5ccccc56)cc4)ccc32)cc1 ; c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1]

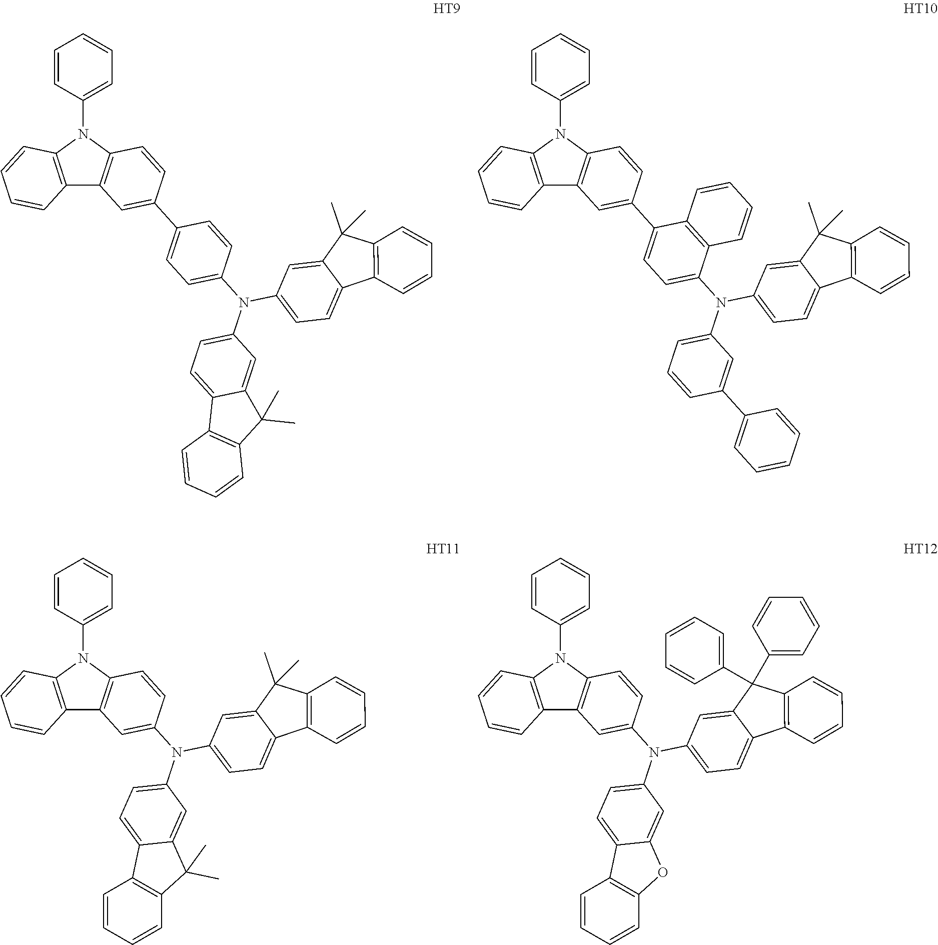 [SMILES: CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21 ; CC1(C)c2ccccc2-c2ccc(N(c3cccc(-c4ccccc4)c3)c3ccc(-c4ccc5c(c4)c4ccccc4n5-c4ccccc4)c4ccccc34)cc21 ; CC1(C)c2ccccc2-c2ccc(N(c3ccc4c(c3)C(C)(C)c3ccccc3-4)c3ccc4c(c3)c3ccccc3n4-c3ccccc3)cc21 ; c1ccc(-n2c3ccccc3c3cc(N(c4ccc5c(c4)C(c4ccccc4)(c4ccccc4)c4ccccc4-5)c4ccc5c(c4)oc4ccccc45)ccc32)cc1]

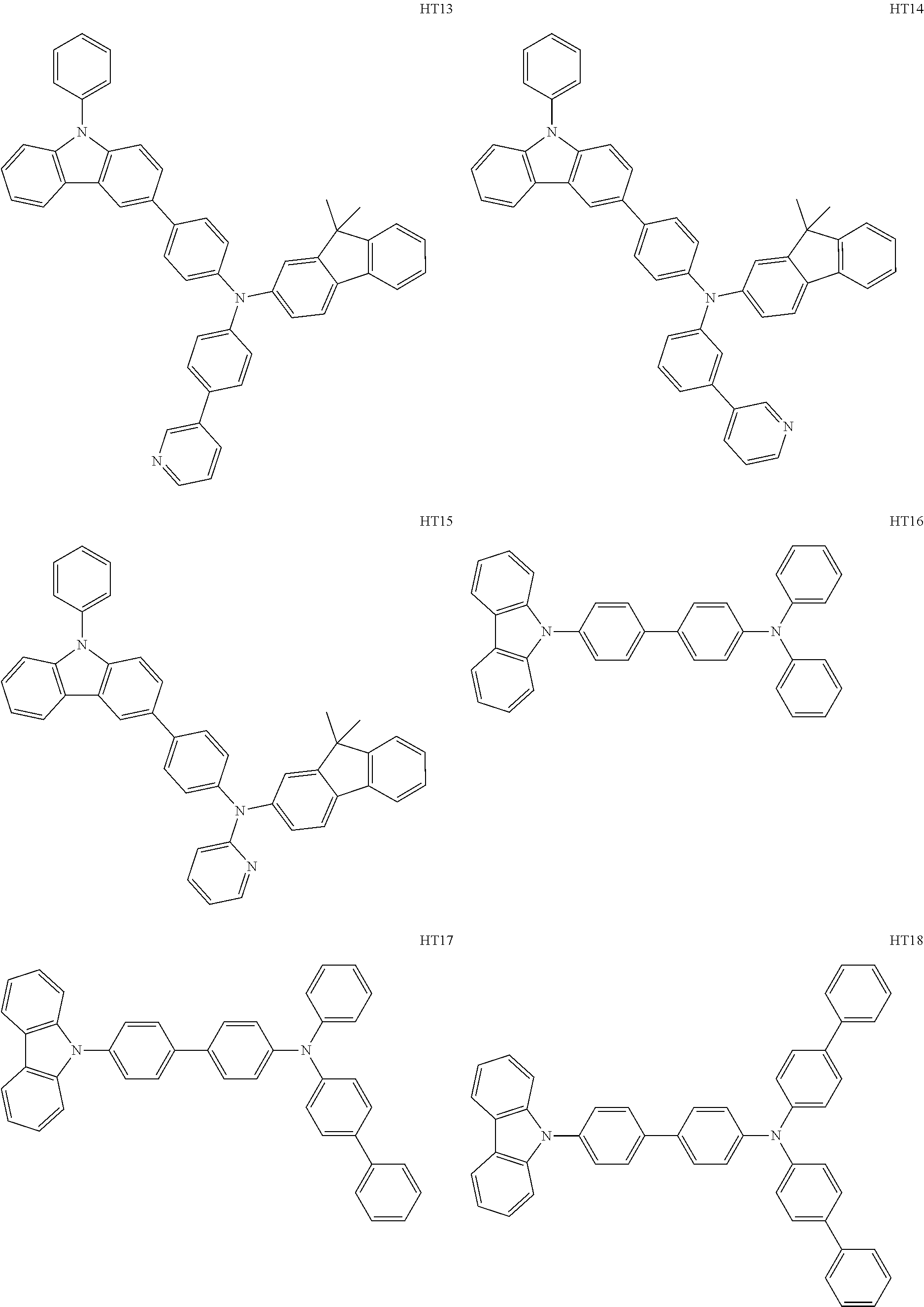 [SMILES: CC1(C)c2ccccc2-c2ccc(N(c3ccc(-c4cccnc4)cc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4cccnc4)c3)cc21 ; CC1(C)c2ccccc2-c2ccc(N(c3ccc(-c4ccc5c(c4)c4ccccc4n5-c4ccccc4)cc3)c3ccccn3)cc21 ; c1ccc(N(c2ccccc2)c2ccc(-c3ccc(-n4c5ccccc5c5ccccc54)cc3)cc2)cc1 ; c1ccc(-c2ccc(N(c3ccccc3)c3ccc(-c4ccc(-n5c6ccccc6c6ccccc65)cc4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1]

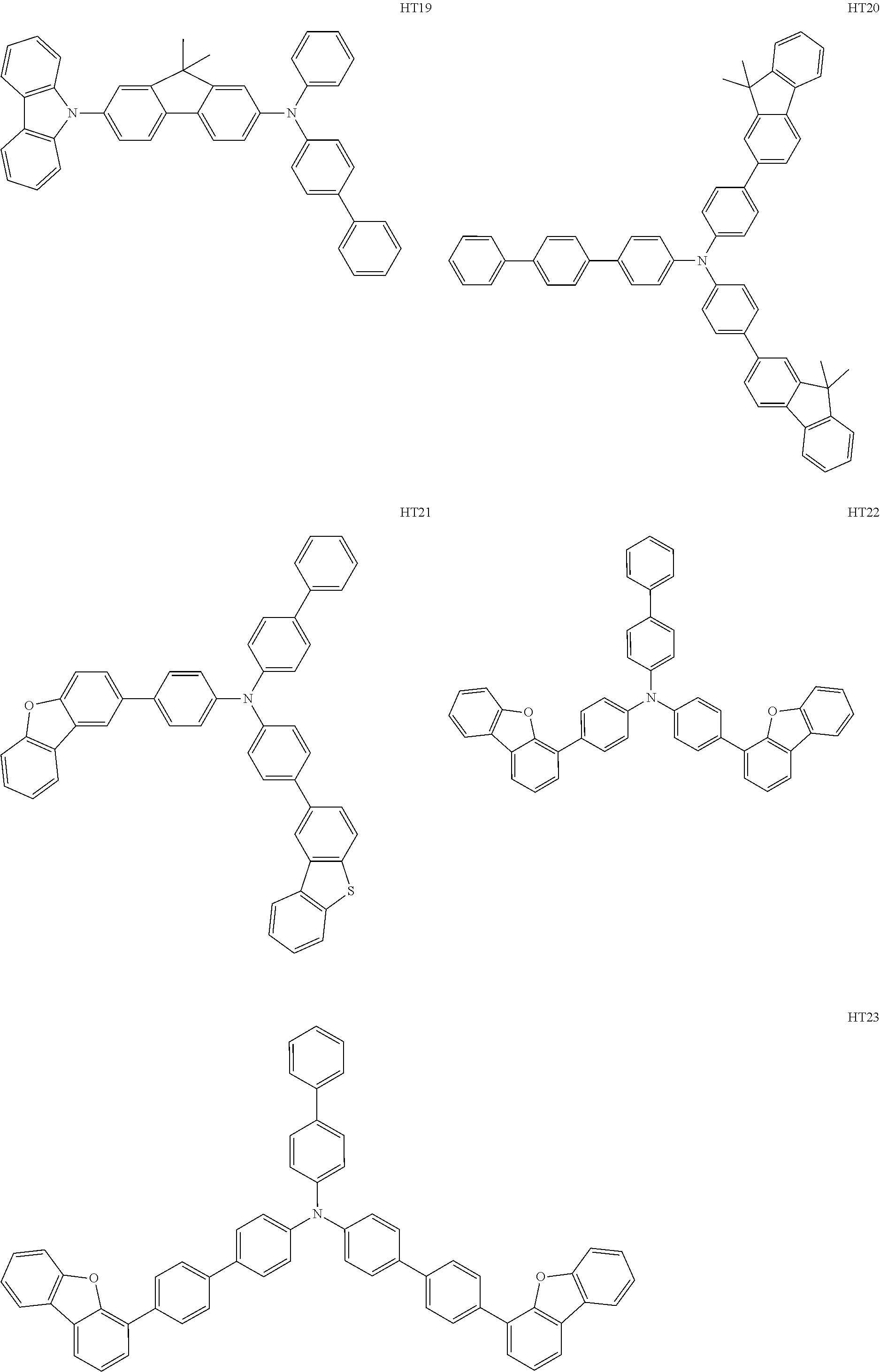 [SMILES: CC1(C)c2cc(N(c3ccccc3)c3ccc(-c4ccccc4)cc3)ccc2-c2ccc(-n3c4ccccc4c4ccccc43)cc21 ; CC1(C)c2ccccc2-c2ccc(-c3ccc(N(c4ccc(-c5ccc(-c6ccccc6)cc5)cc4)c4ccc(-c5ccc6c(c5)C(C)(C)c5ccccc5-6)cc4)cc3)cc21 ; c1ccc(-c2ccc(N(c3ccc(-c4ccc5oc6ccccc6c5c4)cc3)c3ccc(-c4ccc5sc6ccccc6c5c4)cc3)cc2)cc1 ; c1ccc(-c2ccc(N(c3ccc(-c4cccc5c4oc4ccccc45)cc3)c3ccc(-c4cccc5c4oc4ccccc45)cc3)cc2)cc1 ; c1ccc(-c2ccc(N(c3ccc(-c4ccc(-c5cccc6c5oc5ccccc56)cc4)cc3)c3ccc(-c4ccc(-c5cccc6c5oc5ccccc56)cc4)cc3)cc2)cc1]

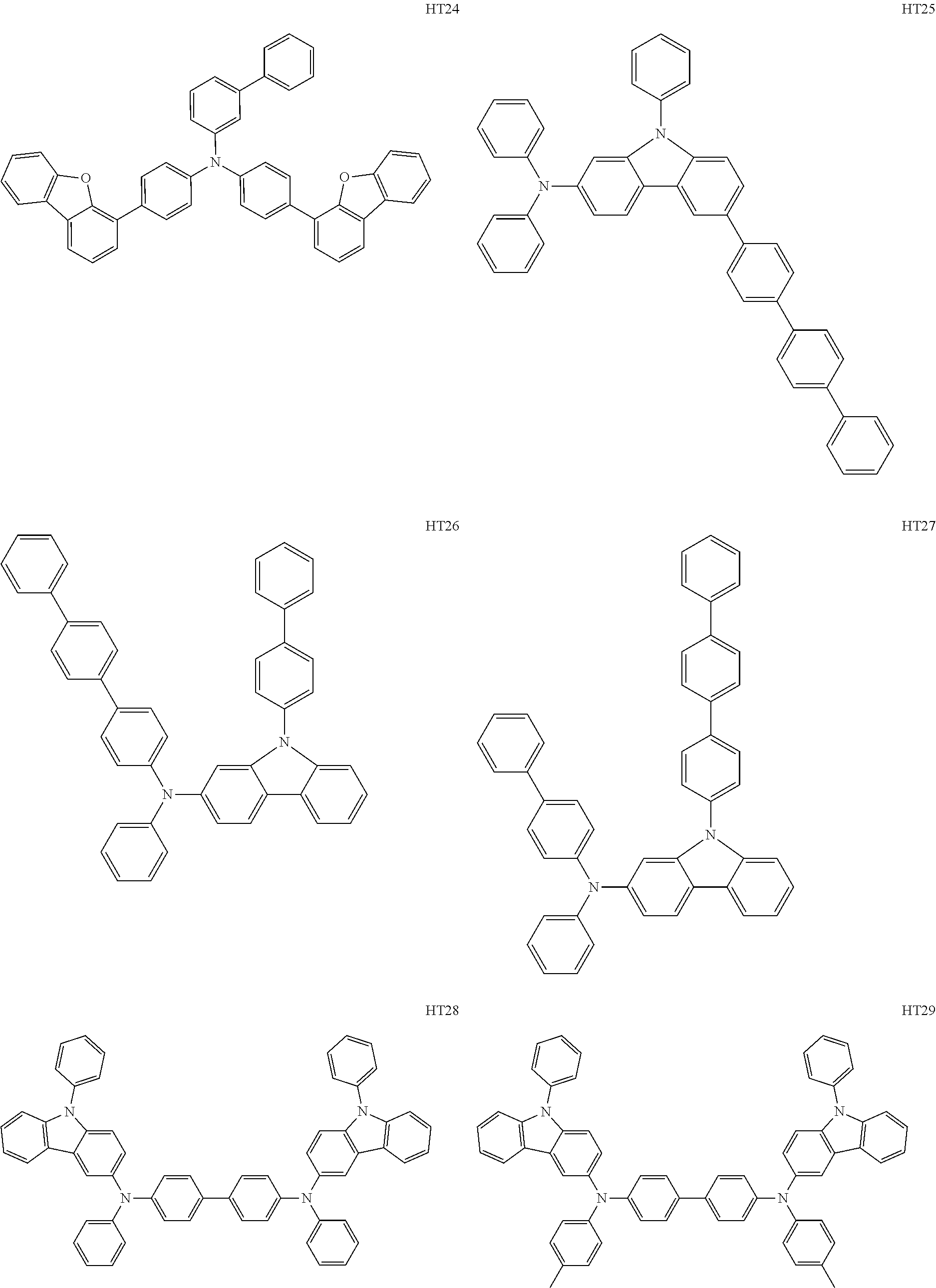

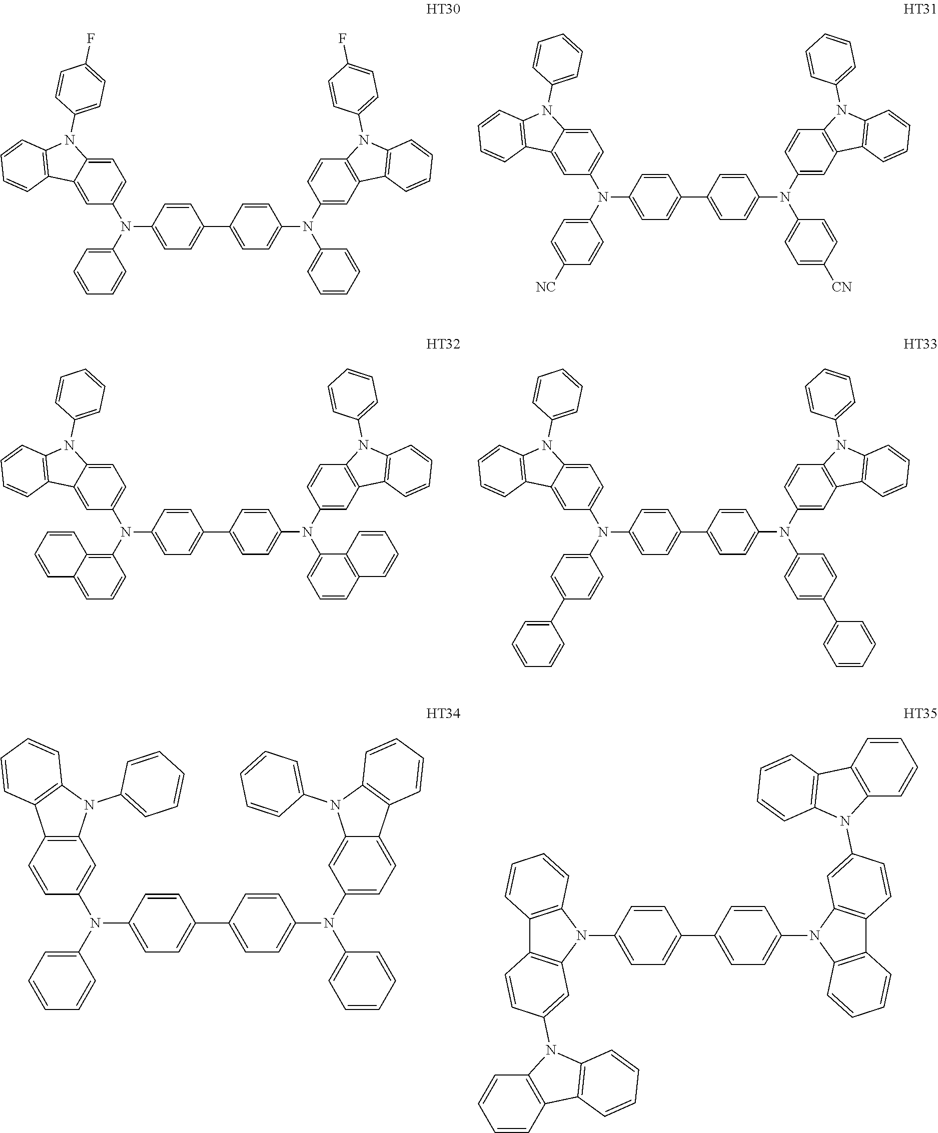

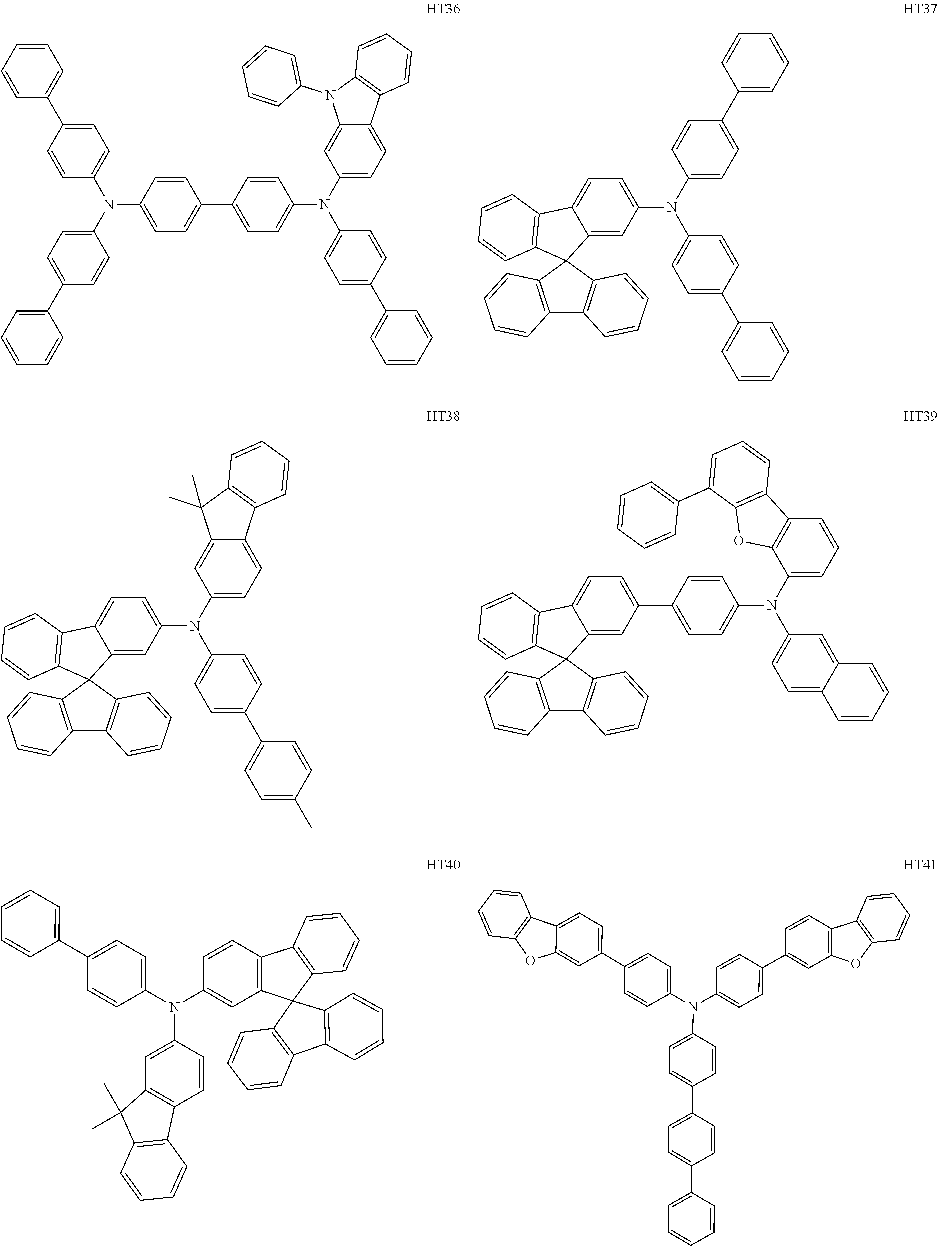

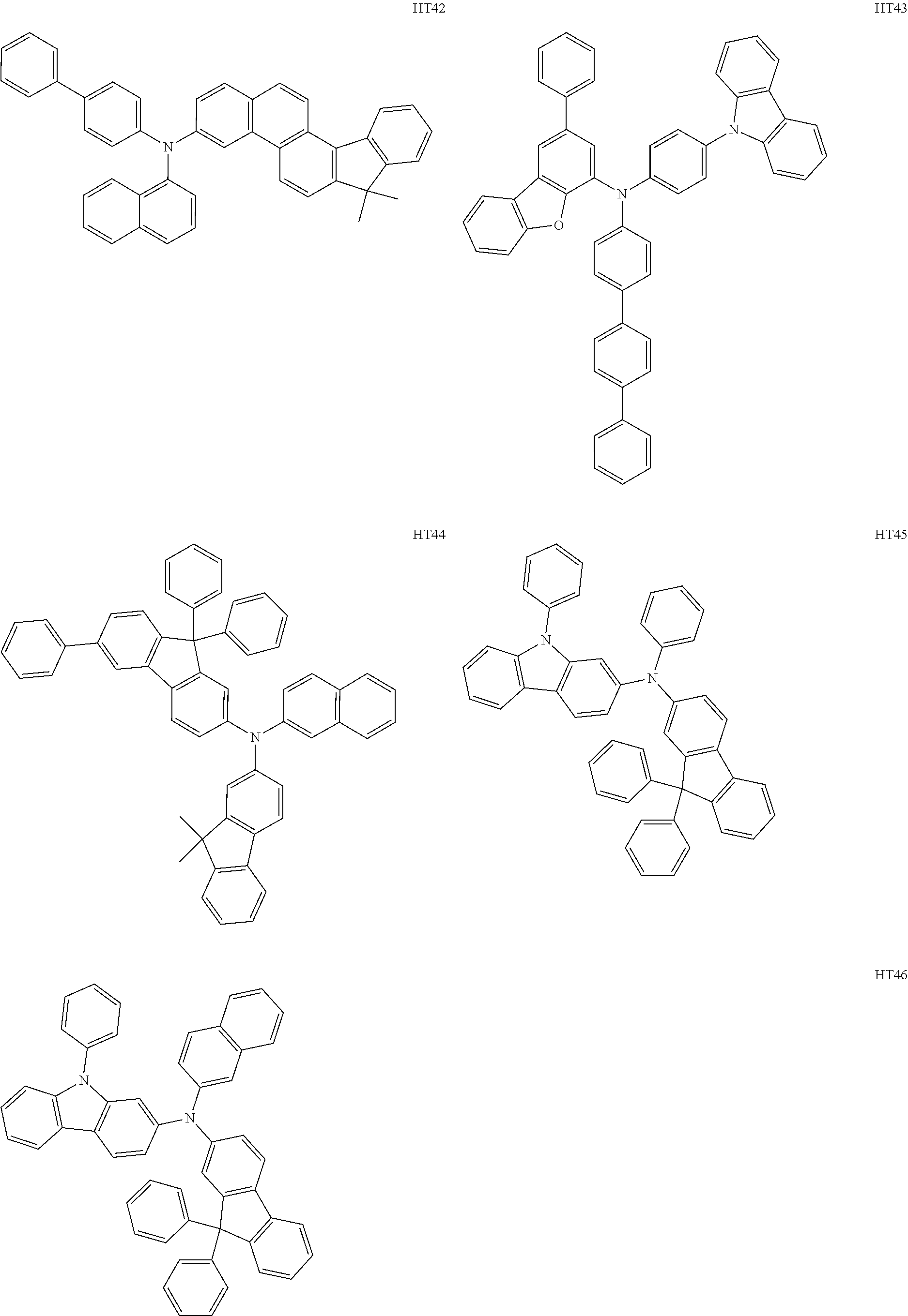 [SMILES: CC1(C)c2ccccc2-c2c1ccc1c2ccc2ccc(N(c3ccc(-c4ccccc4)cc3)c3cccc4ccccc34)cc21 ; c1ccc(-c2ccc(-c3ccc(N(c4ccc(-n5c6ccccc6c6ccccc65)cc4)c4cc(-c5ccccc5)cc5c4oc4ccccc45)cc3)cc2)cc1 ; CC1(C)c2ccccc2-c2ccc(N(c3ccc4c(c3)C(c3ccccc3)(c3ccccc3)c3ccc(-c5ccccc5)cc3-4)c3ccc4ccccc4c3)cc21 ; c1ccc(N(c2ccc3c(c2)C(c2ccccc2)(c2ccccc2)c2ccccc2-3)c2ccc3c4ccccc4n(-c4ccccc4)c3c2)cc1 ; c1ccc(-n2c3ccccc3c3ccc(N(c4ccc5c(c4)C(c4ccccc4)(c4ccccc4)c4ccccc4-5)c4ccc5ccccc5c4)cc32)cc1]

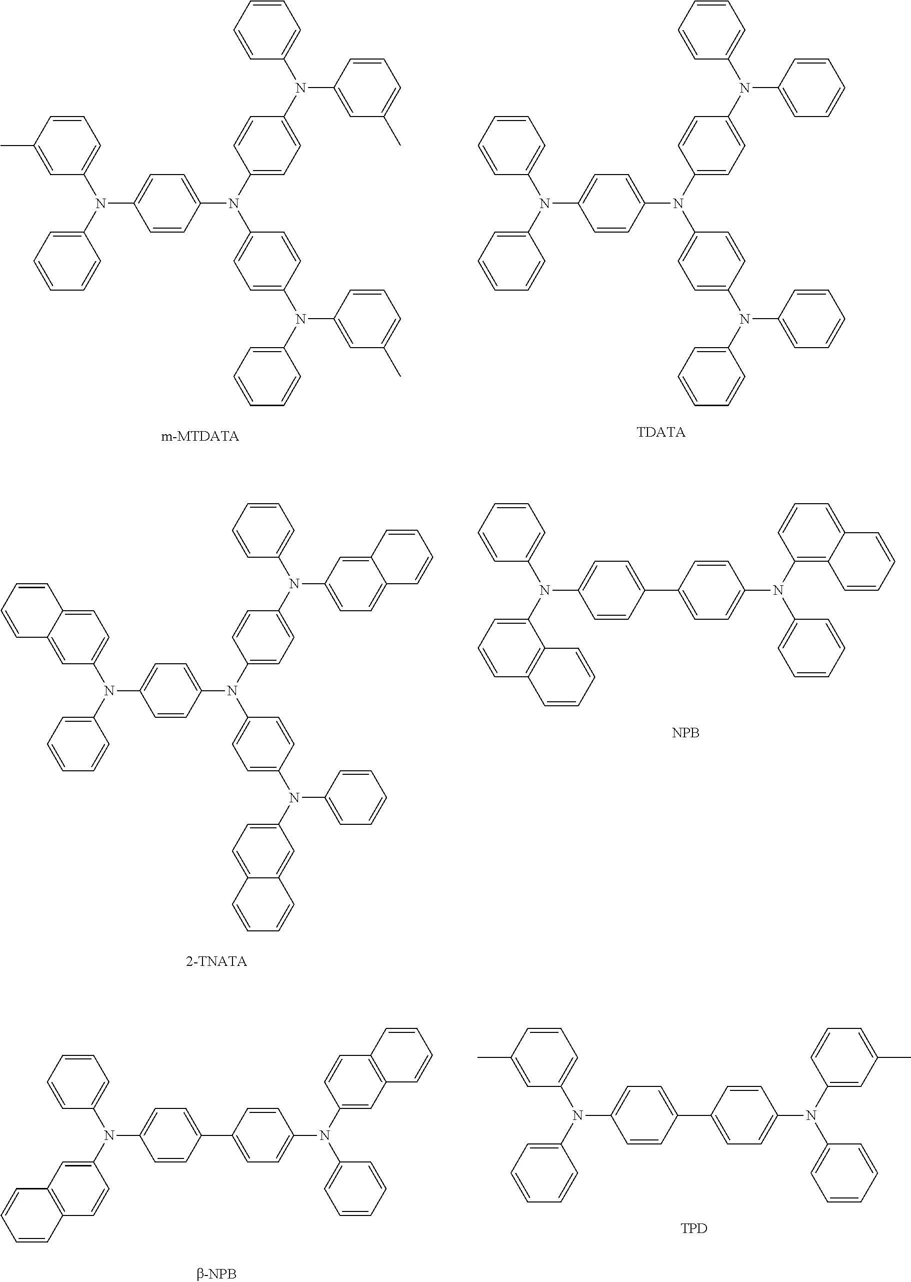

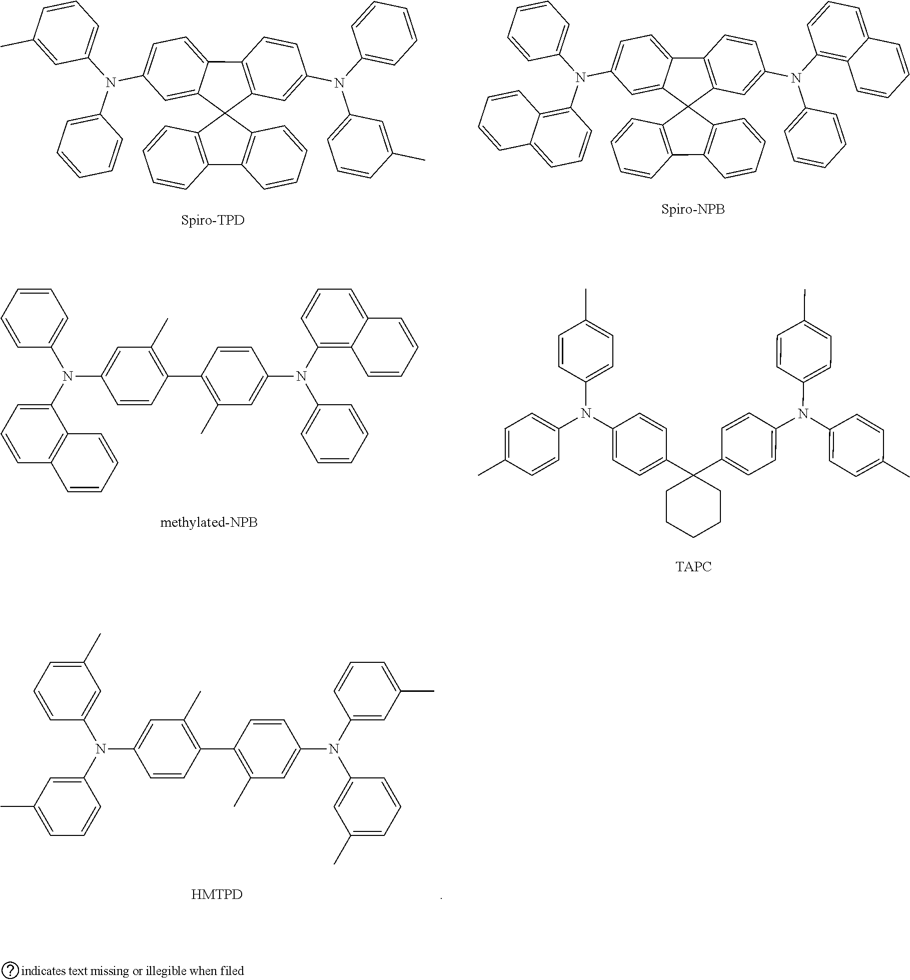

A thickness of the hole transport region may be in a range of about 50 Å to about 10,000 Å. For example, the thickness of the hole transport region may be in a range of about 100 Å to about 4,000 Å. When the hole transport region includes a hole injection layer, a hole transport layer, or any combination thereof, a thickness of the hole injection layer may be in a range of about 100 Å to about 9,000 Å, and a thickness of the hole transport layer may be in a range of about 50 Å to about 2,000 Å. For example, the thickness of the hole injection region may be in a range of about 100 Å to about 1,000 Å. For example, the thickness of the hole transport layer may be in a range of about 100 Å to about 1,500 Å. When the thicknesses of the hole transport region, the hole injection layer, and the hole transport layer are within these ranges, satisfactory hole transporting characteristics may be obtained without a substantial increase in driving voltage.

The emission auxiliary layer may increase light-emission efficiency by compensating for an optical resonance distance according to the wavelength of light emitted by an emission layer, and the electron-blocking layer may block the leakage of electrons from an emission layer to a hole transport region. Materials that may be included in the hole transport region may be included in the emission auxiliary layer and the electron-blocking layer.

[p-Dopant]

The hole transport region may further include, in addition to these materials, a charge-generation material for the improvement of conductive properties. The charge-generation material may be uniformly or non-uniformly dispersed in the hole transport region (for example, in the form of a single layer consisting of a charge-generation material).

The charge-generation material may be, for example, a p-dopant.

In an embodiment, a lowest unoccupied molecular orbital (LUMO) energy level of the p-dopant may be equal to or less than about −3.5 eV.

In embodiments, the p-dopant may include a quinone derivative, a cyano group-containing compound, a compound including element EL1 and element EL2, or any combination thereof.

Examples of a quinone derivative may include TCNQ, F4-TCNQ, etc.

Examples of a cyano group-containing compound may include HAT-CN, and a compound represented by Formula 221:

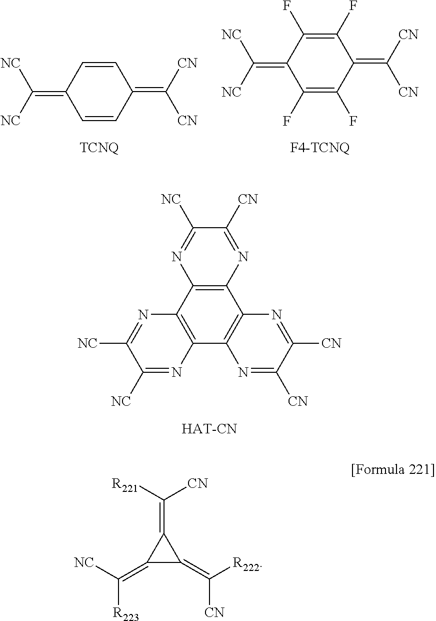

In Formula 221,
R₂₂₁ to R₂₂₃ may each independently be a C₃-C₆₀ carbocyclic group unsubstituted or substituted with at least one R₁₀ₐ or a C₁-C₆₀ heterocyclic group unsubstituted or substituted with at least one R₁₀ₐ, and
at least one of R₂₂₁ to R₂₂₃ may each independently be a C₃-C₆₀ carbocyclic group or a C₁-C₆₀ heterocyclic group, each substituted with a cyano group; —F; —Cl; —Br; —I; a C₁-C₂₀ alkyl group substituted with a cyano group, —F, —Cl, —Br, —I, or any combination thereof; or any combination thereof.

In the compound including element EL1 and element EL2, element EL1 may be a metal, a metalloid, or any combination thereof, and element EL2 may be a non metal, a metalloid, or any combination thereof.

Examples of a metal may include: an alkali metal (for example, lithium (Li), sodium (Na), potassium (K), rubidium (Rb), cesium (Cs), etc.); an alkaline earth metal (for example, beryllium (Be), magnesium (Mg), calcium (Ca), strontium (Sr), barium (Ba), etc.); a transition metal (for example, titanium (Ti), zirconium (Zr), hafnium (Hf), vanadium (V), niobium (Nb), tantalum (Ta), chromium (Cr), molybdenum (Mo), tungsten (W), manganese (Mn), technetium (Tc), rhenium (Re), iron (Fe), ruthenium (Ru), osmium (Os), cobalt (Co), rhodium (Rh), iridium (Ir), nickel (Ni), palladium (Pd), platinum (Pt), copper (Cu), silver (Ag), gold (Au), etc.); a post-transition metal (for example, zinc (Zn), indium (In), tin (Sn), etc.); and a lanthanide metal (for example, lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), etc.).

Examples of a metalloid may include silicon (Si), antimony (Sb), and tellurium (Te).

Examples of a non-metal may include oxygen (O) and a halogen (for example, F, Cl, Br, I, etc.).

Examples of the compound including element EL1 and element EL2 may include a metal oxide, a metal halide (for example, a metal fluoride, a metal chloride, a metal bromide, or a metal iodide), a metalloid halide (for example, a metalloid fluoride, a metalloid chloride, a metalloid bromide, or a metalloid iodide), a metal telluride, or any combination thereof.

Examples of a metal oxide may include a tungsten oxide (for example, WO, W₂O₃, WO₂, WO₃, W₂O₅, etc.), a vanadium oxide (for example, VO, V₂O₃, VO₂, V₂O₅, etc.), a molybdenum oxide (MoO, Mo₂O₃, MoO₂, MoO₃, MO₂O₅, etc.), and a rhenium oxide (for example, ReO₃, etc.).

Examples of a metal halide may include an alkali metal halide, an alkaline earth metal halide, a transition metal halide, a post-transition metal halide, and a lanthanide metal halide.

Examples of an alkali metal halide may include LiF, NaF, KF, RbF, CsF, LiCl, NaCl, KCl, RbCl, CsCl, LiBr, NaBr, KBr, RbBr, CsBr, LiI, NaI, KI, RbI, and CsI.

Examples of an alkaline earth metal halide may include BeF₂, MgF₂, CaF₂, SrF₂, BaF₂, BeCl₂, MgCl₂, CaCl₂), SrCl₂, BaCl₂, BeBr₂, MgBr₂, CaBr₂, SrBr₂, BaBr₂, Belt, MgI₂, CaI₂, SrI₂, and Bale.

Examples of a transition metal halide may include a titanium halide (for example, TiF₄, TiCl₄, TiBr₄, TiI₄, etc.), a zirconium halide (for example, ZrF₄, ZrCl₄, ZrBr₄, ZrI₄, etc.), a hafnium halide (for example, HfF₄, HfCl₄, HfBr₄, HfI₄, etc.), a vanadium halide (for example, VF₃, VCl₃, VBr₃, VI₃, etc.), a niobium halide (for example, NbF₃, NbCl₃, NbBr₃, NbI₃, etc.), a tantalum halide (for example, TaF₃, TaCl₃, TaBr₃, TaI₃, etc.), a chromium halide (for example, CrF₃, CrCl₃, CrBr₃, CrI₃, etc.), a molybdenum halide (for example, MoF₃, MoCl₃, MoBr₃, MoI₃, etc.), a tungsten halide (for example, WF₃, WCl₃, WBr₃, WI₃, etc.), a manganese halide (for example, MnF₂, MnCl₂, MnBr₂, MnI₂, etc.), a technetium halide (for example, TcF₂, TcCl₂, TcBr₂, TCl₂, etc.), a rhenium halide (for example, ReF₂, ReCl₂, ReBr₂, ReI₂, etc.), an iron halide (for example, FeF₂, FeCl₂, FeBr₂, Felt, etc.), a ruthenium halide (for example, RuF₂, RuCl₂, RuBr₂, Rule, etc.), an osmium halide (for example, OsF₂, OsCl₂, OsBr₂, OsI₂, etc.), a cobalt halide (for example, CoF₂, CoCl₂, CoBr₂, Cole, etc.), a rhodium halide (for example, RhF₂, RhCl₂, RhBr₂, RhI₂, etc.), an iridium halide (for example, IrF₂, IrCl₂, IrBr₂, IrI₂, etc.), a nickel halide (for example, NiF₂, NiCl₂, NiBr₂, NiI₂, etc.), a palladium halide (for example, PdF₂, PdCl₂, PdBr₂, PdI₂, etc.), a platinum halide (for example, PtF₂, PtCl₂, PtBr₂, PtI₂, etc.), a copper halide (for example, CuF, CuCl, CuBr, CuI, etc.), a silver halide (for example, AgF, AgCl, AgBr, AgI, etc.), and a gold halide (for example, AuF, AuCl, AuBr, AuI, etc.).

Examples of a post-transition metal halide may include a zinc halide (for example, ZnF₂, ZnCl₂, ZnBr₂, ZnI₂, etc.), an indium halide (for example, InI₃, etc.), and a tin halide (for example, SnI₂, etc.).

Examples of a lanthanide metal halide may include YbF, YbF₂, YbF₃, SmF₃, YbCl, YbCl₂, YbCl₃, SmCl₃, YbBr, YbBr₂, YbBr₃, SmBr₃, YbI, YbI₂, YbI₃, and SmI₃.

Examples of a metalloid halide may include an antimony halide (for example, SbCl₅ and the like) and the like.

Examples of a metal telluride may include an alkali metal telluride (for example, Li₂Te, Na₂Te, K₂Te, Rb₂Te, Cs₂Te, etc.), an alkaline earth metal telluride (for example, BeTe, MgTe, CaTe, SrTe, BaTe, etc.), a transition metal telluride (for example, TiTe₂, ZrTe₂, HfTe₂, V₂Te₃, Nb₂Te₃, Ta₂Te₃, Cr₂Te₃, Mo₂Te₃, W₂Te₃, MnTe, TcTe, ReTe, FeTe, RuTe, OsTe, CoTe, RhTe, IrTe, NiTe, PdTe, PtTe, Cu₂Te, CuTe, Ag₂Te, AgTe, Au₂Te, etc.), a post-transition metal telluride (for example, ZnTe, etc.), and a lanthanide metal telluride (for example, LaTe, CeTe, PrTe, NdTe, PmTe, EuTe, GdTe, TbTe, DyTe, HoTe, ErTe, TmTe, YbTe, LuTe, etc.).

[Emission Layer in Interlayer 130]

When the light-emitting device 10 is a full-color light-emitting device, the emission layer may be patterned into a red emission layer, a green emission layer, and/or a blue emission layer, according to a subpixel. In embodiments, the emission layer may have a stacked structure of two or more layers of a red emission layer, a green emission layer, and a blue emission layer, in which the two or more layers may contact each other or may be separated from each other to emit white light. In embodiments, the emission layer may include two or more materials of a red light-emitting material, a green light-emitting material, and a blue light-emitting material, in which the two or more materials are mixed with each other in a single layer to emit white light.

The emission layer may include a host and a dopant. The dopant may include a phosphorescent dopant, a fluorescent dopant, or any combination thereof.

The amount of the dopant in the emission layer may be in a range of about 0.01 parts by weight to about 15 parts by weight based on 100 parts by weight of the host.

In embodiments, the emission layer may include a quantum dot.

The emission layer may include a delayed fluorescence material. The delayed fluorescence material may serve as a host or as a dopant in the emission layer.

A thickness of the emission layer may be in a range of about 100 Å to about 1,000 Å. For example, the thickness of the emission layer may be in a range of about 200 Å to about 600 Å. When the thickness of the emission layer is within these ranges, excellent light-emission characteristics may be obtained without a substantial increase in driving voltage.

[Host]

The host may further include a compound represented by Formula 301:

[Ar₃₀₁]_{xb11}-[(L₃₀₁)_{xb1}-R₃₀₁]_{xb21}. [Formula 301]

In Formula 301,
Ar₃₀₁ and L₃₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xb11 may be 1, 2, or 3,
xb1 may be an integer from 0 to 5,
R₃₀₁ may be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₃₀₁)(Q₃₀₂)(Q₃₀₃), —N(Q₃₀₁)(Q₃₀₂), —B(Q₃₀₁)(Q₃₀₂), —C(═O)(Q₃₀₁), —S(═O)₂(Q₃₀₁), or —P(═O)(Q₃₀₁)(Q₃₀₂),
xb21 may be an integer from 1 to 5, and
Q₃₀₁ to Q₃₀₃ may each independently be the same as described herein with respect to Q₁.

In an embodiment, in Formula 301, when xb11 is 2 or more, two or more of Ar₃₀₁(s) may be linked to each other via a single bond.

In embodiments, the host may include a compound represented by Formula 301-1, a compound represented by Formula 301-2, or any combination thereof:

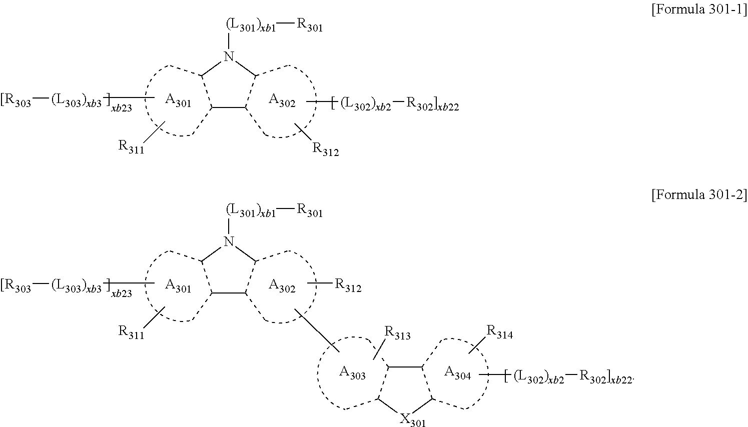

In Formulae 301-1 and 301-2,
ring A₃₀₁ to ring A₃₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
X₃₀₁ may be O, S, N-[(L₃₀₄)_{xb4}-R₃₀₄], C(R₃₀₄)(R₃₀₅), or Si(R₃₀₄)(R₃₀₅),
xb22 and xb23 may each independently be 0, 1, or 2,
L₃₀₁, xb1, and R₃₀₁ may each independently be the same as described herein,

L₃₀₂ to L₃₀₄ may each independently be the same as described herein with respect to with L₃₀₁,
xb2 to xb4 may each independently be the same as described herein with respect to xb1, and
R₃₀₂ to R₃₀₅ and R₃₁₁ to R₃₁₄ may each independently be the same as described herein with respect to R₃₀₁.

In embodiments, the host may include an alkali earth metal complex, a post-transition metal complex, or any combination thereof. For example, the host may include a Be complex (for example, Compound H55), an Mg complex, a Zn complex, or any combination thereof.

In embodiments, the host may include one of Compounds H1 to H128, 9,10-di(2-naphthyl)anthracene (ADN), 2-methyl-9,10-bis(naphthalen-2-yl)anthracene (MADN), 9,10-di-(2-naphthyl)-2-t-butyl-anthracene (TBADN), 4,4′-bis(N-carbazolyl)-1,1′-biphenyl (CBP), 1,3-di-9-carbazolylbenzene (mCP), 1,3,5-tri(carbazol-9-yl)benzene (TCP), or any combination thereof:

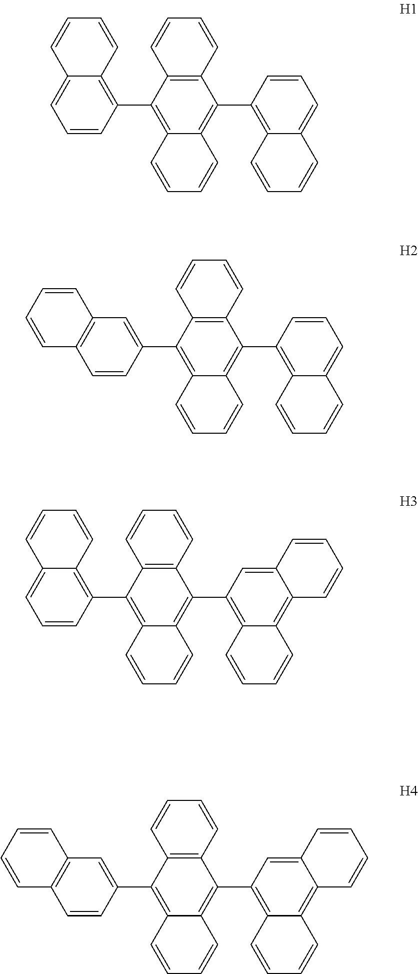

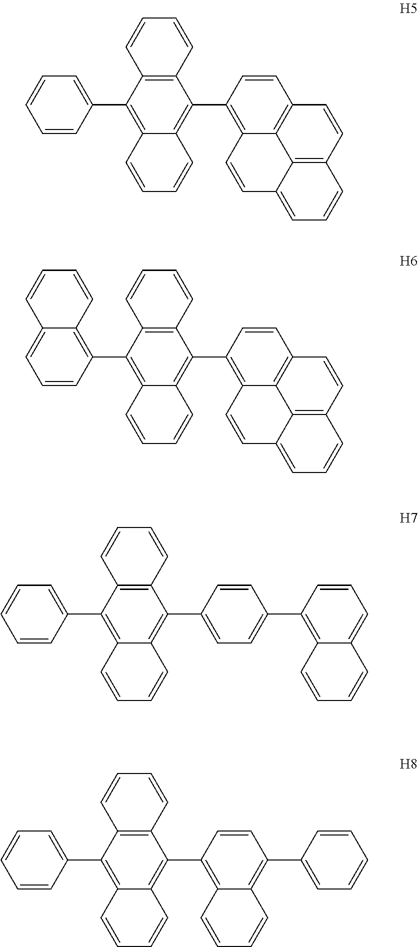

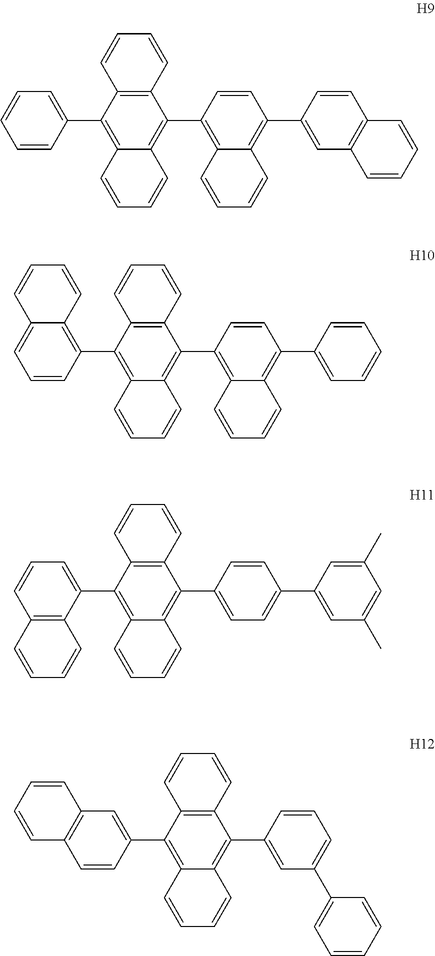

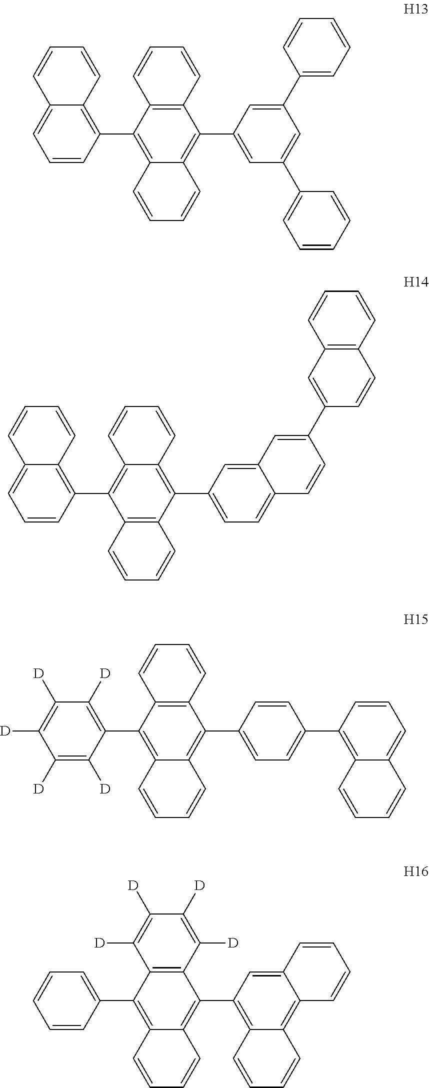

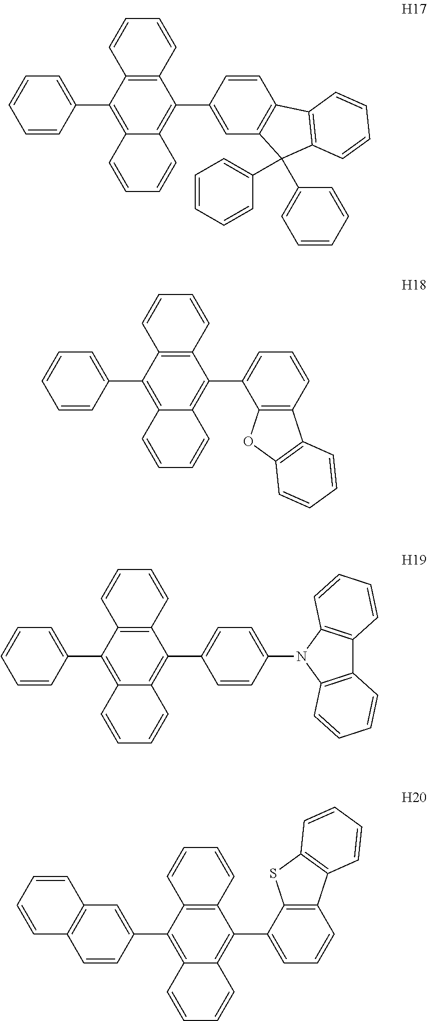

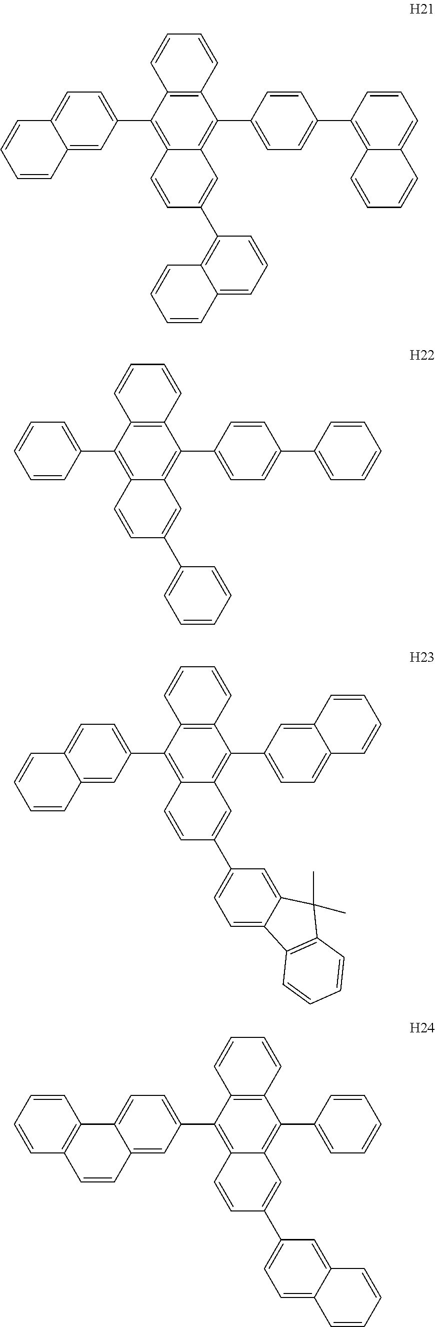

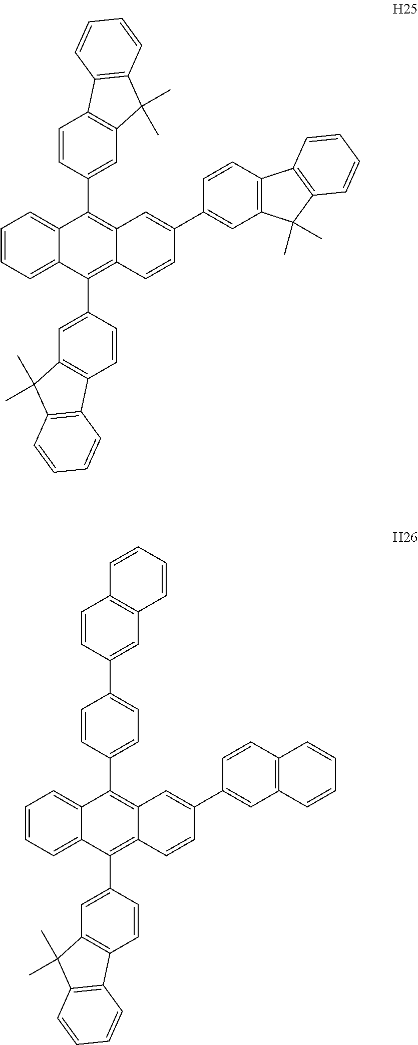

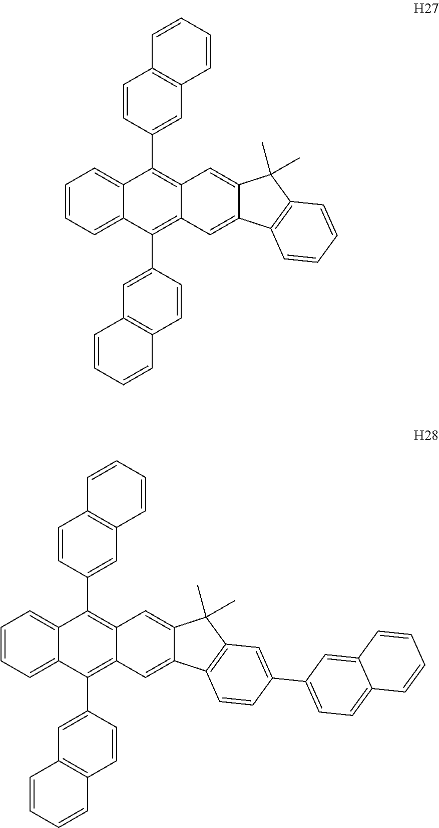

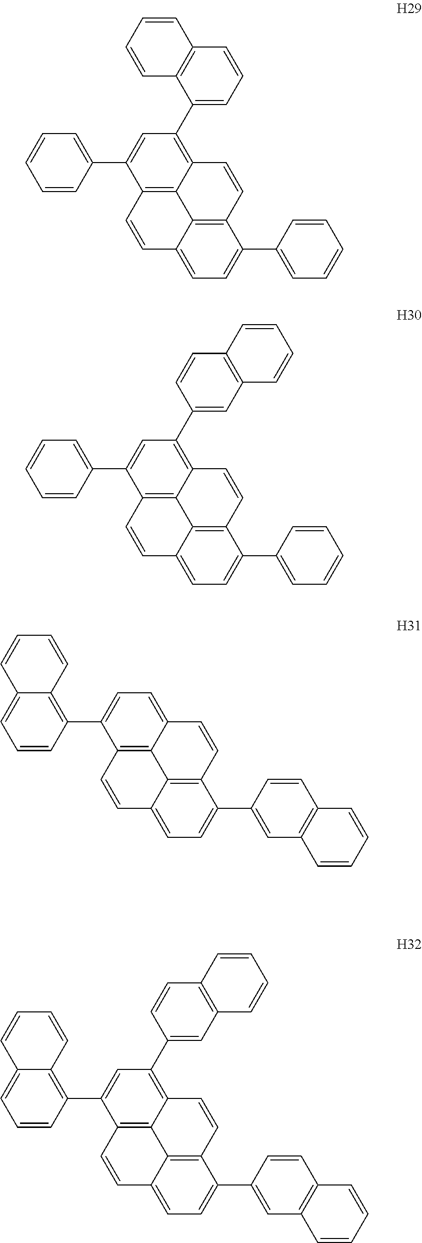

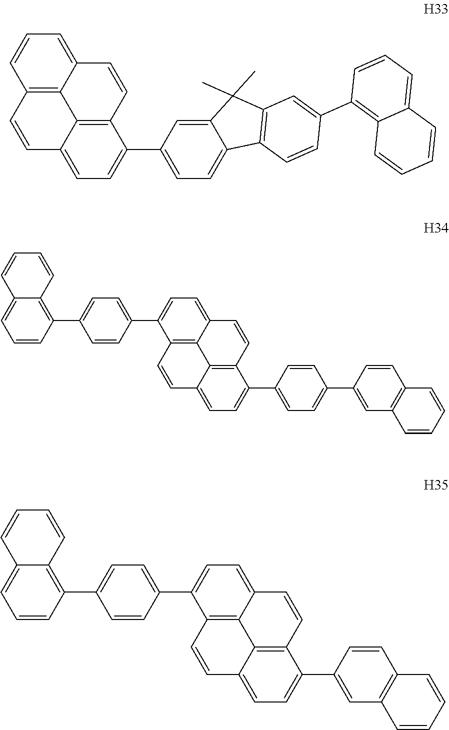

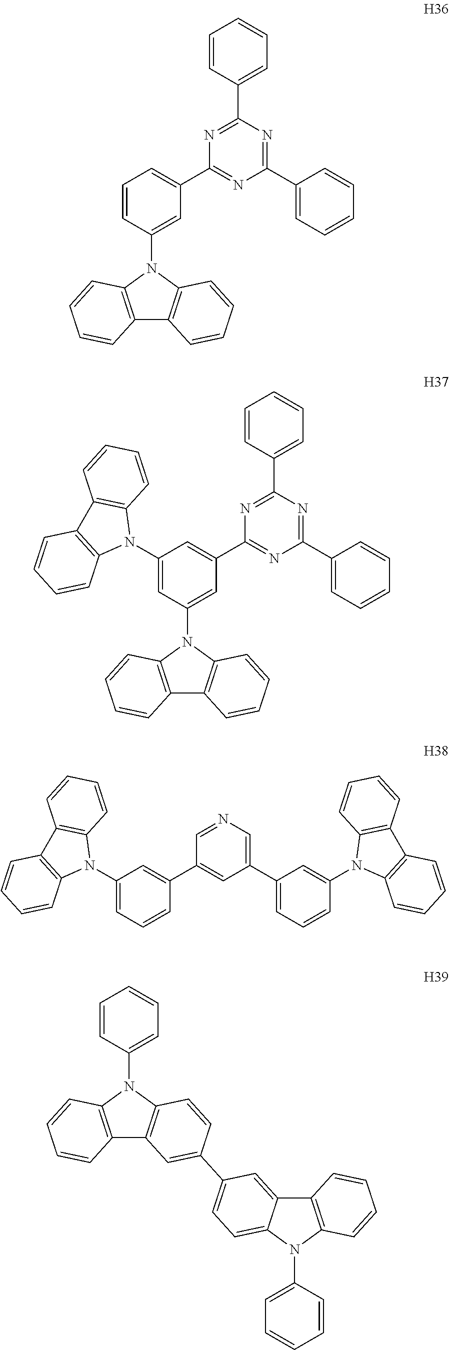 [SMILES: c1ccc(-c2nc(-c3ccccc3)nc(-c3cccc(-n4c5ccccc5c5ccccc54)c3)n2)cc1 ; c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1 ; c1ccc(-n2c3ccccc3c3cc(-c4ccc5c(c4)c4ccccc4n5-c4ccccc4)ccc32)cc1]

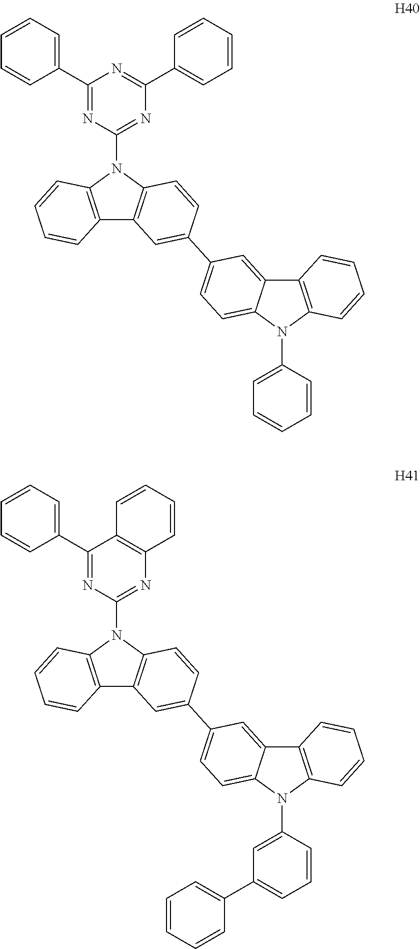 [SMILES: c1ccc(-c2nc(-c3ccccc3)nc(-n3c4ccccc4c4cc(-c5ccc6c(c5)c5ccccc5n6-c5ccccc5)ccc43)n2)cc1 ; c1ccc(-c2cccc(-n3c4ccccc4c4cc(-c5ccc6c(c5)c5ccccc5n6-c5nc(-c6ccccc6)c6ccccc6n5)ccc43)c2)cc1]

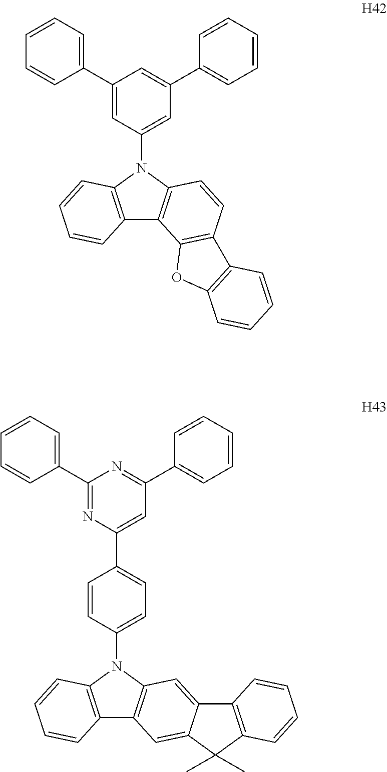 [SMILES: c1ccc(-c2cc(-c3ccccc3)cc(-n3c4ccccc4c4c5oc6ccccc6c5ccc43)c2)cc1 ; CC1(C)c2ccccc2-c2cc3c(cc21)c1ccccc1n3-c1ccc(-c2cc(-c3ccccc3)nc(-c3ccccc3)n2)cc1]

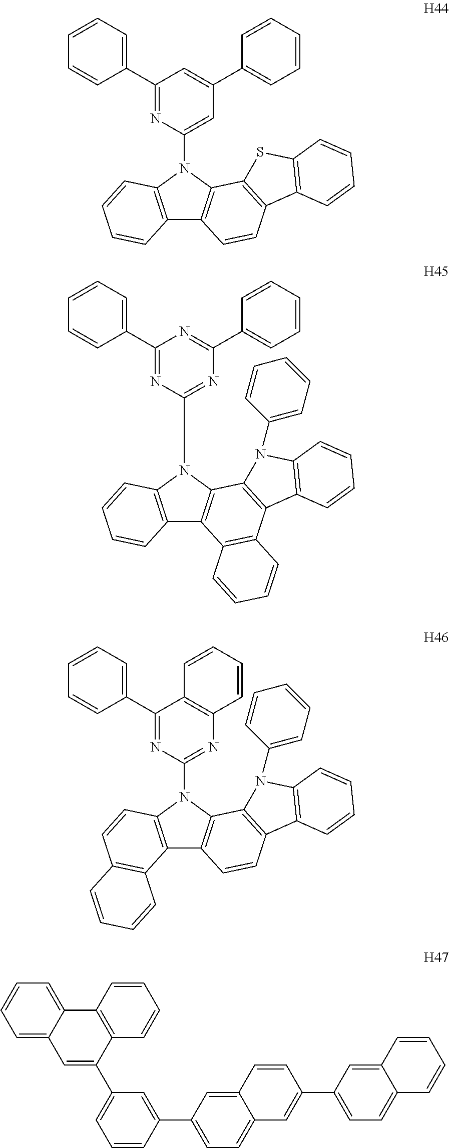

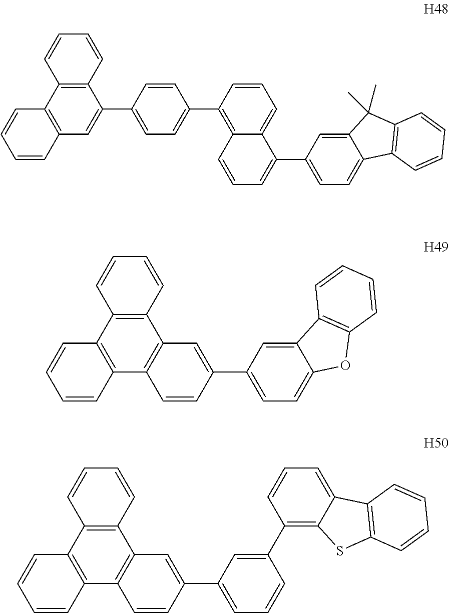

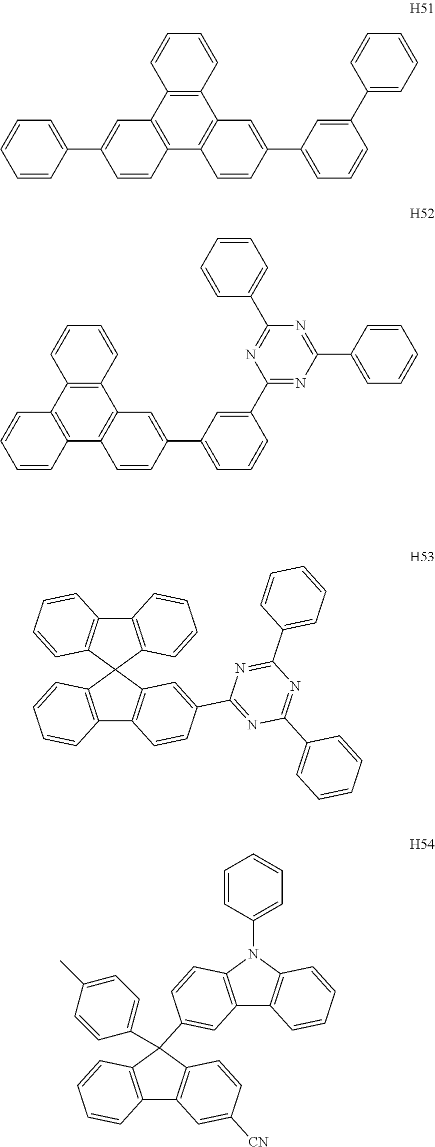

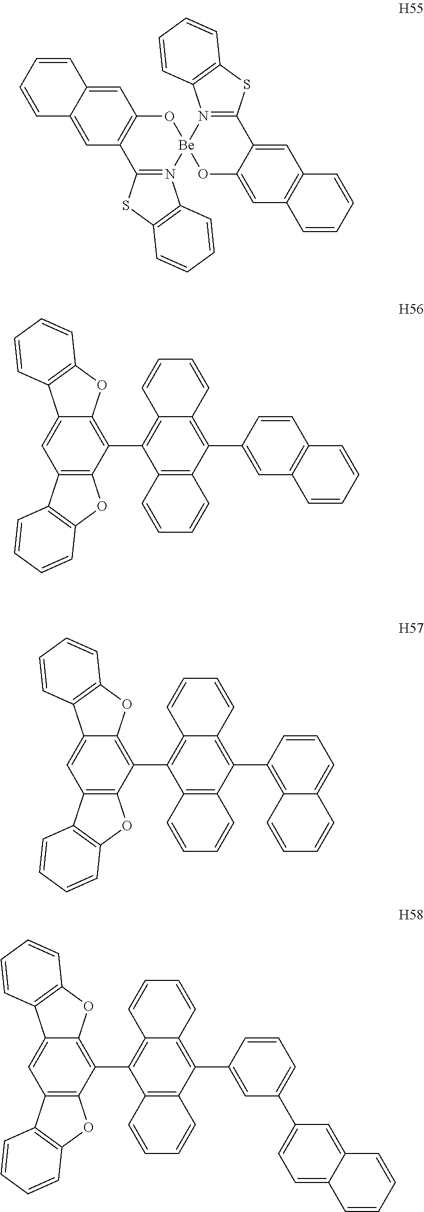

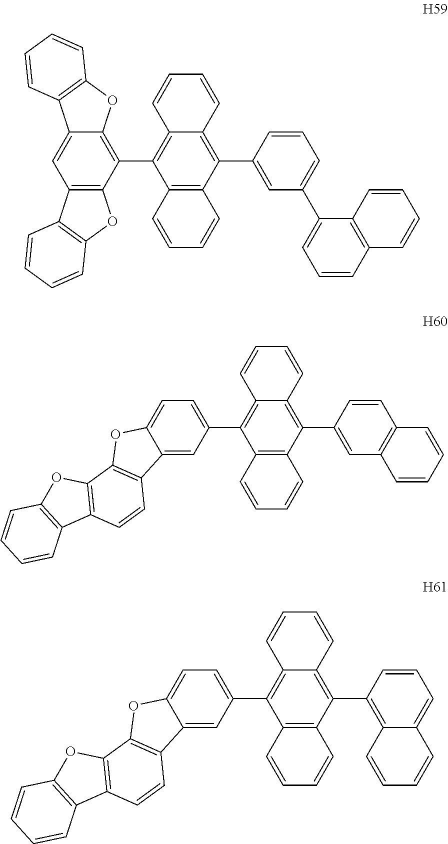 [SMILES: c1cc(-c2cccc3ccccc23)cc(-c2c3ccccc3c(-c3c4oc5ccccc5c4cc4c3oc3ccccc34)c3ccccc23)c1 ; c1ccc2cc(-c3c4ccccc4c(-c4ccc5oc6c(ccc7c8ccccc8oc76)c5c4)c4ccccc34)ccc2c1 ; c1ccc2c(-c3c4ccccc4c(-c4ccc5oc6c(ccc7c8ccccc8oc76)c5c4)c4ccccc34)cccc2c1]

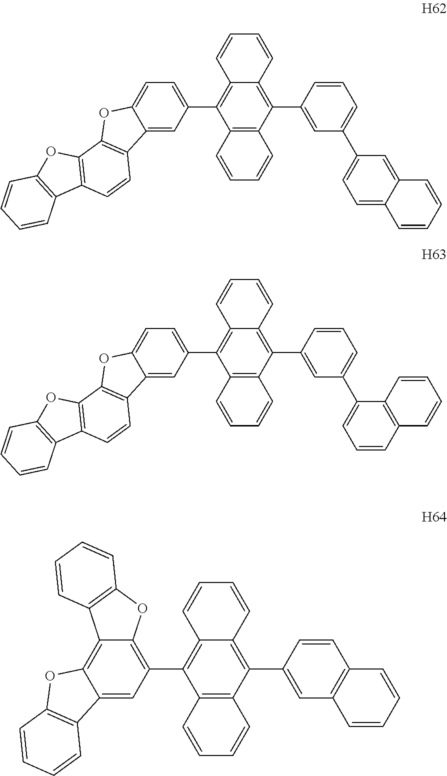 [SMILES: c1cc(-c2ccc3ccccc3c2)cc(-c2c3ccccc3c(-c3ccc4oc5c(ccc6c7ccccc7oc65)c4c3)c3ccccc23)c1 ; c1cc(-c2cccc3ccccc23)cc(-c2c3ccccc3c(-c3ccc4oc5c(ccc6c7ccccc7oc65)c4c3)c3ccccc23)c1 ; c1ccc2cc(-c3c4ccccc4c(-c4cc5c6ccccc6oc5c5c4oc4ccccc45)c4ccccc34)ccc2c1]

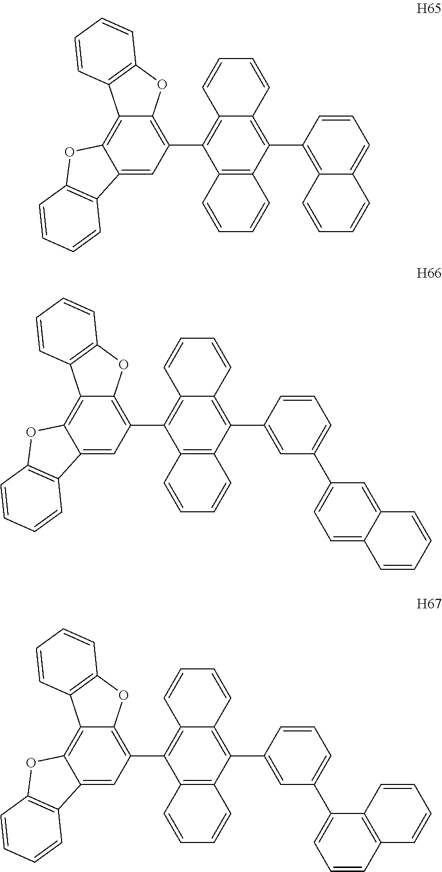 [SMILES: c1ccc2c(-c3c4ccccc4c(-c4cc5c6ccccc6oc5c5c4oc4ccccc45)c4ccccc34)cccc2c1 ; c1cc(-c2ccc3ccccc3c2)cc(-c2c3ccccc3c(-c3cc4c5ccccc5oc4c4c3oc3ccccc34)c3ccccc23)c1 ; c1cc(-c2cccc3ccccc23)cc(-c2c3ccccc3c(-c3cc4c5ccccc5oc4c4c3oc3ccccc34)c3ccccc23)c1]

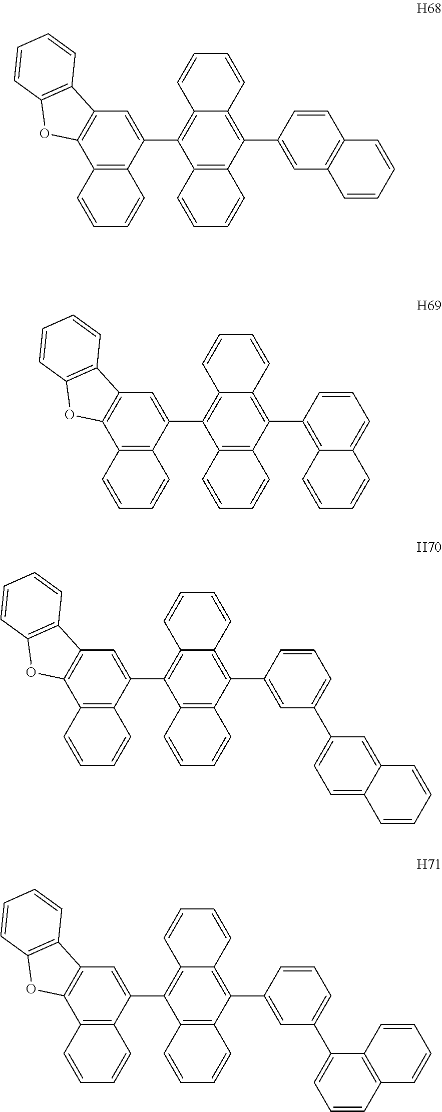 [SMILES: c1ccc2cc(-c3c4ccccc4c(-c4cc5c6ccccc6oc5c5ccccc45)c4ccccc34)ccc2c1 ; c1ccc2c(-c3c4ccccc4c(-c4cc5c6ccccc6oc5c5ccccc45)c4ccccc34)cccc2c1 ; c1cc(-c2ccc3ccccc3c2)cc(-c2c3ccccc3c(-c3cc4c5ccccc5oc4c4ccccc34)c3ccccc23)c1 ; c1cc(-c2cccc3ccccc23)cc(-c2c3ccccc3c(-c3cc4c5ccccc5oc4c4ccccc34)c3ccccc23)c1]

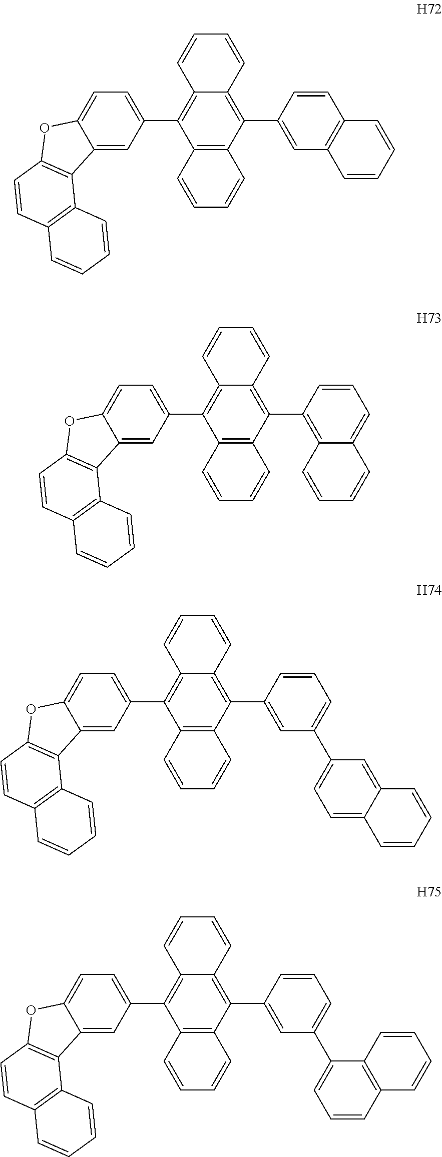 [SMILES: c1ccc2cc(-c3c4ccccc4c(-c4ccc5oc6ccc7ccccc7c6c5c4)c4ccccc34)ccc2c1 ; c1ccc2c(-c3c4ccccc4c(-c4ccc5oc6ccc7ccccc7c6c5c4)c4ccccc34)cccc2c1 ; c1cc(-c2ccc3ccccc3c2)cc(-c2c3ccccc3c(-c3ccc4oc5ccc6ccccc6c5c4c3)c3ccccc23)c1 ; c1cc(-c2cccc3ccccc23)cc(-c2c3ccccc3c(-c3ccc4oc5ccc6ccccc6c5c4c3)c3ccccc23)c1]

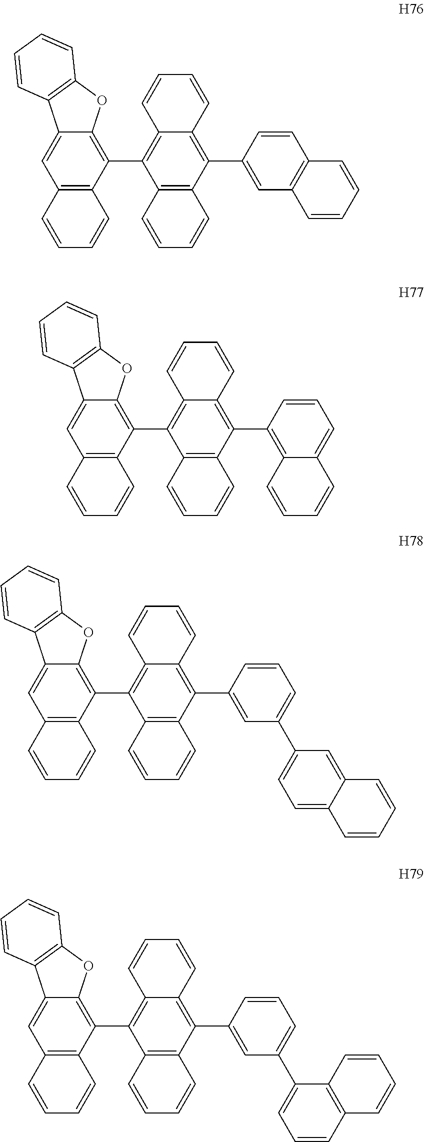 [SMILES: c1ccc2cc(-c3c4ccccc4c(-c4c5ccccc5cc5c4oc4ccccc45)c4ccccc34)ccc2c1 ; c1ccc2c(-c3c4ccccc4c(-c4c5ccccc5cc5c4oc4ccccc45)c4ccccc34)cccc2c1 ; c1cc(-c2ccc3ccccc3c2)cc(-c2c3ccccc3c(-c3c4ccccc4cc4c3oc3ccccc34)c3ccccc23)c1 ; c1cc(-c2cccc3ccccc23)cc(-c2c3ccccc3c(-c3c4ccccc4cc4c3oc3ccccc34)c3ccccc23)c1]

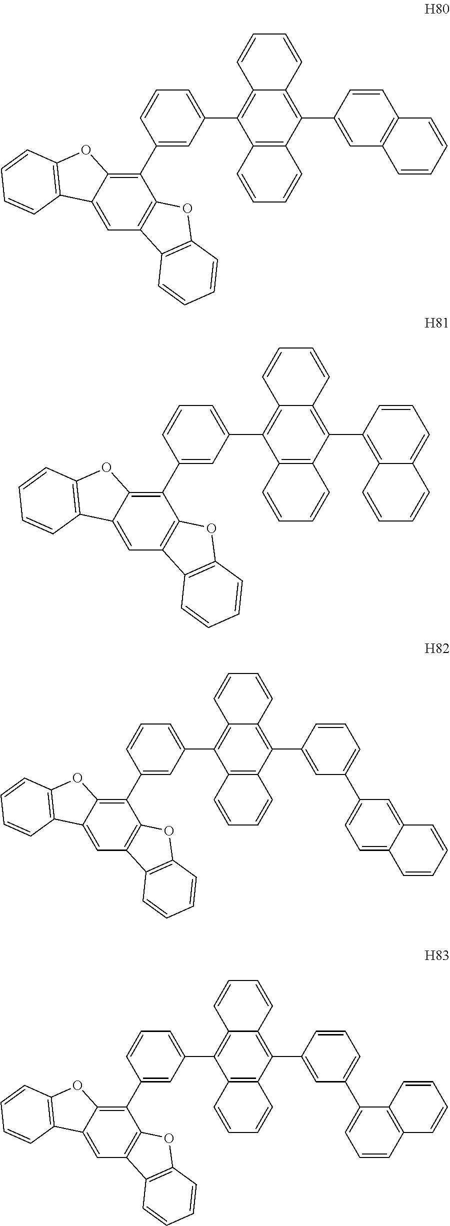 [SMILES: c1cc(-c2c3ccccc3c(-c3ccc4ccccc4c3)c3ccccc23)cc(-c2c3oc4ccccc4c3cc3c2oc2ccccc23)c1 ; c1cc(-c2c3ccccc3c(-c3cccc4ccccc34)c3ccccc23)cc(-c2c3oc4ccccc4c3cc3c2oc2ccccc23)c1 ; c1cc(-c2ccc3ccccc3c2)cc(-c2c3ccccc3c(-c3cccc(-c4c5oc6ccccc6c5cc5c4oc4ccccc45)c3)c3ccccc23)c1 ; c1cc(-c2cccc3ccccc23)cc(-c2c3ccccc3c(-c3cccc(-c4c5oc6ccccc6c5cc5c4oc4ccccc45)c3)c3ccccc23)c1]

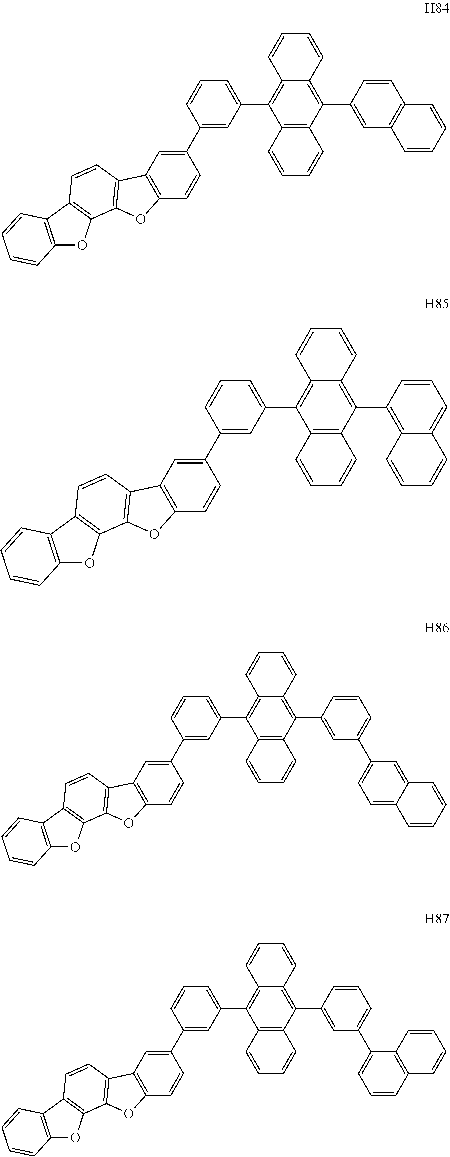 [SMILES: c1cc(-c2ccc3oc4c(ccc5c6ccccc6oc54)c3c2)cc(-c2c3ccccc3c(-c3ccc4ccccc4c3)c3ccccc23)c1 ; c1cc(-c2ccc3oc4c(ccc5c6ccccc6oc54)c3c2)cc(-c2c3ccccc3c(-c3cccc4ccccc34)c3ccccc23)c1 ; c1cc(-c2ccc3ccccc3c2)cc(-c2c3ccccc3c(-c3cccc(-c4ccc5oc6c(ccc7c8ccccc8oc76)c5c4)c3)c3ccccc23)c1 ; c1cc(-c2ccc3oc4c(ccc5c6ccccc6oc54)c3c2)cc(-c2c3ccccc3c(-c3cccc(-c4cccc5ccccc45)c3)c3ccccc23)c1]

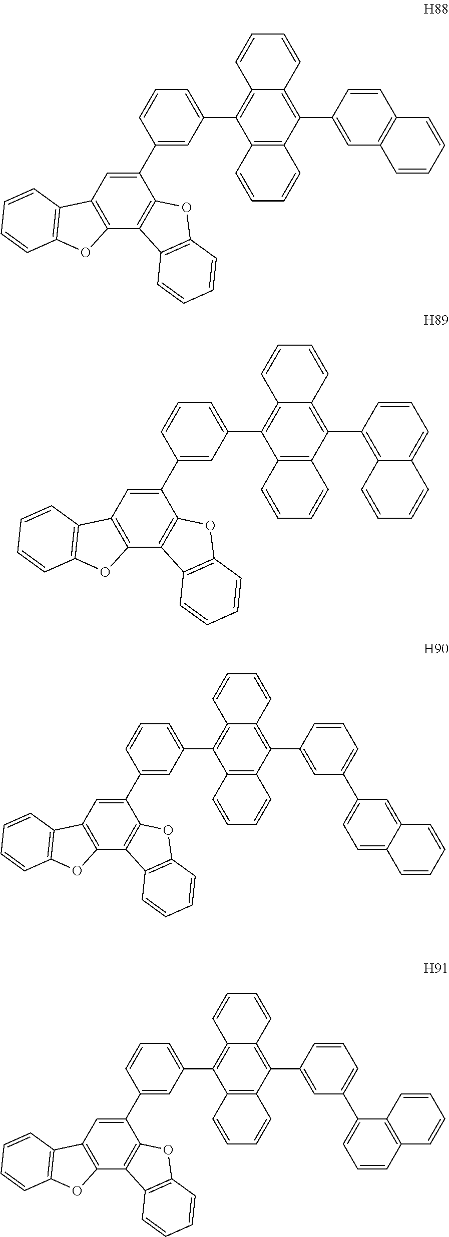 [SMILES: c1cc(-c2c3ccccc3c(-c3ccc4ccccc4c3)c3ccccc23)cc(-c2cc3c4ccccc4oc3c3c2oc2ccccc23)c1 ; c1cc(-c2c3ccccc3c(-c3cccc4ccccc34)c3ccccc23)cc(-c2cc3c4ccccc4oc3c3c2oc2ccccc23)c1 ; c1cc(-c2ccc3ccccc3c2)cc(-c2c3ccccc3c(-c3cccc(-c4cc5c6ccccc6oc5c5c4oc4ccccc45)c3)c3ccccc23)c1 ; c1cc(-c2cccc3ccccc23)cc(-c2c3ccccc3c(-c3cccc(-c4cc5c6ccccc6oc5c5c4oc4ccccc45)c3)c3ccccc23)c1]

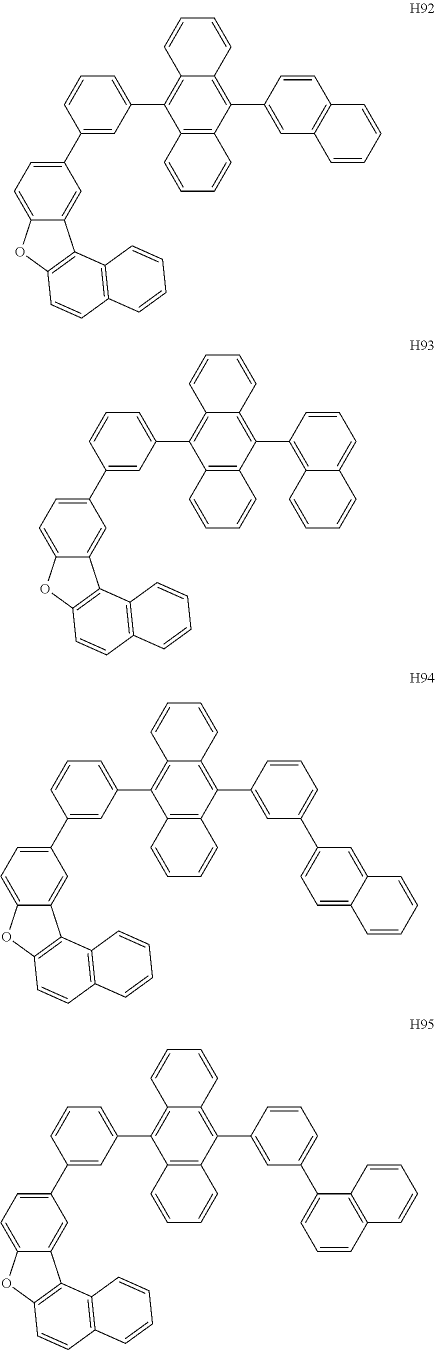 [SMILES: c1cc(-c2ccc3oc4ccc5ccccc5c4c3c2)cc(-c2c3ccccc3c(-c3ccc4ccccc4c3)c3ccccc23)c1 ; c1cc(-c2ccc3oc4ccc5ccccc5c4c3c2)cc(-c2c3ccccc3c(-c3cccc4ccccc34)c3ccccc23)c1 ; c1cc(-c2ccc3ccccc3c2)cc(-c2c3ccccc3c(-c3cccc(-c4ccc5oc6ccc7ccccc7c6c5c4)c3)c3ccccc23)c1 ; c1cc(-c2ccc3oc4ccc5ccccc5c4c3c2)cc(-c2c3ccccc3c(-c3cccc(-c4cccc5ccccc45)c3)c3ccccc23)c1]

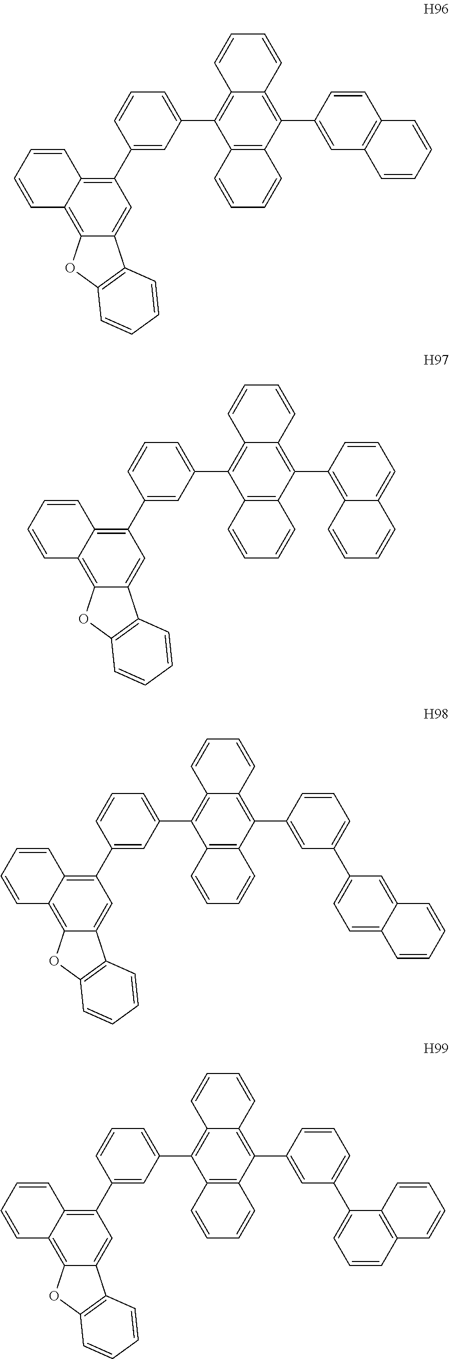 [SMILES: c1cc(-c2c3ccccc3c(-c3ccc4ccccc4c3)c3ccccc23)cc(-c2cc3c4ccccc4oc3c3ccccc23)c1 ; c1cc(-c2c3ccccc3c(-c3cccc4ccccc34)c3ccccc23)cc(-c2cc3c4ccccc4oc3c3ccccc23)c1 ; c1cc(-c2ccc3ccccc3c2)cc(-c2c3ccccc3c(-c3cccc(-c4cc5c6ccccc6oc5c5ccccc45)c3)c3ccccc23)c1 ; c1cc(-c2cccc3ccccc23)cc(-c2c3ccccc3c(-c3cccc(-c4cc5c6ccccc6oc5c5ccccc45)c3)c3ccccc23)c1]

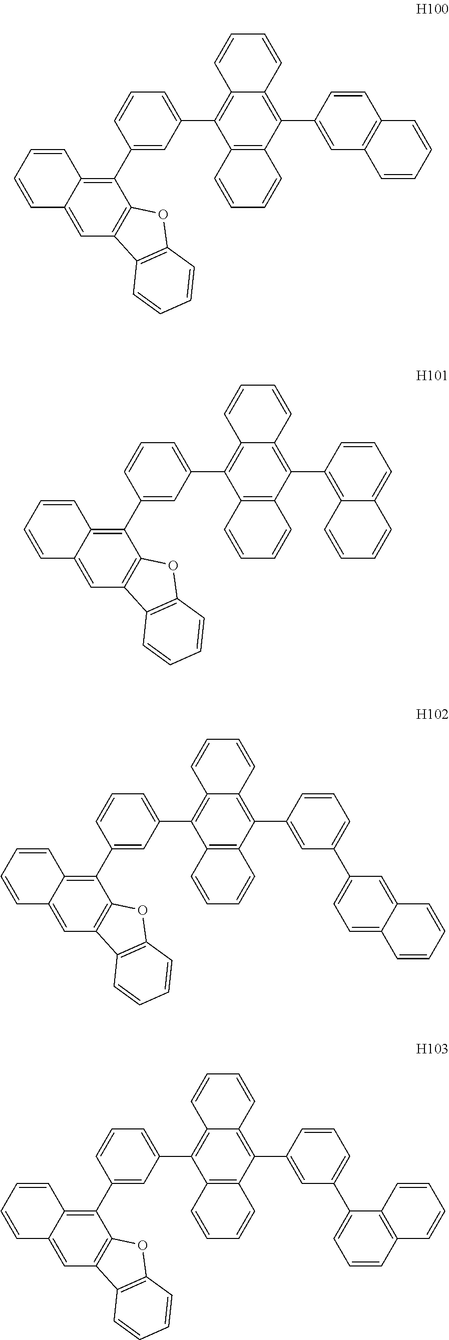 [SMILES: c1cc(-c2c3ccccc3c(-c3ccc4ccccc4c3)c3ccccc23)cc(-c2c3ccccc3cc3c2oc2ccccc23)c1 ; c1cc(-c2c3ccccc3c(-c3cccc4ccccc34)c3ccccc23)cc(-c2c3ccccc3cc3c2oc2ccccc23)c1 ; c1cc(-c2ccc3ccccc3c2)cc(-c2c3ccccc3c(-c3cccc(-c4c5ccccc5cc5c4oc4ccccc45)c3)c3ccccc23)c1 ; c1cc(-c2cccc3ccccc23)cc(-c2c3ccccc3c(-c3cccc(-c4c5ccccc5cc5c4oc4ccccc45)c3)c3ccccc23)c1]

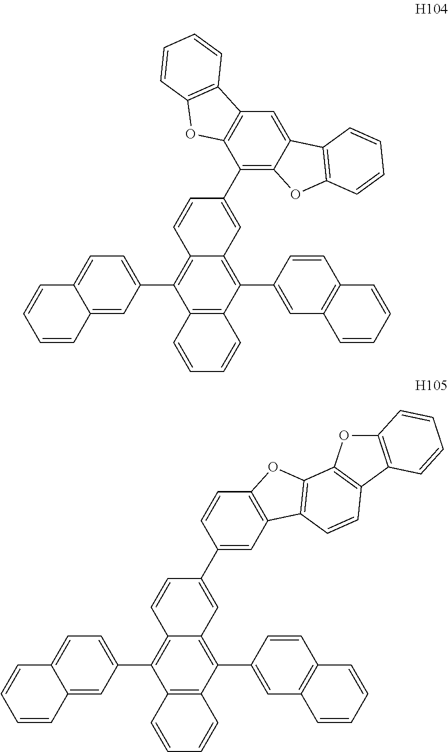 [SMILES: c1ccc2cc(-c3c4ccccc4c(-c4ccc5ccccc5c4)c4cc(-c5c6oc7ccccc7c6cc6c5oc5ccccc56)ccc34)ccc2c1 ; c1ccc2cc(-c3c4ccccc4c(-c4ccc5ccccc5c4)c4cc(-c5ccc6oc7c(ccc8c9ccccc9oc87)c6c5)ccc34)ccc2c1]

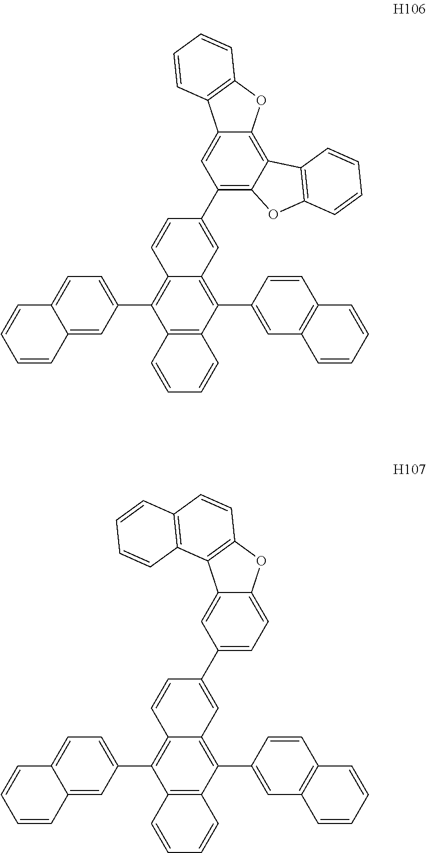 [SMILES: c1ccc2cc(-c3c4ccccc4c(-c4ccc5ccccc5c4)c4cc(-c5cc6c7ccccc7oc6c6c5oc5ccccc56)ccc34)ccc2c1 ; c1ccc2cc(-c3c4ccccc4c(-c4ccc5ccccc5c4)c4cc(-c5ccc6oc7ccc8ccccc8c7c6c5)ccc34)ccc2c1]

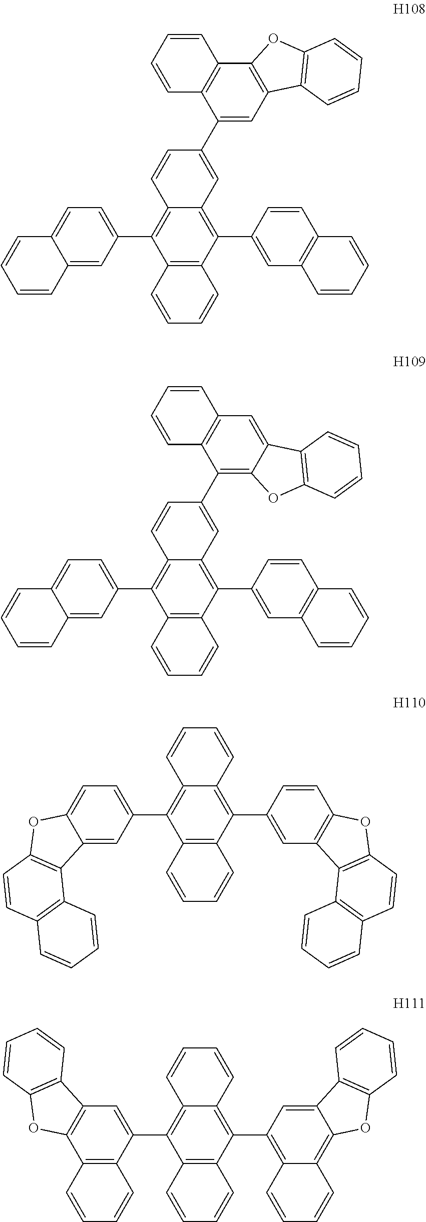 [SMILES: c1ccc2cc(-c3c4ccccc4c(-c4ccc5ccccc5c4)c4cc(-c5cc6c7ccccc7oc6c6ccccc56)ccc34)ccc2c1 ; c1ccc2cc(-c3c4ccccc4c(-c4ccc5ccccc5c4)c4cc(-c5c6ccccc6cc6c5oc5ccccc56)ccc34)ccc2c1 ; c1ccc2c(c1)ccc1oc3ccc(-c4c5ccccc5c(-c5ccc6oc7ccc8ccccc8c7c6c5)c5ccccc45)cc3c12 ; c1ccc2c(c1)oc1c3ccccc3c(-c3c4ccccc4c(-c4cc5c6ccccc6oc5c5ccccc45)c4ccccc34)cc21]

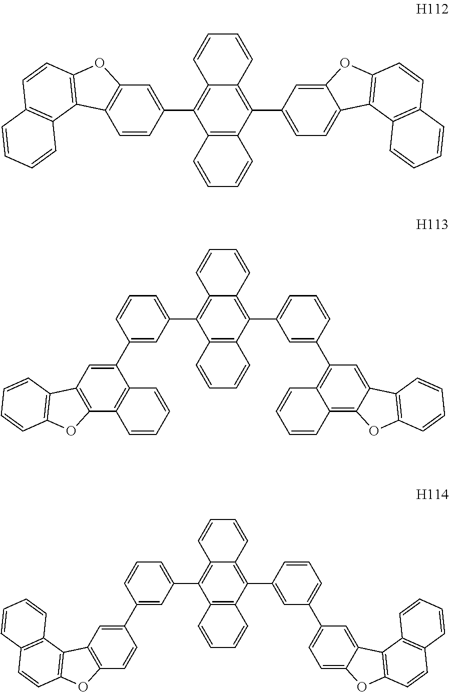 [SMILES: c1ccc2c(c1)ccc1oc3cc(-c4c5ccccc5c(-c5ccc6c(c5)oc5ccc7ccccc7c56)c5ccccc45)ccc3c12 ; c1cc(-c2c3ccccc3c(-c3cccc(-c4cc5c6ccccc6oc5c5ccccc45)c3)c3ccccc23)cc(-c2cc3c4ccccc4oc3c3ccccc23)c1 ; c1cc(-c2ccc3oc4ccc5ccccc5c4c3c2)cc(-c2c3ccccc3c(-c3cccc(-c4ccc5oc6ccc7ccccc7c6c5c4)c3)c3ccccc23)c1]

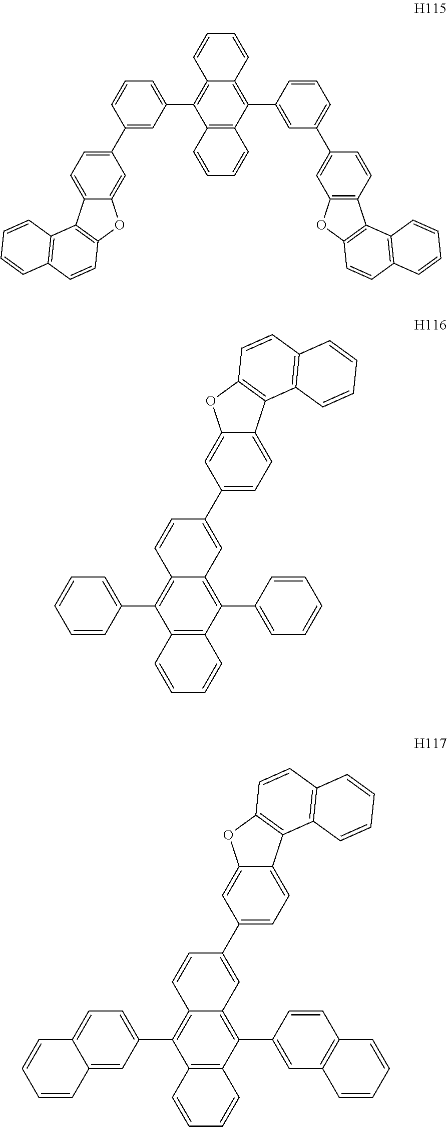 [SMILES: c1cc(-c2ccc3c(c2)oc2ccc4ccccc4c23)cc(-c2c3ccccc3c(-c3cccc(-c4ccc5c(c4)oc4ccc6ccccc6c45)c3)c3ccccc23)c1 ; c1ccc(-c2c3ccccc3c(-c3ccccc3)c3cc(-c4ccc5c(c4)oc4ccc6ccccc6c45)ccc23)cc1 ; c1ccc2cc(-c3c4ccccc4c(-c4ccc5ccccc5c4)c4cc(-c5ccc6c(c5)oc5ccc7ccccc7c56)ccc34)ccc2c1]

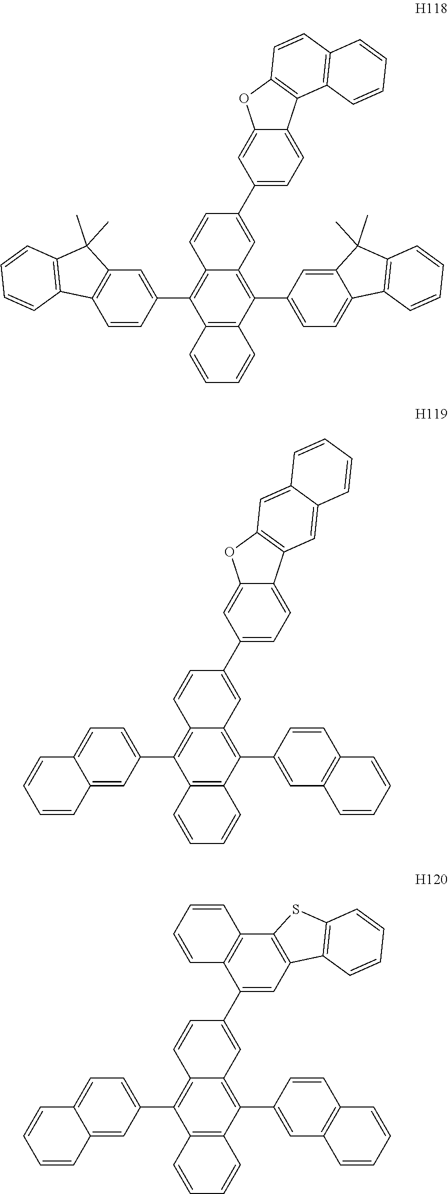 [SMILES: CC1(C)c2ccccc2-c2ccc(-c3c4ccccc4c(-c4ccc5c(c4)C(C)(C)c4ccccc4-5)c4cc(-c5ccc6c(c5)oc5ccc7ccccc7c56)ccc34)cc21 ; c1ccc2cc(-c3c4ccccc4c(-c4ccc5ccccc5c4)c4cc(-c5ccc6c(c5)oc5cc7ccccc7cc56)ccc34)ccc2c1 ; c1ccc2cc(-c3c4ccccc4c(-c4ccc5ccccc5c4)c4cc(-c5cc6c7ccccc7sc6c6ccccc56)ccc34)ccc2c1]

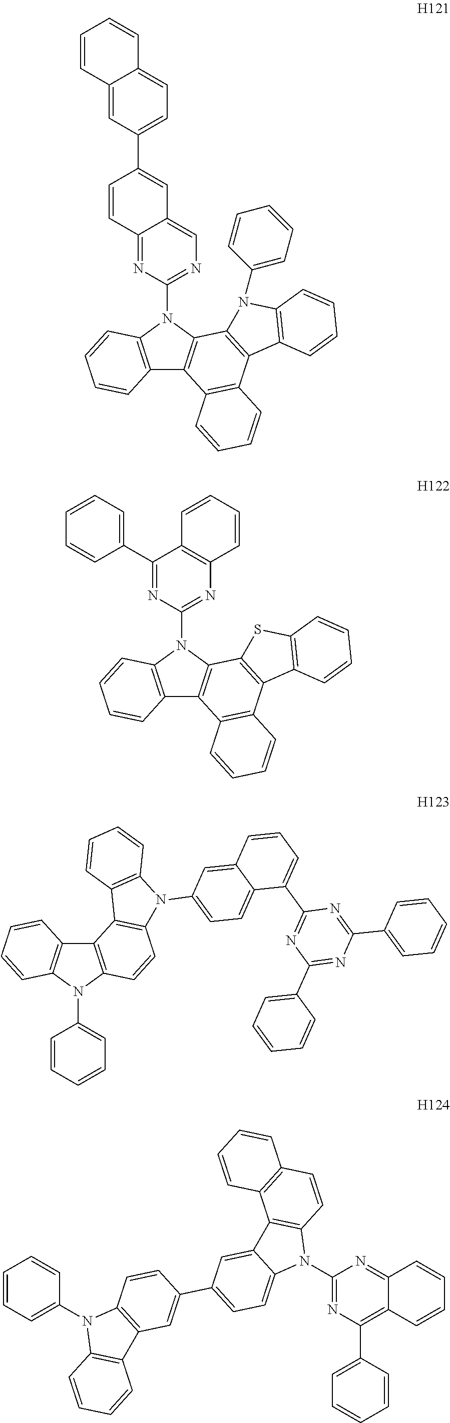 [SMILES: c1ccc(-n2c3ccccc3c3c4ccccc4c4c5ccccc5n(-c5ncc6cc(-c7ccc8ccccc8c7)ccc6n5)c4c32)cc1 ; c1ccc(-c2nc(-n3c4ccccc4c4c5ccccc5c5c6ccccc6sc5c43)nc3ccccc23)cc1 ; c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1]

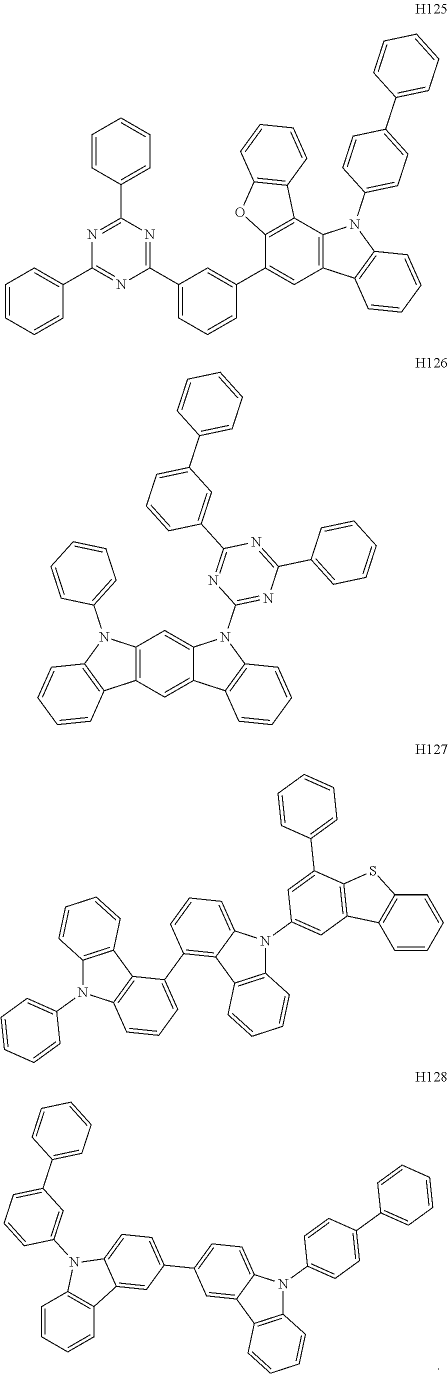

[Phosphorescent Dopant]

In embodiments, the phosphorescent dopant may include at least one transition metal as a central metal.

The phosphorescent dopant may include a monodentate ligand, a bidentate ligand, a tridentate ligand, a tetradentate ligand, a pentadentate ligand, a hexadentate ligand, or any combination thereof.

The phosphorescent dopant may be electrically neutral.

For example, the phosphorescent dopant may include an organometallic compound represented by Formula 401:

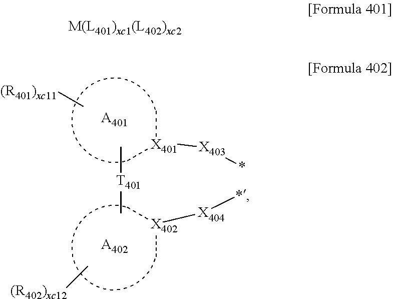

In Formulae 401 and 402,
M may be a transition metal (for example, iridium (Ir), platinum (Pt), palladium (Pd), osmium (Os), titanium (Ti), gold (Au), hafnium (Hf), europium (Eu), terbium (Tb), rhodium (Rh), rhenium (Re), or thulium (Tm)),
L₄₀₁ may be a ligand represented by Formula 402, and xc1 may be 1, 2, or 3, wherein when xc1 is two or more, two or more of L₄₀₁(s) may be identical to or different from each other,
L₄₀₂ may be an organic ligand, and xc2 may be 0, 1, 2, 3, or 4, and when xc2 is 2 or more, two or more of L₄₀₂(s) may be identical to or different from each other,
X₄₀₁ and X₄₀₂ may each independently be nitrogen or carbon,
ring A₄₀₁ and ring A₄₀₂ may each independently be a C₃-C₆₀ carbocyclic group or a C₁-C₆₀ heterocyclic group,
T₄₀₁ may be a single bond, *—O—*′, *—S—*′, *—C(═O)—*′, *—N(Q₄₁₁)-*′, *—C(Q₄₁₁)(Q₄₁₂)—*′, *′C(Q₄₁₁)═C(Q₄₁₂)—*′, *′C(Q₄₁₁)=*′, or *═C═*′,
X₄₀₃ and X₄₀₄ may each independently be a chemical bond (for example, a covalent bond or a coordination bond), O, S, N(Q₄₁₃), B(Q₄₁₃), P(Q₄₁₃), C(Q₄₁₃)(Q₄₁₄), or Si(Q₄₁₃)(Q₄₁₄),
Q₄₁₁ to Q₄₁₄ may each independently be the same as described herein with respect to Q₁,
R₄₀₁ and R₄₀₂ may each independently be hydrogen, deuterium, —F, —Cl, —Br, —I, a hydroxyl group, a cyano group, a nitro group, a C₁-C₂₀ alkyl group unsubstituted or substituted with at least one R₁₀ₐ, a C₁-C₂₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁ 0a, —Si(Q₄₀₁)(Q₄₀₂)(Q₄₀₃), —N(Q₄₀₁)(Q₄₀₂), —B(Q₄₀₁)(Q₄₀₂), —C(═O)(Q₄₀₁), —S(═O)₂(Q₄₀₁), or —P(═O)(Q₄₀₁)(Q₄₀₂),
Q₄₀₁ to Q₄₀₃ may each independently be the same as described herein with respect to Q₁,
xc11 and xc12 may each independently be an integer from 0 to 10, and
* and *′ in Formula 402 may each indicate a binding site to M in Formula 401.

In an embodiment, in Formula 402, X₄₀₁ may be nitrogen, and X₄₀₂ may be carbon, or X₄₀₁ and X₄₀₂ may each be nitrogen.

In embodiments, in Formula 402, when xc1 402 is 2 or more, two ring A₄₀₁(s) in two or more of L₄₀₁(s) may be optionally linked to each other via T₄₀₂, which is a linking group, or two ring A₄₀₂(s) may be optionally linked to each other via T₄₀₃, which is a linking group (see Compounds PD1 to PD4 and PD7). T₄₀₂ and T₄₀₃ may each be the same as described herein with respect to T₄₀₁.

In Formula 401, L₄₀₂ may be an organic ligand. For example, L₄₀₂ may include a halogen group, a diketone group (for example, an acetylacetonate group), a carboxylic acid group (for example, a picolinate group), —C(═O), an isonitrile group, —CN group, a phosphorus group (for example, a phosphine group, a phosphite group, etc.), or any combination thereof.

The phosphorescent dopant may include, for example, one of Compounds PD1 to PD39, or any combination thereof:

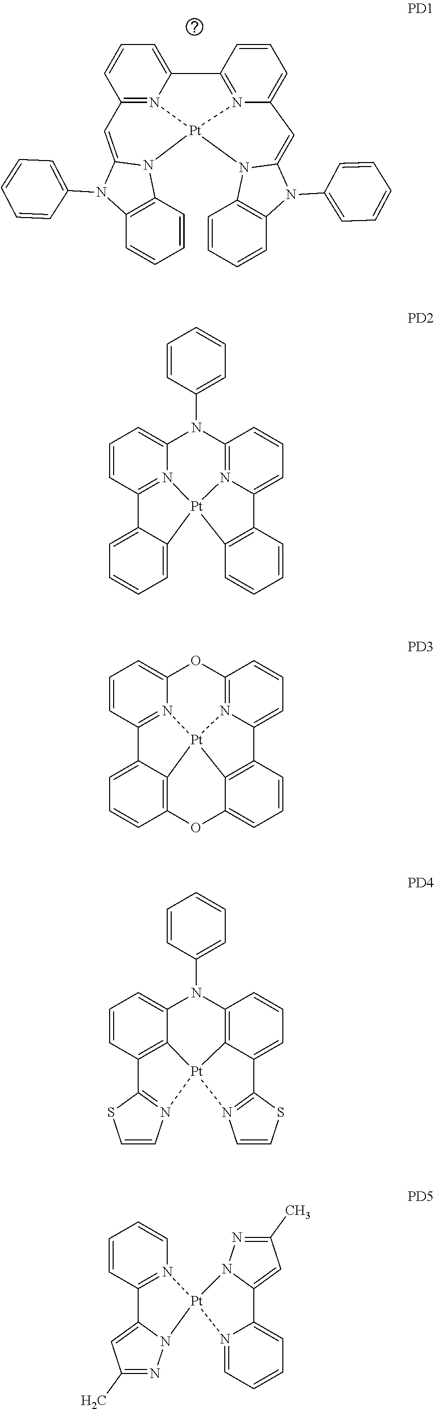

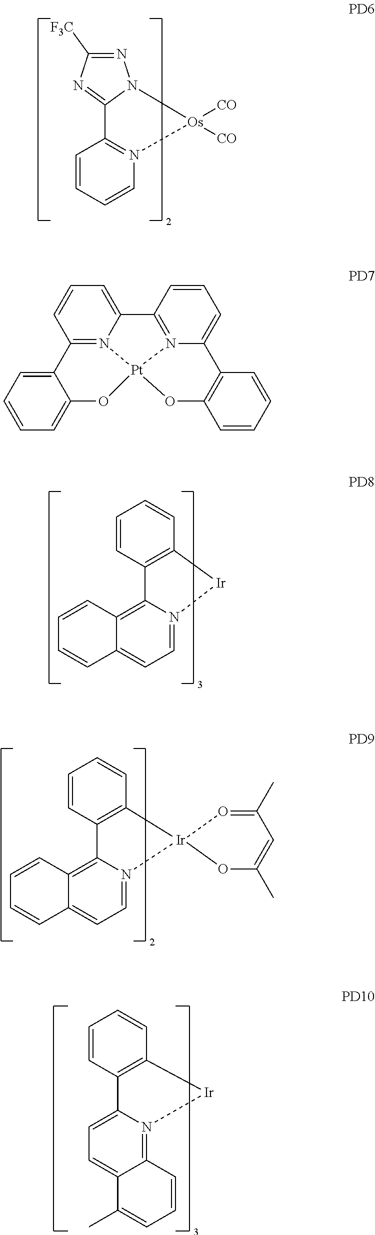

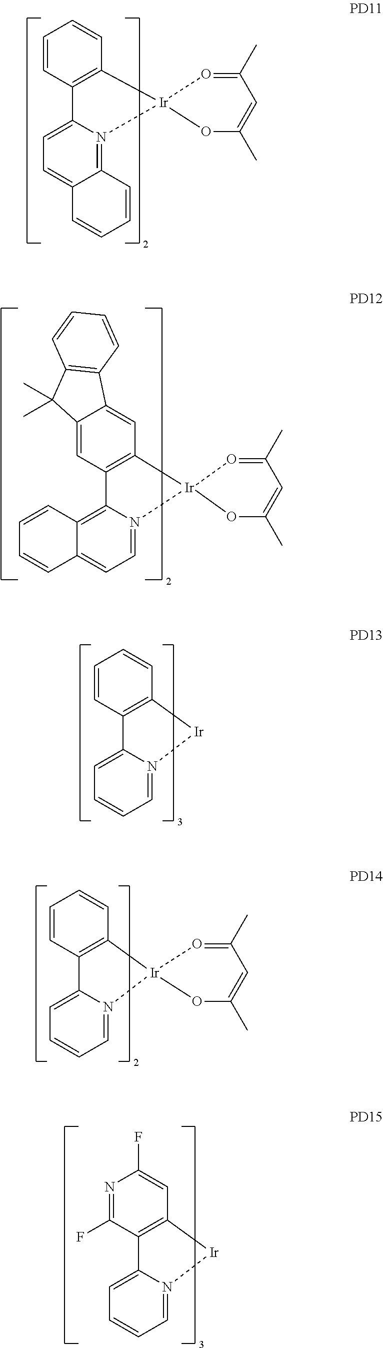

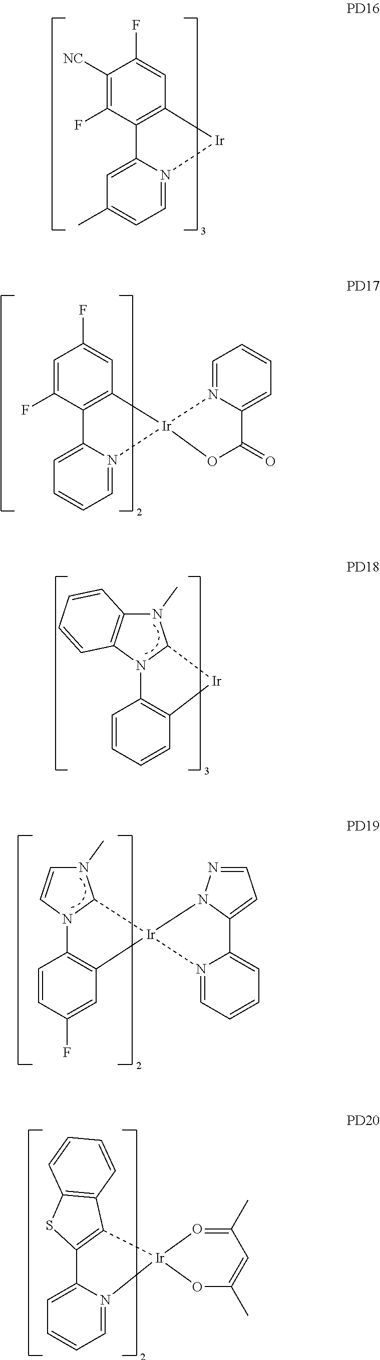

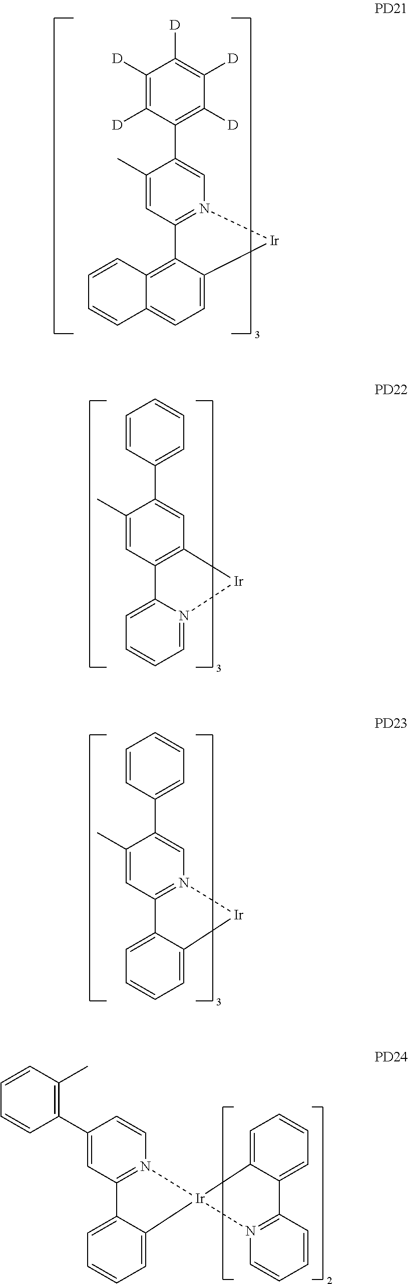

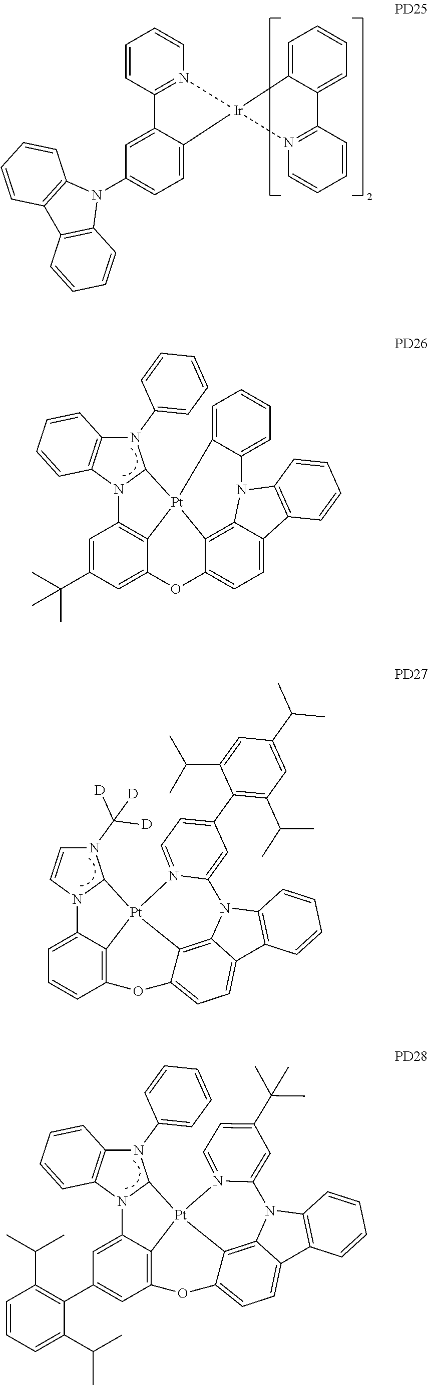

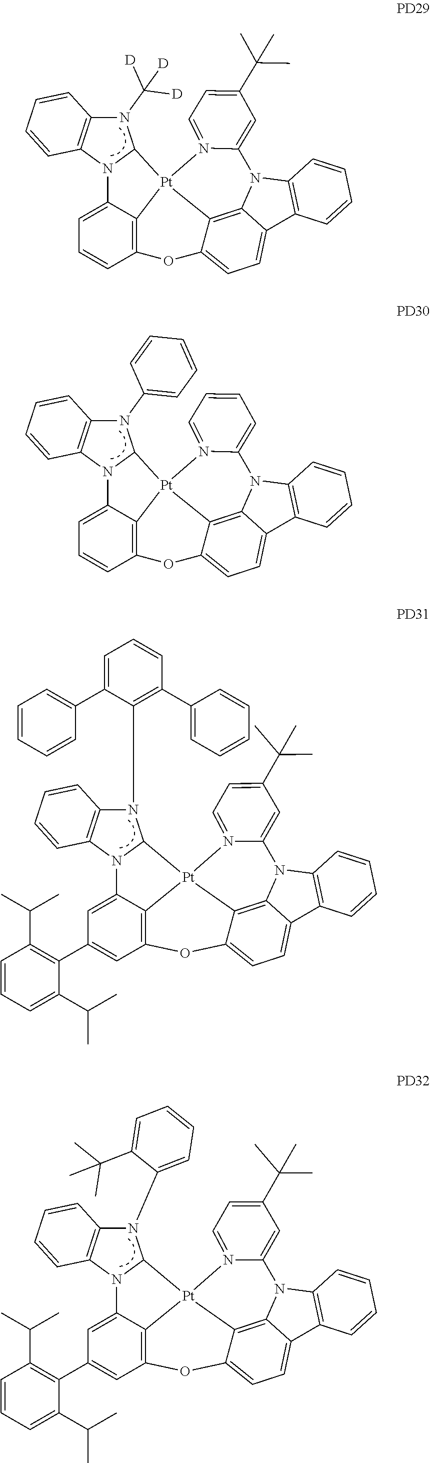

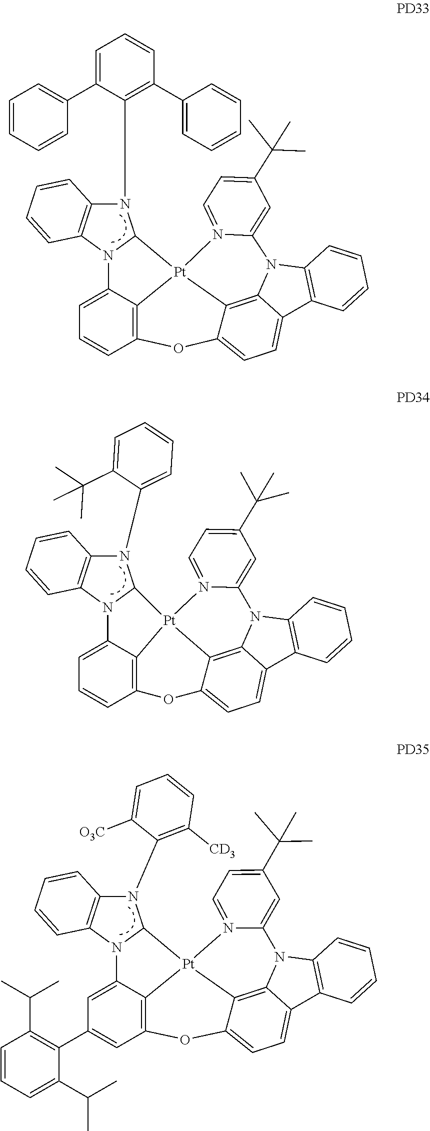

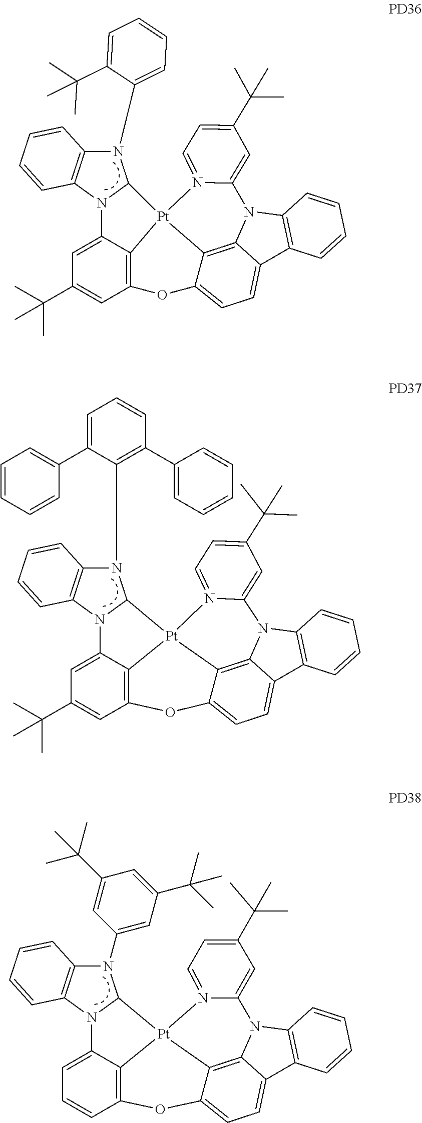

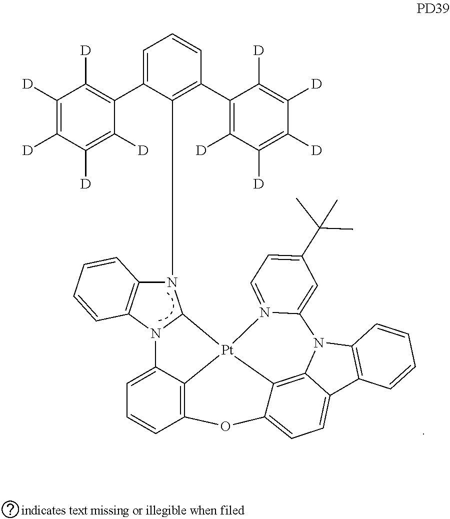

[Fluorescent Dopant]

The fluorescent dopant may include an amine group-containing compound, a styryl group-containing compound, or any combination thereof:

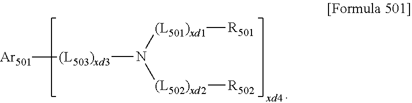

In Formula 501,
Ar₅₀₁, L₅₀₁ to L₅₀₃, R₅₀₁, and R₅₀₂ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xd1 to xd3 may each independently be 0, 1, 2, or 3, and
xd4 may be 1, 2, 3, 4, 5, or 6.

In an embodiment, in Formula 501, Ar₅₀₁ may be a condensed cyclic group (for example, an anthracene group, a chrysene group, or a pyrene group) in which three or more monocyclic groups are condensed together.

In embodiments, in Formula 501, xd4 may be 2.

In an embodiment, the fluorescent dopant may include: one of Compounds FD1 to FD37; DPVBi; DPAVBi; or any combination thereof:

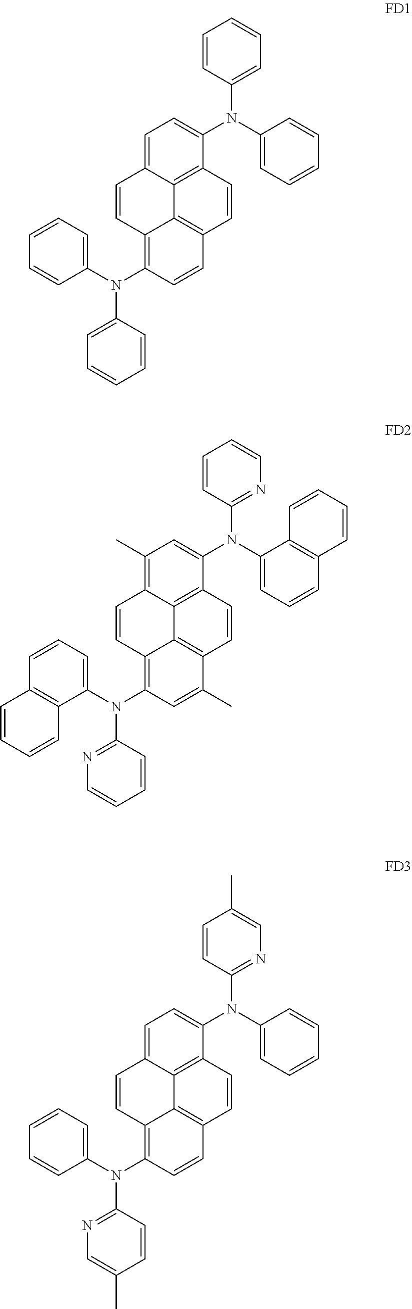 [SMILES: c1ccc(N(c2ccccc2)c2ccc3ccc4c(N(c5ccccc5)c5ccccc5)ccc5ccc2c3c54)cc1 ; Cc1cc(N(c2ccccn2)c2cccc3ccccc23)c2ccc3c(C)cc(N(c4ccccn4)c4cccc5ccccc45)c4ccc1c2c34 ; Cc1ccc(N(c2ccccc2)c2ccc3ccc4c(N(c5ccccc5)c5ccc(C)cn5)ccc5ccc2c3c54)nc1]

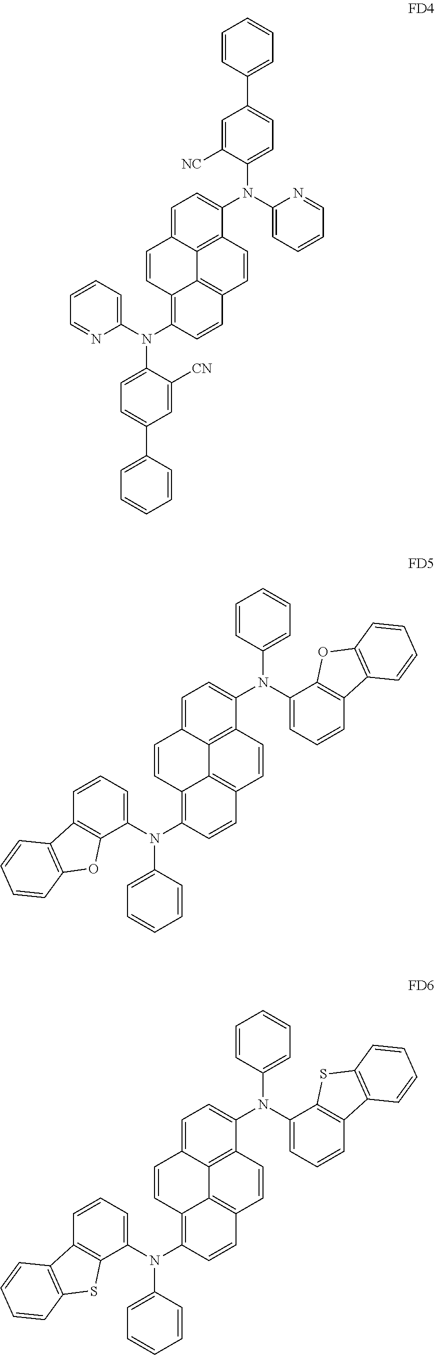 [SMILES: N#Cc1cc(-c2ccccc2)ccc1N(c1ccccn1)c1ccc2ccc3c(N(c4ccccn4)c4ccc(-c5ccccc5)cc4C#N)ccc4ccc1c2c43 ; c1ccc(N(c2ccc3ccc4c(N(c5ccccc5)c5cccc6c5oc5ccccc56)ccc5ccc2c3c54)c2cccc3c2oc2ccccc23)cc1 ; c1ccc(N(c2ccc3ccc4c(N(c5ccccc5)c5cccc6c5sc5ccccc56)ccc5ccc2c3c54)c2cccc3c2sc2ccccc23)cc1]

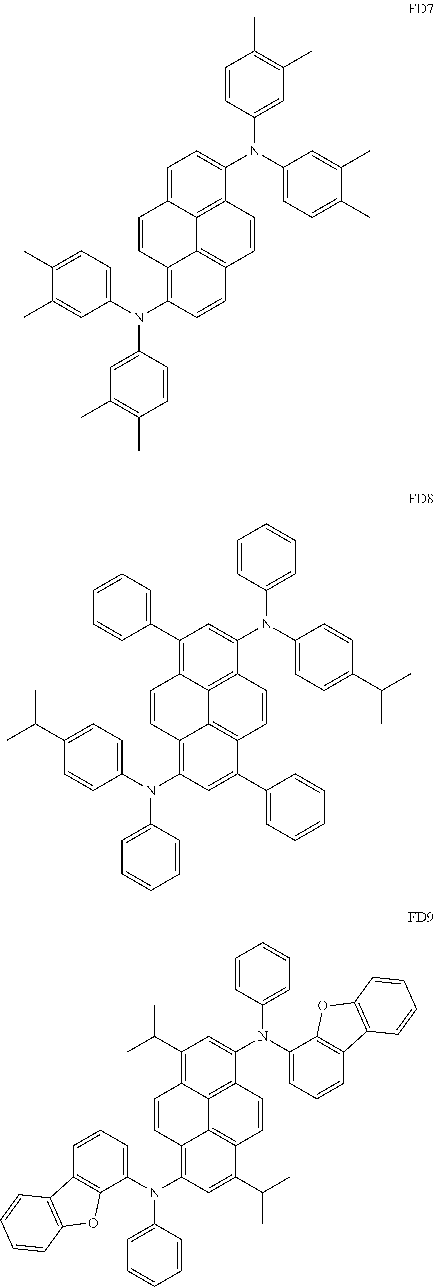

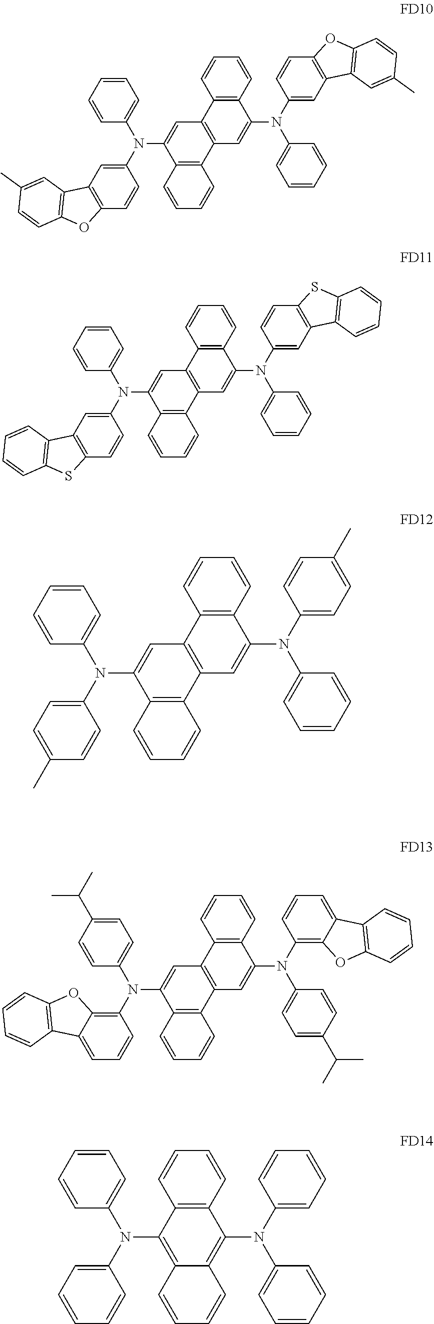

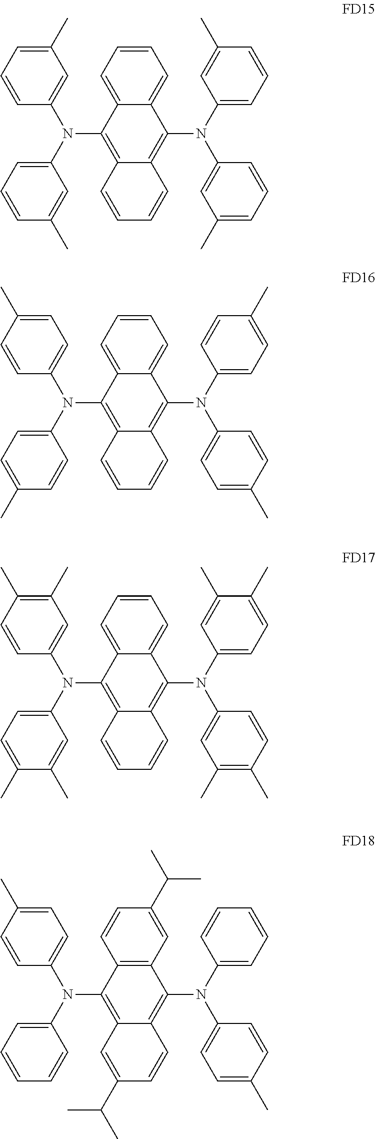 [SMILES: Cc1cccc(N(c2cccc(C)c2)c2c3ccccc3c(N(c3cccc(C)c3)c3cccc(C)c3)c3ccccc23)c1 ; Cc1ccc(N(c2ccc(C)cc2)c2c3ccccc3c(N(c3ccc(C)cc3)c3ccc(C)cc3)c3ccccc23)cc1 ; Cc1ccc(N(c2ccc(C)c(C)c2)c2c3ccccc3c(N(c3ccc(C)c(C)c3)c3ccc(C)c(C)c3)c3ccccc23)cc1C ; Cc1ccc(N(c2ccccc2)c2c3ccc(C(C)C)cc3c(N(c3ccccc3)c3ccc(C)cc3)c3ccc(C(C)C)cc23)cc1]

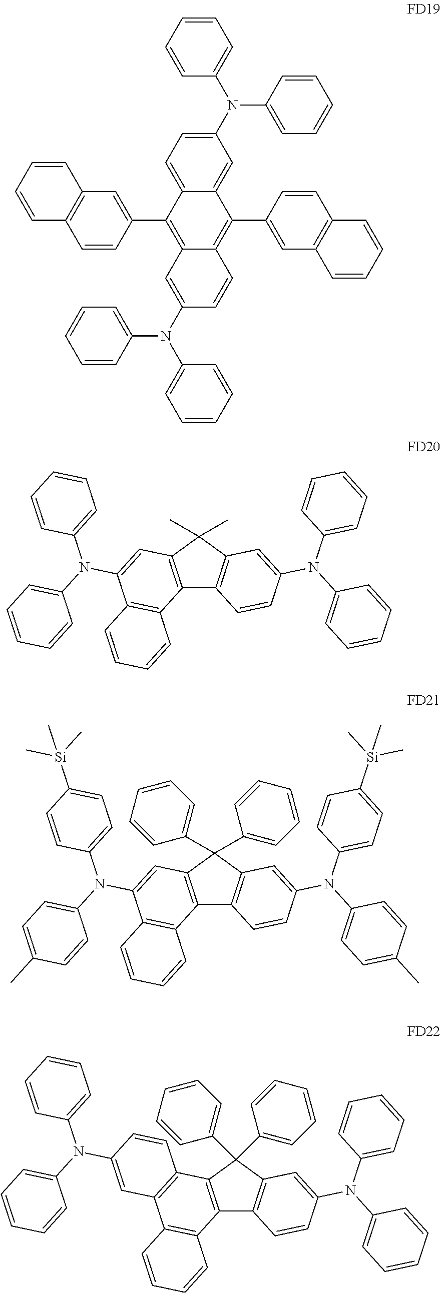

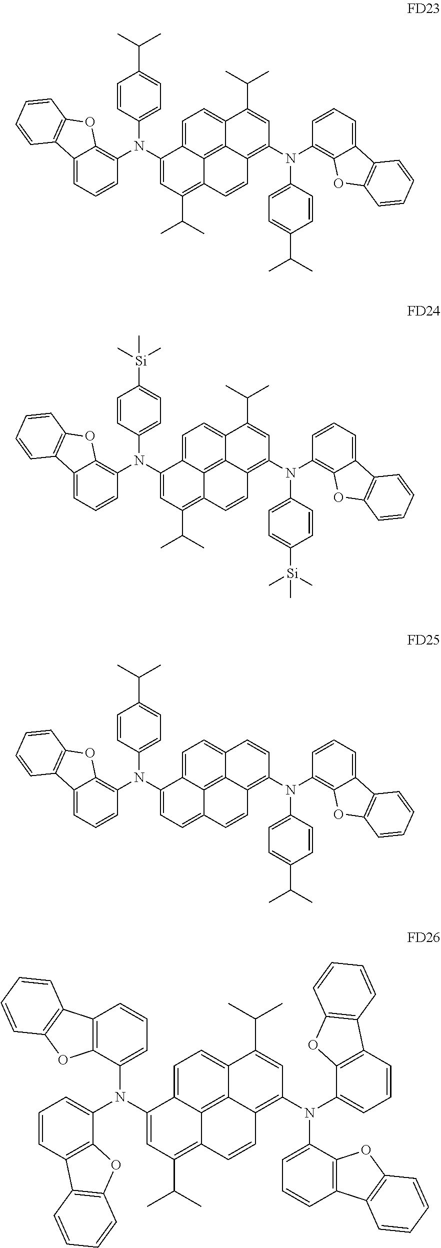 [SMILES: CC(C)c1ccc(N(c2cc(C(C)C)c3ccc4c(N(c5ccc(C(C)C)cc5)c5cccc6c5oc5ccccc56)cc(C(C)C)c5ccc2c3c54)c2cccc3c2oc2ccccc23)cc1 ; CC(C)c1cc(N(c2ccc([Si](C)(C)C)cc2)c2cccc3c2oc2ccccc23)c2ccc3c(C(C)C)cc(N(c4ccc([Si](C)(C)C)cc4)c4cccc5c4oc4ccccc45)c4ccc1c2c34 ; CC(C)c1ccc(N(c2ccc3ccc4c(N(c5ccc(C(C)C)cc5)c5cccc6c5oc5ccccc56)ccc5ccc2c3c54)c2cccc3c2oc2ccccc23)cc1 ; CC(C)c1cc(N(c2cccc3c2oc2ccccc23)c2cccc3c2oc2ccccc23)c2ccc3c(C(C)C)cc(N(c4cccc5c4oc4ccccc45)c4cccc5c4oc4ccccc45)c4ccc1c2c34]

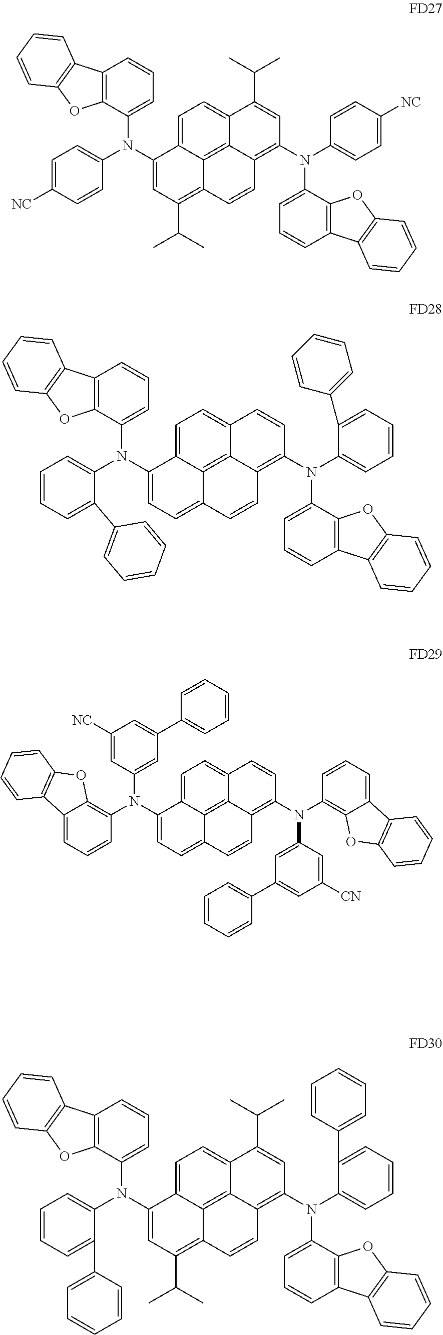

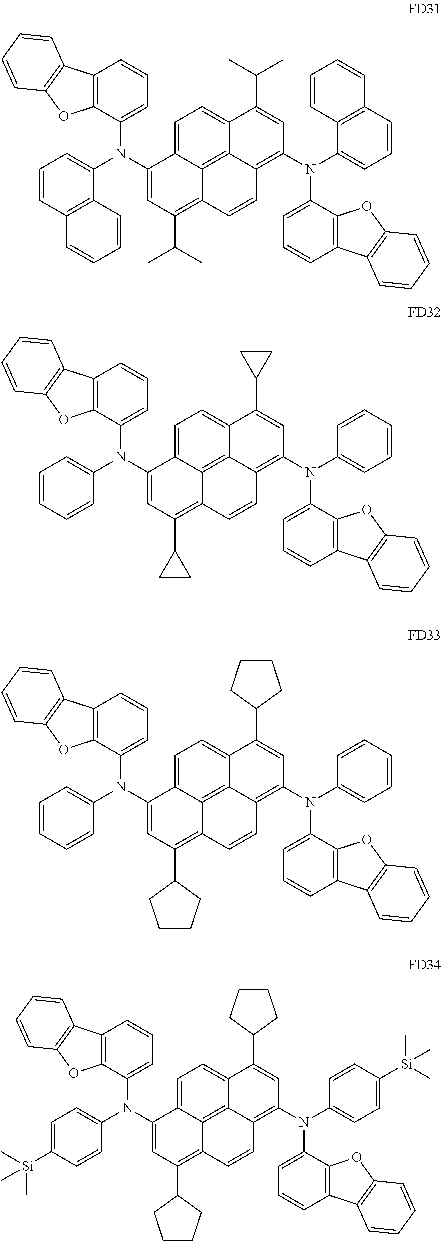

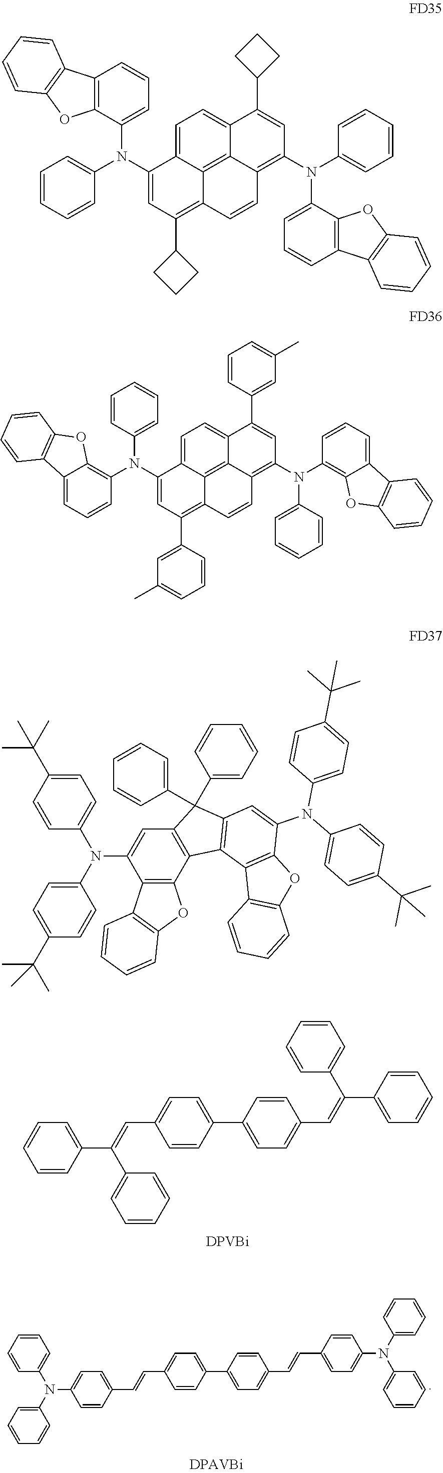

### [Delayed Fluorescence Material]

The emission layer may include a delayed fluorescence material.

In the specification, the delayed fluorescence material may be selected from compounds capable of emitting delayed fluorescent light based on a delayed fluorescence emission mechanism.

The delayed fluorescence material included in the emission layer may serve as a host or as a dopant depending on the types of other materials included in the emission layer.

In embodiments, a difference between a triplet energy level (eV) of the delayed fluorescence material and a singlet energy level (eV) of the delayed fluorescence material may be greater than or equal to 0 eV and less than or equal to 0.5 eV. When the difference between the triplet energy level (eV) of the delayed fluorescence material and the singlet energy level (eV) of the delayed fluorescence material satisfies the above-described range, up-conversion from the triplet state to the singlet state of the delayed fluorescence materials may effectively occur, and thus, the luminescence efficiency of the light-emitting device 10 may be improved.

In an embodiment, the delayed fluorescence material may include: a material including at least one electron donor (for example, a π electron-rich C₃-C₆₀ cyclic group, such as a carbazole group) and at least one electron acceptor (for example, a sulfoxide group, a cyano group, or air electron-deficient nitrogen-containing C₁-C₆₀ cyclic group); and a material including a C₈-C₆₀ polycyclic group in which two or more cyclic groups are condensed while sharing boron (B).

Examples of the delayed fluorescence material may include at least one of Compounds DF1 to DF14:

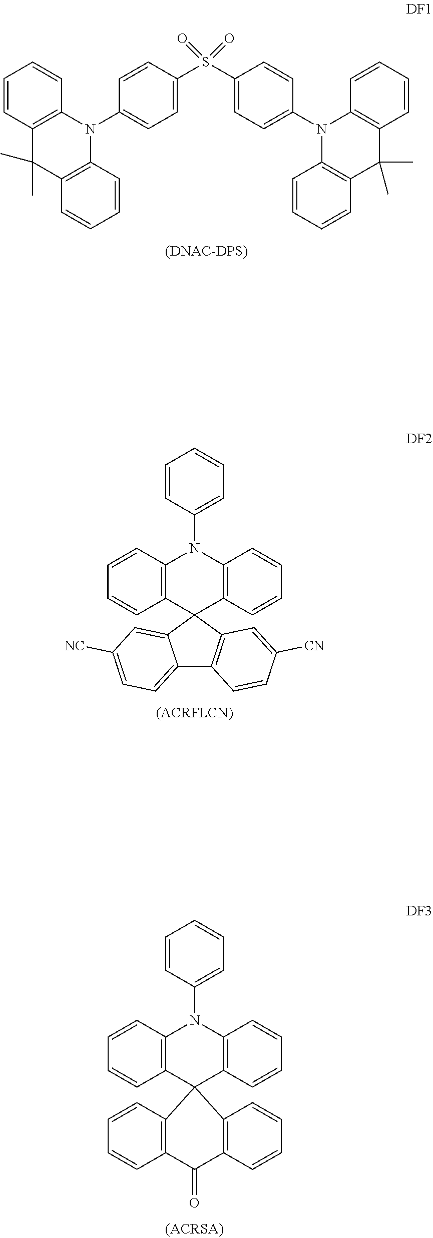

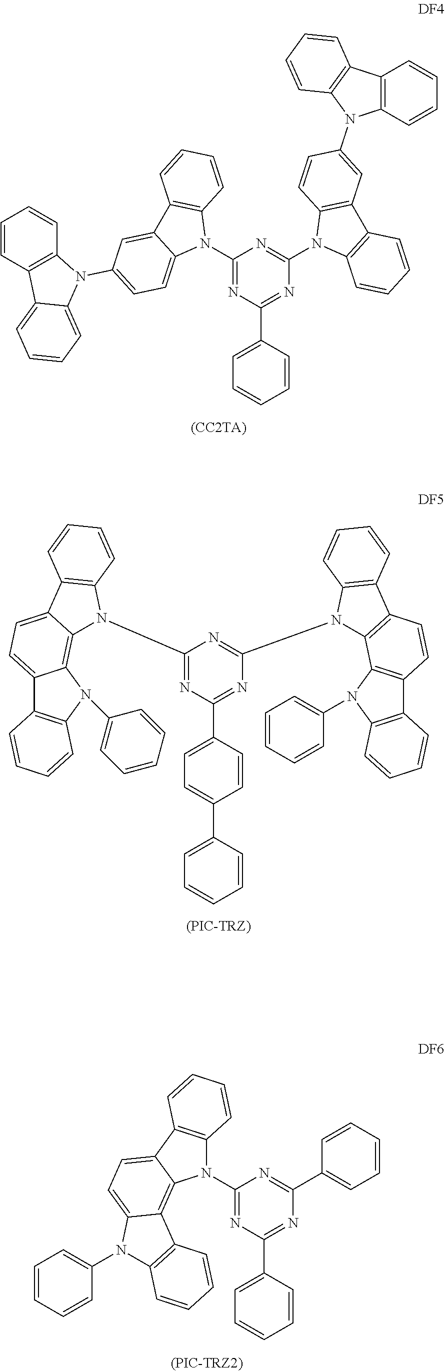 [SMILES: c1ccc(-c2nc(-n3c4ccccc4c4cc(-n5c6ccccc6c6ccccc65)ccc43)nc(-n3c4ccccc4c4cc(-n5c6ccccc6c6ccccc65)ccc43)n2)cc1 ; c1ccc(-c2ccc(-c3nc(-n4c5ccccc5c5ccc6c7ccccc7n(-c7ccccc7)c6c54)nc(-n4c5ccccc5c5ccc6c7ccccc7n(-c7ccccc7)c6c54)n3)cc2)cc1 ; c1ccc(-c2nc(-c3ccccc3)nc(-n3c4ccccc4c4ccc5c(c6ccccc6n5-c5ccccc5)c43)n2)cc1]

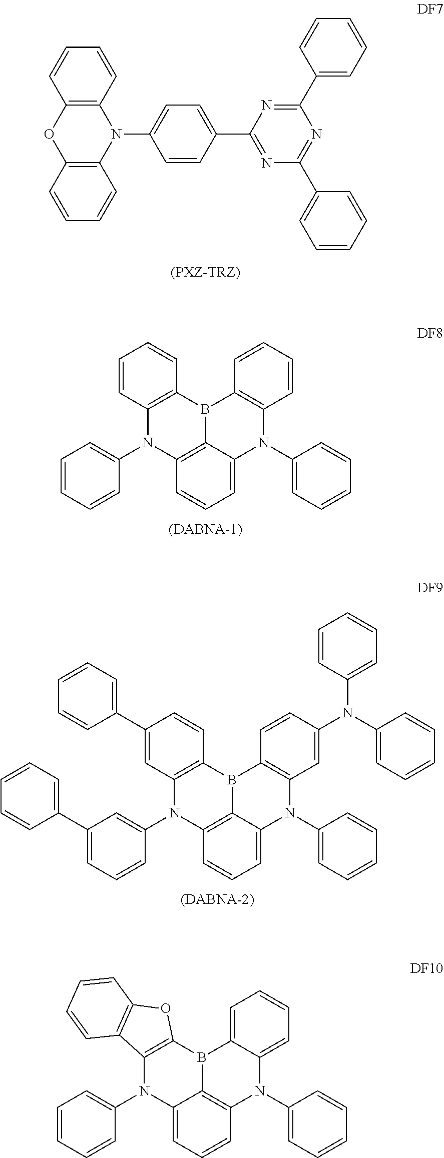

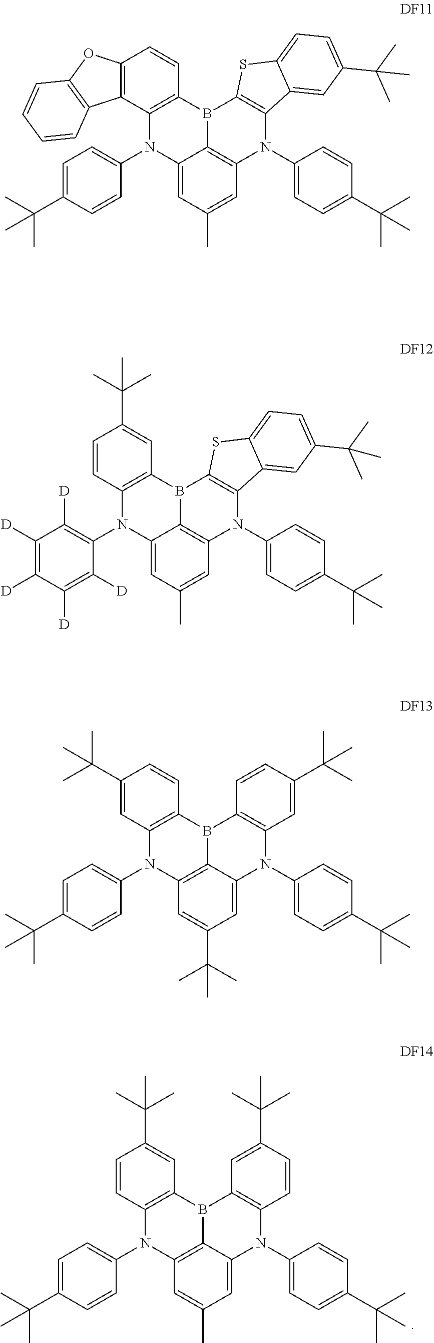

[Quantum Dot]

The emission layer may include a quantum dot.

The term “quantum dots” as used herein may be crystals of a semiconductor compound, and may include any material capable of emitting light of various emission wavelengths according to a size of the crystals.

A diameter of the quantum dot may be, for example, in a range of about 1 nm to about 10 nm.

The quantum dot may be synthesized by a wet chemical process, a metal organic chemical vapor deposition process, a molecular beam epitaxy process, or any process similar thereto.

The wet chemical process is a method that includes mixing a precursor material with an organic solvent and growing a quantum dot particle crystal. When the crystal grows, the organic solvent naturally acts as a dispersant coordinated on the surface of the quantum dot crystal and controls the growth of the crystal so that the growth of quantum dot particles may be controlled through a process which costs less, and may be more readily performed than vapor deposition methods, such as metal organic chemical vapor deposition (MOCVD) or molecular beam epitaxy (MBE).

The quantum dot may include: Group II-VI semiconductor compounds; Group III-V semiconductor compounds; Group III-VI semiconductor compounds; Group I-III-VI semiconductor compounds; Group IV-VI semiconductor compounds; a Group IV element or compound; or any combination thereof.

Examples of a Group II-VI semiconductor compound may include: a binary compound, such as CdS, CdSe, CdTe, ZnS, ZnSe, ZnTe, ZnO, HgS, HgSe, HgTe, MgSe, or MgS; a ternary compound, such as CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, or MgZnS; a quaternary compound, such as CdZnSeS, CdZnSeTe, CdZnSTe, CdHgSeS, CdHgSeTe, CdHgSTe, HgZnSeS, HgZnSeTe, or HgZnSTe; or any combination thereof.

Examples of a Group III-V semiconductor compound may include: a binary compound, such as GaN, GaP, GaAs, GaSb, AlN, AlP, AlAs, AlSb, InN, InP, InAs, or InSb; a ternary compound, such as GaNP, GaNAs, GaNSb, GaPAs, GaPSb, AlNP, AlNAs, AlNSb, AlPAs, AlPSb, InGaP, InNP, InAlP, InNAs, InNSb, InPAs, or InPSb; a quaternary compound, such as GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, or InAlPSb; or any combination thereof. The Group III-V semiconductor compound may further include a Group II element. Examples of the Group III-V semiconductor compound further including a Group II element may include InZnP, InGaZnP, InAlZnP, etc.

Examples of a Group III-VI semiconductor compound may include: a binary compound, such as GaS, GaSe, Ga₂Se₃, GaTe, InS, InSe, In₂S₃, In₂Se₃, or InTe; a ternary compound, such as InGaS₃, or InGaSe₃; or any combination thereof.

Examples of a Group I-III-VI semiconductor compound may include: a ternary compound, such as AgInS, AgInS₂, CuInS, CuInS₂, CuGaO₂, AgGaO₂, or AgAlO₂; or any combination thereof.

Examples of a Group IV-VI semiconductor compound may include: a binary compound, such as SnS, SnSe, SnTe, PbS, PbSe, or PbTe; a ternary compound, such as SnSeS, SnSeTe, SnSTe, PbSeS, PbSeTe, PbSTe, SnPbS, SnPbSe, or SnPbTe; a quaternary compound, such as SnPbSSe, SnPbSeTe, or SnPbSTe; or any combination thereof.

Examples of a Group IV element or compound may include: a single element material, such as Si or Ge; a binary compound, such as SiC or SiGe; or any combination thereof.

Each element included in a multi-element compound such as a binary compound, a ternary compound, and a quaternary compound may be present at a uniform concentration or non-uniform concentration in a particle.

The quantum dot may have a single structure in which the concentration of each element in the quantum dot is uniform, or a core-shell structure. When the quantum dot has a core-shell structure, a material included in the core and a material included in the shell may be different from each other.

The shell of the quantum dot may serve as a protective layer that prevents chemical degeneration of the core to maintain semiconductor characteristics, and/or may serve as a charging layer that imparts electrophoretic characteristics to the quantum dot. The shell may be a single layer or a multi-layer. The interface between the core and the shell may have a concentration gradient in which the concentration of a material that is present in the shell decreases toward the core.

Examples of a shell of the quantum dot may include a metal oxide, a metalloid oxide, or a non-metal oxide, a semiconductor compound, or any combination thereof. Examples of the metal oxide, the metalloid oxide, or the non-metal oxide may include: a binary compound, such as SiO₂, Al₂O₃, TiO₂, ZnO, MnO, Mn₂O₃, Mn₃O₄, CuO, FeO, Fe₂O₃, Fe₃O₄, CoO, Co₃O₄, or NiO; a ternary compound, such as MgAl₂O₄, CoFe₂O₄, NiFe₂O₄, or CoMn₂O₄; and any combination thereof. Examples of the semiconductor compound may include, as described herein, a Group II-VI semiconductor compound, a Group III-V semiconductor compound, a Group III-VI semiconductor compound, a Group I-III-VI semiconductor compound, a Group IV-VI semiconductor compound, or any combination thereof. For example, the semiconductor compound may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, or any combination thereof.

A full width at half maximum (FWHM) of the emission wavelength spectrum of the quantum dot may be equal to or less than about 45 nm. For example, a FWHM of an emission wavelength spectrum of the quantum dot may be equal to or less than about 40 nm. For example, a FWHM of an emission wavelength spectrum of the quantum dot may be equal to or less than about 30 nm. Within these ranges, color purity or color reproducibility may be increased. Light emitted through the quantum dot may be emitted in all directions, so that a wide viewing angle may be improved.

In an embodiment, the quantum dot may be in the form of a spherical particle, a pyramidal particle, a multi-arm particle, a cubic nanoparticle, a nanotube particle, a nanowire particle, a nanofiber particle, or a nanoplate particle.

Since the energy band gap may be adjusted by controlling the size of the quantum dot, light having various wavelength bands may be obtained from a quantum dot emission layer. Accordingly, by using quantum dots of different sizes, a light-emitting device that emits light of various wavelengths may be implemented. In embodiments, the size of the quantum dot may be selected to emit red, green and/or blue light. The size of the quantum dot may be configured to emit white light by combination of light of various colors.

[Electron Transport Region in Interlayer 130]

The electron transport region may have a structure consisting of a layer consisting of a single material, a structure consisting of a layer including different materials, or a structure including multiple layers including different materials.

The electron transport region may include a buffer layer, a hole-blocking layer, an electron control layer, an electron transport layer, an electron injection layer, or any combination thereof.

In an embodiment, the electron transport region may have an electron transport layer/electron injection layer structure, a hole-blocking layer/electron transport layer/electron injection layer structure, an electron control layer/electron transport layer/electron injection layer structure, or a buffer layer/electron transport layer/electron injection layer structure, wherein the layers of each structure may be stacked from an emission layer in its respective stated order, but the structure of the electron transport region is not limited thereto.

In an embodiment, the electron transport region (for example, a buffer layer, a hole-blocking layer, an electron control layer, or an electron transport layer in the electron transport region) may include a metal-free compound including at least one rr electron-deficient nitrogen-containing C₁-C₆₀ cyclic group.

In an embodiment, the electron transport region may include a compound represented by Formula 601:

[Ar₆₀₁]ₓₑ₁₁-[(L₆₀₁)ₓₑ₁-R₆₀₁]ₓₑ₂₁. [Formula 601]

In Formula 601,
Ar₆₀₁ and L₆₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xe11 may be 1, 2, or 3,
xe1 may be 0, 1, 2, 3, 4, or 5,
R₆₀₁ may be a C₃-C₆₀ carbocyclic group unsubstituted or substituted with at least one R₁₀ₐ, a C₁-C₆₀ heterocyclic group unsubstituted or substituted with at least one R₁ 0a, —Si(Q₆₀₁)(Q₆₀₂)(Q₆₀₃), —C(═O)(Q₆₀₁), —S(═O)₂(Q₆₀₁), or —P(═O)(Q₆₀₁)(Q₆₀₂),
Q₆₀₁ to Q₆₀₃ may each independently be the same as described herein with respect to Q₁,
xe21 may be 1, 2, 3, 4, or 5, and
at least one of Ar₆₀₁, L₆₀₁, and R₆₀₁ may each independently be air electron-deficient nitrogen-containing C₁-C₆₀ cyclic group unsubstituted or substituted with at least one R₁₀ₐ.

In an embodiment, in Formula 601 m, when xe11 in is 2 or more, two or more of Ar₆₀₁(s) may be linked to each other via a single bond.

In other embodiments, in Formula 601, Ar₆₀₁ may be a substituted or unsubstituted anthracene group.

In other embodiments, the electron transport region may include a compound represented by Formula 601-1:

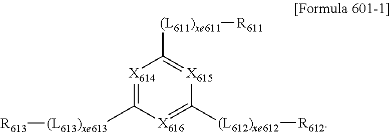

In Formula 601-1,
X₆₁₄ may be N or C(R₆₁₄), X₆₁₅ may be N or C(R₆₁₅), X₆₁₆ may be N or C(R₆₁₆), and at least one of X₆₁₄ to X₆₁₆ may each be N,
L₆₁₁ to L₆₁₃ may each independently be the same as described herein with respect to L₆₀₁,
xe611 to xe613 may each independently be the same as described herein with respect to xe1,
R₆₁₁ to R₆₁₃ may each independently be the same as described herein with respect to R₆₀₁, and
R₆₁₄ to R₆₁₆ may each independently be hydrogen, deuterium, —F, —Cl, —Br, —I, a hydroxyl group, a cyano group, a nitro group, a C₁-C₂₀ alkyl group, a C₁-C₂₀ alkoxy group, a C₃-C₆₀ carbocyclic group unsubstituted or substituted with at least one R₁₀ₐ, or a C₁-C₆₀ heterocyclic group unsubstituted or substituted with at least one R₁₀ₐ.

In an embodiment, in Formulae 601 and 601-1, xe1 and xe611 to xe613 may each independently be 0, 1, or 2.

The electron transport region may include one of Compounds ET1 to ET45, 2,9-dimethyl-4,7-diphenyl-1,10-phenanthroline (BCP), 4,7-diphenyl-1,10-phenanthroline (Bphen), Alq₃, BAlq, TAZ, NTAZ, or any combination thereof:

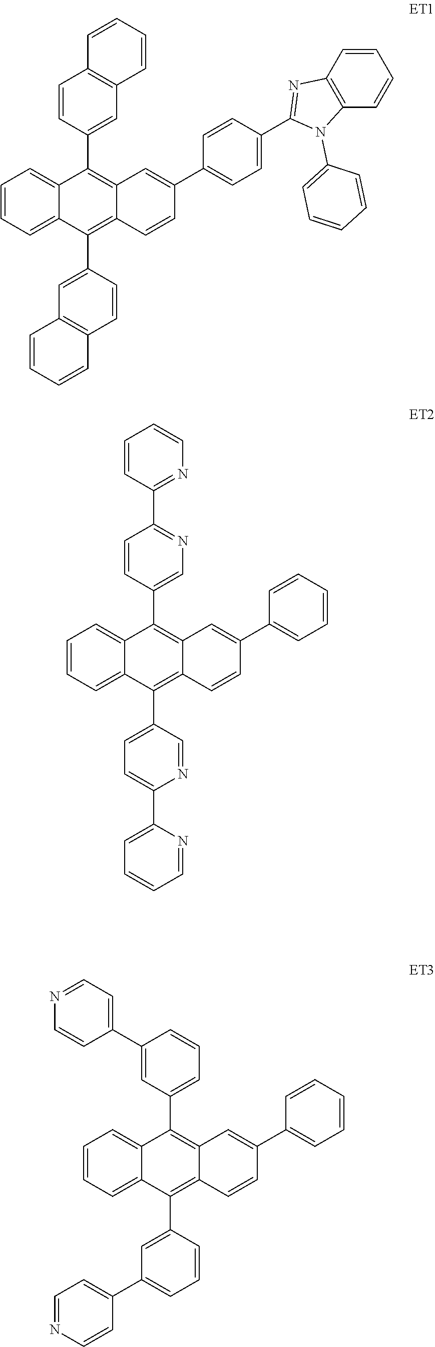 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

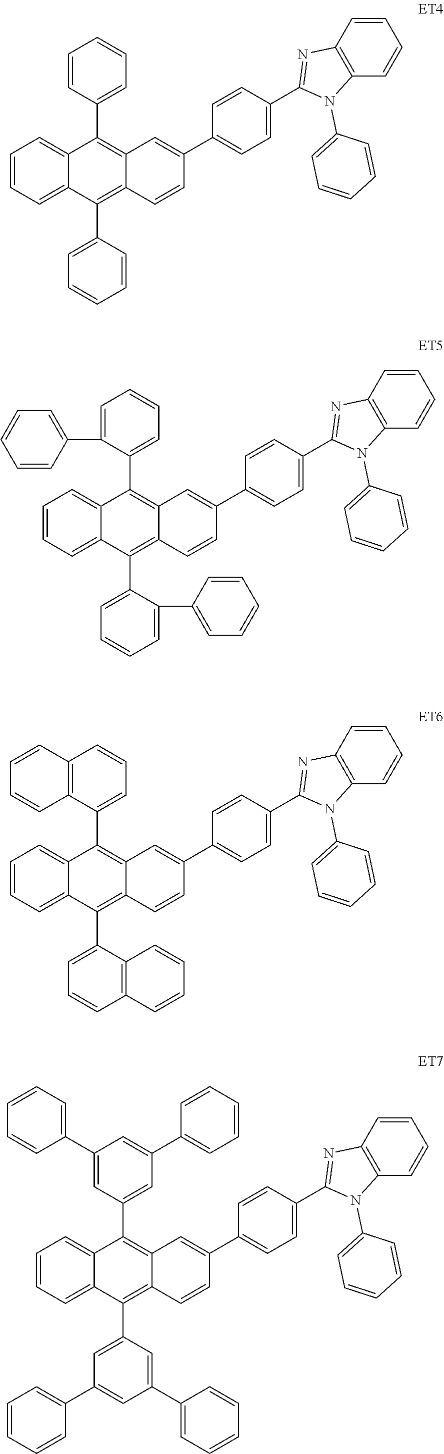 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1 ; c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1]

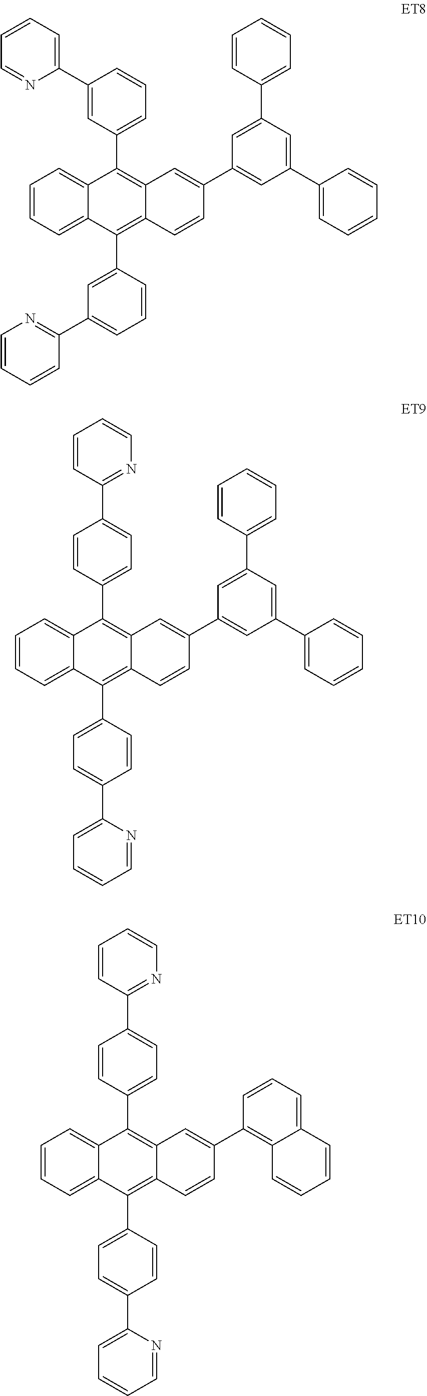 [SMILES: c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1 ; c1ccc(-c2ccc(-c3c4ccccc4c(-c4ccc(-c5ccccn5)cc4)c4cc(-c5cccc6ccccc56)ccc34)cc2)nc1]

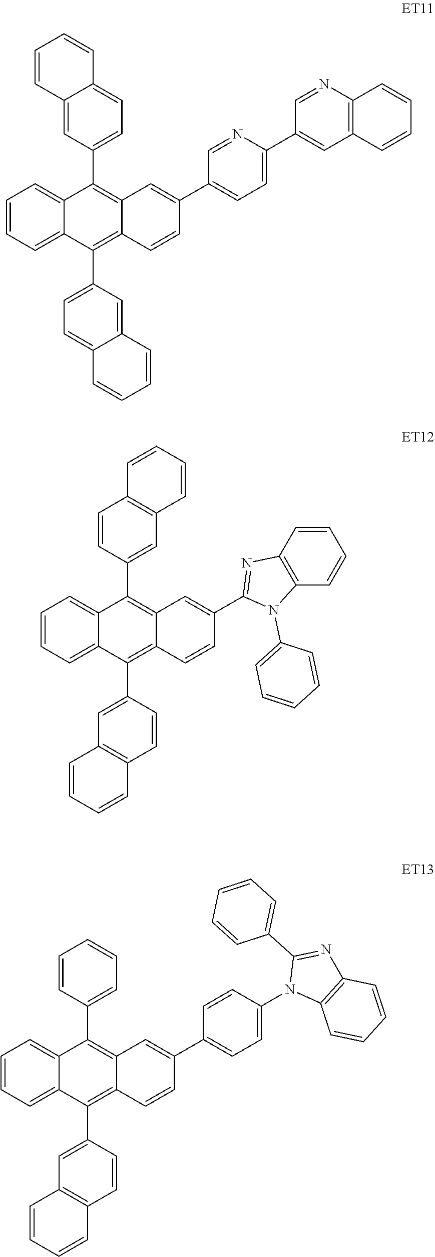 [SMILES: c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1 ; c1ccc(-c2c3ccccc3c(-c3ccc4ccccc4c3)c3ccc(-c4ccc(-n5c(-c6ccccc6)nc6ccccc65)cc4)cc23)cc1]

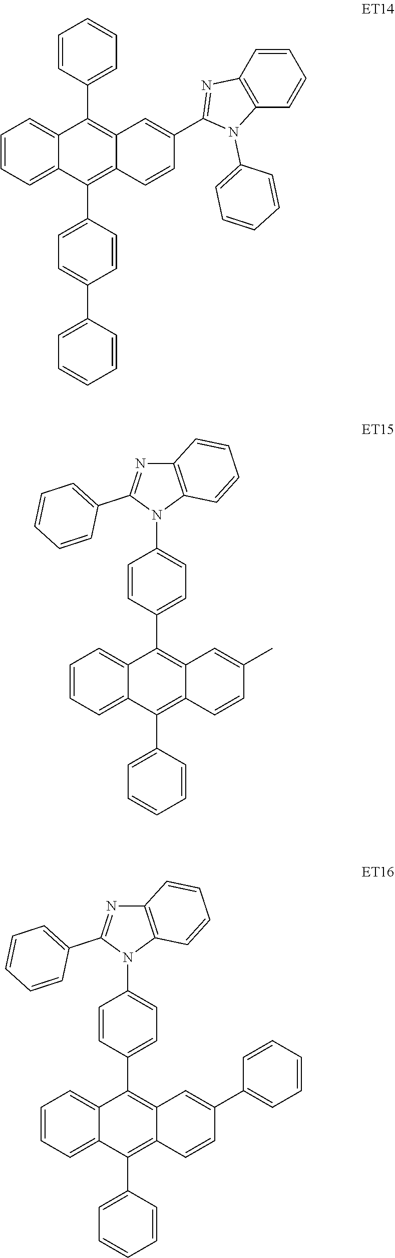 [SMILES: c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1 ; c1ccc(-c2ccc3c(-c4ccccc4)c4ccccc4c(-c4ccc(-n5c(-c6ccccc6)nc6ccccc65)cc4)c3c2)cc1]

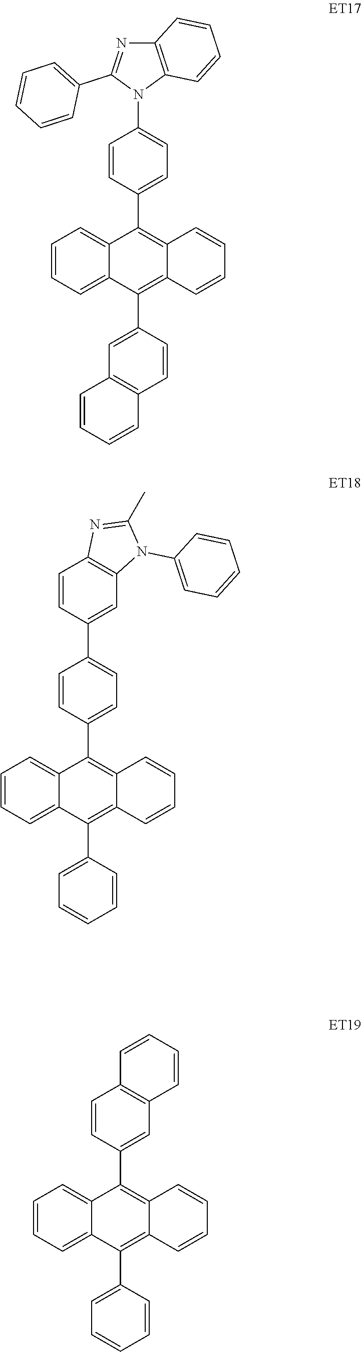

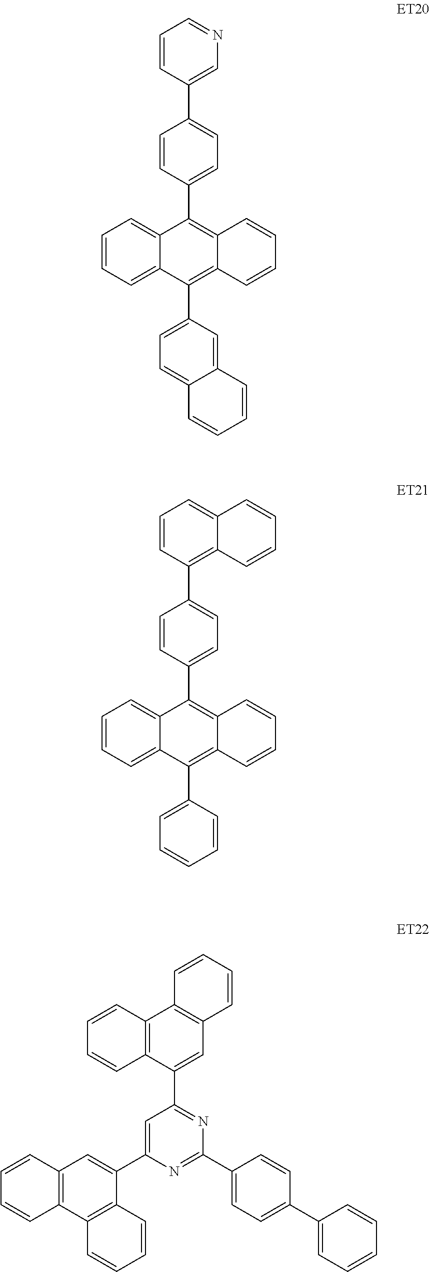

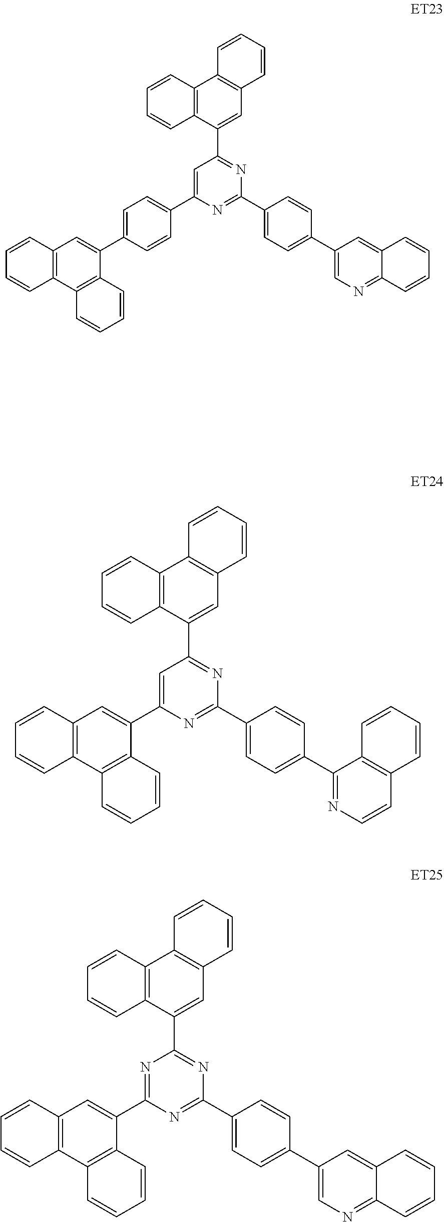 [SMILES: c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1 ; c1ccc2ncc(-c3ccc(-c4nc(-c5cc6ccccc6c6ccccc56)nc(-c5cc6ccccc6c6ccccc56)n4)cc3)cc2c1]

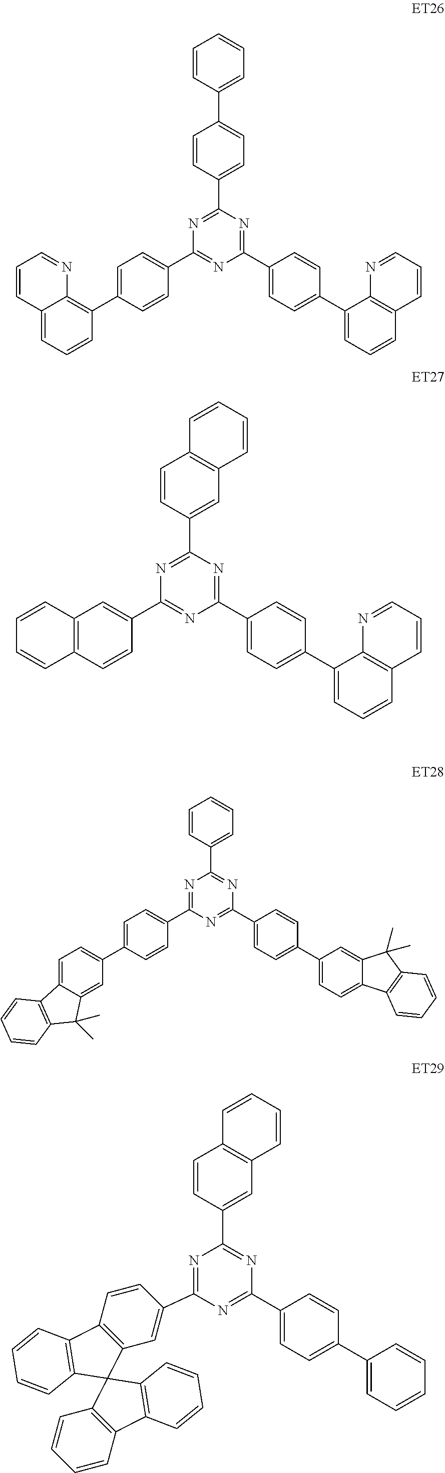 [SMILES: c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1 ; CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3nc(-c4ccc5c(c4)C4(c6ccccc6-c6ccccc64)c4ccccc4-5)nc(-c4ccc5ccccc5c4)n3)cc2)cc1]

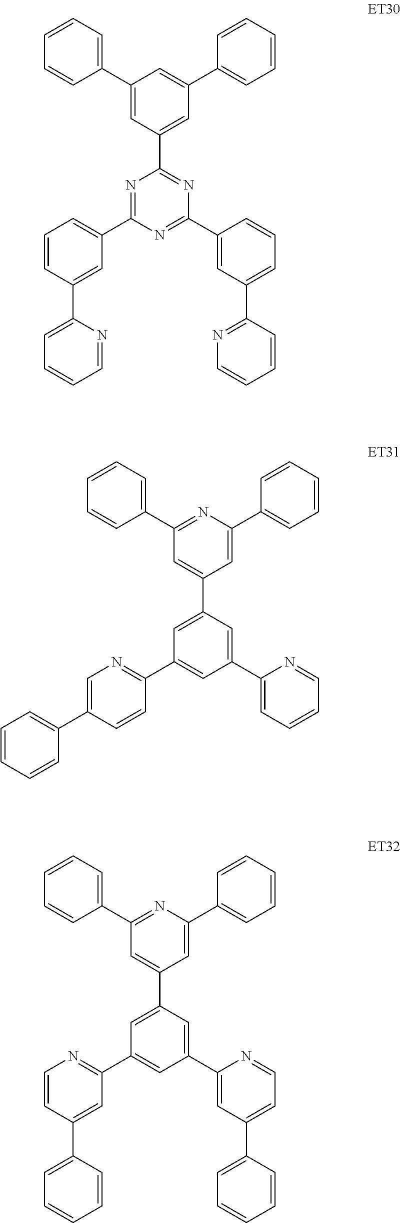 [SMILES: c1ccc(-c2cc(-c3ccccc3)cc(-c3nc(-c4cccc(-c5ccccn5)c4)nc(-c4cccc(-c5ccccn5)c4)n3)c2)cc1 ; c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1]

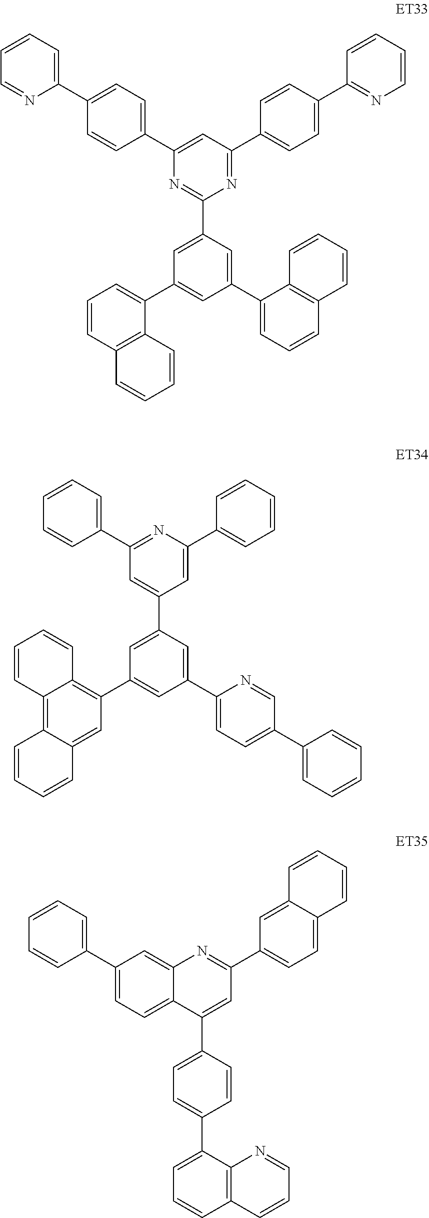 [SMILES: c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1 ; c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1]

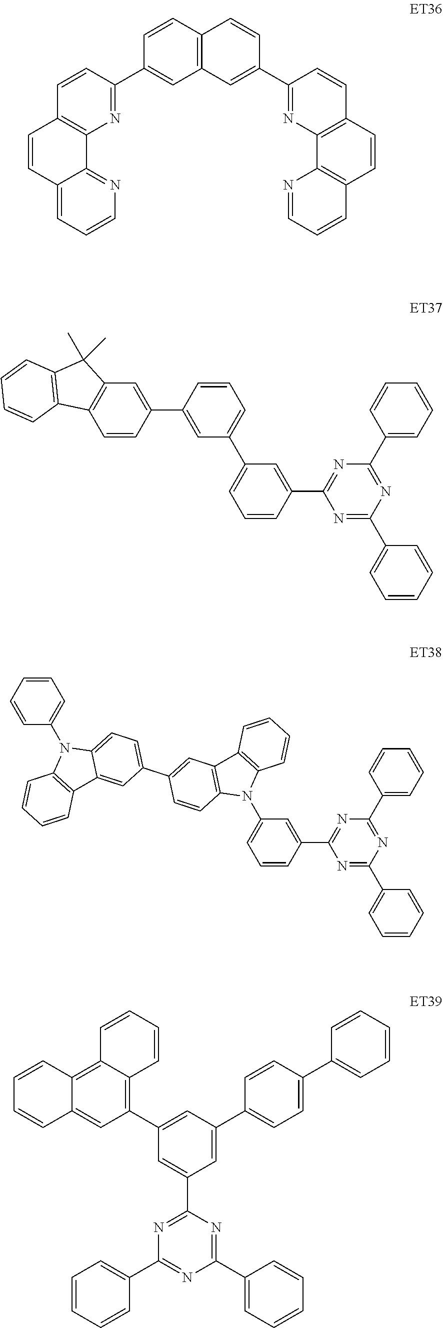 [SMILES: c1cnc2c(c1)ccc1ccc(-c3ccc4ccc(-c5ccc6ccc7cccnc7c6n5)cc4c3)nc12 ; CC1(C)c2ccccc2-c2ccc(-c3cccc(-c4cccc(-c5nc(-c6ccccc6)nc(-c6ccccc6)n5)c4)c3)cc21 ; c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; c1ccc(-c2ccc(-c3cc(-c4nc(-c5ccccc5)nc(-c5ccccc5)n4)cc(-c4cc5ccccc5c5ccccc45)c3)cc2)cc1]

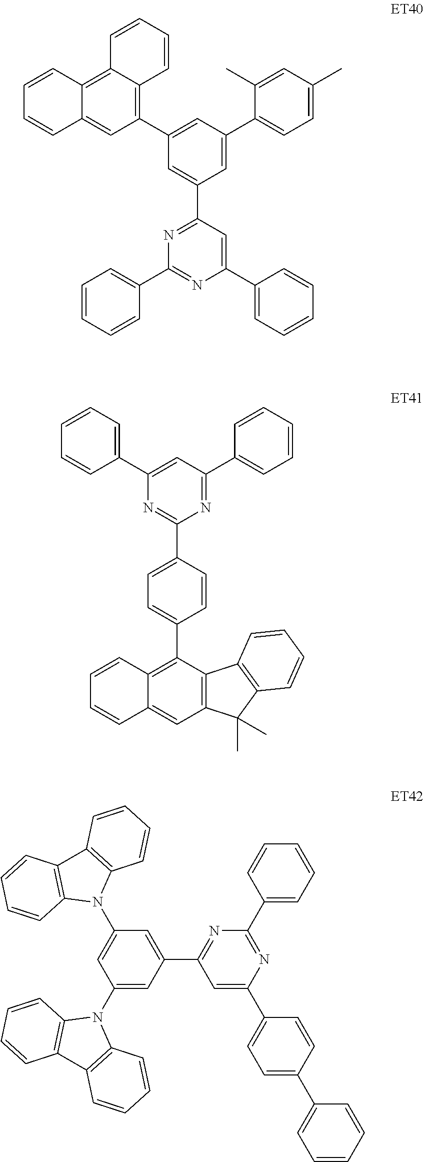 [SMILES: Cc1ccc(-c2cc(-c3cc(-c4ccccc4)nc(-c4ccccc4)n3)cc(-c3cc4ccccc4c4ccccc34)c2)c(C)c1 ; CC1(C)c2ccccc2-c2c1cc1ccccc1c2-c1ccc(-c2nc(-c3ccccc3)cc(-c3ccccc3)n2)cc1 ; c1ccc(-c2ccc(-c3cc(-c4cc(-n5c6ccccc6c6ccccc65)cc(-n5c6ccccc6c6ccccc65)c4)nc(-c4ccccc4)n3)cc2)cc1]

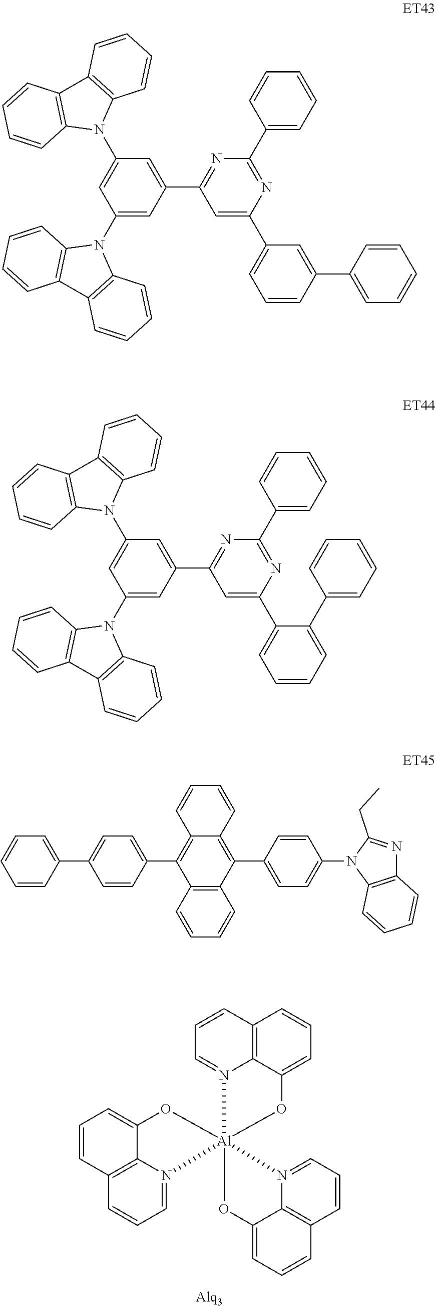

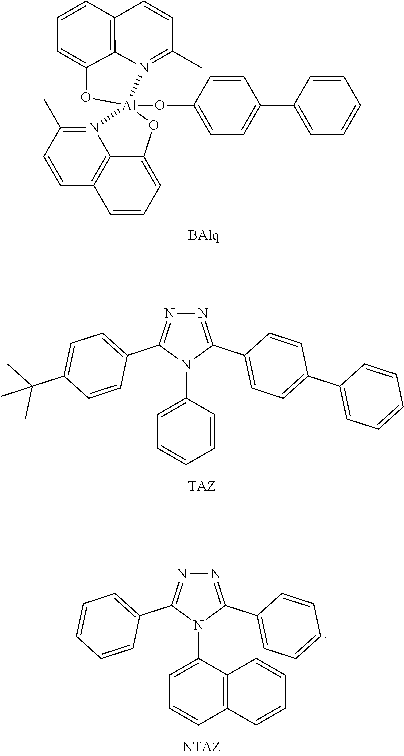

A thickness of the electron transport region may be in a range of about 100 Å to about 5,000 Å. For example, the thickness of the electron transport region may be in a range of about 160 Å to about 4,000 Å. When the electron transport region includes a buffer layer, a hole-blocking layer, an electron control layer, an electron transport layer, or any combination thereof, a thickness of the buffer layer, the hole-blocking layer, or the electron control layer may each independently be in a range of about 20 Å to about 1,000 Å, and the thickness of the electron transport layer may be in a range of about 100 Å to about 1,000 Å. For example, the thickness of the buffer layer, the hole blocking layer, or the electron control layer may each independently be in a range of about 30 Å to about 300 Å. For example, the thickness of the electron transport layer may be in a range of about 150 Å to about 500 Å. When the thickness of the buffer layer, the hole blocking layer, the electron control layer, the electron transport layer, and/or the electron transport region are within these ranges, satisfactory electron transporting characteristics may be obtained without a substantial increase in driving voltage.

The electron transport region (for example, the electron transport layer in the electron transport region) may further include, in addition to the materials described above, a metal-containing material.

The metal-containing material may include an alkali metal complex, an alkaline earth metal complex, or any combination thereof. A metal ion of an alkali metal complex may be a Li ion, a Na ion, a K ion, a Rb ion, or a Cs ion, and a metal ion of an alkaline earth metal complex may be a Be ion, a Mg ion, a Ca ion, a Sr ion, or a Ba ion. A ligand coordinated with the metal ion of the alkali metal complex or with the metal ion of the alkaline earth-metal complex may each independently include a hydroxyquinoline, a hydroxyisoquinoline, a hydroxybenzoquinoline, a hydroxyacridine, a hydroxyphenanthridine, a hydroxyphenyloxazole, a hydroxyphenylthiazole, a hydroxyphenyloxadiazole, a hydroxyphenylthiadiazole, a hydroxyphenylpyridine, a hydroxyphenylbenzimidazole, a hydroxyphenylbenzothiazole, a bipyridine, a phenanthroline, a cyclopentadiene, or any combination thereof.

For example, the metal-containing material may include a Li complex. The Li complex may include, for example, Compound ET-D1 (LiQ) or Compound ET-D2:

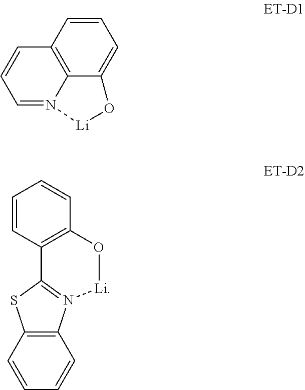

The electron transport region may include an electron injection layer that facilitates the injection of electrons from the second electrode 150. The electron injection layer may directly contact the second electrode 150.

The electron injection layer may have a structure consisting of a layer consisting of a single material, a structure consisting of a layer including different materials, or a structure including multiple layers including different materials.

The electron injection layer may include an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof.

The alkali metal may include Li, Na, K, Rb, Cs, or any combination thereof. The alkaline earth metal may include Mg, Ca, Sr, Ba, or any combination thereof. The rare earth metal may include Sc, Y, Ce, Tb, Yb, Gd, or any combination thereof.

The alkali metal-containing compound, the alkaline earth metal-containing compound, and the rare earth metal-containing compound may be oxides, halides (for example, fluorides, chlorides, bromides, or iodides), or tellurides of the alkali metal, the alkaline earth metal, and the rare earth metal, or any combination thereof.

The alkali metal-containing compound may include: alkali metal oxides, such as Li₂O, Cs₂O, or K₂O; alkali metal halides, such as LiF, NaF, CsF, KF, LiI, NaI, CsI, or KI; or any combination thereof. The alkaline earth metal-containing compound may include an alkaline earth metal compound, such as BaO, SrO, CaO, BaXSr₁₋ₓO (wherein x is a real number satisfying the condition of 0<x<1), BaₓCa₁₋ₓO (wherein x is a real number satisfying the condition of 0<x<1), or the like. The rare earth metal-containing compound may include YbF₃, ScF₃, Sc₂O₃, Y₂O₃, Ce₂O₃, GdF₃, TbF₃, YbI₃, ScI₃, TbI₃, or any combination thereof. In embodiments, the rare earth metal-containing compound may include a lanthanide metal telluride. Examples of the lanthanide metal telluride may include LaTe, CeTe, PrTe, NdTe, PmTe, SmTe, EuTe, GdTe, TbTe, DyTe, HoTe, ErTe, TmTe, YbTe, LuTe, La₂Te₃, Ce₂Te₃, Pr₂Te₃, Nd₂Te₃, Pm₂Te₃, Sm₂Te₃, Eu₂Te₃, Gd₂Te₃, Tb₂Te₃, Dy₂Te₃, Ho₂Te₃, Er₂Te₃, Tm₂Te₃, Yb₂Te₃, and Lu₂Te₃.

The alkali metal complex, the alkaline earth-metal complex, and the rare earth metal complex may include an alkali metal ion, an alkaline earth metal ion, and a rare earth metal ion and a ligand bonded to the metal ion, for example (hydroxyquinoline, hydroxyisoquinoline, hydroxybenzoquinoline, hydroxyacridine, hydroxyphenanthridine, hydroxyphenyloxazole, hydroxyphenylthiazole, hydroxyphenyloxadiazole, hydroxyphenylthiadiazole, hydroxyphenylpyridine, hydroxyphenyl benzimidazole, hydroxyphenylbenzothiazole, bipyridine, phenanthroline, cyclopentadiene, or any combination thereof).

The electron injection layer may consist of an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof, as described above. In embodiments, the electron injection layer may further include an organic material (for example, a compound represented by Formula 601).

In embodiments, the electron injection layer may consist of an alkali metal-containing compound (for example, an alkali metal halide), or an alkali metal-containing compound (for example, an alkali metal halide), and an alkali metal, an alkaline earth metal, a rare earth metal, or any combination thereof. For example, the electron injection layer may be a KI:Yb co-deposited layer, an RbI:Yb co-deposited layer, a LiF:Yb co-deposited layer, or the like.

When the electron injection layer further includes an organic material, an alkali metal, an alkaline earth metal, a rare earth metal, an alkali metal-containing compound, an alkaline earth metal-containing compound, a rare earth metal-containing compound, an alkali metal complex, an alkaline earth-metal complex, a rare earth metal complex, or any combination thereof may be uniformly or non-uniformly dispersed in a matrix including the organic material.

A thickness of the electron injection layer may be in a range of about 1 Å to about 100 Å. For example, the thickness of the electron injection layer may be in a range of about 3 Å to about 90 Å. When the thickness of the electron injection layer is within the ranges described above, satisfactory electron injection characteristics may be obtained without a substantial increase in driving voltage.

[Second Electrode 150]

The second electrode 150 may be located on the interlayer 130 having a structure as described above. The second electrode 150 may be a cathode, which is an electron injection electrode. The second electrode 150 may include a material having a low-work function, for example, a metal, an alloy, an electrically conductive compound, or any combination thereof.

The second electrode 150 may include lithium (Li), silver (Ag), magnesium (Mg), aluminum (Al), aluminum-lithium (Al—Li), calcium (Ca), magnesium-indium (Mg—In), magnesium-silver (Mg—Ag), ytterbium (Yb), silver-ytterbium (Ag—Yb), ITO, IZO, or any combination thereof. The second electrode 150 may be a transmissive electrode, a semi-transmissive electrode, or a reflective electrode.

The second electrode 150 may have a single-layered structure or a multi-layered structure.

[Capping Layer]

The light-emitting device 10 may include a first capping layer outside the first electrode 110, and/or a second capping layer outside the second electrode 150. In an embodiment, the light-emitting device 10 may have a structure in which the first capping layer, the first electrode 110, the interlayer 130, and the second electrode 150 are stacked in the stated order, a structure in which the first electrode 110, the interlayer 130, the second electrode 150, and the second capping layer are stacked in the stated order, or a structure in which the first capping layer, the first electrode 110, the interlayer 130, the second electrode 150, and the second capping layer are stacked in the stated order.

Light generated in an emission layer of the interlayer 130 of the light-emitting device 10 may be extracted toward the outside through the first electrode 110 which may be a semi-transmissive electrode or a transmissive electrode, and through the first capping layer. Light generated in an emission layer of the interlayer 130 of the light-emitting device 10 may be extracted toward the outside through the second electrode 150 which may be a semi-transmissive electrode or a transmissive electrode, and through the second capping layer.

The first capping layer and the second capping layer may increase external emission efficiency according to the principle of constructive interference. Accordingly, the light extraction efficiency of the light-emitting device 10 is increased, so that the luminescence efficiency of the light-emitting device 10 may be improved.

The first capping layer and the second capping layer may each include a material having a refractive index equal to or greater than about 1.6 (with respect to a wavelength of about 589 nm).

The first capping layer and the second capping layer may each independently be an organic capping layer including an organic material, an inorganic capping layer including an inorganic material, or an organic-inorganic composite capping layer including an organic material and an inorganic material.

At least one of the first capping layer and the second capping layer may each independently include carbocyclic compounds, heterocyclic compounds, amine group-containing compounds, porphine derivatives, phthalocyanine derivatives, a naphthalocyanine derivatives, alkali metal complexes, alkaline earth metal complexes, or any combination thereof. Optionally, the carbocyclic compound, the heterocyclic compound, and the amine group-containing compound may each be substituted with a substituent including O, N, S, Se, Si, F, Cl, Br, I, or any combination thereof. In embodiments, at least one of the first capping layer and the second capping layer may each independently include an amine group-containing compound.

For example, at least one of the first capping layer and the second capping layer may each independently include a compound represented by Formula 201, a compound represented by Formula 202, or any combination thereof.

In embodiments, at least one of the first capping layer and the second capping layer may each independently include one of Compounds HT28 to HT33, one of Compounds CP1 to CP6, β-NPB, or any combination thereof:

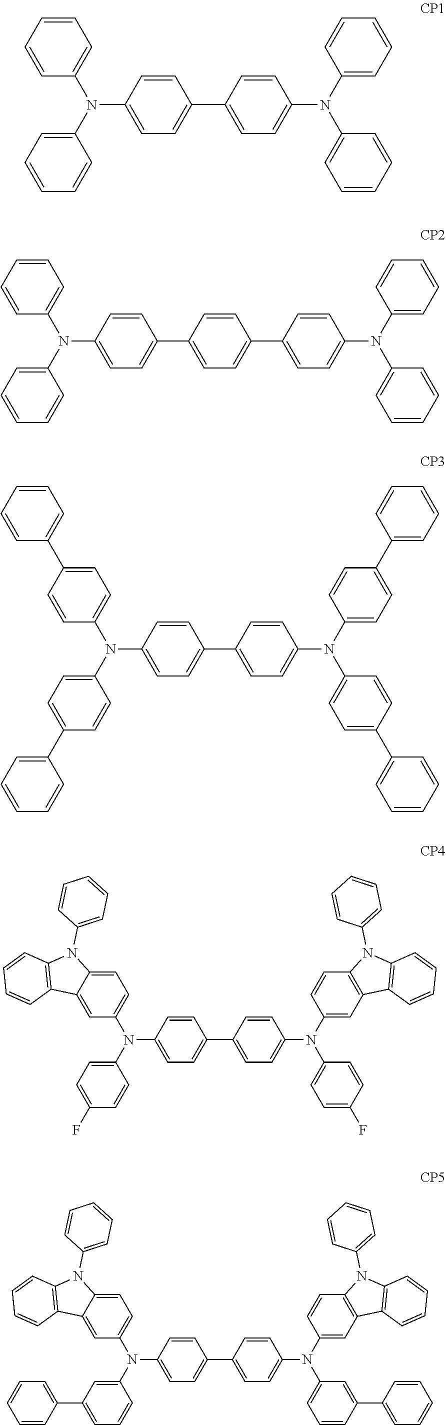 [SMILES: c1ccc(N(c2ccccc2)c2ccc(-c3ccc(N(c4ccccc4)c4ccccc4)cc3)cc2)cc1 ; c1ccc(N(c2ccccc2)c2ccc(-c3ccc(-c4ccc(N(c5ccccc5)c5ccccc5)cc4)cc3)cc2)cc1 ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; Fc1ccc(N(c2ccc(-c3ccc(N(c4ccc(F)cc4)c4ccc5c(c4)c4ccccc4n5-c4ccccc4)cc3)cc2)c2ccc3c(c2)c2ccccc2n3-c2ccccc2)cc1 ; c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1]

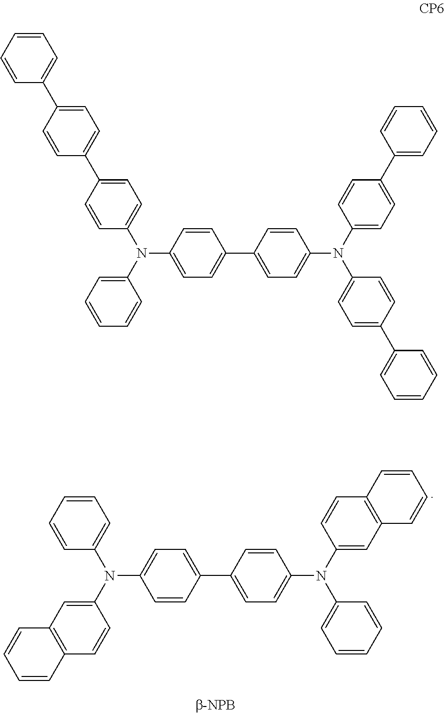 [SMILES: c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1]

[Film]

The heterocyclic compound represented by Formula 1 may be included in various films. Accordingly, another aspect of the disclosure provides a film including the heterocyclic compound represented by Formula 1. The film may be, for example, an optical member (or a light control means) (for example, a color filter, a color conversion member, a capping layer, a light extraction efficiency enhancement layer, a selective light absorbing layer, a polarizing layer, a quantum dot-containing layer, or like), a light-blocking member (for example, a light reflective layer, a light absorbing layer, or the like), or a protective member (for example, an insulating layer, a dielectric layer, or the like).

[Electronic Device]

The light-emitting device may be included in various electronic devices. For example, the electronic device including the light-emitting device may be a light-emitting apparatus, an authentication apparatus, or the like.

The electronic device (for example, a light-emitting apparatus) may further include, in addition to the light-emitting device, a color filter, a color conversion layer, or a color filter and a color conversion layer. The color filter and/or the color conversion layer may be located in at least one direction in which light emitted from the light-emitting device travels. In an embodiment, the light emitted from the light-emitting device may be blue light or white light. The light-emitting device may be the same as described herein. In embodiments, the color conversion layer may include a quantum dot. The quantum dot may be, for example, a quantum dot as described herein.

The electronic device may include a first substrate. The first substrate may include subpixels, the color filter may include color filter areas respectively corresponding to the subpixels, and the color conversion layer may include color conversion areas respectively corresponding to the subpixels.

A pixel-defining film may be located between the subpixels to define each subpixel.

The color filter may further include color filter areas and light-shielding patterns located among the color filter areas, and the color conversion layer may further include color conversion areas and light-shielding patterns located between the color conversion areas.

The color filter areas (or the color conversion areas) may include a first area emitting first color light, a second area emitting second color light, and/or a third area emitting third color light, wherein the first color light, the second color light, and/or the third color light may have different maximum emission wavelengths from one another. In an embodiment, the first color light may be red light, the second color light may be green light, and the third color light may be blue light. The color filter areas (or the color conversion areas) may include quantum dots. For example, the first area may include a red quantum dot, the second area may include a green quantum dot, and the third area may not include a quantum dot. The quantum dot may be the same as described herein. The first area, the second area, and/or the third area may each include a scatter.

For example, the light-emitting device may emit first light, the first area may absorb the first light to emit first-first color light, the second area may absorb the first light to emit second-first color light, and the third area may absorb the first light to emit third-first color light. The first-first color light, the second-first color light, and the third-first color light may have different maximum emission wavelengths. The first light may be blue light, the first-first color light may be red light, the second-first color light may be green light, and the third-first color light may be blue light.

The electronic device may further include a thin-film transistor, in addition to the light-emitting device as described above. The thin-film transistor may include a source electrode, a drain electrode, and an active layer, wherein any one of the source electrode and the drain electrode may be electrically connected to any one of the first electrode and the second electrode of the light-emitting device.

The thin-film transistor may further include a gate electrode, a gate insulating film, or the like.

The active layer may include crystalline silicon, amorphous silicon, an organic semiconductor, an oxide semiconductor, or the like.

The electronic device may further include a sealing portion for sealing the light-emitting device. The sealing portion may be located between the color filter and/or the color conversion layer and the light-emitting device. The sealing portion allows light from the light-emitting device to be extracted to the outside, and prevents ambient air and moisture from penetrating into the light-emitting device. The sealing portion may be a sealing substrate including a transparent glass substrate or a plastic substrate. The sealing portion may be a thin-film encapsulation layer including at least one layer of an organic layer and/or an inorganic layer. When the sealing portion is a thin film encapsulation layer, the electronic device may be flexible.

Various functional layers may be additionally located on the sealing portion, in addition to the color filter and/or the color conversion layer, according to the use of the electronic device. Examples of the functional layers may include a touch screen layer, a polarizing layer, and the like. The touch screen layer may be a pressure-sensitive touch screen layer, a capacitive touch screen layer, or an infrared touch screen layer. The authentication apparatus may be, for example, a biometric authentication apparatus that authenticates an individual by using biometric information of a living body (for example, fingertips, pupils, etc.).

The authentication apparatus may further include, in addition to the light-emitting device as described above, a biometric information collector.

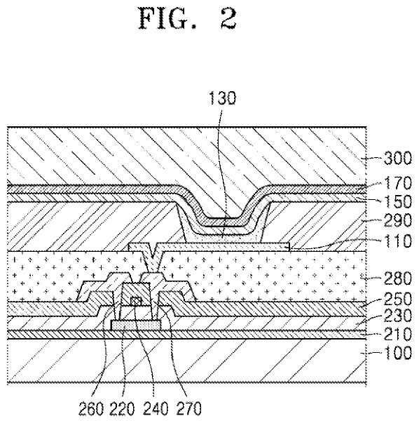

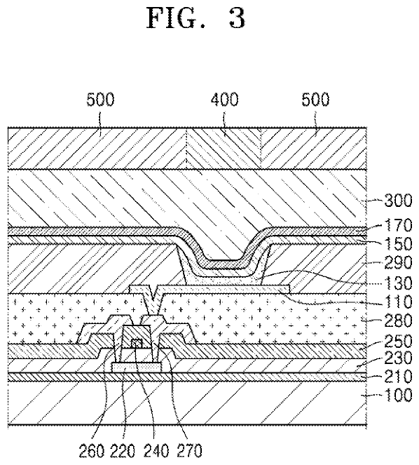

The electronic device may be applied to various displays, light sources, lighting, personal computers (for example, a mobile personal computer), mobile phones, digital cameras, electronic organizers, electronic dictionaries, electronic game machines, medical instruments (for example, electronic thermometers, sphygmomanometers, blood glucose meters, pulse measurement devices, pulse wave measurement devices, electrocardiogram displays, ultrasonic diagnostic devices, or endoscope displays), fish finders, various measuring instruments, meters (for example, meters for a vehicle, an aircraft, and a vessel), projectors, and the like.

[Description of FIGS. 2 and 3]

FIG. 2 is a schematic cross-sectional view showing an electronic device according to an embodiment of the disclosure.

The electronic device (for example, a light-emitting apparatus) of FIG. 2 includes a substrate 100, a thin-film transistor (TFT), a light-emitting device, and an encapsulation portion 300 that seals the light-emitting device.

The substrate 100 may be a flexible substrate, a glass substrate, or a metal substrate. A buffer layer 210 may be located on the substrate 100. The buffer layer 210 may prevent penetration of impurities through the substrate 100 and may provide a flat surface on the substrate 100.

A TFT may be located on the buffer layer 210. The TFT may include an active layer 220, a gate electrode 240, a source electrode 260, and a drain electrode 270.

The active layer 220 may include an inorganic semiconductor such as silicon or polysilicon, an organic semiconductor, or an oxide semiconductor, and may include a source region, a drain region, and a channel region.

A gate insulating film 230 for insulating the activation layer 220 from the gate electrode 240 may be located on the active layer 220, and the gate electrode 240 may be located on the gate insulating film 230.

An interlayer insulating film 250 may be located on the gate electrode 240. The interlayer insulating film 250 may be located between the gate electrode 240 and the source electrode 260 to insulate the gate electron 240 from the source electrode 260 and between the gate electrode 240 and the drain electrode 270, to insulate the gate electrode 240 from the drain electrode 270.

The source electrode 260 and the drain electrode 270 may be located on the interlayer insulating film 250. The interlayer insulating film 250 and the gate insulating film 230 may be formed to expose a source region and a drain region of the active layer 220, and the source electrode 260 and the drain electrode 270 may respectively contact the exposed portions of the source region and the drain region of the active layer 220.

The TFT may be electrically connected to a light-emitting device to drive the light-emitting device, and may be covered and protected by a passivation layer 280. The passivation layer 280 may include an inorganic insulating film, an organic insulating film, or any combination thereof. A light-emitting device may be provided on the passivation layer 280. The light-emitting device may include a first electrode 110, an interlayer 130, and a second electrode 150.

The first electrode 110 may be located on the passivation layer 280. The passivation layer 280 may be located to expose a portion of the drain electrode 270, and may not fully cover the drain electrode 270. The first electrode 110 may be located to be electrically connected to the exposed portion of the drain electrode 270.

A pixel defining layer 290 including an insulating material may be located on the first electrode 110. The pixel defining layer 290 may expose a region of the first electrode 110, and an interlayer 130 may be formed in the exposed region of the first electrode 110. The pixel defining layer 290 may be a polyimide or polyacrylic organic film. Although not shown in FIG. 2, at least some layers of the interlayer 130 may extend beyond the upper portion of the pixel defining layer 290 to be provided in the form of a common layer.

The second electrode 150 may be located on the interlayer 130, and a capping layer 170 may be further included on the second electrode 150. The capping layer 170 may be formed to cover the second electrode 150.

The encapsulation portion 300 may be located on the capping layer 170. The encapsulation portion 300 may be located on a light-emitting device to protect the light-emitting device from moisture or oxygen. The encapsulation portion 300 may include: an inorganic film including silicon nitride (SiNx), silicon oxide (SiOx), indium tin oxide, indium zinc oxide, or any combination thereof; an organic film including polyethylene terephthalate, polyethylene naphthalate, polycarbonate, polyimide, polyethylene sulfonate, polyoxymethylene, polyarylate, hexamethyldisiloxane, an acrylic resin (for example, polymethyl methacrylate, polyacrylic acid, or the like), an epoxy-based resin (for example, aliphatic glycidyl ether (AGE), or the like), or any combination thereof; or any combination of the inorganic films and the organic films.

FIG. 3 shows a schematic cross-sectional view showing an electronic device according to an embodiment of the disclosure.

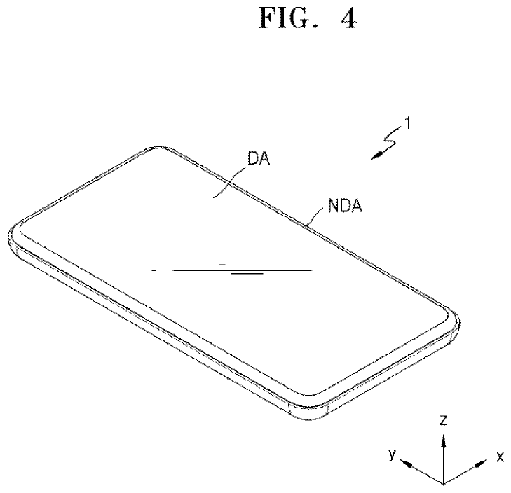

The electronic device (for example, a light-emitting apparatus) of FIG. 3 may differ from the electronic device of FIG. 2, at least in that a light-shielding pattern 500 and a functional region 400 are further included on the encapsulation portion 300. The functional region 400 may be a color filter area, a color conversion area, or a combination of the color filter area and the color conversion area. In an embodiment, the light-emitting device included in the electronic device of FIG. 3 may be a tandem light-emitting device.

[Description of FIG. 4]

FIG. 4 is a schematic perspective view of an electronic apparatus 1 including a light-emitting device according to an embodiment.

The electronic apparatus 1, which may be an apparatus that displays a moving image or still image, may be not only a portable electronic equipment, such as a mobile phone, a smartphone, a tablet personal computer (PC), a mobile communication terminal, an electronic notebook, an electronic book, a portable multimedia player (PMP), a navigation device, or a ultra mobile PC (UMPC), but may also be various products, such as a television, a laptop computer, a monitor, a billboard, or an Internet of things (IOT). The electronic apparatus 1 may be such a product as described above, or may be a part thereof.

The electronic apparatus 1 may be a wearable device, such as a smart watch, a watch phone, a glasses-type display, or a head mounted display (HMD), or may be a part of the wearable device. However, embodiments of the disclosure are not limited thereto.

In an embodiment, the electronic apparatus 1 may be a center information display (CID) on an instrument panel and a center fascia or dashboard of a vehicle, a room mirror display instead of a side mirror of a vehicle, an entertainment display for the rear seat of a car or a display placed on the back of the front seat, head up display (HUD) installed in front of a vehicle or projected on a front window glass, or a computer generated hologram augmented reality head up display (CGH AR HUD). FIG. 4 illustrates an embodiment in which the electronic apparatus 1 is a smartphone, for convenience of explanation.

The electronic apparatus 1 may include a display area DA and a non-display area NDA outside the display area DA. A display device may implement an image through a two-dimensional array of pixels that are arranged in the display area DA.

The non-display area NDA is an area that does not display an image, and may surround the display area DA. On the non-display area NDA, a driver for providing electrical signals or power to display devices arranged on the display area DA may be arranged. On the non-display area NDA, a pad, which is an area to which an electronic element or a printing circuit board may be electrically connected, may be arranged.

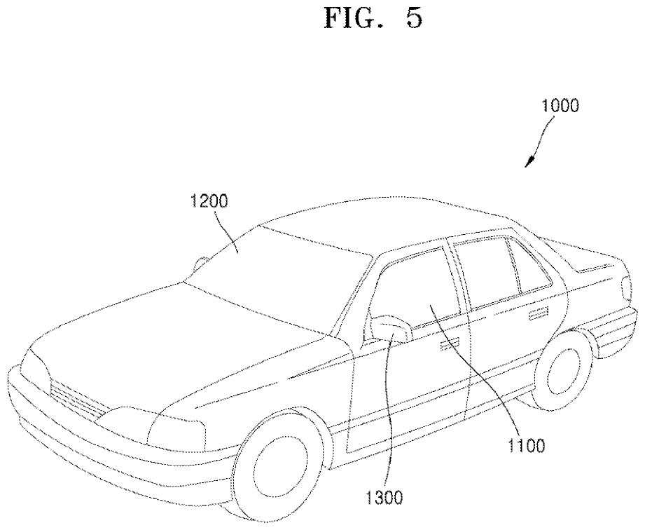

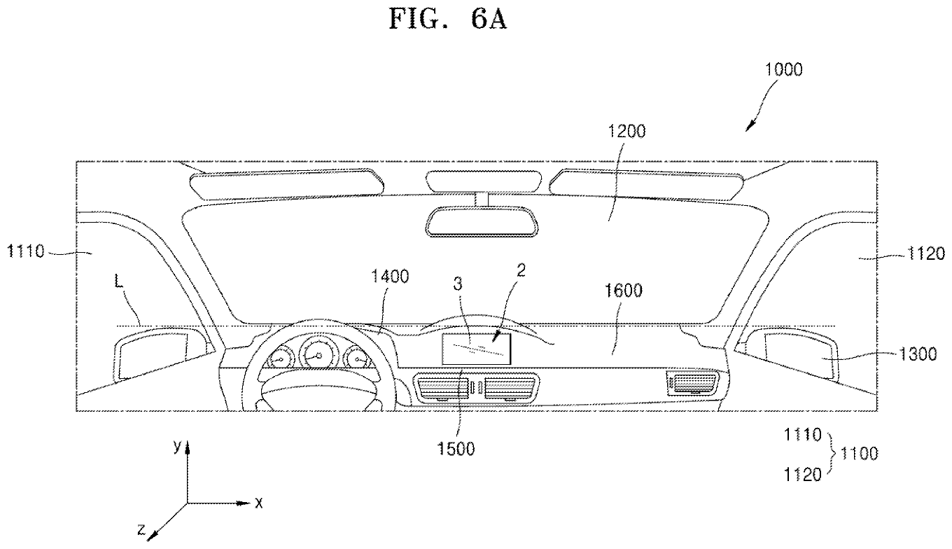

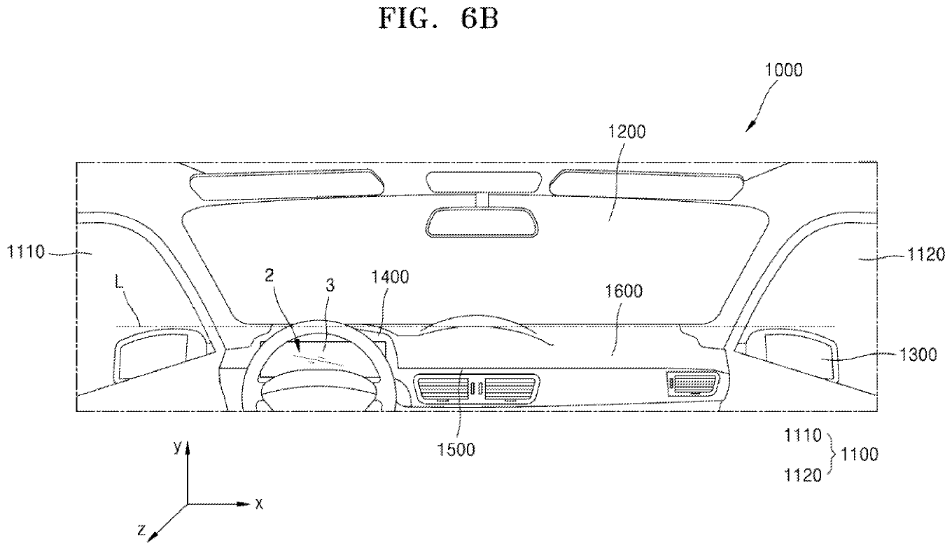

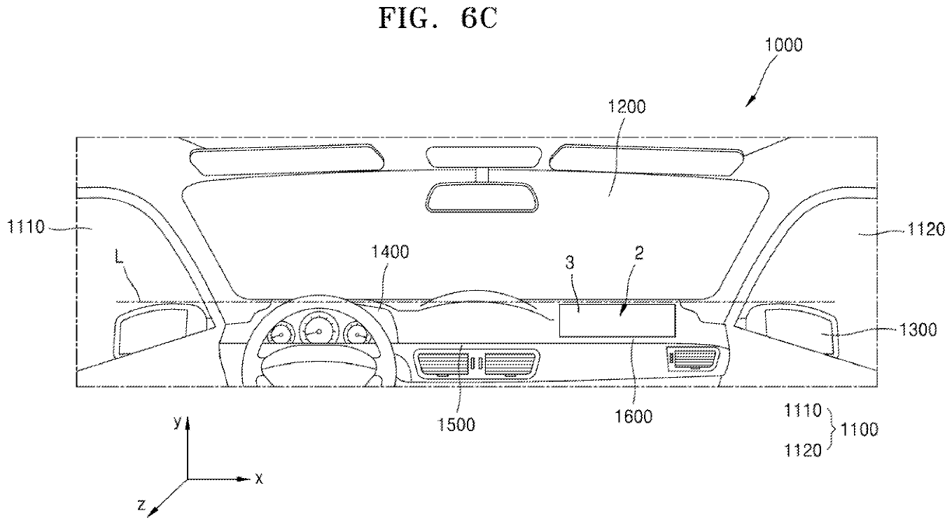

In the electronic apparatus 1, a length in the x-axis direction and a length in the y-axis direction may be different from each other. For example, as shown in FIG. 4, a length in the x-axis direction may be shorter than a length in the y-axis direction. In embodiments, a length in the x-axis direction may be the same as a length in the y-axis direction. In embodiments, a length in the x-axis direction may be longer than a length in the y-axis direction.

[Description of FIGS. 5 and 6A to 6C]

FIG. 5 is a schematic perspective view of exterior of a vehicle 1000 as an electronic apparatus including a light-emitting device according to an embodiment. FIGS. 6A to 6C are each a schematic diagram illustrating an interior of the vehicle 1000 according to embodiments.

Referring to FIGS. 5, 6A, 6B, and 6C, the vehicle 1000 may refer to various apparatuses for moving a subject to be transported, such as a human, an object, or an animal, from a departure point to a destination. Examples of the vehicle 1000 may include a vehicle traveling on a road or track, a vessel moving over a sea or river, an airplane flying in the sky using the action of air, and the like.

The vehicle 1000 may travel on a road or a track. The vehicle 1000 may move in a direction (e.g., a predetermined direction or a selectable direction) according to the rotation of at least one wheel. For example, the vehicle 1000 may include a three-wheeled or four-wheeled vehicle, a construction machine, a two-wheeled vehicle, a prime mover device, a bicycle, and a train running on a track.

The vehicle 1000 may include a body having an interior and an exterior, and a chassis that is a portion excluding the body in which mechanical apparatuses necessary for driving are installed. The exterior of the vehicle body may include a front panel, a bonnet, a roof panel, a rear panel, a trunk, a pillar provided at a boundary between doors, and the like. The chassis of the vehicle 1000 may include a power generating device, a power transmitting device, a driving device, a steering device, a braking device, a suspension device, a transmission device, a fuel device, front and rear wheels, left and right wheels, and the like.

The vehicle 1000 may include a side window glass 1100, a front window glass 1200, a side mirror 1300, a cluster 1400, a center fascia 1500, a passenger seat dashboard 1600, and a display device 2.

The side window glass 1100 and the front window glass 1200 may be partitioned by a pillar arranged between the side window glass 1100 and the front window glass 1200.

The side window glass 1100 may be installed on a side of the vehicle 1000. In an embodiment, the side window glass 1100 may be installed on a door of the vehicle 1000. Multiple side window glasses 1100 may be provided and may face each other. In an embodiment, the side window glass 1100 may include a first side window glass 1110 and a second side window glass 1120. In an embodiment, the first side window glass 1110 may be arranged adjacent to the cluster 1400, and the second side window glass 1120 may be arranged adjacent to the passenger seat dashboard 1600.

In an embodiment, the side window glasses 1100 may be spaced apart from each other in the x-direction or the −x-direction. For example, the first side window glass 1110 and the second side window glass 1120 may be spaced apart from each other in the x direction or the −x direction. For example, an imaginary straight line L connecting the side window glasses 1100 may extend in the x-direction or the −x-direction. For example, an imaginary straight line L connecting the first side window glass 1110 and the second side window glass 1120 to each other may extend in the x direction or the −x direction.

The front window glass 1200 may be installed on the front of the vehicle 1000. The front window glass 1200 may be arranged between the side window glasses 1100 facing each other.

The side mirror 1300 may provide a rear view of the vehicle 1000. The side mirror 1300 may be installed on the exterior of the vehicle body. In one embodiment, multiple side mirrors 1300 may be provided. Any one of the side mirrors 1300 may be arranged outside the first side window glass 1110. Another of the side mirrors 1300 may be arranged outside the second side window glass 1120.

The cluster 1400 may be arranged in front of the steering wheel. The cluster 1400 may include a tachometer, a speedometer, a coolant thermometer, a fuel gauge turn indicator, a high beam indicator, a warning lamp, a seat belt warning lamp, an odometer, a hodometer, an automatic shift selector indicator lamp, a door open warning lamp, an engine oil warning lamp, and/or a low fuel warning light.

The center fascia 1500 may include a control panel on which buttons for adjusting an audio device, an air conditioning device, and a seat heater are disposed. The center fascia 1500 may be arranged on one side of the cluster 1400.

A passenger seat dashboard 1600 may be spaced apart from the cluster 1400 with the center fascia 1500 arranged therebetween. In an embodiment, the cluster 1400 may be arranged to correspond to a driver seat (not shown), and the passenger seat dashboard 1600 may be disposed to correspond to a passenger seat (not shown). In an embodiment, the cluster 1400 may be adjacent to the first side window glass 1110, and the passenger seat dashboard 1600 may be adjacent to the second side window glass 1120.

In an embodiment, the display device 2 may include a display panel 3, and the display panel 3 may display an image. The display device 2 may be arranged inside the vehicle 1000. In an embodiment, the display device 2 may be arranged between the side window glasses 1100 facing each other. The display device 2 may be arranged on at least one of the cluster 1400, the center fascia 1500, and the passenger seat dashboard 1600.

The display device 2 may include an organic light-emitting display device, an inorganic electroluminescent (EL) display device, a quantum dot display device, and the like. Hereinafter, as the display device 2 according to an embodiment of the disclosure, an organic light-emitting display device display including the light-emitting device according to the disclosure will be described as an example, but various types of display devices as described above may be used in embodiments of the disclosure.

Referring to FIG. 6A, the display device 2 may be arranged on the center fascia 1500. In an embodiment, the display device 2 may display navigation information. In an embodiment, the display device 2 may display information regarding audio, video, or vehicle settings.

Referring to FIG. 6B, the display device 2 may be arranged on the cluster 1400. When the display device 2 is arranged on the cluster 1400, the cluster 1400 may display driving information and the like through the display device 2. For example, the cluster 1400 may digitally implement driving information. The digital cluster 1400 may display vehicle information and driving information as images. For example, a needle and a gauge of a tachometer and various warning lights or icons may be displayed by a digital signal.

Referring to FIG. 6C, the display device 2 may be arranged on the dashboard 1600 of the passenger seat. The display device 2 may be embedded in the passenger seat dashboard 1600 or arranged on the passenger seat dashboard 1600. In an embodiment, the display device 2 arranged on the dashboard 1600 for the passenger seat may display an image related to information displayed on the cluster 1400 and/or information displayed on the center fascia 1500. In embodiments, the display device 2 arranged on the passenger seat dashboard 1600 may display information that is different from the information displayed on the cluster 1400 and/or the information displayed on the center fascia 1500.

[Manufacturing Method]

Respective layers included in the hole transport region, the emission layer, and respective layers included in the electron transport region may be formed in a certain region by using one or more suitable methods selected from vacuum deposition, spin coating, casting, Langmuir-Blodgett (LB) deposition, ink-jet printing, laser-printing, and laser-induced thermal imaging.

When layers constituting the hole transport region, an emission layer, and layers constituting the electron transport region are formed by vacuum deposition, the deposition may be performed at a deposition temperature of about 100° C. to about 500° C., a vacuum degree of about 10⁻⁸ torr to about 10⁻³ torr, and a deposition speed of about 0.01 Å/sec to about 100 Å/sec, depending on a material to be included in a layer to be formed and the structure of a layer to be formed.

### Definitions of Terms

The term “C₃-C₆₀ carbocyclic group” as used herein may be a cyclic group consisting of carbon atoms as the only ring-forming atoms and having three to sixty carbon atoms, and the term “C₁-C₆₀ heterocyclic group” as used herein may be a cyclic group that has one to sixty carbon atoms and further has, in addition to a carbon atom, at least one heteroatom as a ring-forming atom. The C₃-C₆₀ carbocyclic group and the C₁-C₆₀ heterocyclic group may each be a monocyclic group consisting of one ring or a polycyclic group in which two or more rings are condensed with each other. For example, a C₁-C₆₀ heterocyclic group may have 3 to 61 ring-forming atoms.

The “cyclic group” as used herein may be a C₃-C₆₀ carbocyclic group, or a C₁-C₆₀ heterocyclic group.

The term “π electron-rich C₃-C₆₀ cyclic group” as used herein may be a cyclic group that has three to sixty carbon atoms and may not include *—N═*′ as a ring-forming moiety, and the term “π electron-deficient nitrogen-containing C₁-C₆₀ cyclic group” as used herein may be a heterocyclic group that has one to sixty carbon atoms and may include *—N=*′ as a ring-forming moiety.

In embodiments,
a C₃-C₆₀ carbocyclic group may be a Ti group or a group in which two or more Ti groups are condensed with each other (for example, a cyclopentadiene group, an adamantane group, a norbornane group, a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, or an indenoanthracene group),
a C₁-C₆₀ heterocyclic group may be a T2 group, a group in which two or more T2 groups are condensed with each other, or a group in which at least one T2 group and at least one Ti group are condensed with each other (for example, a pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, etc.),
a π electron-rich C₃-C₆₀ cyclic group may be a T1 group, a group in which two or more T1 groups are condensed with each other, a T3 group, a group in which two or more T3 groups are condensed with each other, or a group in which at least one T3 group and at least one Ti group are condensed with each other (for example, a C₃-C₆₀ carbocyclic group, a 1H-pyrrole group, a silole group, a borole group, a 2H-pyrrole group, a 3H-pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, etc.),
a π electron-deficient nitrogen-containing C₁-C₆₀ cyclic group may be a T4 group, a group in which two or more T4 groups are condensed with each other, a group in which at least one T4 group and at least one Ti group are condensed with each other, a group in which at least one T4 group and at least one T3 group are condensed with each other, or a group in which at least one T4 group, at least one Ti group, and at least one T3 group are condensed with one another (for example, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, etc.), wherein

the T1 group may be a cyclopropane group, a cyclobutane group, a cyclopentane group, a cyclohexane group, a cycloheptane group, a cyclooctane group, a cyclobutene group, a cyclopentene group, a cyclopentadiene group, a cyclohexene group, a cyclohexadiene group, a cycloheptene group, an adamantane group, a norbornane (or a bicyclo[2.2.1]heptane) group, a norbornene group, a bicyclo[1.1.1]pentane group, a bicyclo[2.1.1]hexane group, a bicyclo[2.2.2]octane group, or a benzene group,
the T2 group may be a furan group, a thiophene group, a 1H-pyrrole group, a silole group, a borole group, a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, a tetrazine group, a pyrrolidine group, an imidazolidine group, a dihydropyrrole group, a piperidine group, a tetrahydropyridine group, a dihydropyridine group, a hexahydropyrimidine group, a tetrahydropyrimidine group, a dihydropyrimidine group, a piperazine group, a tetrahydropyrazine group, a dihydropyrazine group, a tetrahydropyridazine group, or a dihydropyridazine group,
the T3 group may be a furan group, a thiophene group, a 1H-pyrrole group, a silole group, or a borole group, and
the T4 group may be a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, or a tetrazine group.

The terms “cyclic group”, “C₃-C₆₀ carbocyclic group”, “C₁-C₆₀ heterocyclic group”, “π electron-rich C₃-C₆₀ cyclic group”, or “π electron-deficient nitrogen-containing C₁-C₆₀ cyclic group” as used herein each may be a group condensed to any cyclic group, a monovalent group, or a polyvalent group (for example, a divalent group, a trivalent group, a tetravalent group, etc.) according to the structure of a formula for which the corresponding term is used. For example, a “benzene group” may be a benzo group, a phenyl group, a phenylene group, or the like, which may be readily understood by one of ordinary skill in the art according to the structure of a formula including the “benzene group.”

Examples of a monovalent C₃-C₆₀ carbocyclic group or a monovalent C₁-C₆₀ heterocyclic group may include a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group. Examples of a divalent C₃-C₆₀ carbocyclic group or a monovalent C₁-C₆₀ heterocyclic group may include a C₃-C₁₀ cycloalkylene group, a C₁-C₁₀ heterocycloalkylene group, a C₃-C₁₀ cycloalkenylene group, a C₁-C₁₀ heterocycloalkenylene group, a C₆-C₆₀ arylene group, a C₁-C₆₀ heteroarylene group, a divalent non-aromatic condensed polycyclic group, and a substituted or unsubstituted divalent non-aromatic condensed heteropolycyclic group.

The term “C₁-C₆₀ alkyl group” as used herein may be a linear or branched aliphatic hydrocarbon monovalent group that has one to sixty carbon atoms, and examples thereof may include a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, and a tert-decyl group. The term “C₁-C₆₀ alkylene group” as used herein may be a divalent group having a same structure as the C₁-C₆₀ alkyl group.

The term “C₂-C₆₀ alkenyl group” as used herein may be a monovalent hydrocarbon group having at least one carbon-carbon double bond in the middle or at a terminus of a C₂-C₆₀ alkyl group, and examples thereof may include an ethenyl group, a propenyl group, and a butenyl group. The term “C₂-C₆₀ alkenylene group” as used herein may be a divalent group having a same structure as the C₂-C₆₀ alkenyl group.

The term “C₂-C₆₀ alkynyl group” as used herein may be a monovalent hydrocarbon group having at least one carbon-carbon triple bond in the middle or at a terminus of a C₂-C₆₀ alkyl group, and examples thereof may include an ethynyl group, a propynyl group, and the like. The term “C₂-C₆₀ alkynylene group” as used herein may be a divalent group having a same structure as the C₂-C₆₀ alkynyl group.

The term “C₁-C₆₀ alkoxy group” as used herein may be a monovalent group represented by —O(A₁₀₁) (wherein A₁₀₁ may be a the C₁-C₆₀ alkyl group), and examples thereof may include a methoxy group, an ethoxy group, and an isopropyloxy group.

The term “C₃-C₁₀ cycloalkyl group” as used herein may be a monovalent saturated hydrocarbon cyclic group having 3 to 10 carbon atoms, and examples thereof may include a cyclopropyl group, a cyclobutyl group, a cyclopentyl group, a cyclohexyl group, a cycloheptyl group, a cyclooctyl group, an adamantanyl group, a norbornanyl group (or bicyclo[2.2.1]heptyl group), a bicyclo[1.1.1]pentyl group, a bicyclo[2.1.1]hexyl group, and a bicyclo[2.2.2]octyl group. The term “C₃-C₁₀ cycloalkylene group” as used herein may be a divalent group having a same structure as the C₃-C₁₀ cycloalkyl group.

The term “C₁-C₁₀ heterocycloalkyl group” as used herein may be a monovalent cyclic group of 1 to 10 carbon atoms, further including, in addition to carbon atoms, at least one heteroatom, as ring-forming atoms, and examples may include a 1,2,3,4-oxatriazolidinyl group, a tetrahydrofuranyl group, and a tetrahydrothiophenyl group. The term “C₁-C₁₀ heterocycloalkylene group” as used herein may be a divalent group having a same structure as the C₁-C₁₀ heterocycloalkyl group.

The term C₃-C₁₀ cycloalkenyl group used herein may be a monovalent cyclic group that has three to ten carbon atoms and at least one carbon-carbon double bond in the ring thereof and no aromaticity, and examples thereof may include a cyclopentenyl group, a cyclohexenyl group, and a cycloheptenyl group. The term “C₃-C₁₀ cycloalkenylene group” as used herein may be a divalent group having a same structure as the C₃-C₁₀ cycloalkenyl group.

The term “C₁-C₁₀ heterocycloalkenyl group” as used herein may be a monovalent cyclic group of 1 to 10 carbon atoms, further including, in addition to carbon atoms, at least one heteroatom, as ring-forming atoms, and having at least one carbon-carbon double bond in the cyclic structure thereof. Examples of a C₁-C₁₀ heterocycloalkenyl group may include a 4,5-dihydro-1,2,3,4-oxatriazolyl group, a 2,3-dihydrofuranyl group, and a 2,3-dihydrothiophenyl group. The term “C₁-C₁₀ heterocycloalkenylene group” as used herein may be a divalent group having a same structure as the C₁-C₁₀ heterocycloalkenyl group.

The term “C₆-C₆₀ aryl group” as used herein may be a monovalent group having a carbocyclic aromatic system of 6 to 60 carbon atoms, and the term “C₆-C₆₀ arylene group” as used herein may be a divalent group having a carbocyclic aromatic system of 6 to 60 carbon atoms. Examples of a C₆-C₆₀ aryl group may include a phenyl group, a pentalenyl group, a naphthyl group, an azulenyl group, an indacenyl group, an acenaphthyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a heptalenyl group, a naphthacenyl group, a picenyl group, a hexacenyl group, a pentacenyl group, a rubicenyl group, a coronenyl group, and an ovalenyl group. When the C₆-C₆₀ aryl group and the C₆-C₆₀ arylene group each include two or more rings, the respective rings may be condensed with each other.

The term “C₁-C₆₀ heteroaryl group” as used herein may be a monovalent group having a heterocyclic aromatic system of 1 to 60 carbon atoms, further including, in addition to carbon atoms, at least one heteroatom, as ring-forming atoms. The term “C₁-C₆₀ heteroarylene group” as used herein may be a divalent group having a heterocyclic aromatic system of 1 to 60 carbon atoms, further including, in addition to carbon atoms, at least one heteroatom, as ring-forming atoms. Examples of a C₁-C₆₀ heteroaryl group may include a pyridinyl group, a pyrimidinyl group, a pyrazinyl group, a pyridazinyl group, a triazinyl group, a quinolinyl group, a benzoquinolinyl group, an isoquinolinyl group, a benzoisoquinolinyl group, a quinoxalinyl group, a benzoquinoxalinyl group, a quinazolinyl group, a benzoquinazolinyl group, a cinnolinyl group, a phenanthrolinyl group, a phthalazinyl group, and a naphthyridinyl group. When the C₁-C₆₀ heteroaryl group and the C₁-C₆₀ heteroarylene group each include two or more rings, the respective rings may be condensed with each other.

The term “monovalent non-aromatic condensed polycyclic group” as used herein may be a monovalent group (for example, having 8 to 60 carbon atoms) having two or more rings condensed to each other, only carbon atoms as ring-forming atoms, and no aromaticity in its entire molecular structure. Examples of a monovalent non-aromatic condensed polycyclic group may include an indenyl group, a fluorenyl group, a spiro-bifluorenyl group, a benzofluorenyl group, an indenophenanthrenyl group, and an indeno anthracenyl group. The term “divalent non-aromatic condensed polycyclic group” as used herein may be a divalent group having a same structure as the monovalent non-aromatic condensed polycyclic group described above.

The term “monovalent non-aromatic condensed heteropolycyclic group” as used herein may be a monovalent group (for example, having 1 to 60 carbon atoms) having two or more rings condensed to each other, further including, in addition to carbon atoms, at least one heteroatom, as ring-forming atoms, and having non-aromaticity in its entire molecular structure. Examples of a monovalent non-aromatic condensed heteropolycyclic group may include a pyrrolyl group, a thiophenyl group, a furanyl group, an indolyl group, a benzoindolyl group, a naphthoindolyl group, an isoindolyl group, a benzoisoindolyl group, a naphthoisoindolyl group, a benzosilolyl group, a benzothiophenyl group, a benzofuranyl group, a carbazolyl group, a dibenzosilolyl group, a dibenzothiophenyl group, a dibenzofuranyl group, an azacarbazolyl group, an azafluorenyl group, an azadibenzosilolyl group, an azadibenzothiophenyl group, an azadibenzofuranyl group, a pyrazolyl group, an imidazolyl group, a triazolyl group, a tetrazolyl group, an oxazolyl group, an isoxazolyl group, a thiazolyl group, an isothiazolyl group, an oxadiazolyl group, a thiadiazolyl group, a benzopyrazolyl group, a benzimidazolyl group, a benzoxazolyl group, a benzothiazolyl group, a benzoxadiazolyl group, a benzothiadiazolyl group, an imidazopyridinyl group, an imidazopyrimidinyl group, an imidazotriazinyl group, an imidazopyrazinyl group, an imidazopyridazinyl group, an indeno carbazolyl group, an indolocarbazolyl group, a benzofurocarbazolyl group, a benzothienocarbazolyl group, a benzosilolocarbazolyl group, a benzoindolocarbazolyl group, a benzocarbazolyl group, a benzonaphthofuranyl group, a benzonaphthothiophenyl group, a benzonaphtho silolyl group, a benzofurodibenzofuranyl group, a benzofurodibenzothiophenyl group, and a benzothienodibenzothiophenyl group. The term “divalent non-aromatic condensed heteropolycyclic group” as used herein may be a divalent group having a same structure as the monovalent non-aromatic condensed heteropolycyclic group described above.

The term “C₆-C₆₀ aryloxy group” as used herein may be a group represented by —O(A₁₀₂) (wherein A₁₀₂ may be a C₆-C₆₀ aryl group), and the term “C₆-C₆₀ arylthio group” as used herein may be a group represented by —S(A₁₀₃) (wherein A₁₀₃ may be a C₆-C₆₀ aryl group).

The term “C₇-C₆₀ arylalkyl group” used herein may be a group represented by -(A₁₀₄)(A₁₀₅) (where A₁₀₄ may be a C₁-C₅₄ alkylene group, and A₁₀₅ may be a C₆-C₅₉ aryl group), and the term C₂-C₆₀ heteroarylalkyl group” used herein may be a group represented by -(A₁₀₆)(A₁₀₇) (where A₁₀₆ may be a C₁-C₅₉ alkylene group, and A₁₀₇ may be a C₁-C₅₉ heteroaryl group).

In the specification, the group “R₁₀ₐ” as used herein may be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂).

In the specification, Q₁ to Q₃, Q₁₁ to Q₁₃, Q₂₁ to Q₂₃ and Q₃₁ to Q₃₃ may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof; a C₇-C₆₀ arylalkyl group; or a C₂-C₆₀ heteroarylalkyl group.

The term “heteroatom” as used herein may be any atom other than a carbon atom or a hydrogen atom. Examples of a heteroatom may include O, S, N, P, Si, B, Ge, Se, or any combinations thereof.

The term “third-row transition metal” as used herein may be hafnium (Hf), tantalum (Ta), tungsten (W), rhenium (Re), osmium (Os), iridium (Ir), platinum (Pt), gold (Au), and the like.

The term “Ph” refers to a phenyl group, the term “Me” refers to a methyl group, the term “Et” refers to an ethyl group, the term “tert-Bu” or “But” each refer to a tert-butyl group, and the term “OMe” refers to a methoxy group.

The term “biphenyl group” as used herein may be “a phenyl group substituted with a phenyl group”. For example, the “biphenyl group” may be a substituted phenyl group having a C₆-C₆₀ aryl group as a substituent.

The term “terphenyl group” as used herein may be “a phenyl group substituted with a biphenyl group”. For example, the “terphenyl group” may be a substituted phenyl group having, as a substituent, a C₆-C₆₀ aryl group substituted with a C₆-C₆₀ aryl group.

The symbols * and *′ as used herein, unless defined otherwise, each refer to a binding site to a neighboring atom in a corresponding formula or moiety.

In the specification, “x-axis”, “y-axis”, and “z-axis” are not limited to three axes in an orthogonal coordinate system (for example, a Cartesian coordinate system), and may be interpreted in a broader sense than the aforementioned three axes in an orthogonal coordinate system. For example, the x-axis, y-axis, and z-axis may be axes that are orthogonal to each other, or may be axes that are in different directions that are not orthogonal to each other.

Hereinafter, compounds according to embodiments and light-emitting devices according to embodiments will be described in detail with reference to the following Synthesis Examples and Examples. The wording “B was used instead of A” used in describing Synthesis Examples refers to that an identical molar equivalent of B was used in place of A.

### EXAMPLES

### Synthesis of Intermediate Compounds 61-A, 61-B, 87-A, 125-A, 125-B, 142-A, 142-B, 154-A, 154-B, and 187-A

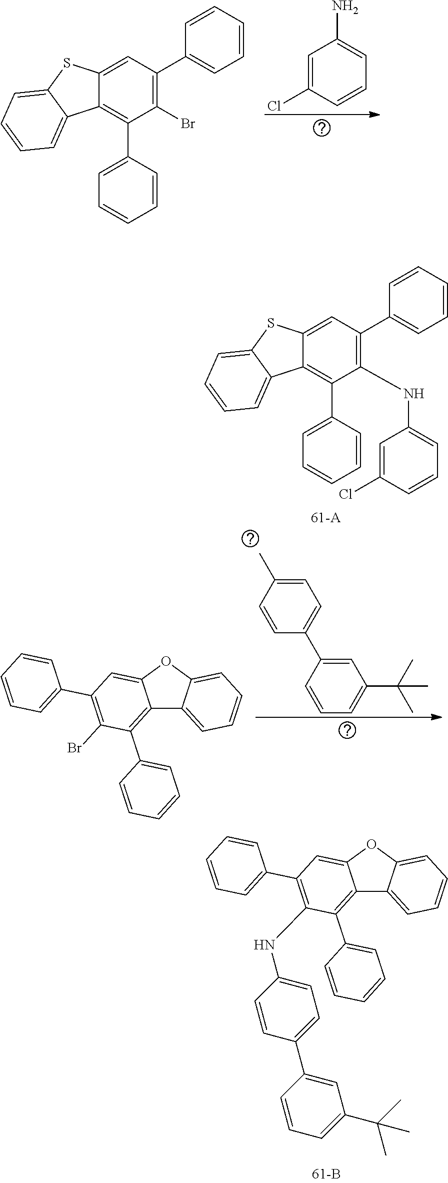

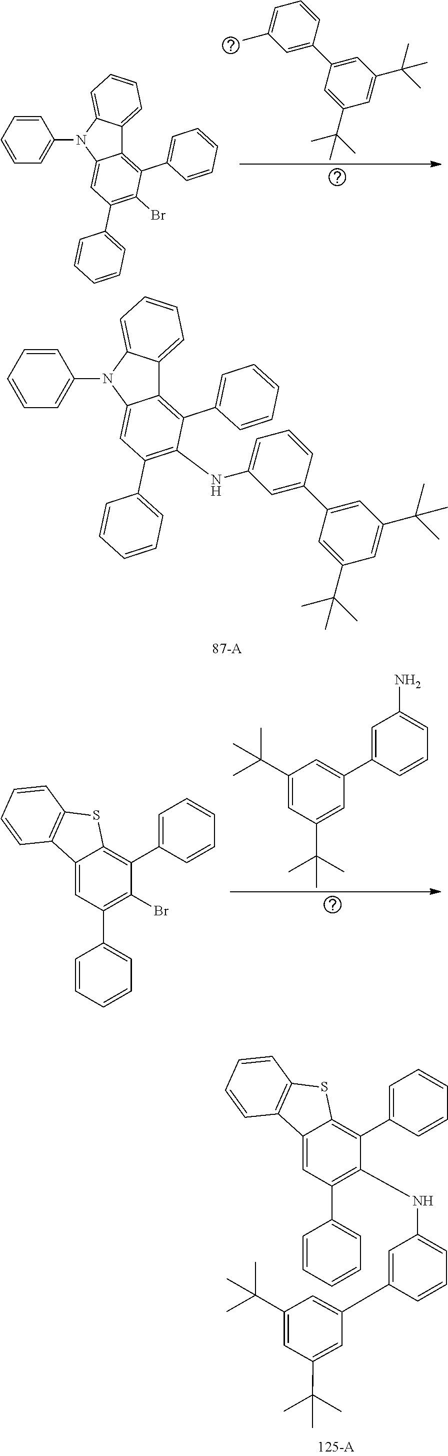

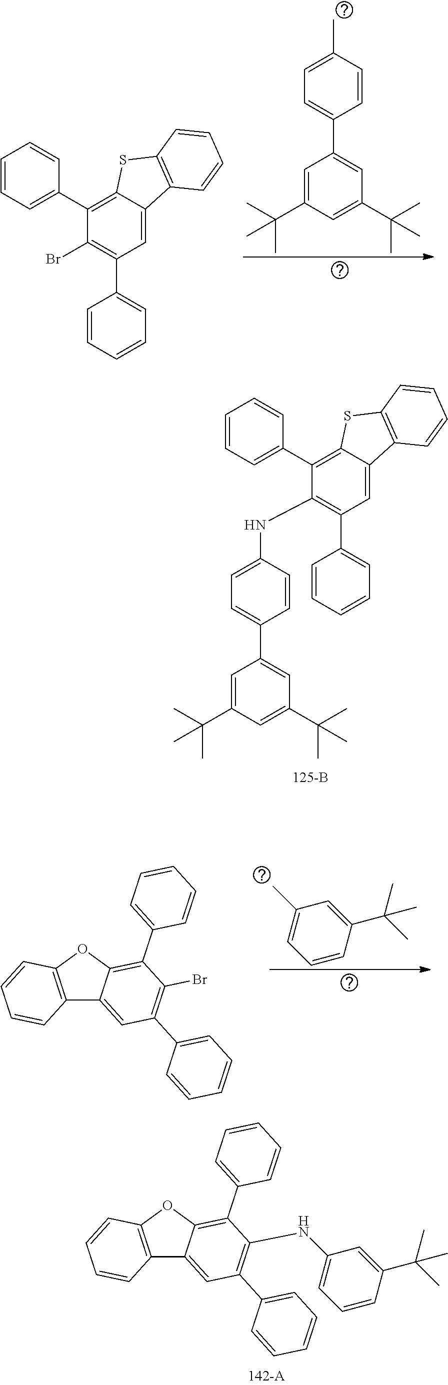

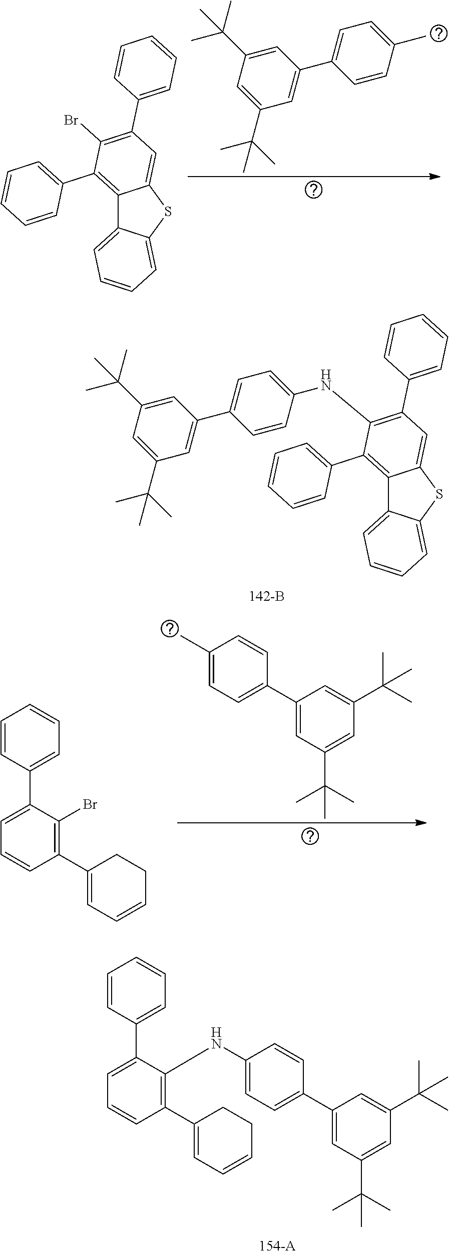

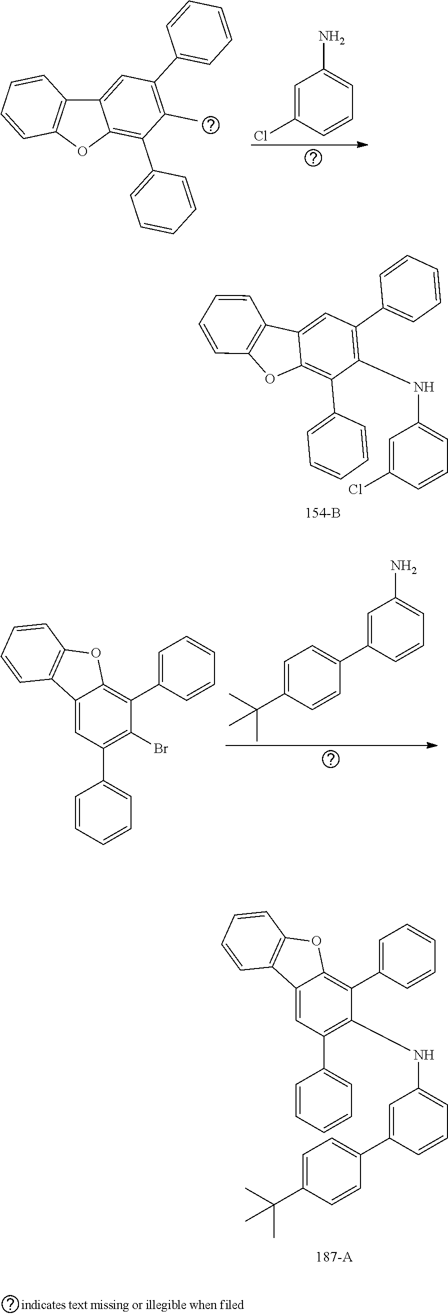

Each of the Intermediate Compounds was prepared by the Buchwald-Hartwig organic reaction, and the obtained compounds were identified by ESI-LCMS. For synthesis of each Intermediate Compound, each starting compound (1 eq), each nitrogen-containing compound (1 eq), tris(dibenzylideneacetone)dipalladium(0) (0.05 eq), tri-tert-butylphosphine (0.10 eq), and sodium tert-butoxide (1.7 eq) were dissolved in xylene, followed by stirring in a nitrogen atmosphere at 85° C. to 90° C. for 10 hours to 12 hours. The mixture was cooled and dried under reduced pressure to remove o-xylene. A washing process was performed thereon three times by using ethyl acetate and water, and an organic layer thus obtained was dried by using MgSO₄ under reduced pressure. The resulting product was purified by column chromatography and recrystallized (dichloromethane:n-Hexane) to obtain each of Intermediate Compounds 61-A, 61-B, 87-A, 125-A, 125-B, 142-A, 142-B, 154-A, 154-B, and 187-A. (Yield: 60% to 70%).

### Synthesis Example 1 (Synthesis of Compound 61)

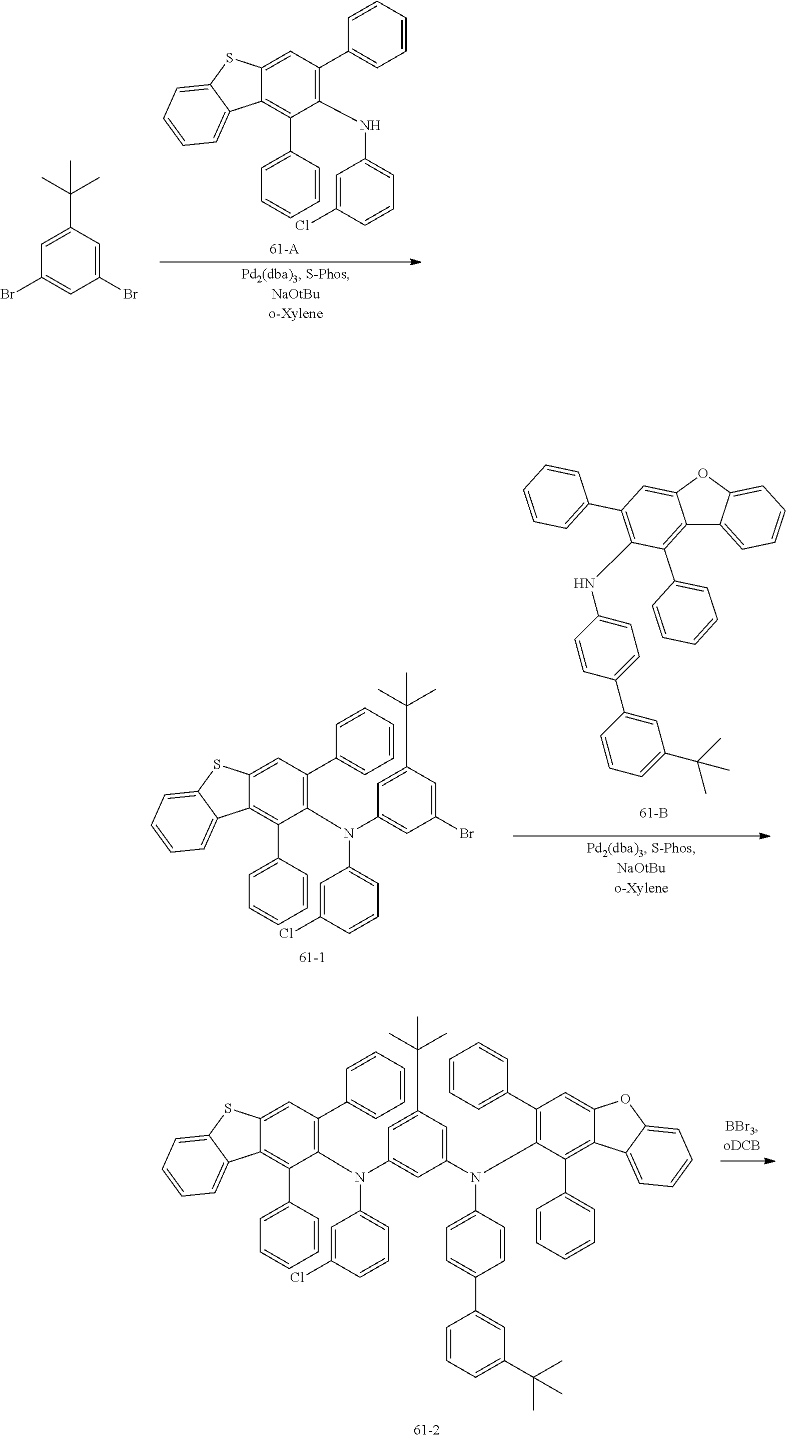 [SMILES: CC(C)(C)c1cc(Br)cc(Br)c1 ; Clc1cccc(Nc2c(-c3ccccc3)cc3sc4ccccc4c3c2-c2ccccc2)c1 ; CC(C)(C)c1cc(Br)cc(N(c2cccc(Cl)c2)c2c(-c3ccccc3)cc3sc4ccccc4c3c2-c2ccccc2)c1 ; CC(C)(C)c1cccc(-c2ccc(Nc3c(-c4ccccc4)cc4oc5ccccc5c4c3-c3ccccc3)cc2)c1 ; CC(C)(C)c1cccc(-c2ccc(N(c3cc(N(c4cccc(Cl)c4)c4c(-c5ccccc5)cc5sc6ccccc6c5c4-c4ccccc4)cc(C(C)(C)C)c3)c3c(-c4ccccc4)cc4oc5ccccc5c4c3-c3ccccc3)cc2)c1]

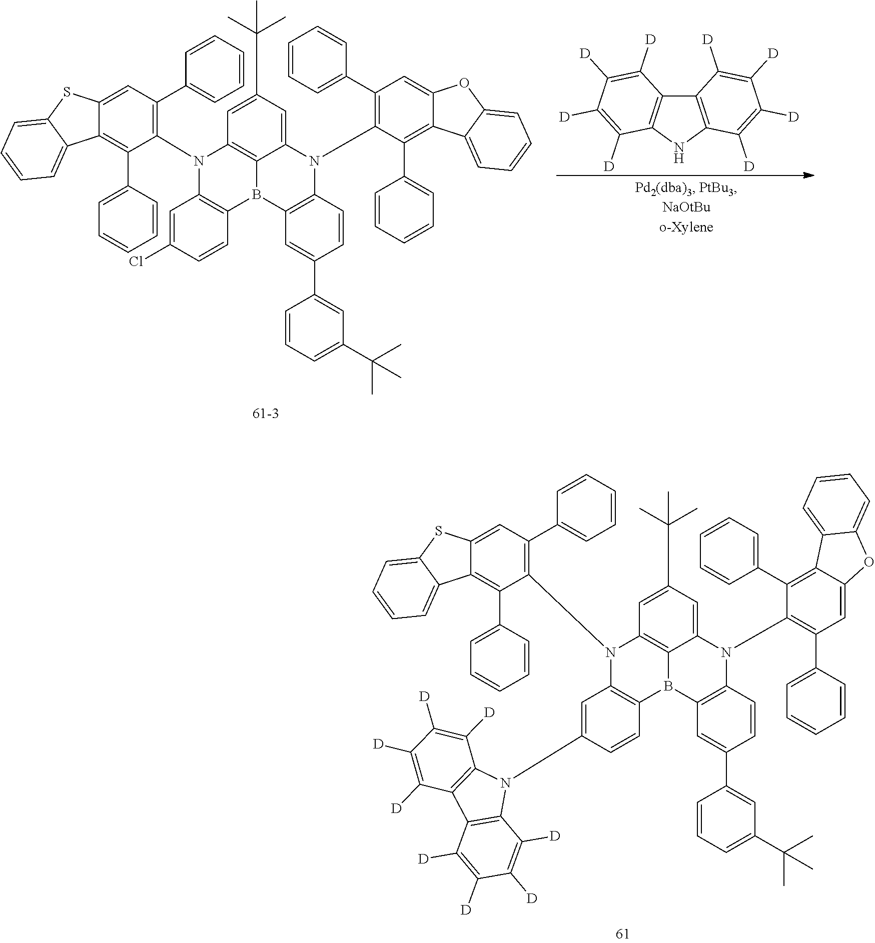

### Synthesis of Intermediate Compound 61-1

1,3-dibromo-5-(tert-butyl)benzene (1 eq), Intermediate Compound 61-A (1 eq), tris(dibenzylideneacetone)dipalladium(0) (0.05 eq), 2-dicyclohexylphosphino-2′,6′-dimethoxybiphenyl (S-Phos, 0.10 eq), and sodium tert-butoxide (1.5 eq) were dissolved in xylene, followed by stirring in a nitrogen atmosphere at 150° C. for 12 hours in a high-pressure reactor. The mixture was cooled and dried under reduced pressure to remove o-xylene. A washing process was performed thereon three times by using ethyl acetate and water, and an organic layer thus obtained was dried by using MgSO₄ under reduced pressure. The resulting product was purified by column chromatography and recrystallized (dichloromethane:n-Hexane) to obtain Intermediate Compound 61-1. The yield was 62%.

### Synthesis of Intermediate Compound 61-2

Intermediate Compound 61-1 (1 eq), Intermediate Compound 61-B (1 eq), tris(dibenzylideneacetone)dipalladium(0) (0.05 eq), 2-dicyclohexylphosphino-2′,6′-dimethoxybiphenyl (S-Phos, 0.10 eq), and sodium tert-butoxide (1.5 eq) were dissolved in xylene, followed by stirring in a nitrogen atmosphere at 150° C. for 10 hours in a high-pressure reactor. The mixture was cooled and dried under reduced pressure to remove o-xylene. A washing process was performed thereon three times by using ethyl acetate and water, and an organic layer thus obtained was dried by using MgSO₄ under reduced pressure. The resulting product was purified by column chromatography and recrystallized (dichloromethane:n-Hexane) to obtain Intermediate Compound 61-2. The yield was 67%.

### Synthesis of Intermediate Compound 61-3

After Intermediate Compound 61-2 (1 eq) was dissolved in ortho dichlorobenzene, the flask was cooled to 0° C. in a nitrogen atmosphere, and boron tribromide (BBr₃, 2.5 eq) dissolved in ortho dichlorobenzene was slowly injected thereto. After termination of the dropwise addition, the temperature was raised to 190° C., followed by stirring for 24 hours. After cooling to 0° C., triethylamine was slowly added dropwise to the flask to terminate the reaction until the exotherm stopped, and then, n-hexane and methanol were added thereto to cause precipitation and a solid was obtained therefrom by filtration. The obtained solid was purified by silica filtration, and purified by MC/Hex recrystallization again to obtain Intermediate Compound 61-3. Final purification was performed thereon by using column chromatography and recrystallized (dichloromethane:n-Hexane) to obtain Intermediate Compound 61-3. The yield was 12%.

### Synthesis of Compound 61

Intermediate Compound 61-3 (1 eq), 9H-carbazole-1,2,3,4,5,6,7,8-d8 (1.5 eq), tris(dibenzylideneacetone)dipalladium(0) (0.05 eq), tri-tert-butylphosphine (0.10 eq), and sodium tert-butoxide (1.5 eq) were dissolved in xylene, followed by stirring in a nitrogen atmosphere at 150° C. for 10 hours. The mixture was cooled and dried under reduced pressure to remove o-xylene. A washing process was performed thereon three times by using ethyl acetate and water, and an organic layer thus obtained was dried by using MgSO₄ under reduced pressure. The resulting product was purified by column chromatography (dichloromethane:n-Hexane), and recrystallized to obtain Compound 61. The yield was 60%.

The compound was finally purified by sublimation purification. By ESI-LCMS, the obtained compound was identified as Compound 61.

ESI-LCMS: [M]⁺: C₉₂H₆₀N₃. 1282.9

### Synthesis Example 2 (Synthesis of Compound 87)

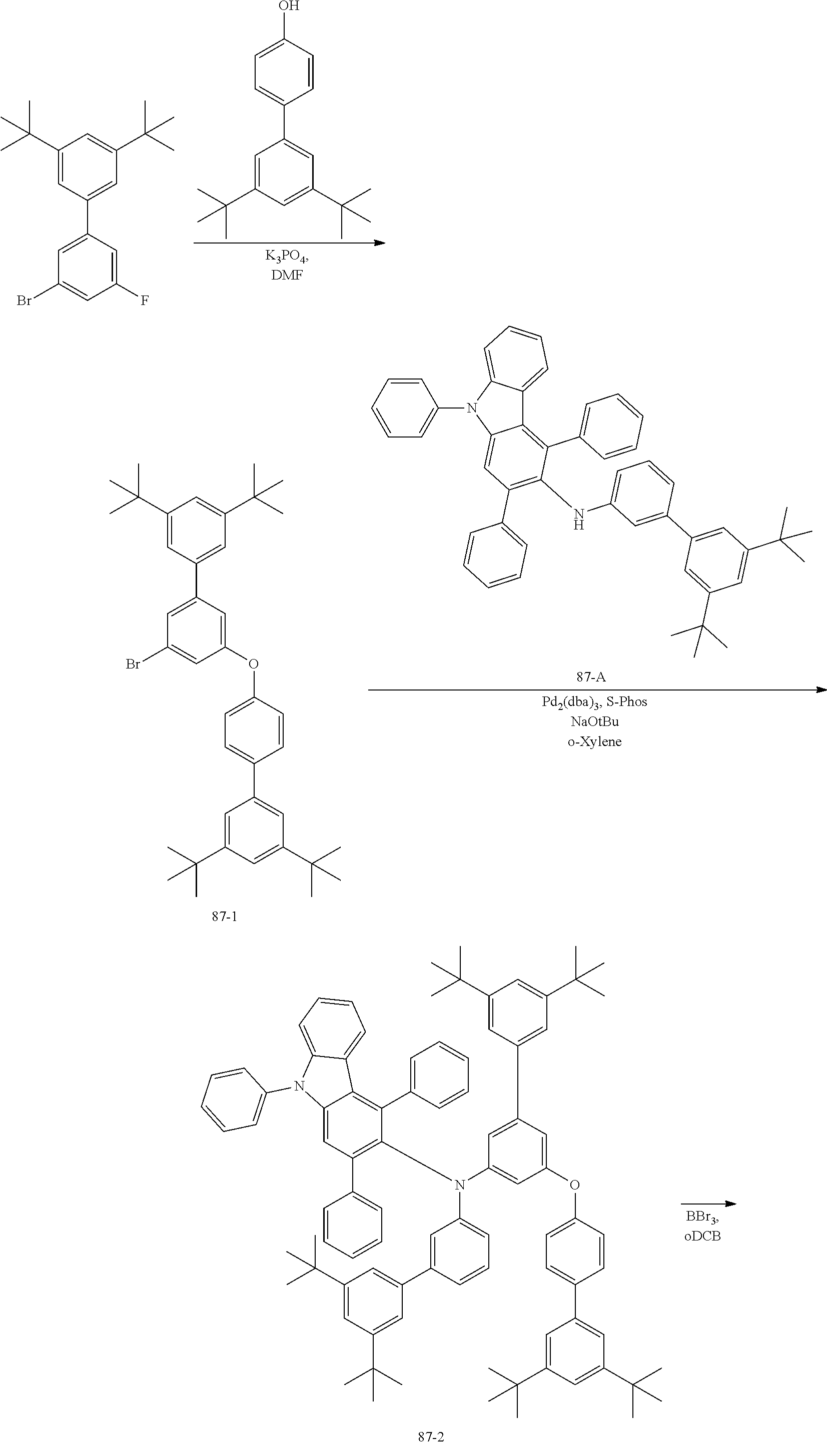

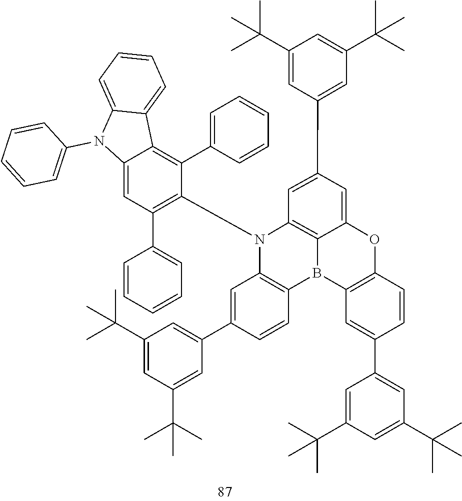 [SMILES: CC(C)(C)c1cc(-c2ccc3c(c2)B2c4ccc(-c5cc(C(C)(C)C)cc(C(C)(C)C)c5)cc4N(c4c(-c5ccccc5)cc5c(c4-c4ccccc4)c4ccccc4n5-c4ccccc4)c4cc(-c5cc(C(C)(C)C)cc(C(C)(C)C)c5)cc(c42)O3)cc(C(C)(C)C)c1]

### Synthesis of Intermediate Compound 87-1

3-bromo-3′,5′-di-tert-butyl-5-fluoro-1,1′-biphenyl (1 eq), 3′,5′-di-tert-butyl-[1,1′-biphenyl]-4-ol (1 eq), potassium phosphate, and tribasic (3 eq) were dissolved in DMF, followed by stirring in a nitrogen atmosphere at 150° C. for 10 hours. The mixture was cooled and dried under reduced pressure to remove DMF. A washing process was performed thereon three times by using ethyl acetate and water, and an organic layer thus obtained was dried by using MgSO₄ under reduced pressure. The resulting product was purified by column chromatography and recrystallized (dichloromethane:n-Hexane) to obtain Intermediate Compound 87-1. The yield was 71%.

### Synthesis of Intermediate Compound 87-2

Intermediate Compound 87-1 (1 eq), N-(3′,5′-di-tert-butyl-[1,1′-biphenyl]-3-yl)-2,4,9-triphenyl-9H-carbazol-3-amine (1 eq), 2-dicyclohexylphosphino-2′,6′-dimethoxybiphenyl (S-Phos, 0.10 eq), and sodium tert-butoxide (2 eq) were dissolved in o-Xylene, followed by stirring in a nitrogen atmosphere at 150° C. for 20 hours in a high-pressure reactor. The mixture was cooled and dried under reduced pressure to remove o-xylene. A washing process was performed thereon three times by using ethyl acetate and water, and an organic layer thus obtained was dried by using MgSO₄ under reduced pressure. The resulting product was purified by column chromatography and recrystallized (dichloromethane:n-Hexane) to obtain Intermediate Compound 87-2. The yield was 79%.

### Synthesis of Compound 87

After Intermediate Compound 87-2 (1 eq) was dissolved in ortho dichlorobenzene, the flask was cooled to 0° C. in a nitrogen atmosphere, and BBr₃ (2.5 eq) dissolved in ortho dichlorobenzene was slowly injected thereto. After termination of the dropwise addition, the temperature was raised to 190° C., followed by stirring for 24 hours. After cooling to 0° C., triethylamine was slowly added dropwise to the flask to terminate the reaction until the exotherm stopped, and methanol was added thereto to cause precipitation and a solid was obtained therefrom by filtration. The obtained solid was purified by silica pad filtration, and purified by MC/Hex recrystallization again to obtain Compound 87. Purification was performed by column chromatography and recrystallized (dichloromethane:n-Hexane), followed by final sublimation purification. The yield was 15%. By ESI-LCMS, the obtained compound was identified as Compound 87.

ESI-LCMS: [M]⁺: C₉₀H₉₁BN₂O. 1227.9

### Synthesis Example 3 (Synthesis of Compound 125)

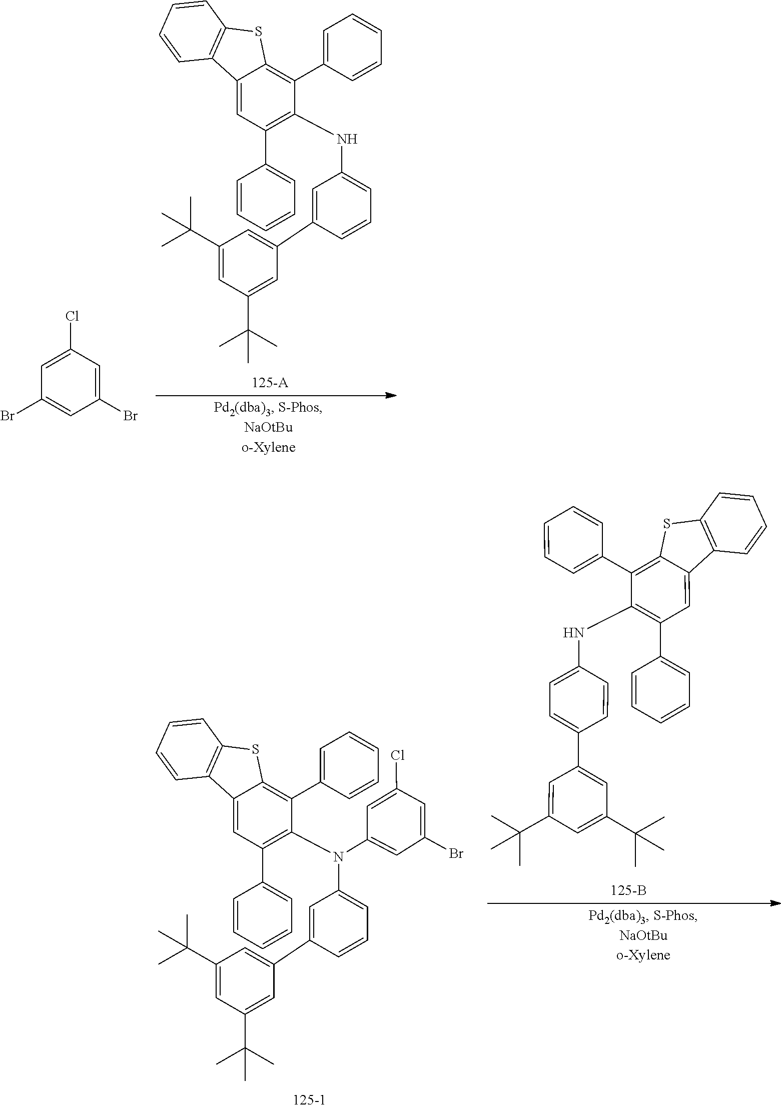

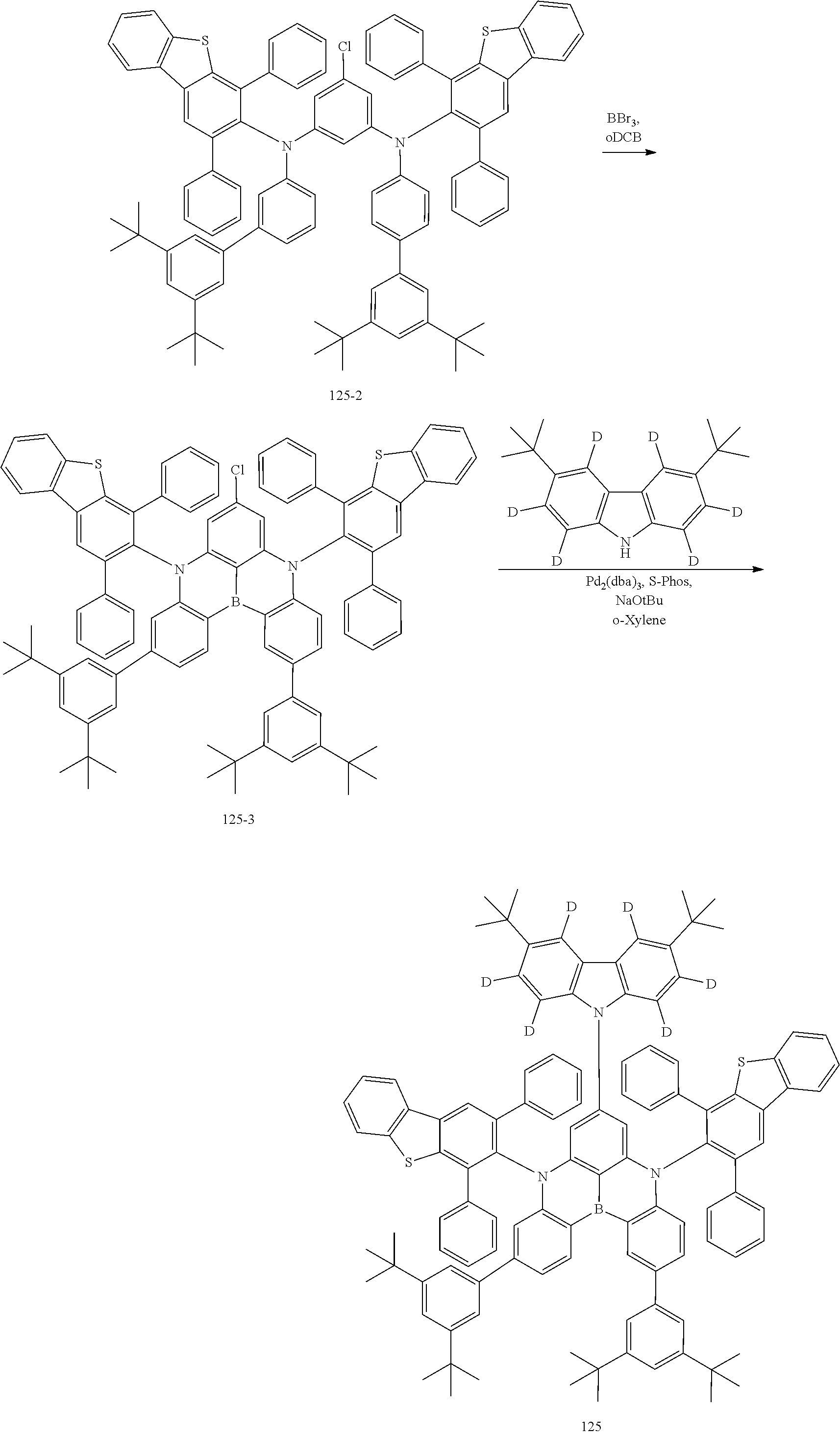

### Synthesis of Intermediate Compound 125-1

1,3-dibromo-5-chlorobenzene (1.1 eq), Intermediate Compound 125-A (1 eq), tris(dibenzylideneacetone)dipalladium(0) (0.05 eq), 2-dicyclohexylphosphino-2′,6′-dimethoxybiphenyl (S-Phos, 0.10 eq), and sodium tert-butoxide (2 eq) were dissolved in o-Xylene, followed by stirring in a nitrogen atmosphere at 150° C. for 20 hours in a high-pressure reactor. The mixture was cooled and dried under reduced pressure to remove o-xylene. A washing process was performed thereon three times by using ethyl acetate and water, and an organic layer thus obtained was dried by using MgSO₄ under reduced pressure. The resulting product was purified by column chromatography and recrystallized (dichloromethane:n-Hexane) to obtain Intermediate Compound 125-1. The yield was 69%.

### Synthesis of Intermediate Compound 125-2

Intermediate Compound 125-1 (1 eq), Intermediate Compound 125-B (1.1 eq), tris(dibenzylideneacetone)dipalladium(0) (0.05 eq), 2-dicyclohexylphosphino-2′,6′-dimethoxybiphenyl (S-Phos, 0.10 eq), and sodium tert-butoxide (2 eq) were dissolved in xylene, followed by stirring in a nitrogen atmosphere at 150° C. for 20 hours in a high-pressure reactor. The mixture was cooled and dried under reduced pressure to remove o-xylene. A washing process was performed thereon three times by using ethyl acetate and water, and an organic layer thus obtained was dried by using MgSO₄ under reduced pressure. The resulting product was purified by column chromatography and recrystallized (dichloromethane:n-Hexane) to obtain Intermediate Compound 125-2. The yield was 75%.

### Synthesis of Intermediate Compound 125-3

After Intermediate Compound 125-2 (1 eq) was dissolved in ortho dichlorobenzene, the flask was cooled to 0° C. in a nitrogen atmosphere, and boron tribromide (BBr₃, 2.5 eq) dissolved in ortho dichlorobenzene was slowly injected thereto. After termination of the dropwise addition, the temperature was raised to 190° C., followed by stirring for 24 hours. After cooling to 0° C., triethylamine was slowly added dropwise to the flask to terminate the reaction until the exotherm stopped, and methanol was added thereto to cause precipitation and a solid was obtained therefrom by filtration. The obtained solid was purified by silica pad filtration, and purified by MC/Hex recrystallization again to obtain Intermediate Compound 125-3. Final purification was performed thereon by using column chromatography and recrystallized (dichloromethane:n-Hexane). The yield was 10%.

### Synthesis of Compound 125

Intermediate Compound 125-3 (1 eq), 3,6-di-tert-butyl-9H-carbazole-1,2,4,5,7,8-d6 (1.3 eq), tris(dibenzylideneacetone)dipalladium(0) (0.05 eq), tri-tert-butylphosphine (0.10 eq), and sodium tert-butoxide (1.5 eq) were dissolved in xylene, followed by stirring in a nitrogen atmosphere at 150° C. for 10 hours. The mixture was cooled and dried under reduced pressure to remove o-xylene. A washing process was performed thereon three times by using ethyl acetate and water, and an organic layer thus obtained was dried by using MgSO₄ under reduced pressure. The resulting product was purified by column chromatography (dichloromethane:n-Hexane), and recrystallized to obtain Compound 125. The yield was 51%. The final purification was performed by sublimation purification, and the obtained compound was identified as Compound 125 by ESI-LCMS.

ESI-LCMS: [M]⁺: C₁₁₄H₉₈D₆BN₃S₂. 1597.5

### Synthesis Example 4 (Synthesis of Compound 1421

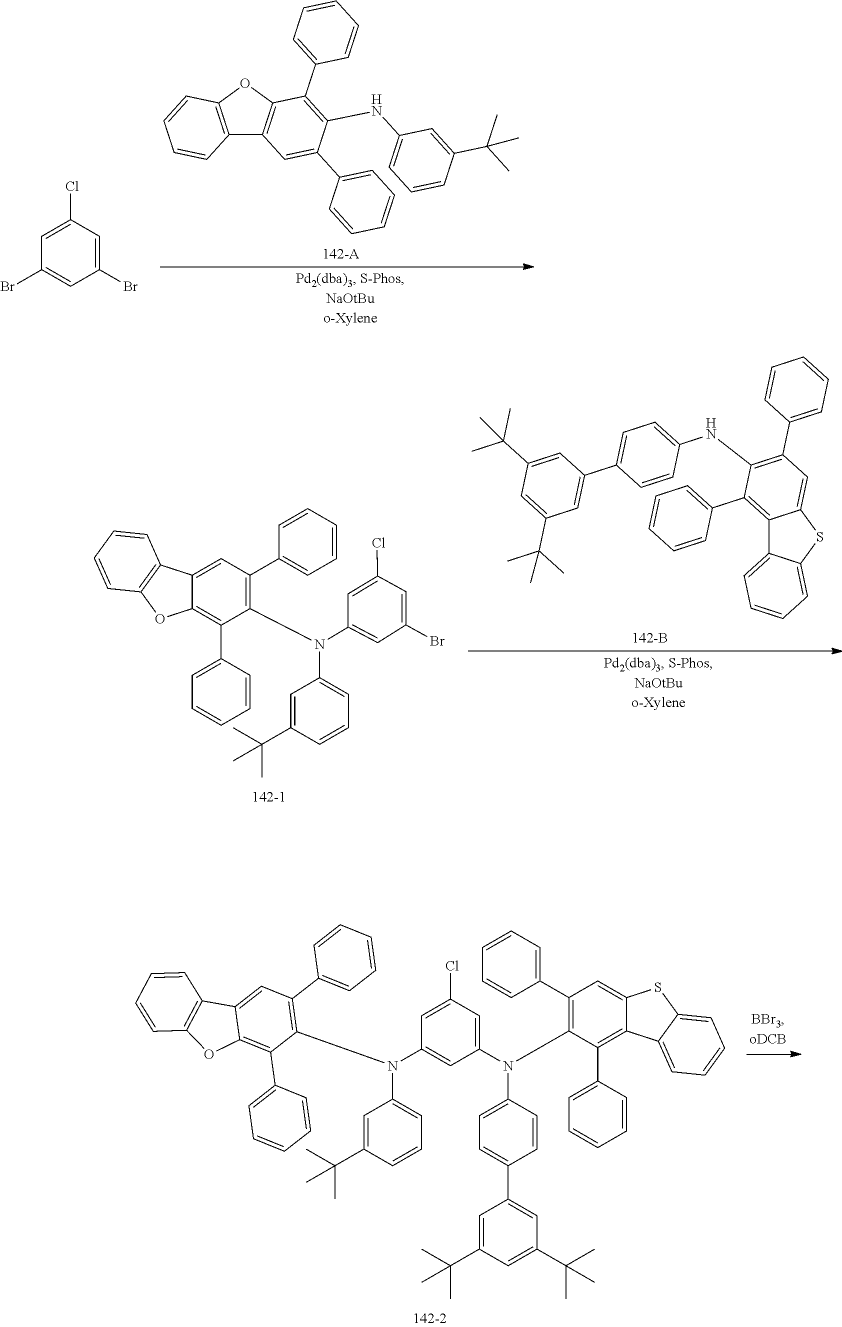 [SMILES: Clc1cc(Br)cc(Br)c1 ; CC(C)(C)c1cccc(Nc2c(-c3ccccc3)cc3c(oc4ccccc43)c2-c2ccccc2)c1 ; CC(C)(C)c1cccc(N(c2cc(Cl)cc(Br)c2)c2c(-c3ccccc3)cc3c(oc4ccccc43)c2-c2ccccc2)c1 ; CC(C)(C)c1cc(-c2ccc(Nc3c(-c4ccccc4)cc4sc5ccccc5c4c3-c3ccccc3)cc2)cc(C(C)(C)C)c1 ; CC(C)(C)c1cccc(N(c2cc(Cl)cc(N(c3ccc(-c4cc(C(C)(C)C)cc(C(C)(C)C)c4)cc3)c3c(-c4ccccc4)cc4sc5ccccc5c4c3-c3ccccc3)c2)c2c(-c3ccccc3)cc3c(oc4ccccc43)c2-c2ccccc2)c1]

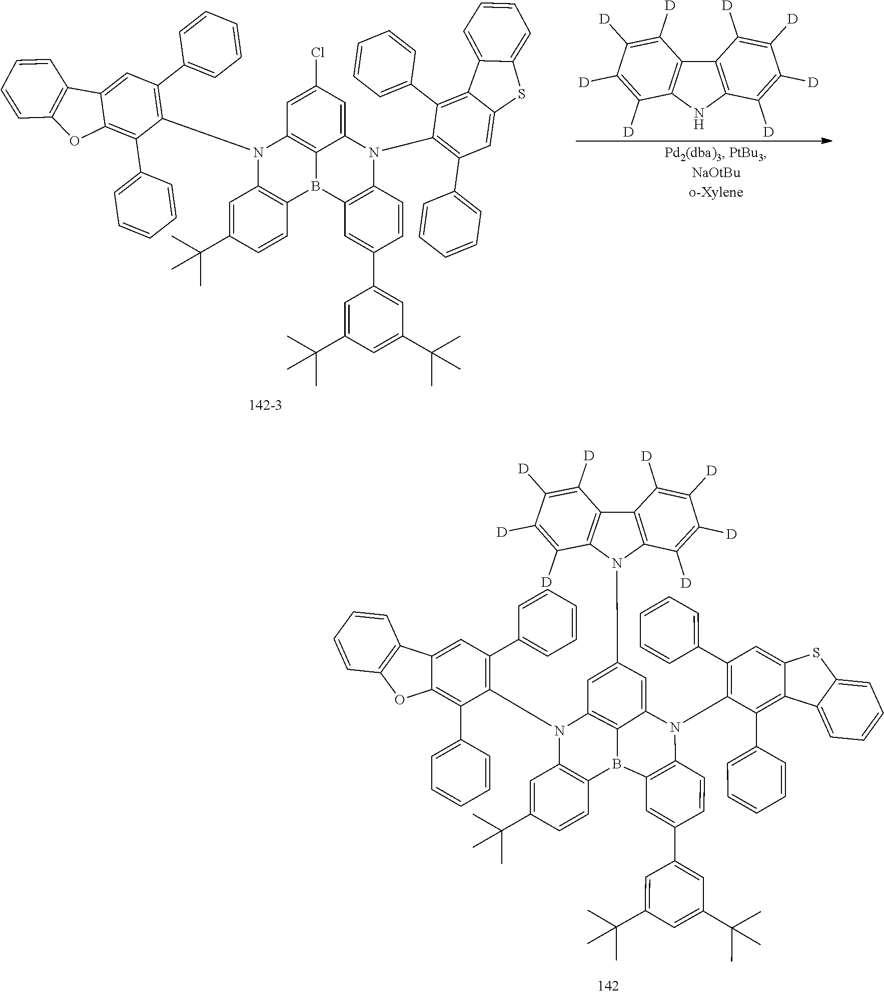 [SMILES: CC(C)(C)c1cc(-c2ccc3c(c2)B2c4ccc(C(C)(C)C)cc4N(c4c(-c5ccccc5)cc5c(oc6ccccc65)c4-c4ccccc4)c4cc(Cl)cc(c42)N3c2c(-c3ccccc3)cc3sc4ccccc4c3c2-c2ccccc2)cc(C(C)(C)C)c1 ; [2H]c1c([2H])c([2H])c2c([nH]c3c([2H])c([2H])c([2H])c([2H])c32)c1[2H] ; [2H]c1c([2H])c([2H])c2c(c1[2H])c1c([2H])c([2H])c([2H])c([2H])c1n2-c1cc2c3c(c1)N(c1c(-c4ccccc4)cc4sc5ccccc5c4c1-c1ccccc1)c1ccc(-c4cc(C(C)(C)C)cc(C(C)(C)C)c4)cc1B3c1ccc(C(C)(C)C)cc1N2c1c(-c2ccccc2)cc2c(oc3ccccc32)c1-c1ccccc1]

### Synthesis of Intermediate Compound 142-1

1,3-dibromo-5-chlorobenzene (1.1 eq), Intermediate Compound 142-A (1 eq), tris(dibenzylideneacetone)dipalladium(0) (0.05 eq), 2-dicyclohexylphosphino-2′,6′-dimethoxybiphenyl (S-Phos, 0.10 eq), and sodium tert-butoxide (2 eq) were dissolved in o-Xylene, followed by stirring in a nitrogen atmosphere at 150° C. for 20 hours in a high-pressure reactor. The mixture was cooled and dried under reduced pressure to remove o-xylene. A washing process was performed thereon three times by using ethyl acetate and water, and an organic layer thus obtained was dried by using MgSO₄ under reduced pressure. The resulting product was purified by column chromatography and recrystallized (dichloromethane:n-Hexane) to obtain Intermediate Compound 142-1. The yield was 61%.

### Synthesis of Intermediate Compound 142-2

Intermediate Compound 142-1 (1.1 eq), Intermediate Compound 142-B (1 eq), tris(dibenzylideneacetone)dipalladium(0) (0.05 eq), 2-dicyclohexylphosphino-2′,6′-dimethoxybiphenyl (S-Phos, 0.10 eq), and sodium tert-butoxide (2 eq) were dissolved in 0-Xylene, followed by stirring in a nitrogen atmosphere at 150° C. for 20 hours in a high-pressure reactor. The mixture was cooled and dried under reduced pressure to remove o-xylene. A washing process was performed thereon three times by using ethyl acetate and water, and an organic layer thus obtained was dried by using MgSO₄ under reduced pressure. The resulting product was purified by column chromatography and recrystallized (dichloromethane:n-Hexane) to obtain Intermediate Compound 142-2. The yield was 68%.

### Synthesis of Intermediate Compound 142-3

After Intermediate Compound 142-2 (1 eq) was dissolved in ortho dichlorobenzene, the flask was cooled to 0° C. in a nitrogen atmosphere, and boron tribromide (BBr₃, 2.5 eq) dissolved in ortho dichlorobenzene was slowly injected thereto. After termination of the dropwise addition, the temperature was raised to 190° C., followed by stirring for 24 hours. After cooling to 0° C., triethylamine was slowly added dropwise to the flask to terminate the reaction until the exotherm stopped, and then, methanol was added thereto to cause precipitation and a solid was obtained therefrom by filtration. The obtained solid was purified by silica pad filtration, and purified by MC/Hex recrystallization again to obtain Intermediate Compound 142-3. Final purification was performed thereon by using column chromatography and recrystallized (dichloromethane:n-Hexane). The yield was 13%.

### Synthesis of Compound 142

Intermediate Compound 142-3 (1 eq), 9H-carbazole-1,2,3,4,5,6,7,8-d8 (1.3 eq), tris(dibenzylideneacetone)dipalladium(0) (0.05 eq), tri-tert-butylphosphine (0.10 eq), and sodium tert-butoxide (1.5 eq) were dissolved in o-Xylene, followed by stirring in a nitrogen atmosphere at 150° C. for 10 hours. The mixture was cooled and dried under reduced pressure to remove o-xylene. A washing process was performed thereon three times by using ethyl acetate and water, and an organic layer thus obtained was dried by using MgSO₄ under reduced pressure. The resulting product was purified by column chromatography (dichloromethane:n-Hexane), and recrystallized to obtain Compound 142. The yield was 54%. The final purification was performed by sublimation purification, and the obtained compound was identified as Compound 142 by ESI-LCMS.

ESI-LCMS: [M]⁺: C₉₂H₆₀N₃O. 1282.9

### Synthesis Example 5 (Synthesis of Compound 154)

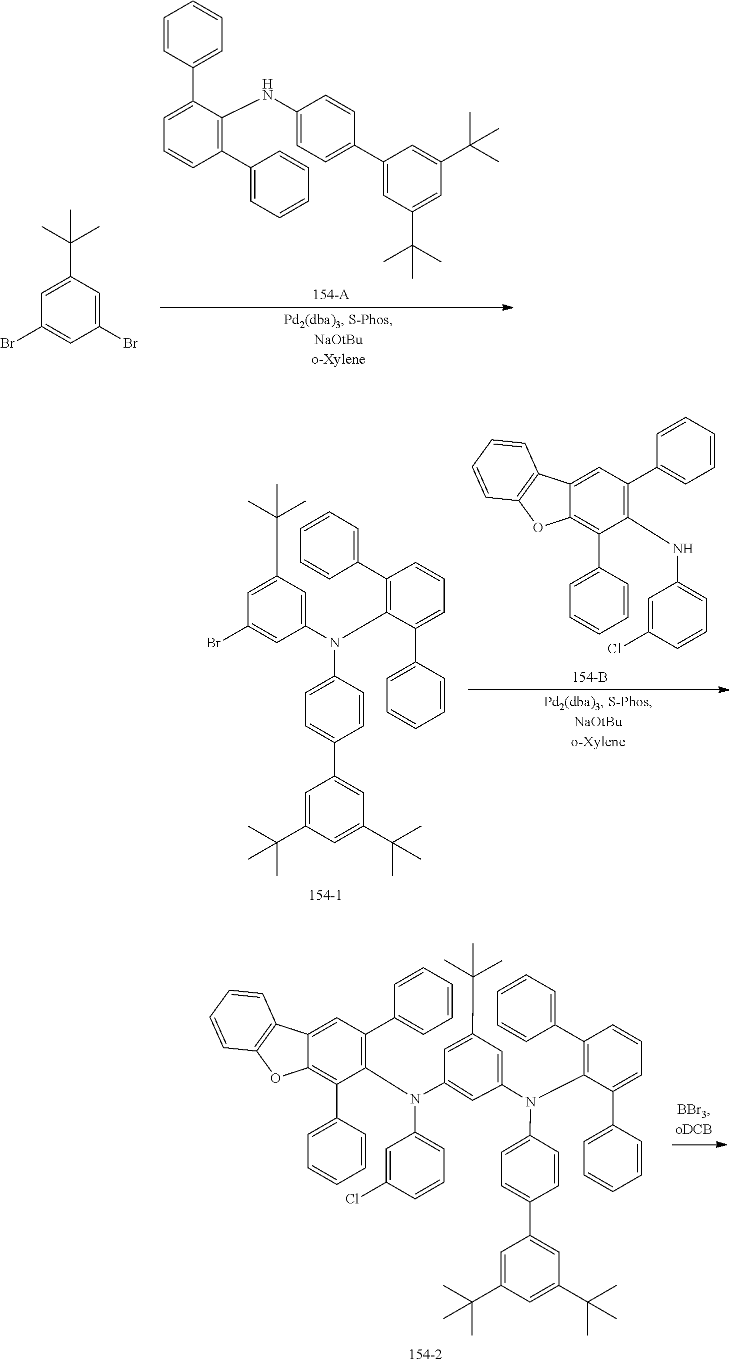 [SMILES: CC(C)(C)c1cc(Br)cc(Br)c1 ; CC(C)(C)c1cc(-c2ccc(Nc3c(-c4ccccc4)cccc3-c3ccccc3)cc2)cc(C(C)(C)C)c1 ; CC(C)(C)c1cc(Br)cc(N(c2ccc(-c3cc(C(C)(C)C)cc(C(C)(C)C)c3)cc2)c2c(-c3ccccc3)cccc2-c2ccccc2)c1 ; Clc1cccc(Nc2c(-c3ccccc3)cc3c(oc4ccccc43)c2-c2ccccc2)c1 ; CC(C)(C)c1cc(N(c2ccc(-c3cc(C(C)(C)C)cc(C(C)(C)C)c3)cc2)c2c(-c3ccccc3)cccc2-c2ccccc2)cc(N(c2cccc(Cl)c2)c2c(-c3ccccc3)cc3c(oc4ccccc43)c2-c2ccccc2)c1]

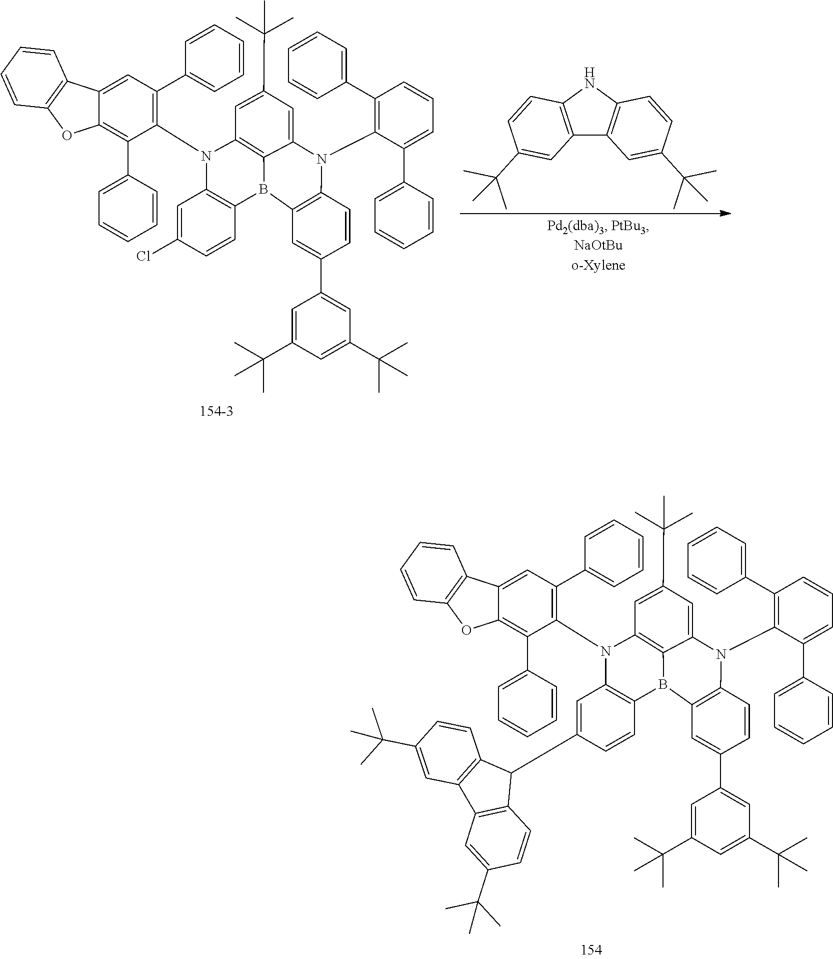

### Synthesis of Intermediate Compound 154-1

1,3-dibromo-5-(tert-butyl)benzene (1.1 eq), Intermediate Compound 154-A (1 eq), tris(dibenzylideneacetone)dipalladium(0) (0.05 eq), 2-dicyclohexylphosphino-2′,6′-dimethoxybiphenyl (S-Phos, 0.10 eq), and sodium tert-butoxide (2 eq) were dissolved in o-Xylene, followed by stirring in a nitrogen atmosphere at 150° C. for 20 hours in a high-pressure reactor. The mixture was cooled and dried under reduced pressure to remove o-xylene. A washing process was performed thereon three times by using ethyl acetate and water, and an organic layer thus obtained was dried by using MgSO₄ under reduced pressure. The resulting product was purified by column chromatography and recrystallized (dichloromethane:n-Hexane) to obtain Intermediate Compound 154-1. The yield was 66%.

### Synthesis of Intermediate Compound 154-2

Intermediate Compound 154-1 (1.1 eq), Intermediate Compound 154-B (1 eq), tris(dibenzylideneacetone)dipalladium(0) (0.05 eq), 2-dicyclohexylphosphino-2′,6′-dimethoxybiphenyl (S-Phos, 0.10 eq), and sodium tert-butoxide (2 eq) were dissolved in 0-Xylene, followed by stirring in a nitrogen atmosphere at 150° C. for 20 hours in a high-pressure reactor. The mixture was cooled and dried under reduced pressure to remove o-xylene. A washing process was performed thereon three times by using ethyl acetate and water, and an organic layer thus obtained was dried by using MgSO₄ under reduced pressure. The resulting product was purified by column chromatography and recrystallized (dichloromethane:n-Hexane) to obtain Intermediate Compound 154-2. The yield was 57%.

### Synthesis of Intermediate Compound 154-3

After Intermediate Compound 154-2 (1 eq) was dissolved in ortho dichlorobenzene, the flask was cooled to 0° C. in a nitrogen atmosphere, and boron tribromide (BBr₃, 2.5 eq) dissolved in ortho dichlorobenzene was slowly injected thereto. After termination of the dropwise addition, the temperature was raised to 190° C., followed by stirring for 24 hours. After cooling to 0° C., triethylamine was slowly added dropwise to the flask to terminate the reaction until the exotherm stopped, and methanol was added thereto to cause precipitation and a solid was obtained therefrom by filtration. The obtained solid was purified by silica pad filtration, and purified by MC/Hex recrystallization again to obtain Intermediate Compound 154-3. Final purification was performed thereon by using column chromatography and recrystallized (dichloromethane:n-Hexane). The yield was 9%.

### Synthesis of Compound 154

Intermediate Compound 154-3 (1 eq), 3,6-di-tert-butyl-9H-carbazole (1.3 eq), tris(dibenzylideneacetone)dipalladium(0) (0.05 eq), tri-tert-butylphosphine (0.10 eq), and sodium tert-butoxide (1.5 eq) were dissolved in o-Xylene, followed by stirring in a nitrogen atmosphere at 150° C. for 10 hours. The mixture was cooled and dried under reduced pressure to remove o-xylene. A washing process was performed thereon three times by using ethyl acetate and water, and an organic layer thus obtained was dried by using MgSO₄ under reduced pressure. The resulting product was purified by column chromatography (dichloromethane:n-Hexane), and recrystallized to obtain Compound 154. The yield was 61%. The final purification was performed by sublimation purification, and the obtained compound was identified as Compound 154 by ESI-LCMS.

ESI-LCMS: [M]⁺: C₉₈H₉₀N₃O. 1337.0

### Synthesis Example 6 (Synthesis of Compound 187)

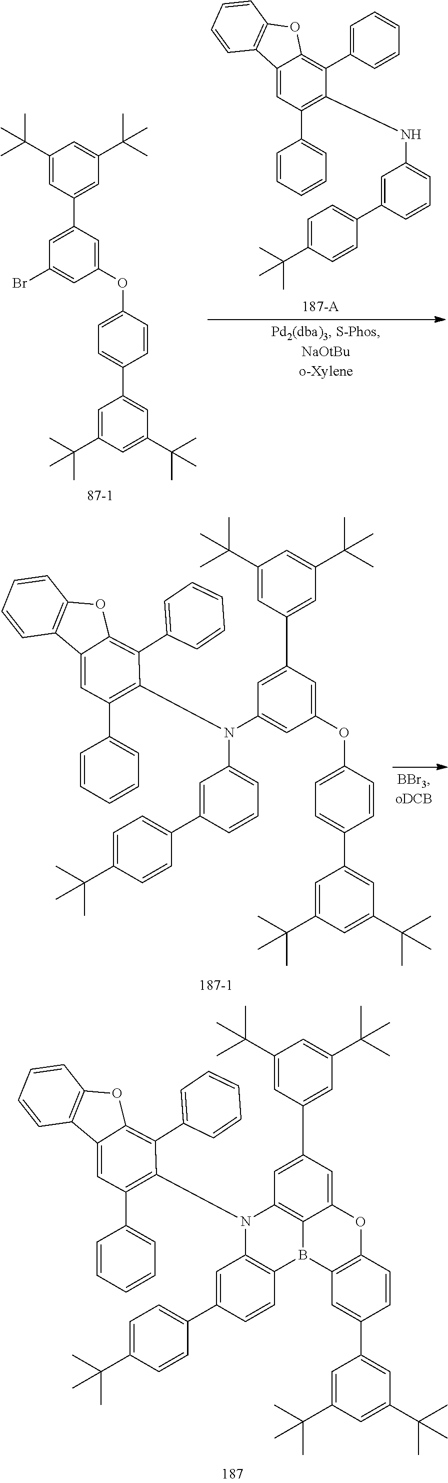 [SMILES: CC(C)(C)c1cc(-c2ccc(Oc3cc(Br)cc(-c4cc(C(C)(C)C)cc(C(C)(C)C)c4)c3)cc2)cc(C(C)(C)C)c1 ; CC(C)(C)c1ccc(-c2cccc(Nc3c(-c4ccccc4)cc4c(oc5ccccc54)c3-c3ccccc3)c2)cc1 ; CC(C)(C)c1ccc(-c2cccc(N(c3cc(Oc4ccc(-c5cc(C(C)(C)C)cc(C(C)(C)C)c5)cc4)cc(-c4cc(C(C)(C)C)cc(C(C)(C)C)c4)c3)c3c(-c4ccccc4)cc4c(oc5ccccc54)c3-c3ccccc3)c2)cc1 ; CC(C)(C)c1ccc(-c2ccc3c(c2)N(c2c(-c4ccccc4)cc4c(oc5ccccc54)c2-c2ccccc2)c2cc(-c4cc(C(C)(C)C)cc(C(C)(C)C)c4)cc4c2B3c2cc(-c3cc(C(C)(C)C)cc(C(C)(C)C)c3)ccc2O4)cc1]

### Synthesis of Intermediate Compound 187-1

Intermediate Compound 87-1 (1.1 eq), Intermediate Compound 187-A (1 eq), tris(dibenzylideneacetone)dipalladium(0) (0.05 eq), 2-dicyclohexylphosphino-2′,6′-dimethoxybiphenyl (S-Phos, 0.10 eq), and sodium tert-butoxide (2 eq) were dissolved in 0-Xylene, followed by stirring in a nitrogen atmosphere at 150° C. for 20 hours in a high-pressure reactor. The mixture was cooled and dried under reduced pressure to remove o-xylene. A washing process was performed thereon three times by using ethyl acetate and water, and an organic layer thus obtained was dried by using MgSO₄ under reduced pressure. The resulting product was purified by column chromatography and recrystallized (dichloromethane:n-Hexane) to obtain Intermediate Compound 187-1. The yield was 72%.

### Synthesis of Compound 187

After Intermediate Compound 187-2 (1 eq) was dissolved in ortho dichlorobenzene, the flask was cooled to 0° C. in a nitrogen atmosphere, and boron tribromide (BBr₃, 2.5 eq) dissolved in ortho dichlorobenzene was slowly injected thereto. After termination of the dropwise addition, the temperature was raised to 190° C., followed by stirring for 24 hours. After cooling to 0° C., triethylamine was slowly added dropwise to the flask to terminate the reaction until the exotherm stopped, and methanol was added thereto to cause precipitation and a solid was obtained therefrom by filtration. The obtained solid was purified by silica pad filtration, and purified by MC/Hex recrystallization again to obtain Compound 187. Final purification was performed thereon by using column chromatography and recrystallized (dichloromethane:n-Hexane). The yield was 12%. The final purification was performed by sublimation purification, and the obtained compound was identified as Compound 187 by ESI-LCMS.

ESI-LCMS: [M]⁺: C₈₀H₇₈NO₂. 1096.7

Synthesis methods of compounds other than the compounds of Synthesis Examples 1 to 6 may be readily recognized by those skilled in the art by referring to the synthesis paths and source materials.

### Example 1-1

As an anode, a Corning 15 Ω/cm² (1,200 Å) ITO glass substrate was cut to a size of 50 mm×50 mm×0.5 mm, sonicated with isopropyl alcohol and pure water each for 5 minutes, and cleaned by exposure to ultraviolet rays and ozone for 30 minutes. The ITO glass substrate was provided to a vacuum deposition apparatus.

NPD was vacuum-deposited on the ITO anode formed on the glass substrate to form a hole injection layer having a thickness of 300 Å, and Compound HT6 was vacuum-deposited on the hole injection layer to form a hole transport layer having a thickness of 200 Å.

A hole-transporting compound CzSi was vacuum-deposited on the hole transport layer to form an emission auxiliary layer having a thickness of 100 Å, and a host including HTH-1 and ETH-1 at a weight ratio of 5:5, PS2 as a sensitizer, and Compound 61 as a dopant were co-deposited at a weight ratio of 82:17:1 to form an emission layer having a thickness of 200 Å.

TSPO1 was deposited on the emission layer to form an electron transport layer having a thickness of 200 Å, and a buffer electron-transporting compound TPBI was deposited on the electron transport layer to form a buffer layer having a thickness of 300 Å.

LiF was deposited on the electron transport layer to form an electron injection layer having a thickness of 10 Å, and Al was vacuum-deposited thereon to form a LiF/Al electrode having a thickness of 3,000 Å (cathode electrode). HT28 was vacuum-deposited on the LiF/Al electrode to form a capping layer having a thickness of 700 Å, thereby manufacturing a light-emitting device.

### Examples 1-2 to 1-6 and Comparative Examples 1-1 to 1-3

Organic electroluminescent light-emitting devices of Examples 1-2 to 1-6 and Comparative Examples 1-1 to 1-3 were manufactured in the same manner as in Example 1-1, except that, when forming the emission layer, combinations of a dopant, a sensitizer, and a host shown in Table 1 were used.

TABLE 1

| | | | Host |
|---|---|---|---|
| | Dopant | Sensitizer | (HTH:ETH = 5:5) |
| Example 1-1 | Compound 61 | PS-2 | HTH-1/ETH-2 |
| Example 1-2 | Compound 87 | PS-1 | HTH-2/ETH-2 |
| Example 1-3 | Compound 125 | PS-2 | HTH-3/ETH-2 |
| Example 1-4 | Compound 142 | PS-1 | HTH-3/ETH-1 |
| Example 1-5 | Compound 154 | PS-2 | HTH-2/ETH-3 |
| Example 1-6 | Compound 187 | PS-1 | HTH-1/ETH-3 |
| Comparative | Comparative | PS-2 | HTH-3/ETH-3 |
| Example 2-1 | Example | | |
| | Compound 1 | | |
| Comparative | Comparative | PS-1 | HTH-1/ETH-1 |
| Example 2-2 | Example | | |
| | Compound 2 | | |
| Comparative | Comparative | PS-1 | HTH-2/ETH-2 |
| Example 2-3 | Example | | |
| | Compound 3 | | |

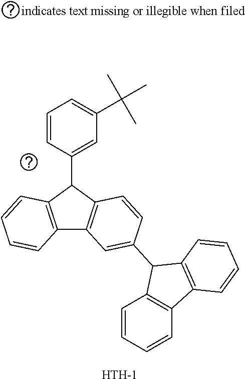 [SMILES: CC(C)(C)c1cccc(C2c3ccccc3-c3cc(C4c5ccccc5-c5ccccc54)ccc32)c1 ; "HTH-1"]

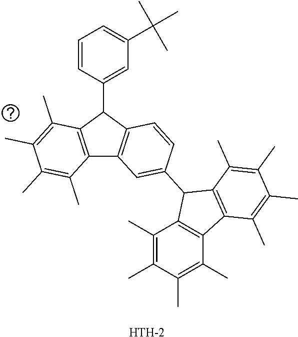

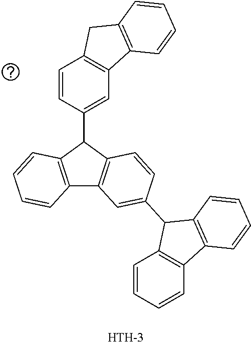 [SMILES: c1ccc2c(c1)Cc1ccc(C3c4ccccc4-c4cc(C5c6ccccc6-c6ccccc65)ccc43)cc1-2 ; "HTH-3"]

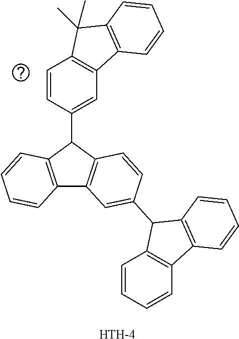 [SMILES: CC1(C)c2ccccc2-c2cc(C3c4ccccc4-c4cc(C5c6ccccc6-c6ccccc65)ccc43)ccc21 ; "HTH-4"]

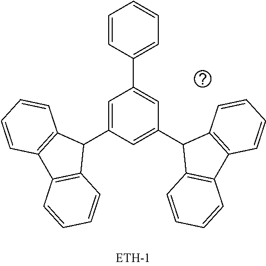 [SMILES: c1ccc(-c2cc(C3c4ccccc4-c4ccccc43)cc(C3c4ccccc4-c4ccccc43)c2)cc1]

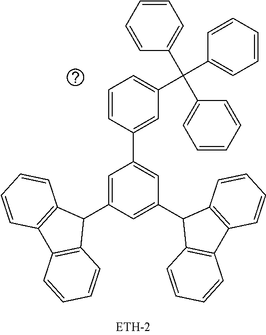 [SMILES: c1ccc(C(c2ccccc2)(c2ccccc2)c2cccc(-c3cc(C4c5ccccc5-c5ccccc54)cc(C4c5ccccc5-c5ccccc54)c3)c2)cc1]

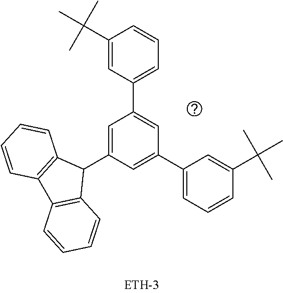 [SMILES: CC(C)(C)c1cccc(-c2cc(-c3cccc(C(C)(C)C)c3)cc(C3c4ccccc4-c4ccccc43)c2)c1]

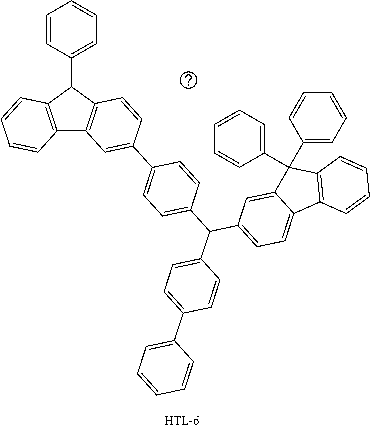 [SMILES: c1ccc(-c2ccc(C(c3ccc(-c4ccc5c(c4)-c4ccccc4C5c4ccccc4)cc3)c3ccc4c(c3)C(c3ccccc3)(c3ccccc3)c3ccccc3-4)cc2)cc1]

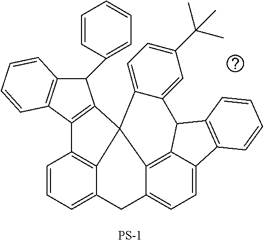

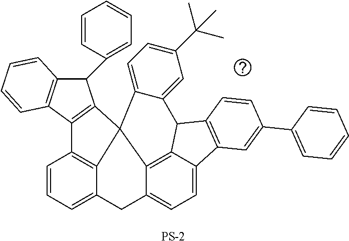

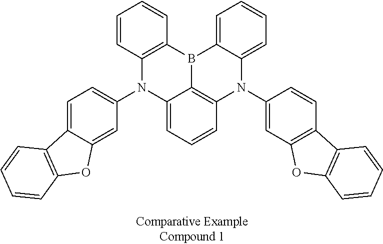 [SMILES: c1ccc2c(c1)B1c3ccccc3N(c3ccc4c(c3)oc3ccccc34)c3cccc(c31)N2c1ccc2c(c1)oc1ccccc12]

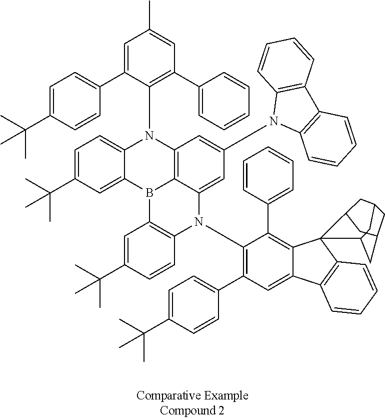 [SMILES: Cc1cc(-c2ccccc2)c(N2c3ccc(C(C)(C)C)cc3B3c4cc(C(C)(C)C)ccc4N(c4c(-c5ccc(C(C)(C)C)cc5)cc5c(c4-c4ccccc4)C4(c6ccccc6-5)C5CC6CC7CC4C7(C6)C5)c4cc(-n5c6ccccc6c6ccccc65)cc2c43)c(-c2ccc(C(C)(C)C)cc2)c1]

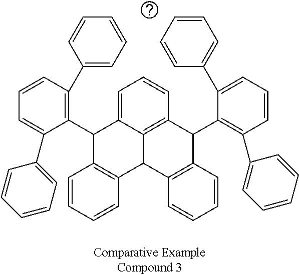 [SMILES: c1ccc(-c2cccc(-c3ccccc3)c2C2c3ccccc3C3c4ccccc4C(c4c(-c5ccccc5)cccc4-c4ccccc4)c4cccc2c43)cc1]

### Examples 2-1 to 2-6 and Comparative Examples 2-1 to 2-3

Organic electroluminescent light-emitting devices of Examples 2-1 to 2-6 and Comparative Examples 2-1 to 2-3 were manufactured in the same manner as in Examples 1-1 to 1-6 and Comparative Examples 1-1 to 1-3, respectively, except that, when forming the emission layer, only a dopant and a host were used.

### Evaluation Example 1

To evaluate characteristics of the organic light-emitting devices manufactured according to Examples 1-1 to 1-6 and Comparative Examples 1-1 to 1-3, the driving voltage at a current density of 10 mA/cm² and maximum quantum efficiency were measured, and results are shown in Table 2. The driving voltage of each of the organic light-emitting devices was measured using a source meter (Keithley Instrument Inc., 2400 series), and the maximum quantum efficiency of each of the organic electroluminescent devices was measured using the external quantum efficiency measurement apparatus C9920-2-12 of Hamamatsu Photonics Inc. In evaluating the maximum quantum efficiency, the luminance/current density was measured utilizing a luminance meter that was calibrated for wavelength sensitivity, and the maximum quantum efficiency was converted by assuming an angular luminance distribution (Lambertian) which introduced a perfect reflecting diffuser. To evaluate the device lifespan, values obtained by comparing a time taken to reach 50% of the initial luminance in Comparative Example 1-1 with those of Examples 1-1 to 1-6 and Comparative Examples 1-2 and 1-3 were calculated.

Similarly, the driving voltage and the maximum quantum efficiency of organic light-emitting devices of Examples 2-1 to 2-6 and Comparative Examples 2-1 to 2-3 were measured, and the result thereof is shown in Table 3. Moreover, to measure the device lifespan, values obtained by comparing a time taken to reach 50% of the initial luminance in Comparative Example 2-1 with those of Examples 2-1 to 2-6 and Comparative Examples 2-2 and 2-3 were calculated.

TABLE 2

| | | | | | | Maximum | | |
|---|---|---|---|---|---|---|---|---|
| | | | | | Maximum | external | | |
| | | | | Driving | luminescence | quantum | | |
| | | | Host | voltage | efficiency | efficiency | Relative | Emission |
| | Dopant | Sensitizer | (HTH:ETH = 5:5) | (V) | (cd/A) | (cd/A) | lifespan (%) | color |
| Example 1-1 | Compound 61 | PS2 | HTH-1/ETH-2 | 4.3 | 25.7 | 24.9 | 305 | Blue |
| Example 1-2 | Compound 87 | PS1 | HTH-2/ETH-2 | 4.4 | 23.8 | 22.8 | 240 | Blue |
| Example 1-3 | Compound 125 | PS2 | HTH-3/ETH-2 | 4.3 | 24.9 | 23.8 | 320 | Blue |
| Example 1-4 | Compound 142 | PS1 | HTH-3/ETH-1 | 4.4 | 24.5 | 23.6 | 280 | Blue |
| Example 1-5 | Compound 154 | PS2 | HTH-2/ETH-3 | 4.3 | 24.3 | 23.4 | 290 | Blue |
| Example 1-6 | Compound 187 | PS1 | HTH-1/ETH-3 | 4.4 | 23.2 | 22.7 | 205 | Blue |
| Comparative | Comparative | PS2 | HTH-3/ETH-3 | 4.9 | 18.1 | 17.5 | 100 | Blue |
| Example 1-1 | Example | | | | | | | |
| | Compound 1 | | | | | | | |
| Comparative | Comparative | PS1 | HTH-1/ETH-1 | 4.7 | 21.5 | 20.7 | 165 | Blue |
| Example 1-2 | Example | | | | | | | |
| | Compound 2 | | | | | | | |
| Comparative | Comparative | PS1 | HTH-2/ETH-2 | 4.8 | 19.5 | 18.8 | 190 | Blue |
| Example 1-3 | Example | | | | | | | |
| | Compound 3 | | | | | | | |

TABLE 3

| | | | | Maximum | |
|---|---|---|---|---|---|
| | | | Maximum | external | |
| | | | luminescence | quantum | |
| | | Host | efficiency | efficiency | Emission |
| | Dopant | (HTH:ETH = 5:5) | (cd/A) | (cd/A) | color |
| Example 2-1 | Compound 61 | HTH-1/ETH-2 | 8.4 | 8.1 | Blue |
| Example 2-2 | Compound 87 | HTH-2/ETH-2 | 7.7 | 7.4 | Blue |
| Example 2-3 | compound 125 | HTH-3/ETH-2 | 8.1 | 7.7 | Blue |
| Example 2-4 | Compound 142 | HTH-3/ETH-1 | 8.0 | 7.7 | Blue |
| Example 2-5 | Compound 154 | HTH-2/ETH-3 | 7.9 | 7.6 | Blue |
| Example 2-6 | Compound 187 | HTH-1/ETH-3 | 7.6 | 7.4 | Blue |
| Comparative | Comparative | HTH-3/ETH-3 | 5.9 | 5.7 | Blue |
| Example 2-1 | Example | | | | |
| | Compound 1 | | | | |
| Comparative | Comparative | HTH-1/ETH-1 | 7.0 | 6.8 | Blue |
| Example 2-2 | Example | | | | |
| | Compound 2 | | | | |
| Comparative | Comparative | HTH-2/ETH-2 | 6.5 | 6.3 | Blue |
| Example 2-3 | Example | | | | |
| | Compound 3 | | | | |

The use of the heterocyclic compound may enable the manufacture of a light-emitting device having high efficiency and a long lifespan and a high-quality electronic device including the light-emitting device.

Embodiments have been disclosed herein, and although terms are employed, they are used and are to be interpreted in a generic and descriptive sense only and not for the purposes of limitation. In some instances, as would be apparent by one of ordinary skill in the art, features, characteristics, and/or elements described in connection with an embodiment may be used singly or in combination with features, characteristics, and/or elements described in connection with other embodiments unless otherwise specifically indicated. Accordingly, it will be understood by those of ordinary skill in the art that various changes in form and details may be made without departing from the spirit and scope of the disclosure.

What is claimed is:

1. A light-emitting device comprising:
a first electrode;
a second electrode facing the first electrode;
an interlayer between the first electrode and the second electrode and comprising an emission layer; and
a heterocyclic compound represented by Formula 1: 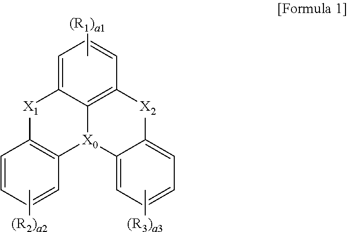
wherein in Formula 1,
X₀ is B, P(═O), or P(═S),
X₁ is N(R₄), O, or S,
X₂ is N(R₅), O, or S,
at least one of X₁ and X₂ is each independently a group represented by one of Formulae 2-1 to 2-4, 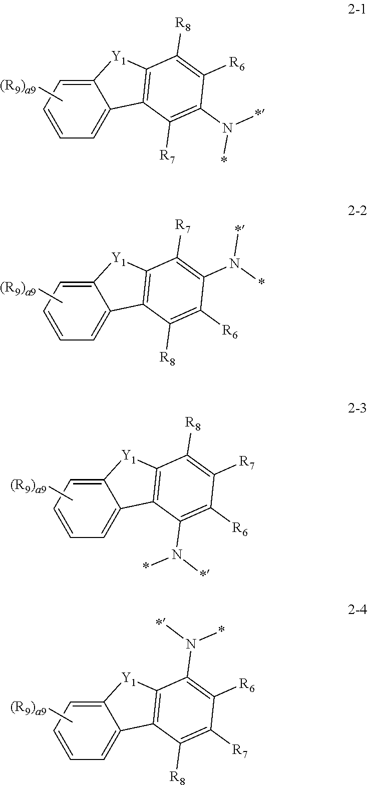
wherein in Formulae 1 and 2-1 to 2-4,
Y₁ is N(R₁₀), O, or S,
a1 to a3 and a9 are each independently an integer from 1 to 4,
* and *′ each independently indicate a binding site to a neighboring carbon atom,
R₁ to R₁₀ are each independently hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
in Formulae 2-1 and 2-2, at least one of R₆ and R₇ is a C₃-C₆₀ carbocyclic group unsubstituted or substituted with at least one R₁₀ₐ or a C₁-C₆₀ heterocyclic group unsubstituted or substituted with at least one R₁₀ₐ, and
in Formulae 2-1 and 2-2, R₆ in is a C₃-C₆₀ carbocyclic group unsubstituted or substituted with at least one R₁₀ₐ or a C₁-C₆₀ heterocyclic group unsubstituted or substituted with at least one R₁₀ₐ,
R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or a combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or a combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ are each independently: hydrogen; deuterium; —F; —Cl; —Br; —I; a hydroxyl group; a cyano group; a nitro group; or a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or a combination thereof.

2. The light-emitting device of claim 1, wherein
the first electrode is an anode,
the second electrode is a cathode,
the interlayer further comprises:
a hole transport region between the first electrode and the emission layer; and
an electron transport region between the emission layer and the second electrode,
the hole transport region comprises a hole injection layer, a hole transport layer, an emission auxiliary layer, an electron blocking layer, or a combination thereof, and
the electron transport region comprises a hole blocking layer, an electron transport layer, an electron injection layer, an electron control layer, or a combination thereof.

3. The light-emitting device of claim 1, wherein the interlayer includes the heterocyclic compound.

4. The light-emitting device of claim 1, wherein the emission layer includes the heterocyclic compound.

5. The light-emitting device of claim 4, wherein the emission layer further comprises at least one of a first host represented by Formula 5, a second host that includes a group represented by Formula 7, and a sensitizer: 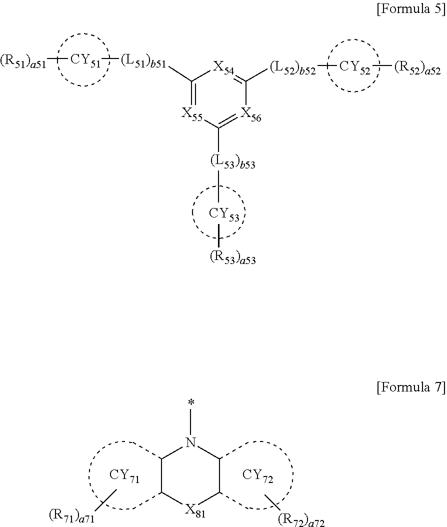
wherein in Formula 5,
X₅₄ to X₅₆ are each independently C(R₅₀), CH, or N,
at least one of X₅₄ to X₅₆ is each N,
ring CY₅₁ to ring CY₅₃ are each independently a C₅-C₆₀ carbocyclic group or a C₁-C₆₀ heterocyclic group,
L₅₁ to L₅₃ are each independently a C₃-C₆₀ carbocyclic group unsubstituted or substituted with at least one R₁₀ₐ or a C₁-C₆₀ heterocyclic group unsubstituted or substituted with at least one R₁₀ₐ,
b51 to b53 are each independently an integer from 0 to 3,
when b51 is 0, *-(L₅₁)_{b51}-*′ is a single bond,
when b52 is 0, *-(L₅₂)_{b52}-*′ is a single bond,
when b53 is 0, *-(L₅₃)_{ab3}-*′ is a single bond,
R₅₀ to R₅₃ are each independently the same as described in connection with R₁ to R₁₀ in Formula 1, and
a51 to a53 are each independently an integer from 0 to 10,
wherein in Formula 7,
ring CY₇₁ and ring CY₇₂ are each independently a C₅-C₆₀ carbocyclic group or a C₁-C₆₀ heterocyclic group,
X₈₁ is a single bond, O, S, N(R₈₁), B(R₈₁), C(R₈₁ₐ)(R_{81b}), or Si(R₈₁ₐ)(R_{81b}),
R₇₁, R₇₂, R₈₁, R₈₁ₐ, and R_{81b} are each independently the same as described in connection with R₁ to R₁₀ in Formula 1,
a71 and a72 are each independently an integer from 0 to 10, and
* indicates a binding site to a neighboring atom.

6. An electronic device comprising the light-emitting device of claim 1.

7. The electronic device of claim 6, further comprising:
a color filter, a color conversion layer, a touch screen layer, a polarizing layer, or a combination thereof.

8. An electronic apparatus comprising the light-emitting device of claim 1.

9. The electronic apparatus of claim 8, wherein the electronic apparatus is a flat panel display, a curved display, a computer monitor, a medical monitor, a television, a billboard, an indoor light, an outdoor light, a signal light, a head-up display, a fully transparent display, a partially transparent display, a flexible display, a rollable display, a foldable display, a stretchable display, a laser printer, a telephone, a portable phone, a tablet personal computer, a phablet, a personal digital assistant (FDA), a wearable device, a laptop computer, a digital camera, a camcorder, a viewfinder, a micro display, a three-dimensional (3D) display, a virtual reality display, an augmented reality display, a vehicle, a video wall with multiple displays tiled together, a theater screen, a stadium screen, a phototherapy device, or a signboard.

10. A heterocyclic compound represented by Formula 1: 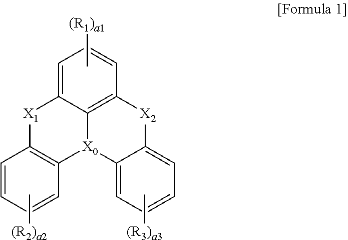
wherein in Formula 1,
X₀ is B, P(═O), or P(═S),
X₁ is N(R₄), O, or S,
X₂ is N(R₅), O, or S,
at least one of X₁ and X₂ is each independently a group represented by one of Formulae 2-1 to 2-4, 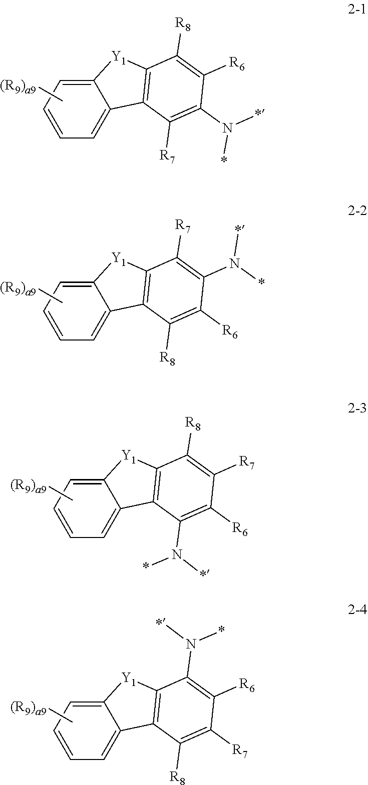
wherein in Formulae 1 and 2-1 to 2-4,
Y₁ is N(R₁₀), O, or S,
a1 to a3 and a9 are each independently an integer from 1 to 4,
* and *′ each independently indicate a binding site to a neighboring carbon atom,
R₁ to R₁₀ are each independently hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
in Formulae 2-1 and 2-2, at least one of R₆ and R₇ in is a C₃-C₆₀ carbocyclic group unsubstituted or substituted with at least one R₁₀ₐ or a C₁-C₆₀ heterocyclic group unsubstituted or substituted with at least one R₁₀ₐ, and
in Formulae 2-3 and 2-4, R₆ in Formulae 2-3 and 2-4 is a C₃-C₆₀ carbocyclic group unsubstituted or substituted with at least one R₁₀ₐ or a C₁-C₆₀ heterocyclic group unsubstituted or substituted with at least one R₁₀ₐ,
R₁₀ₐ is:
deuterium, —F, —Cl, —Br, —I, a hydroxyl group, a cyano group, or a nitro group; a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or a combination thereof;
a C₃-C₆₀ carbocyclic group, a C₁-C₆₀ heterocyclic group, a C₆-C₆₀ aryloxy group,
a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or a combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ are each independently: hydrogen; deuterium; —F; —Cl; —Br; —I; a hydroxyl group; a cyano group; a nitro group; or a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or a combination thereof.

11. The heterocyclic compound of claim 10, wherein X₁ and X₂ are identical to each other.

12. The heterocyclic compound of claim 10, wherein X₁ and X₂ are different from each other.

13. The heterocyclic compound of claim 10, wherein a1 to a3 are each 1.

14. The heterocyclic compound of claim 10, wherein R₁ to R₅, R₉, and R₁₀ are each independently:
hydrogen, deuterium, —F, —Cl, —Br, —I, a cyano group, or a C₁-C₂₀ alkyl group;
a C₁-C₂₀ alkyl group substituted with at least one of deuterium, —F, —Cl, —Br, —I, —CD₃, —CD₂H,-CDH₂, —CF₃, —CF₂H, —CFH₂, a cyano group, a nitro group, a C₁-C₁₀ alkyl group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a naphthyl group, a pyridinyl group, and a pyrimidinyl group;
a phenyl group, a pyridine group, a dibenzofuranyl group, a dibenzothiophenyl group, a carbazolyl group, an azadibenzofuranyl group, an azadibenzothiophenyl group, or an azacarbazolyl group, each unsubstituted or substituted with at least one of deuterium, —F, —Cl, —Br, —I, —CD₃, —CD₂H,-CDH₂, —CF₃, —CF₂H, —CFH₂, a cyano group, a C₁-C₂₀ alkyl group, a phenyl group, a biphenyl group, a C₁-C₁₀ alkylphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzoimidazolyl group, a benzofuranyl group, a benzothiophenyl group, a benzoisothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, —Si(Q₃₁)(Q₃₂)(Q₃₃), —N(Q₃₁)(Q₃₂), or —B(Q₃₁)(Q₃₂); or
Si(Q₁)(Q₂)(Q₃), —N(Q₁)(Q₂), or —B(Q₁)(Q₂), and
Q₁ to Q₃ and Q₃₁ to Q₃₃ are each independently:
CH₃, —CD₃, —CD₂H,-CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, or —CD₂CDH₂; or
an n-propyl group, an isopropyl group, an n-butyl group, an isobutyl group, a sec-butyl group, a tert-butyl group, an n-pentyl group, an isopentyl group, a sec-pentyl group, a tert-pentyl group, a phenyl group, a naphthyl group, a pyridinyl group, a pyrimidinyl group, a pyridazinyl group, a pyrazinyl group, or a triazinyl group, each unsubstituted or substituted with at least one of deuterium, a C₁-C₁₀ alkyl group, a phenyl group, a biphenyl group, a pyridinyl group, a pyrimidinyl group, a pyridazinyl group, a pyrazinyl group, and a triazinyl group.

15. The heterocyclic compound of claim 10, wherein R₁ to R₅, R₉, and R₁₀ are each independently:
a methyl group, an ethyl group, an n-propyl group, an iso-propyl group, an n-butyl group, an isobutyl group, a sec-butyl group, a tert-butyl group, an n-pentyl group, an isopentyl group, a sec-pentyl group, a tert-pentyl group, a 2-methylbutyl group, a 2-2dimethylpropyl group, a 1-ethylpropyl group, or a 1,2-dimethylpropyl group, each unsubstituted or substituted with deuterium, —F, —Cl, —Br, —I, a hydroxyl group, a cyano group, or a nitro group; or
a benzene group, a pyridine group, a dibenzofuran group, a dibenzothiophene group, a carbazole group, an azadibenzofuran group, an azadibenzothiophene group, or an azacarbazole group, each unsubstituted or substituted with a methyl group, an ethyl group, an n-propyl group, an iso-propyl group, an n-butyl group, an isobutyl group, a sec-butyl group, a tert-butyl group, an n-pentyl group, an isopentyl group, a sec-pentyl group, a tert-pentyl group, a 2-methylbutyl group, a 2-2 dimethylpropyl group, 1-ethylpropyl, 1,2-dimethylpropyl, or a phenyl group, each unsubstituted or substituted with deuterium, —F, —Cl, —Br, —I, a hydroxyl group, a cyano group, or a nitro group.

16. The heterocyclic compound of claim 10, wherein the heterocyclic compound is represented by Formula 1-1: 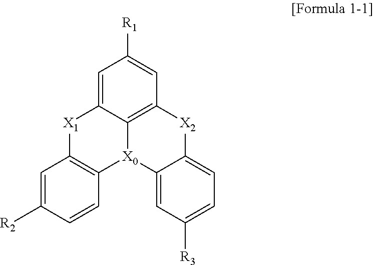
wherein in Formula 1-1,
X₀ to X₂ and R₁ to R₃ are the same as described in Formula 1, and
at least one of R₁ to R₃ is each not hydrogen.

17. The heterocyclic compound of claim 10, wherein at least one of R₆ to R₈ is each independently a C₆-C₆₀ aryl group unsubstituted or substituted with at least one R₁₀ₐ.

18. The heterocyclic compound of claim 10, wherein at least one of R₆ to R₈ is each independently a phenyl group, a pentalenyl group, a naphthyl group, an azulenyl group, an indacenyl group, an acenaphthyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a heptalenyl group, a naphthacenyl group, a picenyl group, a hexacenyl group, a pentacenyl group, a rubicenyl group, a coronenyl group, or an ovalenyl group.

19. The heterocyclic compound of claim 10, wherein in Formula 1, at least one of X₁ and X₂ is each independently a group represented by one of Formulae 3-1 to 3-10: 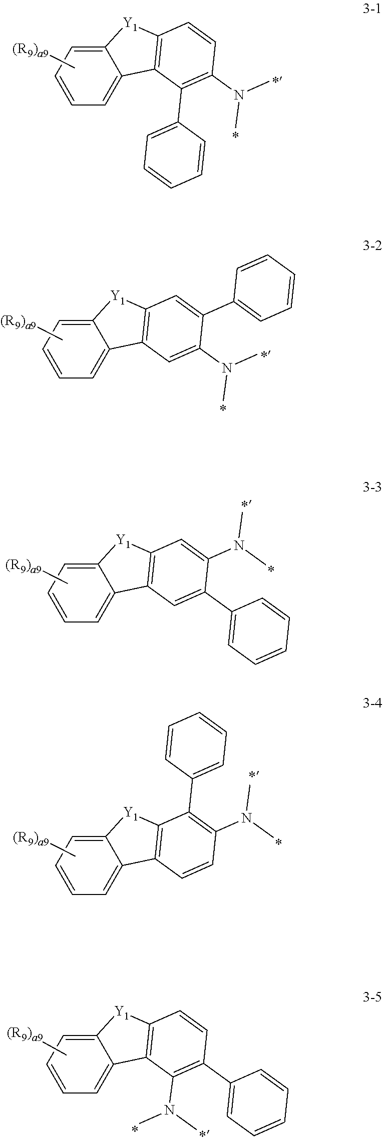 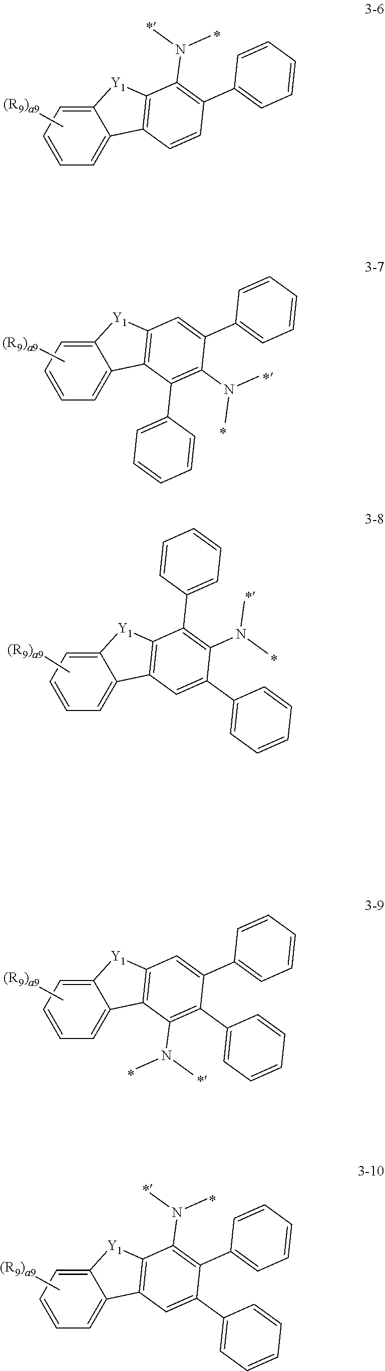
wherein in Formulae 3-1 to 3-10,
Y₁, a9, R₉, *, and *′ are the same as described in Formula 2-1 to 2-4.

20. The heterocyclic compound of claim 10, wherein, the heterocyclic compound represented by Formula 1 is one of Compounds 1 to 192: 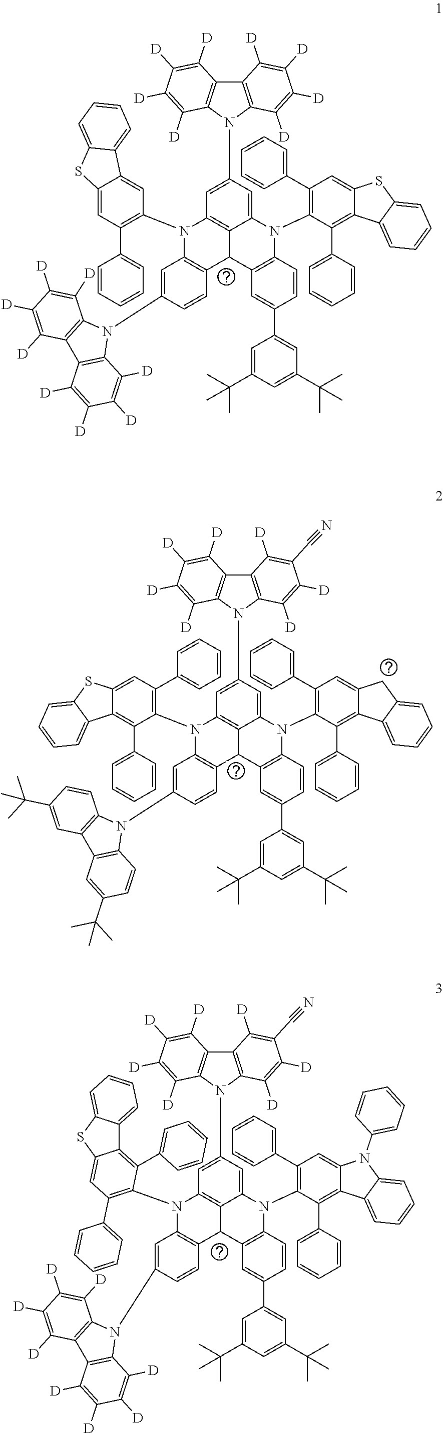 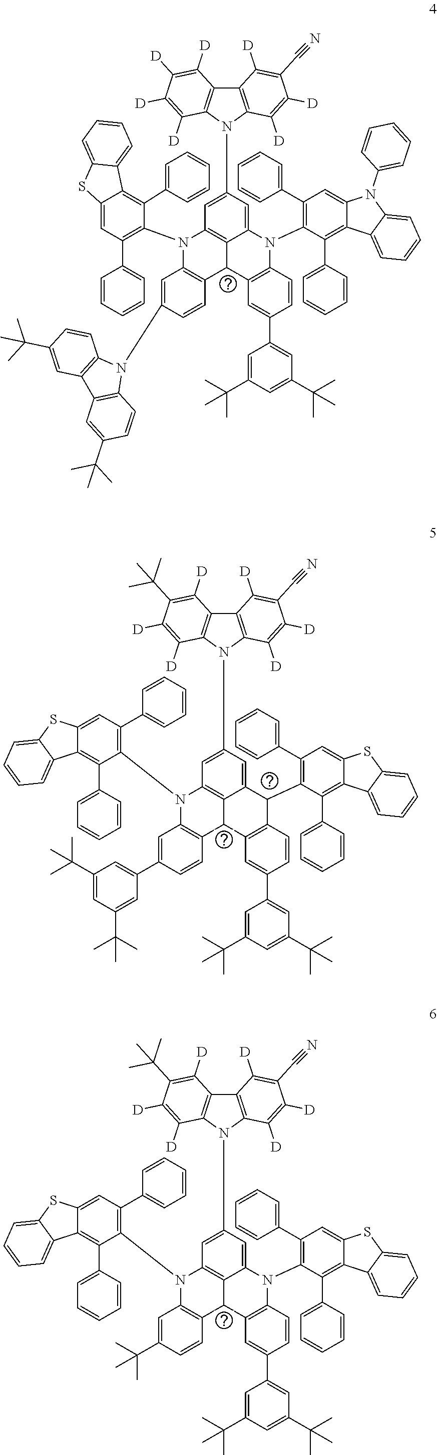 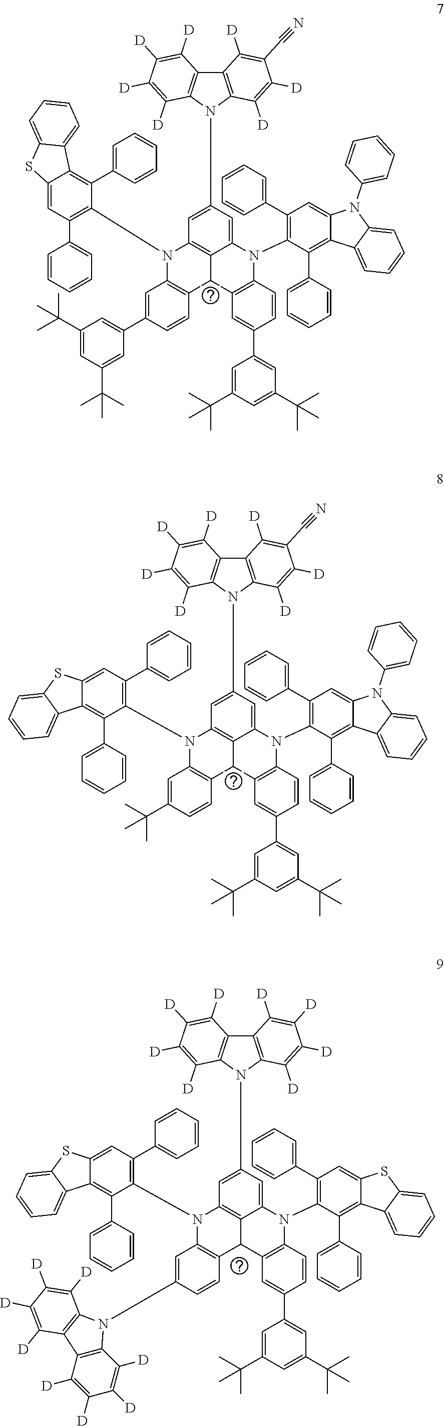 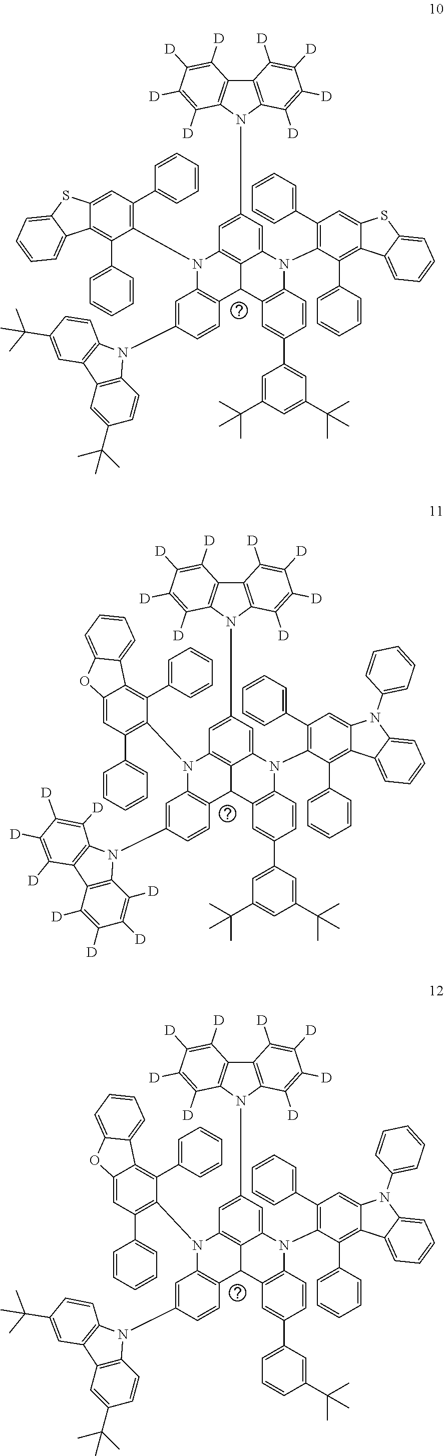 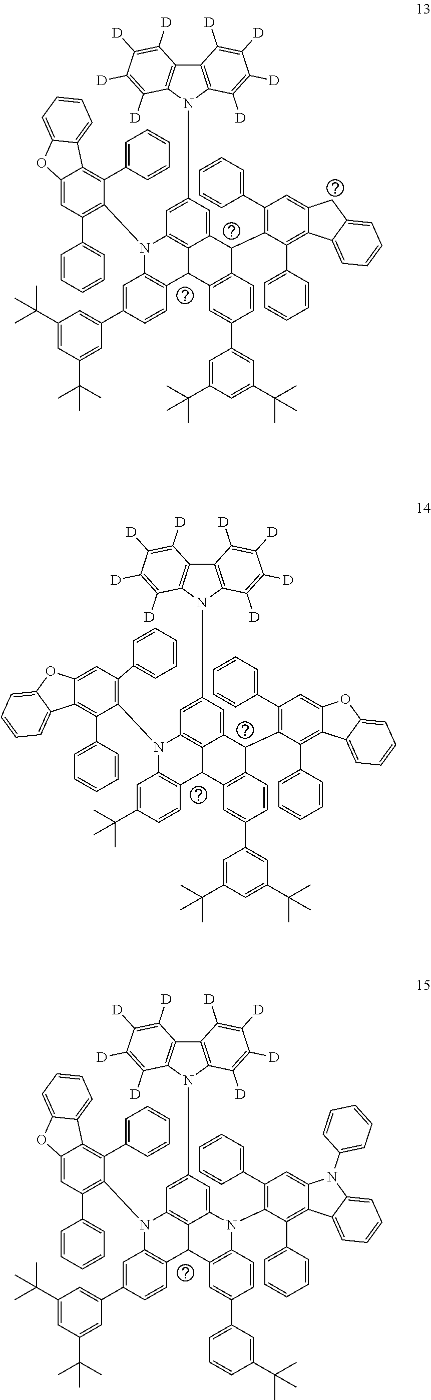 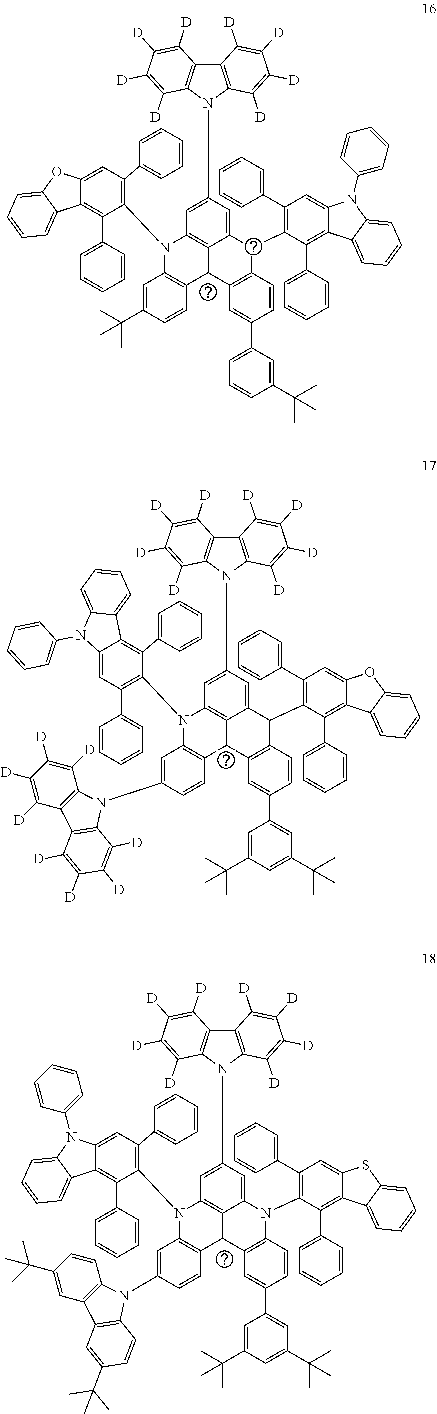 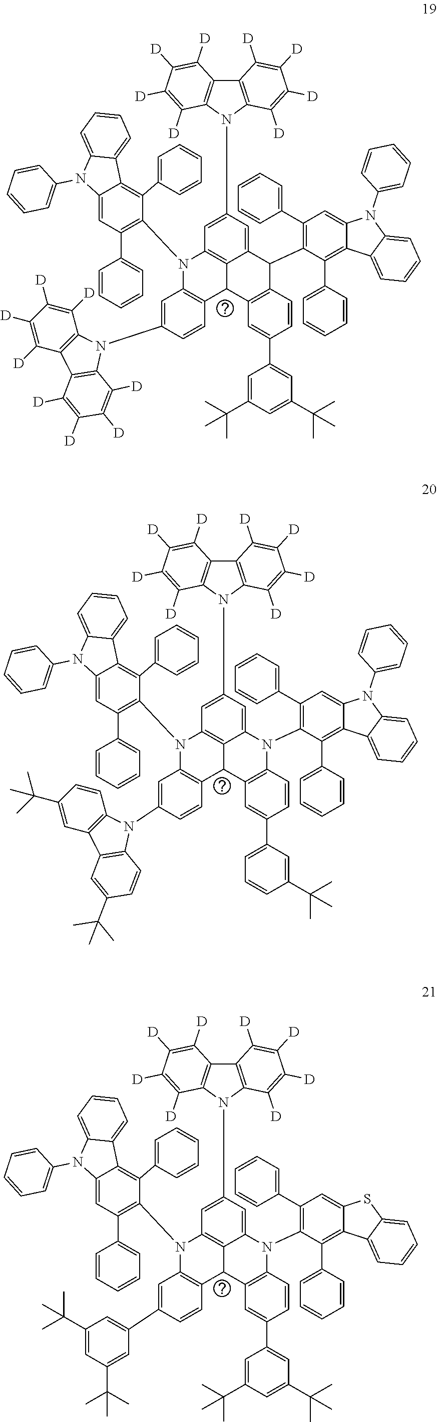 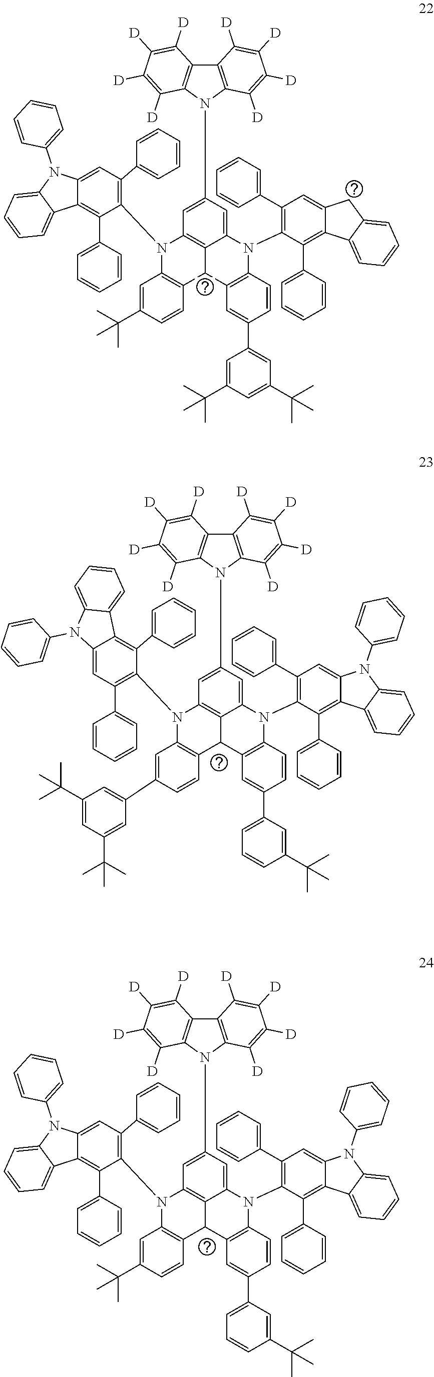 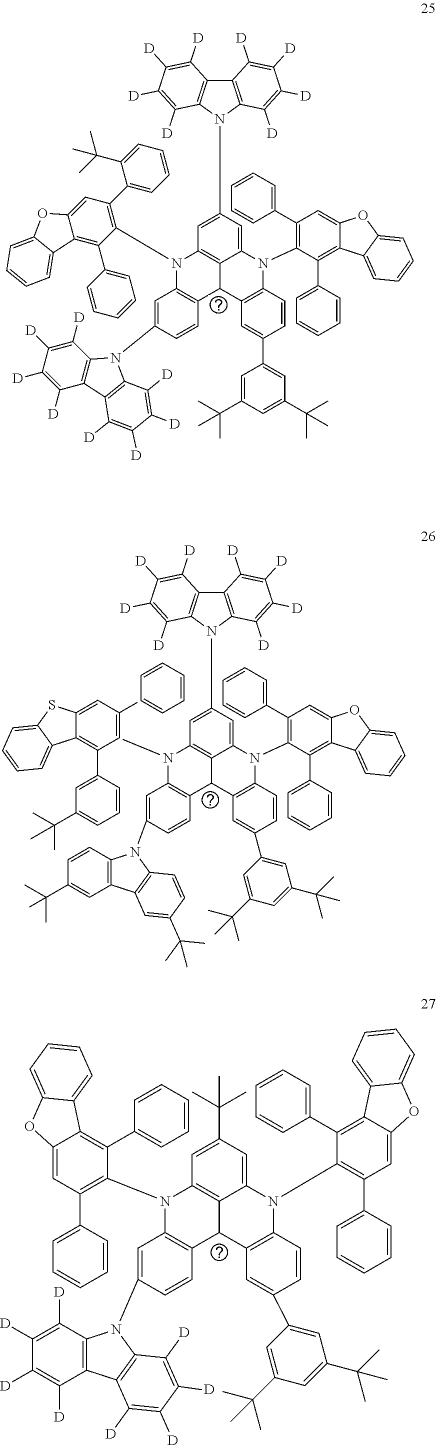 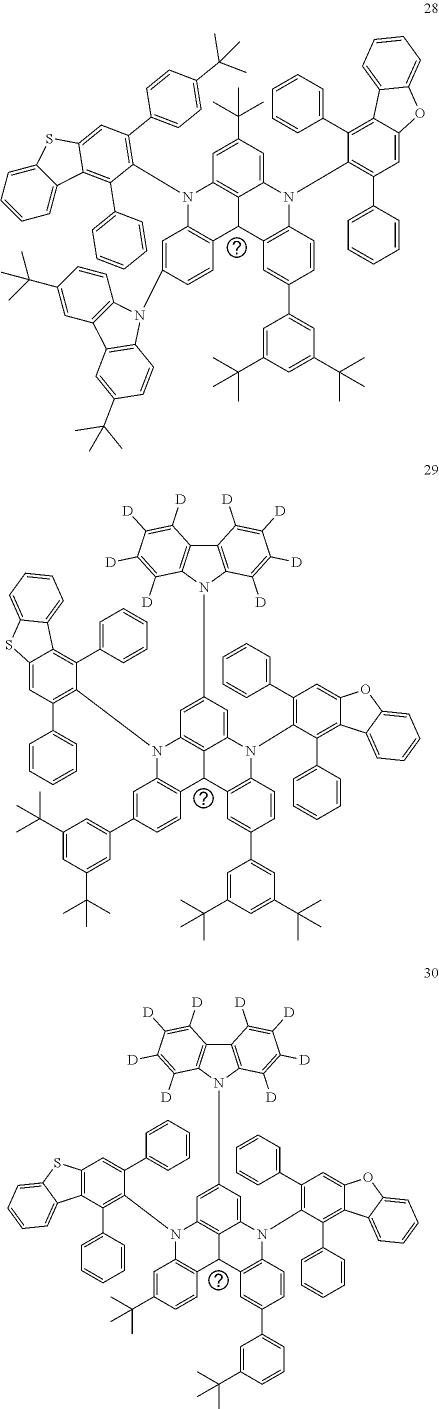 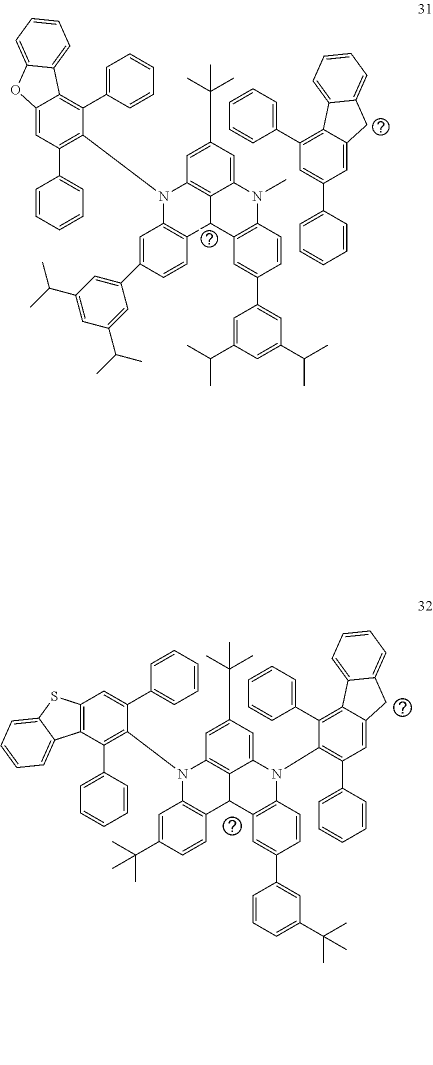 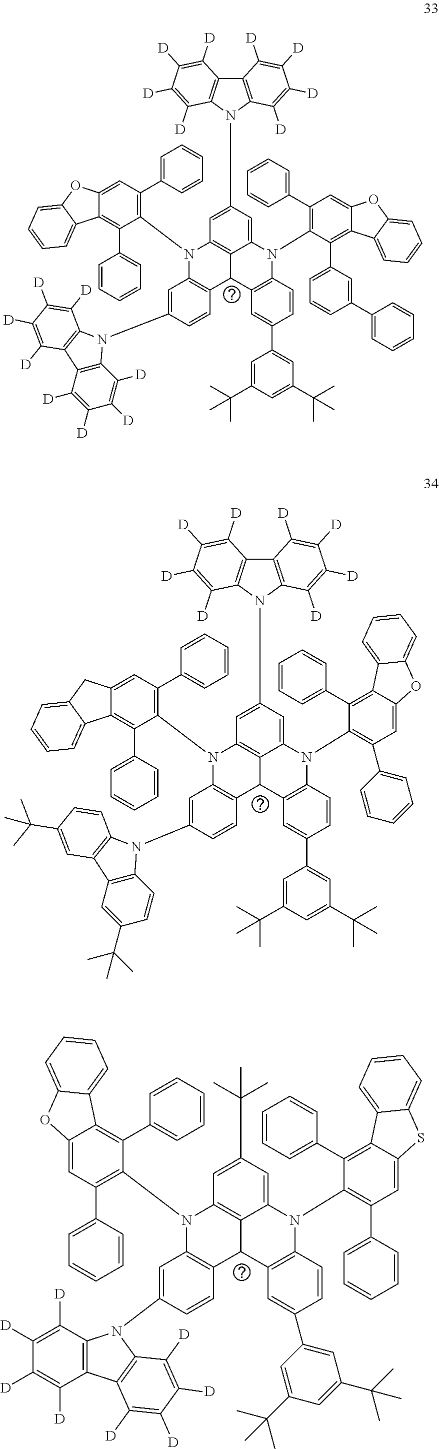 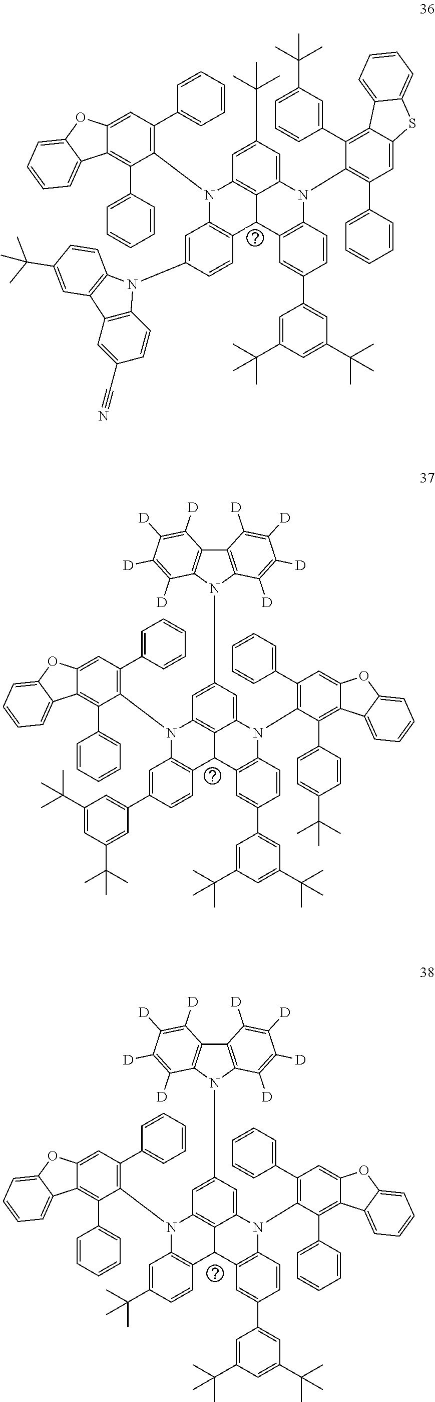 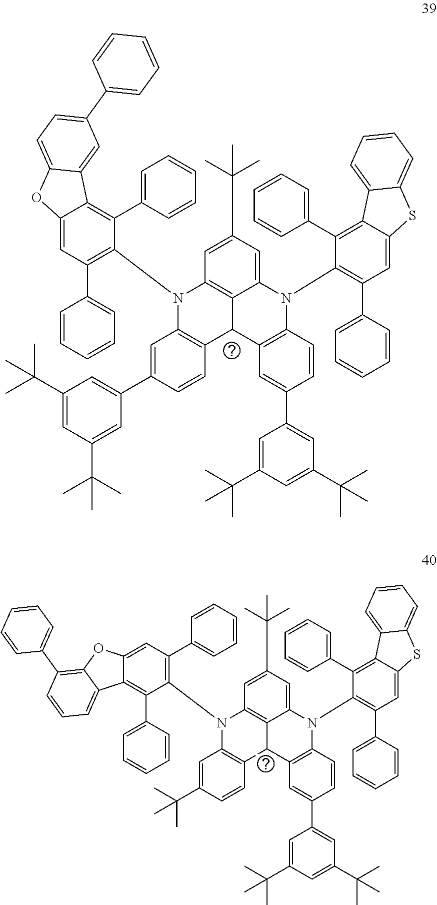 [SMILES: CC(C)(C)c1cc(-c2ccc3c(c2)C2c4ccc(-c5cc(C(C)(C)C)cc(C(C)(C)C)c5)cc4N(c4c(-c5ccccc5)cc5oc6ccc(-c7ccccc7)cc6c5c4-c4ccccc4)c4cc(C(C)(C)C)cc(c42)N3c2c(-c3ccccc3)cc3sc4ccccc4c3c2-c2ccccc2)cc(C(C)(C)C)c1 ; CC(C)(C)c1cc(-c2ccc3c(c2)C2c4ccc(C(C)(C)C)cc4N(c4c(-c5ccccc5)cc5oc6c(-c7ccccc7)cccc6c5c4-c4ccccc4)c4cc(C(C)(C)C)cc(c42)N3c2c(-c3ccccc3)cc3sc4ccccc4c3c2-c2ccccc2)cc(C(C)(C)C)c1] 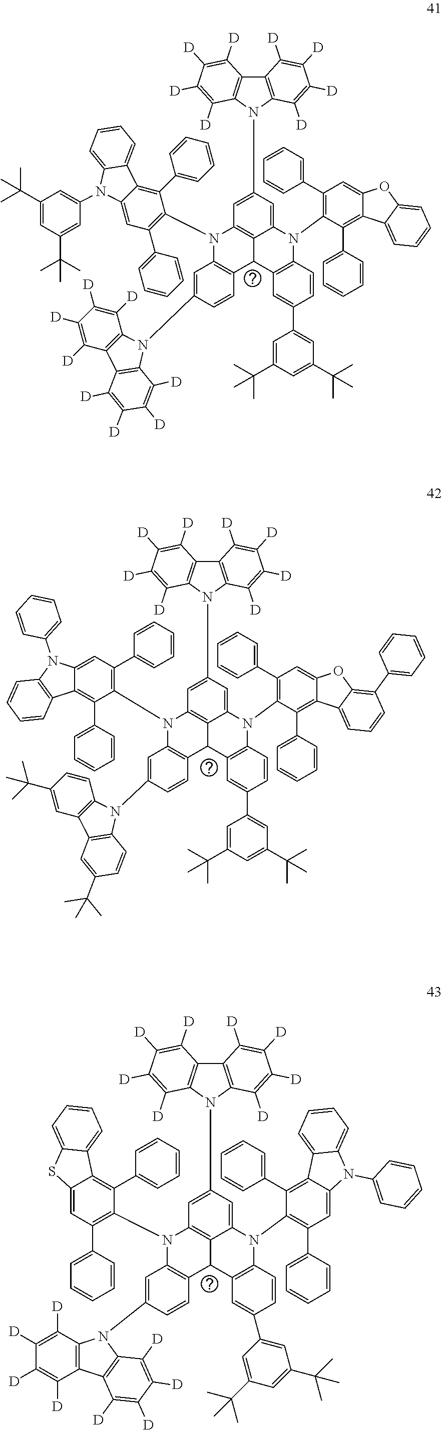 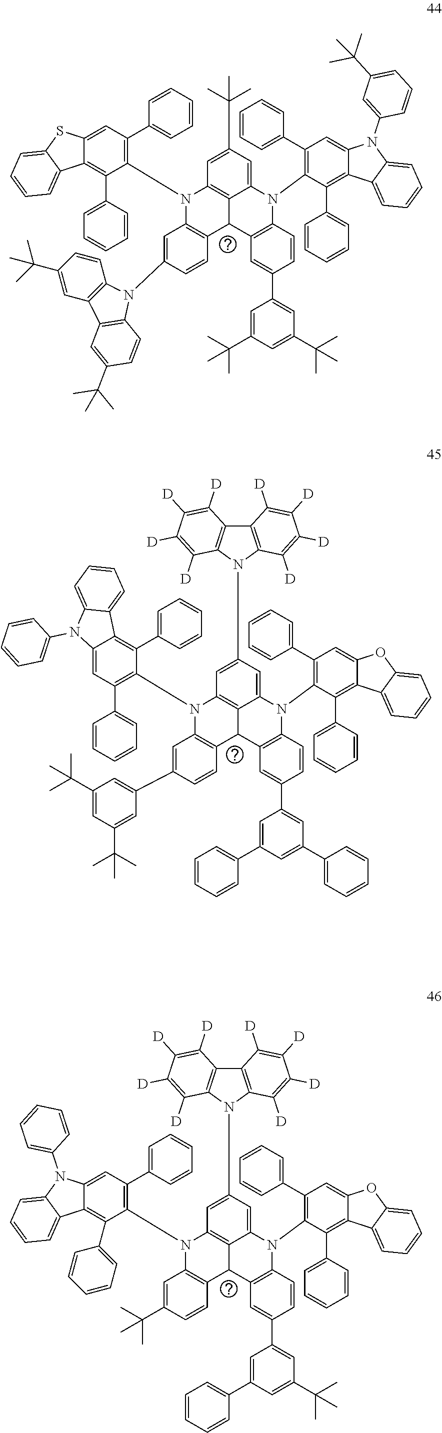 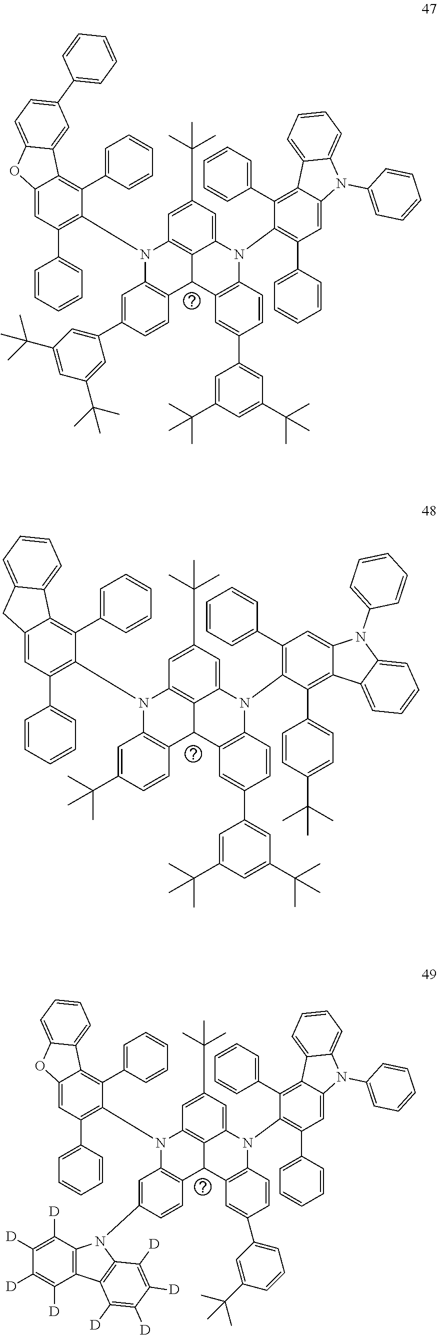 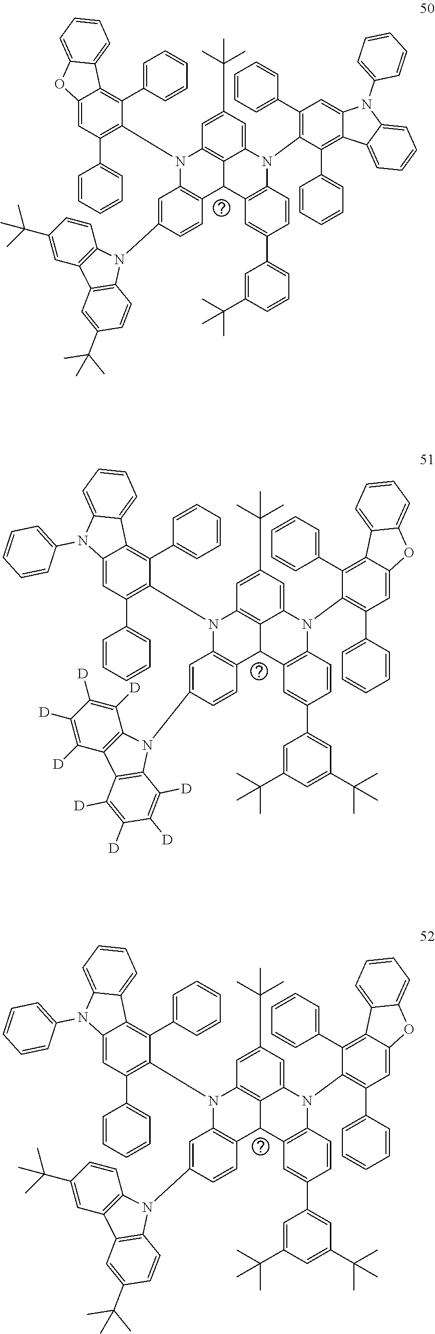 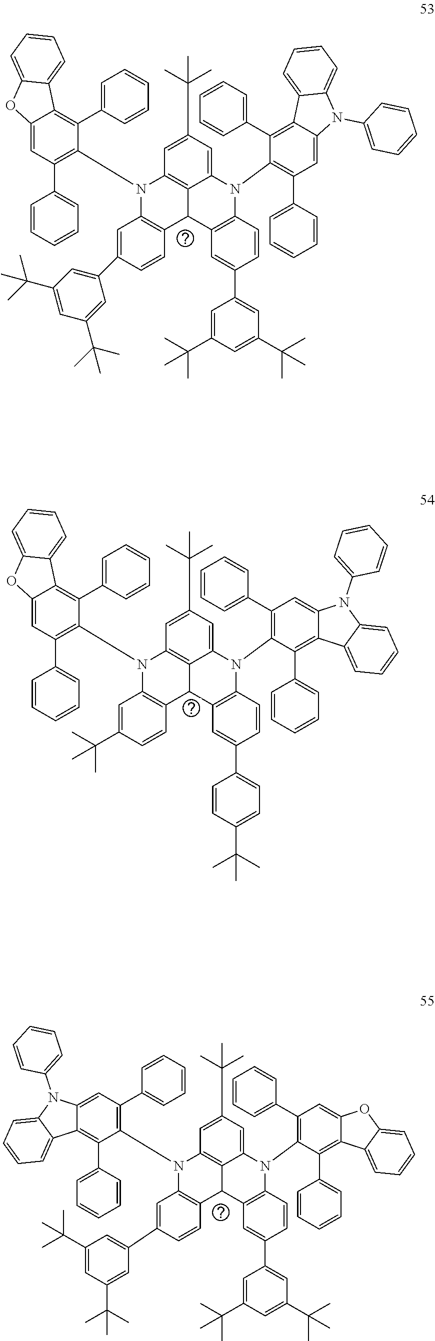 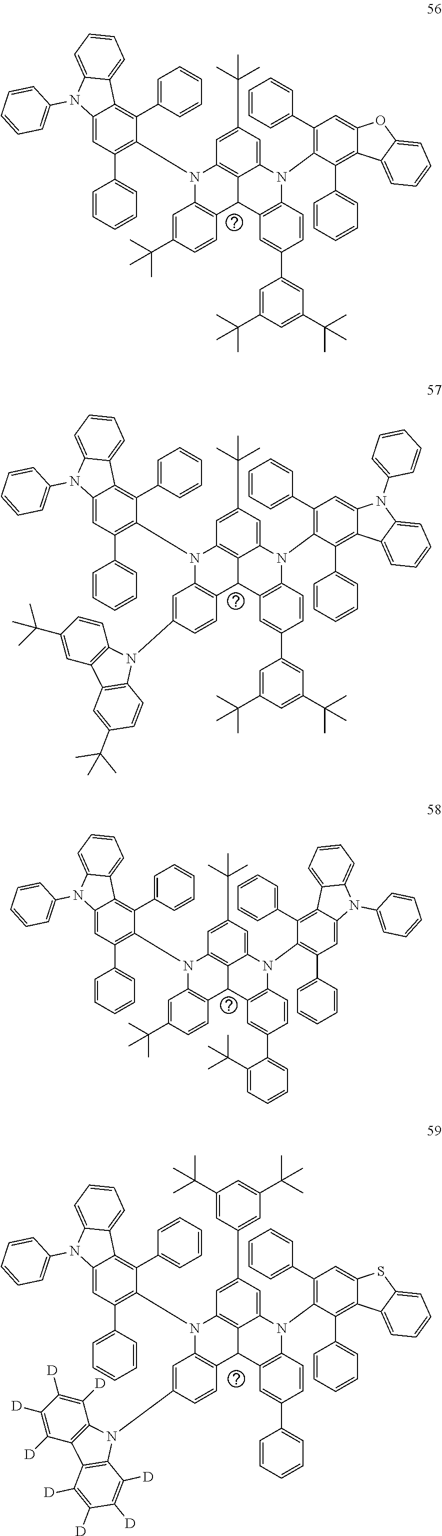 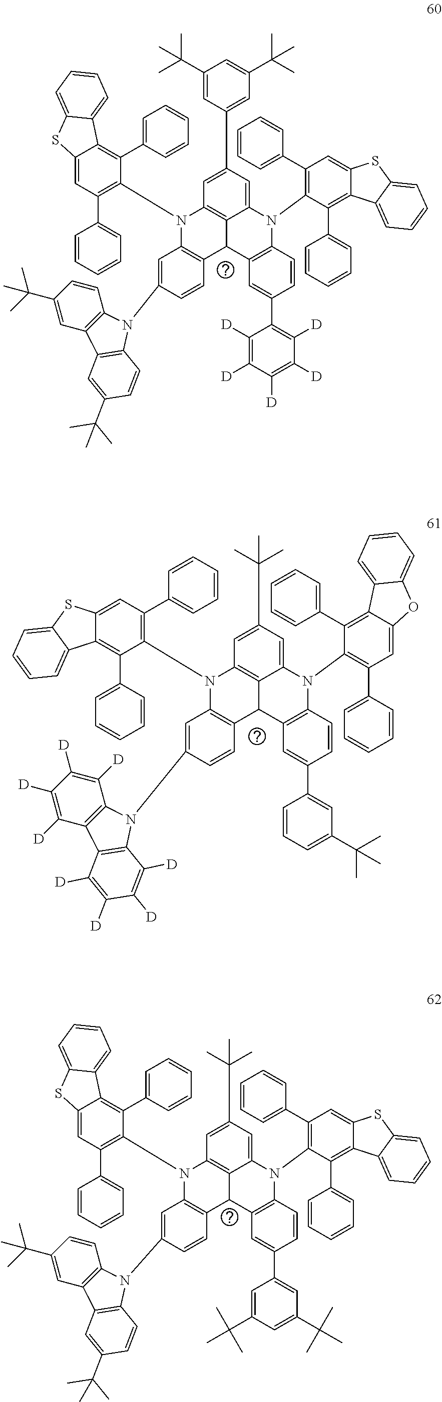 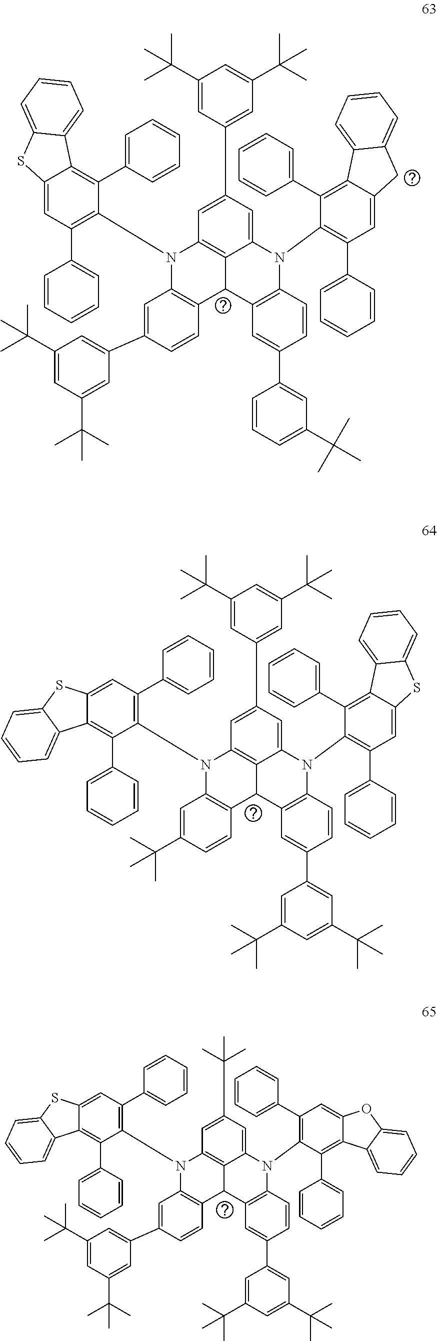 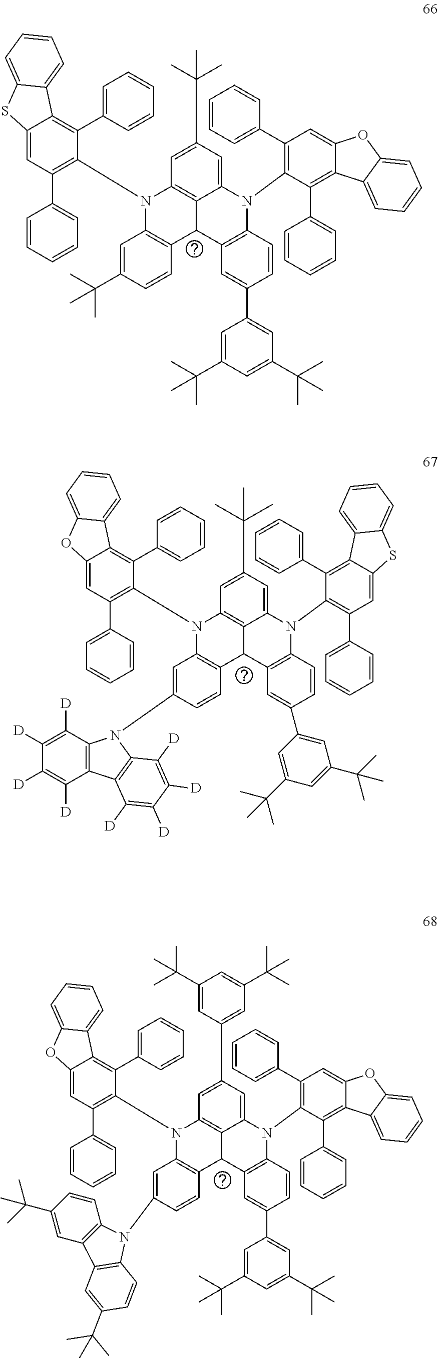 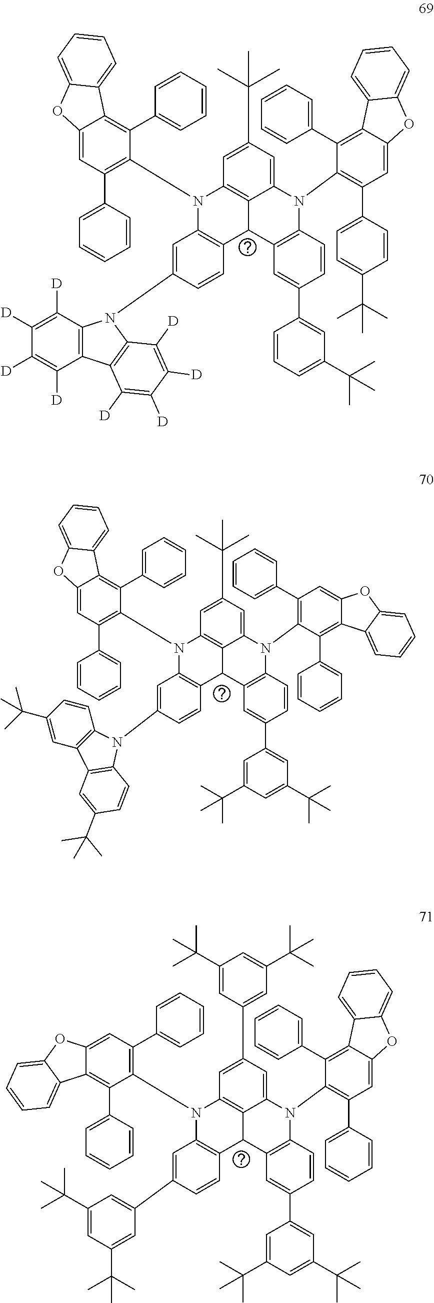 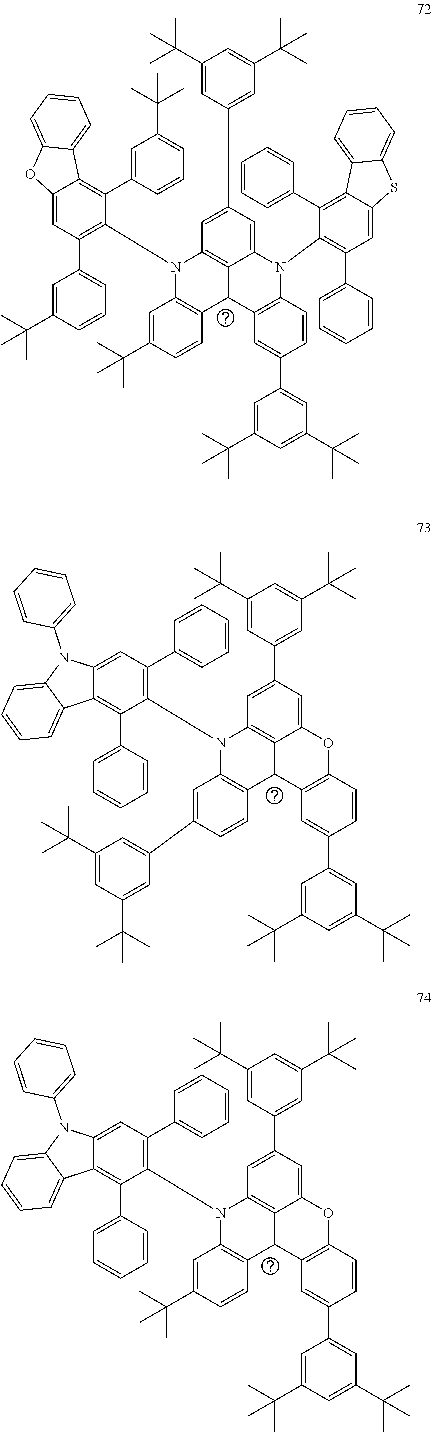 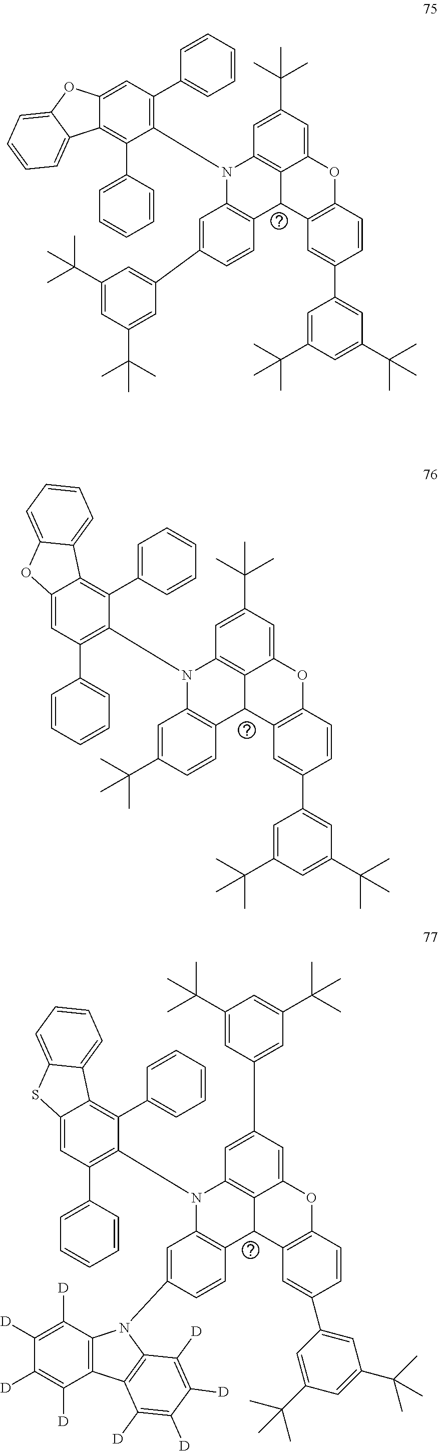 [SMILES: CC(C)(C)c1cc(-c2ccc3c(c2)C2c4ccc(-c5cc(C(C)(C)C)cc(C(C)(C)C)c5)cc4N(c4c(-c5ccccc5)cc5oc6ccccc6c5c4-c4ccccc4)c4cc(C(C)(C)C)cc(c42)O3)cc(C(C)(C)C)c1 ; CC(C)(C)c1cc(-c2ccc3c(c2)C2c4ccc(C(C)(C)C)cc4N(c4c(-c5ccccc5)cc5oc6ccccc6c5c4-c4ccccc4)c4cc(C(C)(C)C)cc(c42)O3)cc(C(C)(C)C)c1 ; [2H]c1c([2H])c([2H])c2c(c1[2H])c1c([2H])c([2H])c([2H])c([2H])c1n2-c1ccc2c(c1)N(c1c(-c3ccccc3)cc3sc4ccccc4c3c1-c1ccccc1)c1cc(-c3cc(C(C)(C)C)cc(C(C)(C)C)c3)cc3c1C2c1cc(-c2cc(C(C)(C)C)cc(C(C)(C)C)c2)ccc1O3] 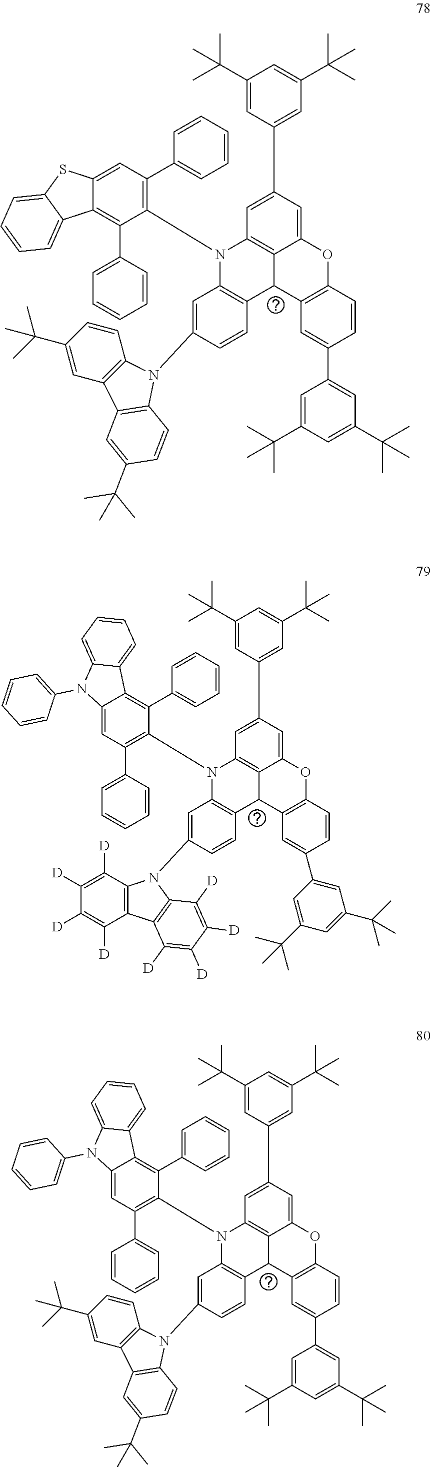 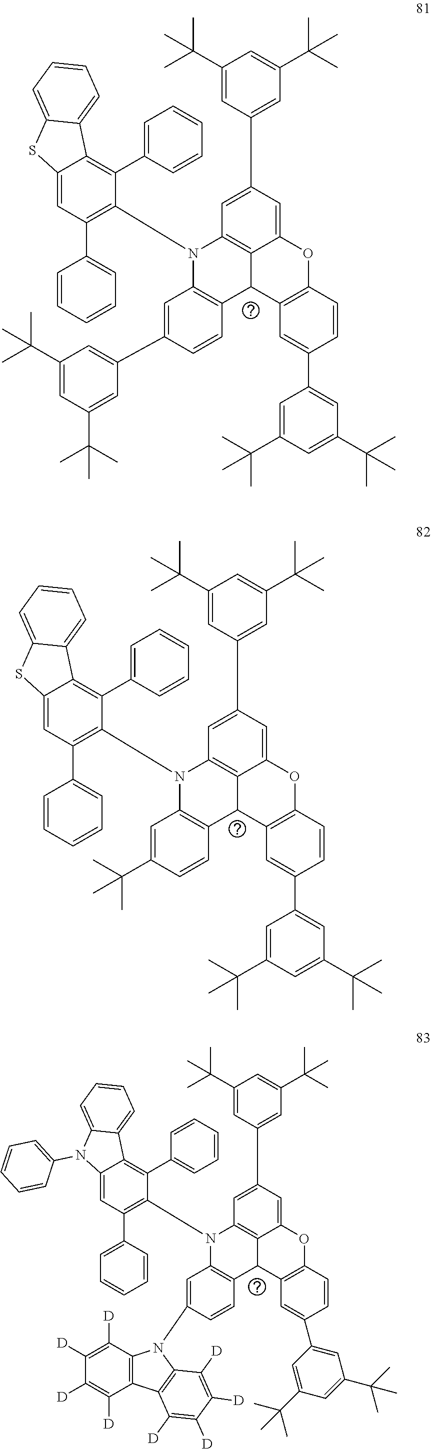 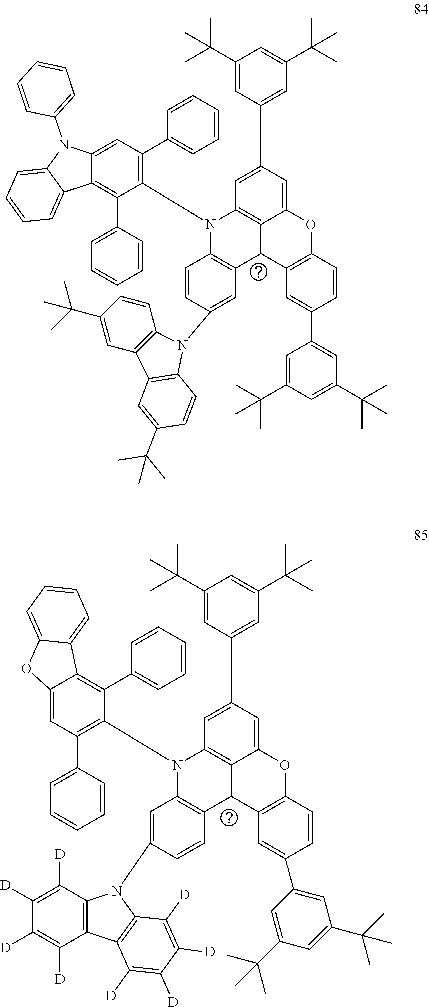 [SMILES: CC(C)(C)c1cc(-c2ccc3c(c2)C2c4cc(-n5c6ccc(C(C)(C)C)cc6c6cc(C(C)(C)C)ccc65)ccc4N(c4c(-c5ccccc5)cc5c(c4-c4ccccc4)c4ccccc4n5-c4ccccc4)c4cc(-c5cc(C(C)(C)C)cc(C(C)(C)C)c5)cc(c42)O3)cc(C(C)(C)C)c1 ; [2H]c1c([2H])c([2H])c2c(c1[2H])c1c([2H])c([2H])c([2H])c([2H])c1n2-c1ccc2c(c1)N(c1c(-c3ccccc3)cc3oc4ccccc4c3c1-c1ccccc1)c1cc(-c3cc(C(C)(C)C)cc(C(C)(C)C)c3)cc3c1C2c1cc(-c2cc(C(C)(C)C)cc(C(C)(C)C)c2)ccc1O3] 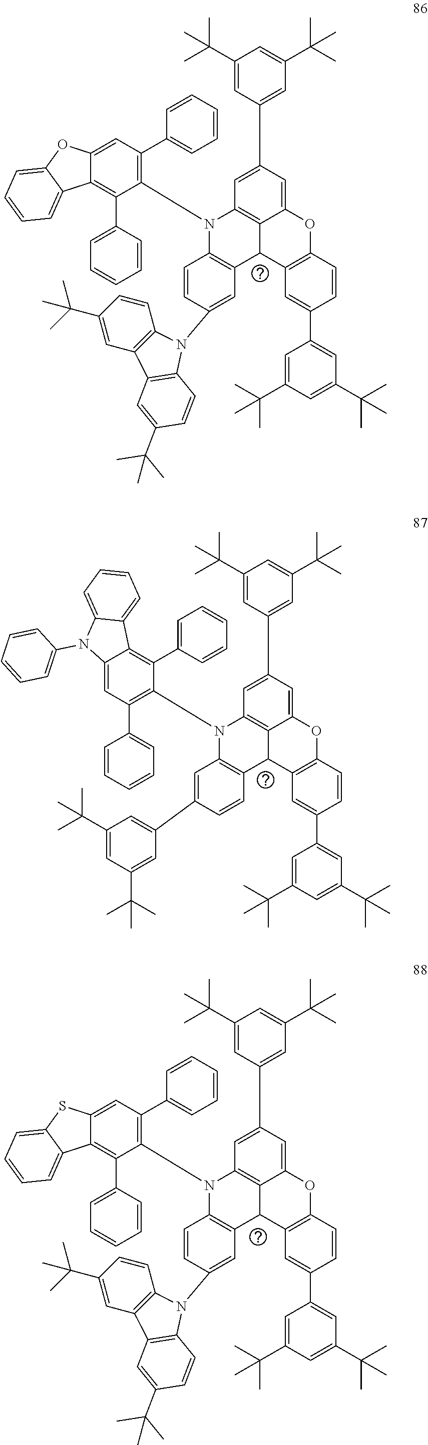 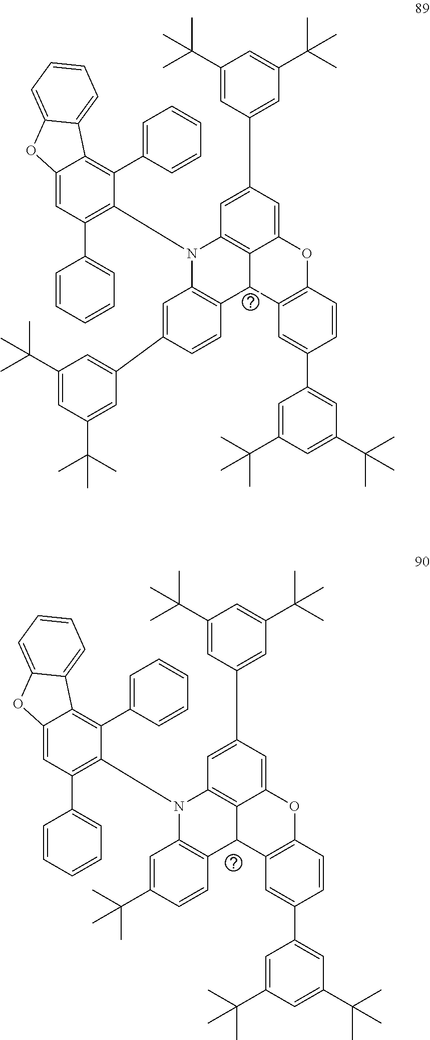 [SMILES: CC(C)(C)c1cc(-c2ccc3c(c2)C2c4ccc(-c5cc(C(C)(C)C)cc(C(C)(C)C)c5)cc4N(c4c(-c5ccccc5)cc5oc6ccccc6c5c4-c4ccccc4)c4cc(-c5cc(C(C)(C)C)cc(C(C)(C)C)c5)cc(c42)O3)cc(C(C)(C)C)c1 ; CC(C)(C)c1cc(-c2ccc3c(c2)C2c4ccc(C(C)(C)C)cc4N(c4c(-c5ccccc5)cc5oc6ccccc6c5c4-c4ccccc4)c4cc(-c5cc(C(C)(C)C)cc(C(C)(C)C)c5)cc(c42)O3)cc(C(C)(C)C)c1] 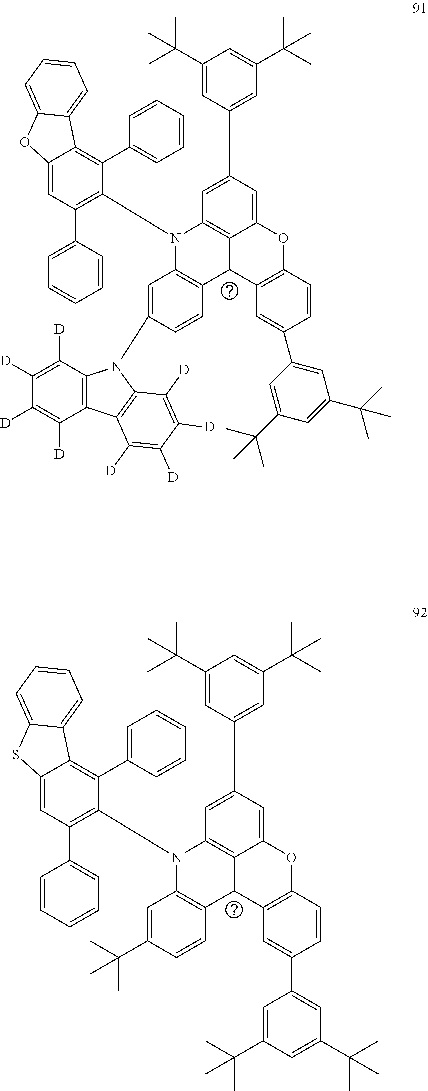 [SMILES: [2H]c1c([2H])c([2H])c2c(c1[2H])c1c([2H])c([2H])c([2H])c([2H])c1n2-c1ccc2c(c1)N(c1c(-c3ccccc3)cc3oc4ccccc4c3c1-c1ccccc1)c1cc(-c3cc(C(C)(C)C)cc(C(C)(C)C)c3)cc3c1C2c1cc(-c2cc(C(C)(C)C)cc(C(C)(C)C)c2)ccc1O3 ; CC(C)(C)c1cc(-c2ccc3c(c2)C2c4ccc(C(C)(C)C)cc4N(c4c(-c5ccccc5)cc5sc6ccccc6c5c4-c4ccccc4)c4cc(-c5cc(C(C)(C)C)cc(C(C)(C)C)c5)cc(c42)O3)cc(C(C)(C)C)c1] 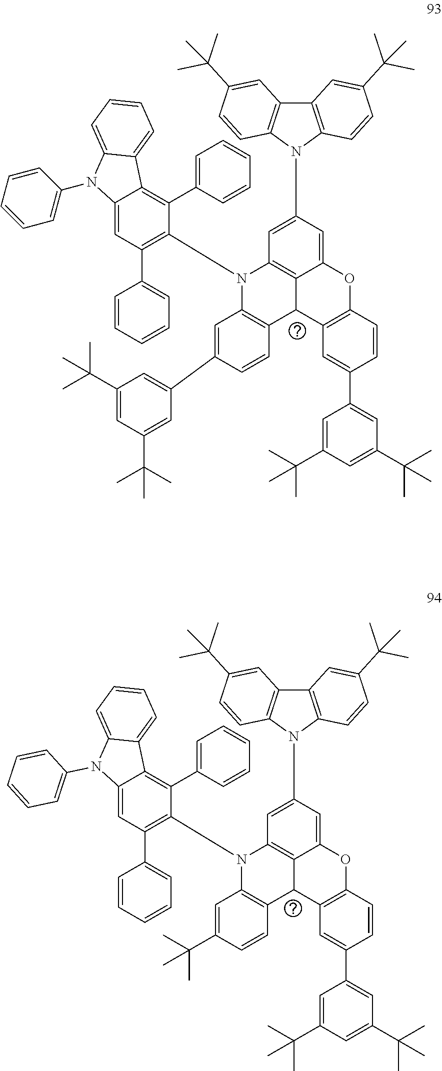 [SMILES: CC(C)(C)c1cc(-c2ccc3c(c2)C2c4ccc(-c5cc(C(C)(C)C)cc(C(C)(C)C)c5)cc4N(c4c(-c5ccccc5)cc5c(c4-c4ccccc4)c4ccccc4n5-c4ccccc4)c4cc(-n5c6ccc(C(C)(C)C)cc6c6cc(C(C)(C)C)ccc65)cc(c42)O3)cc(C(C)(C)C)c1 ; CC(C)(C)c1cc(-c2ccc3c(c2)C2c4ccc(C(C)(C)C)cc4N(c4c(-c5ccccc5)cc5c(c4-c4ccccc4)c4ccccc4n5-c4ccccc4)c4cc(-n5c6ccc(C(C)(C)C)cc6c6cc(C(C)(C)C)ccc65)cc(c42)O3)cc(C(C)(C)C)c1] 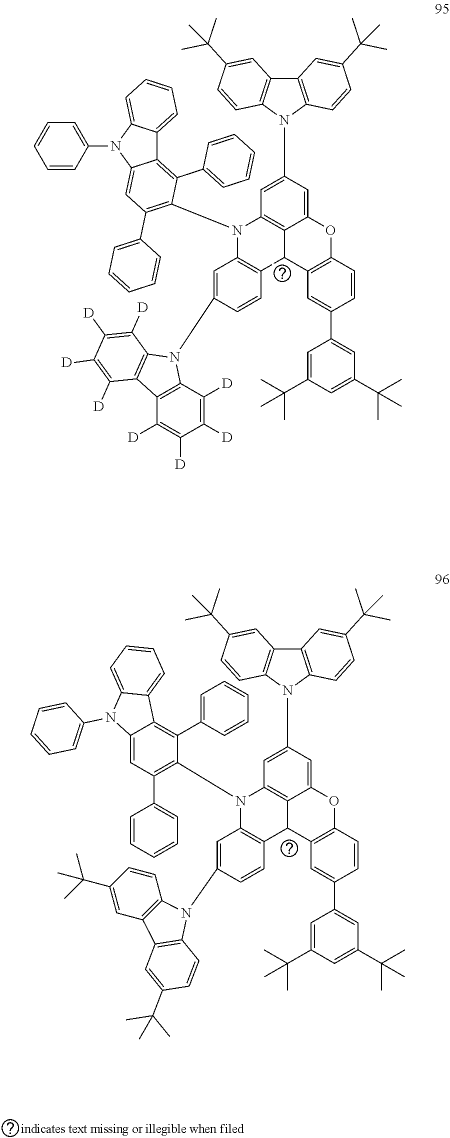 [SMILES: [2H]c1c([2H])c([2H])c2c(c1[2H])c1c([2H])c([2H])c([2H])c([2H])c1n2-c1ccc2c(c1)N(c1c(-c3ccccc3)cc3c(c1-c1ccccc1)c1ccccc1n3-c1ccccc1)c1cc(-n3c4ccc(C(C)(C)C)cc4c4cc(C(C)(C)C)ccc43)cc3c1C2c1cc(-c2cc(C(C)(C)C)cc(C(C)(C)C)c2)ccc1O3 ; CC(C)(C)c1cc(-c2ccc3c(c2)C2c4ccc(-n5c6ccc(C(C)(C)C)cc6c6cc(C(C)(C)C)ccc65)cc4N(c4c(-c5ccccc5)cc5c(c4-c4ccccc4)c4ccccc4n5-c4ccccc4)c4cc(-n5c6ccc(C(C)(C)C)cc6c6cc(C(C)(C)C)ccc65)cc(c42)O3)cc(C(C)(C)C)c1] 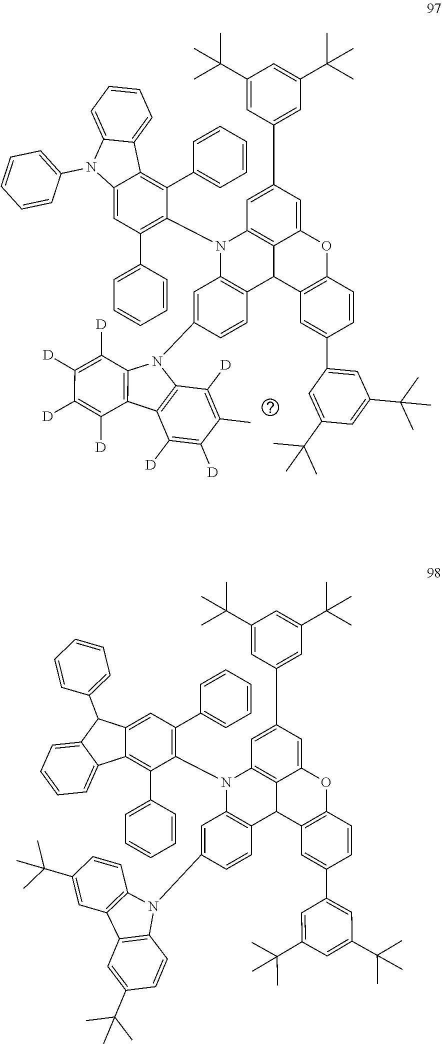 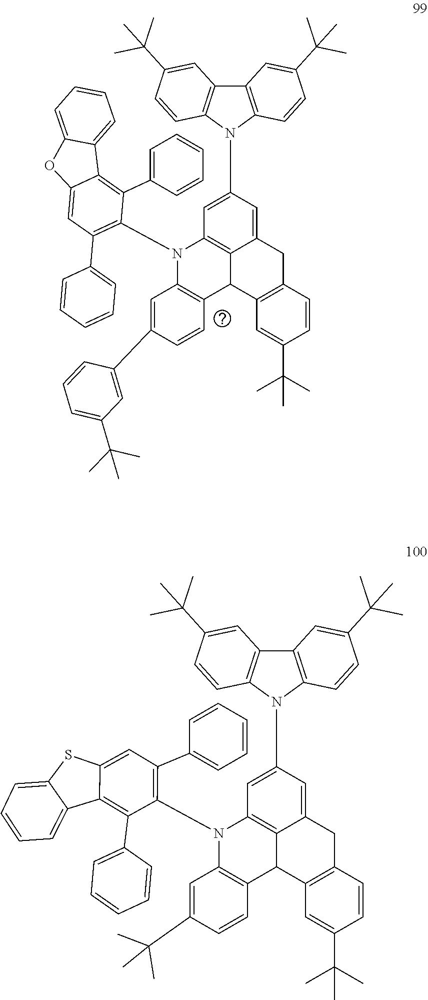 [SMILES: CC(C)(C)c1cccc(-c2ccc3c(c2)N(c2c(-c4ccccc4)cc4oc5ccccc5c4c2-c2ccccc2)c2cc(-n4c5ccc(C(C)(C)C)cc5c5cc(C(C)(C)C)ccc54)cc4c2C3c2cc(C(C)(C)C)ccc2C4)c1 ; CC(C)(C)c1ccc2c(c1)C1c3ccc(C(C)(C)C)cc3N(c3c(-c4ccccc4)cc4sc5ccccc5c4c3-c3ccccc3)c3cc(-n4c5ccc(C(C)(C)C)cc5c5cc(C(C)(C)C)ccc54)cc(c31)C2] 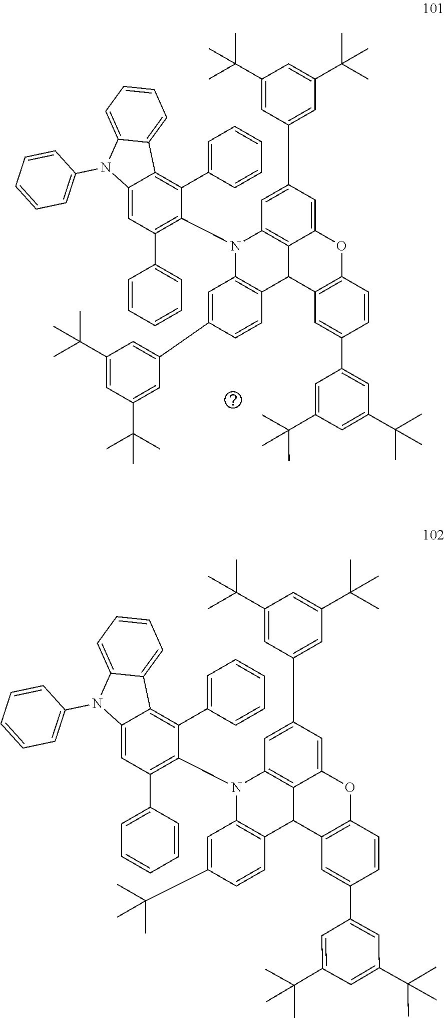 [SMILES: CC(C)(C)c1cc(-c2ccc3c(c2)C2c4ccc(-c5cc(C(C)(C)C)cc(C(C)(C)C)c5)cc4N(c4c(-c5ccccc5)cc5c(c4-c4ccccc4)c4ccccc4n5-c4ccccc4)c4cc(-c5cc(C(C)(C)C)cc(C(C)(C)C)c5)cc(c42)O3)cc(C(C)(C)C)c1 ; CC(C)(C)c1cc(-c2ccc3c(c2)C2c4ccc(C(C)(C)C)cc4N(c4c(-c5ccccc5)cc5c(c4-c4ccccc4)c4ccccc4n5-c4ccccc4)c4cc(-c5cc(C(C)(C)C)cc(C(C)(C)C)c5)cc(c42)O3)cc(C(C)(C)C)c1] 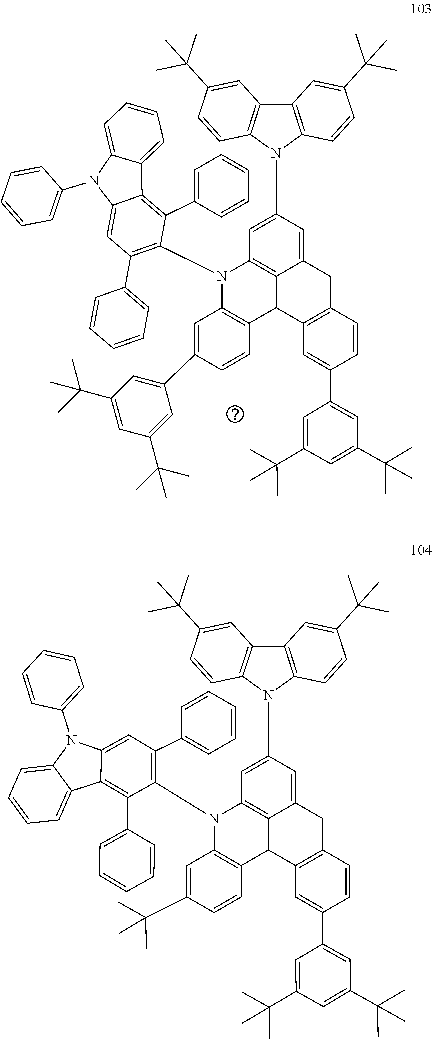 [SMILES: CC(C)(C)c1cc(-c2ccc3c(c2)C2c4ccc(-c5cc(C(C)(C)C)cc(C(C)(C)C)c5)cc4N(c4c(-c5ccccc5)cc5c(c4-c4ccccc4)c4ccccc4n5-c4ccccc4)c4cc(-n5c6ccc(C(C)(C)C)cc6c6cc(C(C)(C)C)ccc65)cc(c42)C3)cc(C(C)(C)C)c1 ; CC(C)(C)c1cc(-c2ccc3c(c2)C2c4ccc(C(C)(C)C)cc4N(c4c(-c5ccccc5)cc5c(c4-c4ccccc4)c4ccccc4n5-c4ccccc4)c4cc(-n5c6ccc(C(C)(C)C)cc6c6cc(C(C)(C)C)ccc65)cc(c42)C3)cc(C(C)(C)C)c1] 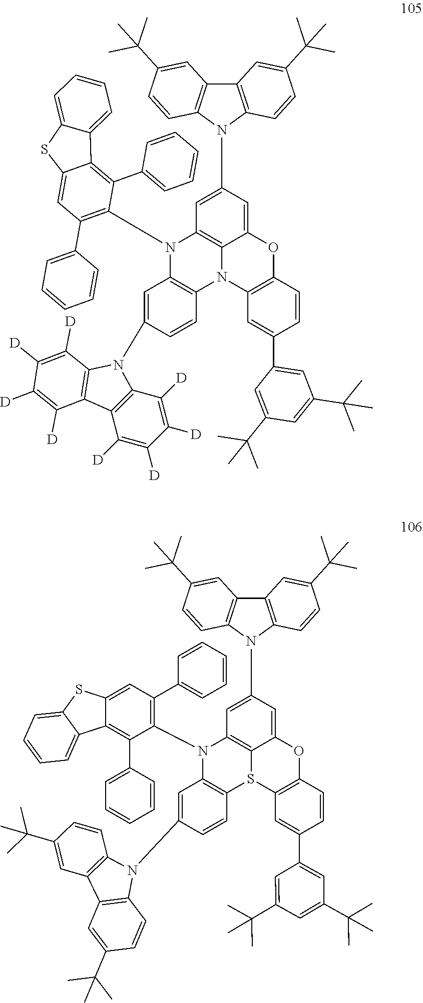 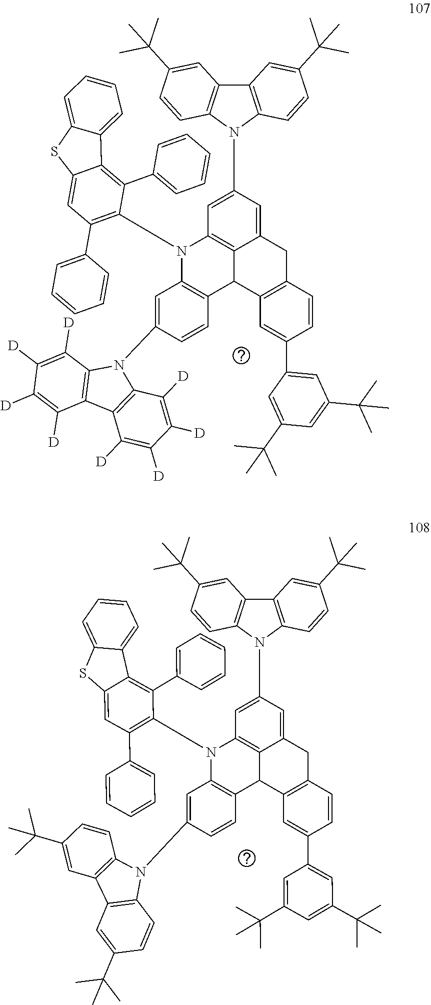 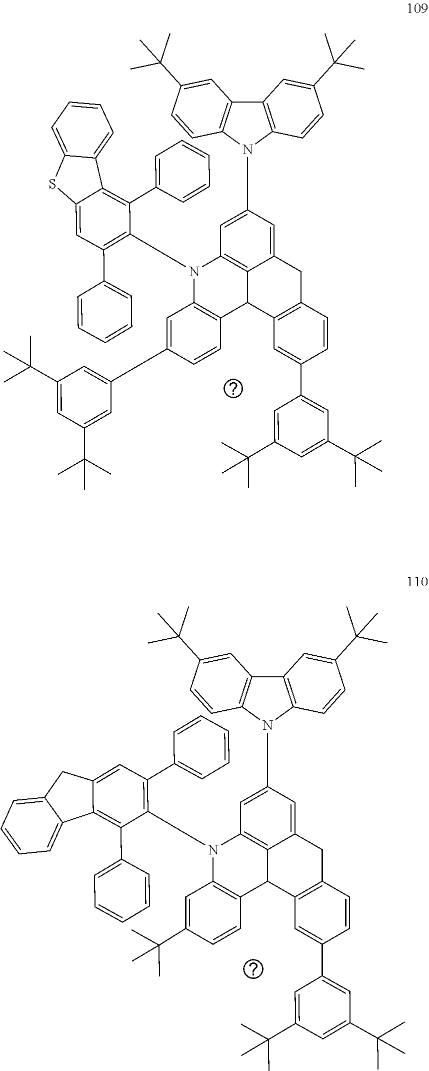 [SMILES: CC(C)(C)c1cc(-c2ccc3c(c2)C2c4ccc(-c5cc(C(C)(C)C)cc(C(C)(C)C)c5)cc4N(c4c(-c5ccccc5)cc5sc6ccccc6c5c4-c4ccccc4)c4cc(-n5c6ccc(C(C)(C)C)cc6c6cc(C(C)(C)C)ccc65)cc(c42)C3)cc(C(C)(C)C)c1 ; CC(C)(C)c1cc(-c2ccc3c(c2)C2c4ccc(C(C)(C)C)cc4N(c4c(-c5ccccc5)cc5c(c4-c4ccccc4)-c4ccccc4C5)c4cc(-n5c6ccc(C(C)(C)C)cc6c6cc(C(C)(C)C)ccc65)cc(c42)C3)cc(C(C)(C)C)c1] 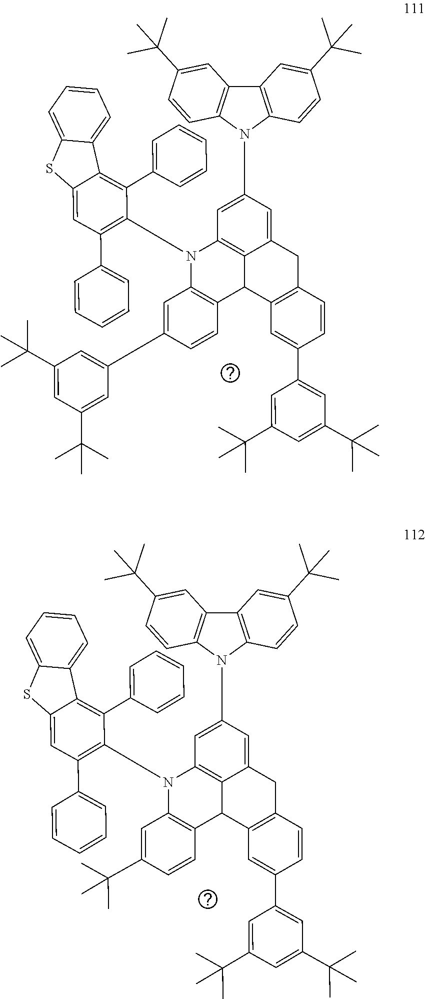 [SMILES: CC(C)(C)c1cc(-c2ccc3c(c2)C2c4ccc(-c5cc(C(C)(C)C)cc(C(C)(C)C)c5)cc4N(c4c(-c5ccccc5)cc5sc6ccccc6c5c4-c4ccccc4)c4cc(-n5c6ccc(C(C)(C)C)cc6c6cc(C(C)(C)C)ccc65)cc(c42)C3)cc(C(C)(C)C)c1 ; CC(C)(C)c1cc(-c2ccc3c(c2)C2c4ccc(C(C)(C)C)cc4N(c4c(-c5ccccc5)cc5sc6ccccc6c5c4-c4ccccc4)c4cc(-n5c6ccc(C(C)(C)C)cc6c6cc(C(C)(C)C)ccc65)cc(c42)C3)cc(C(C)(C)C)c1] 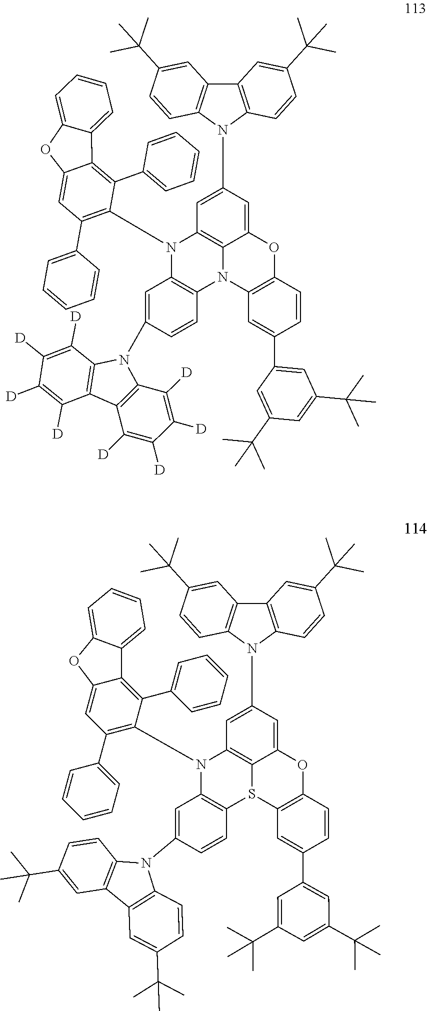 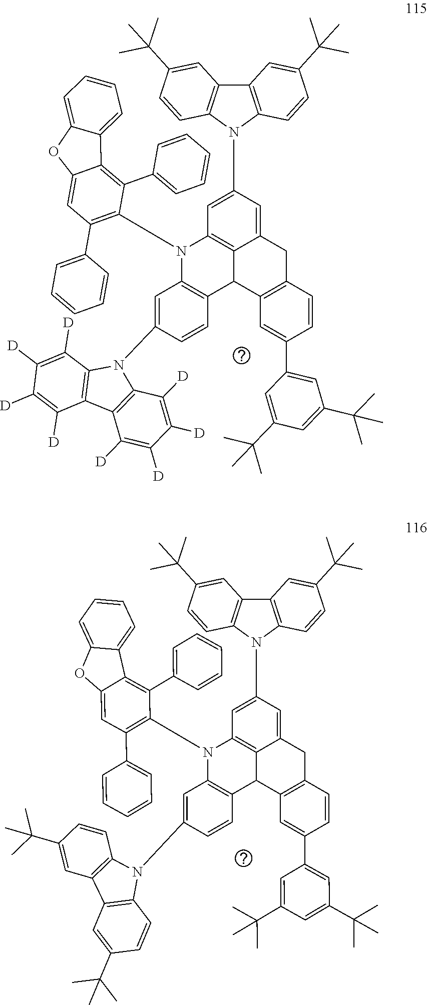 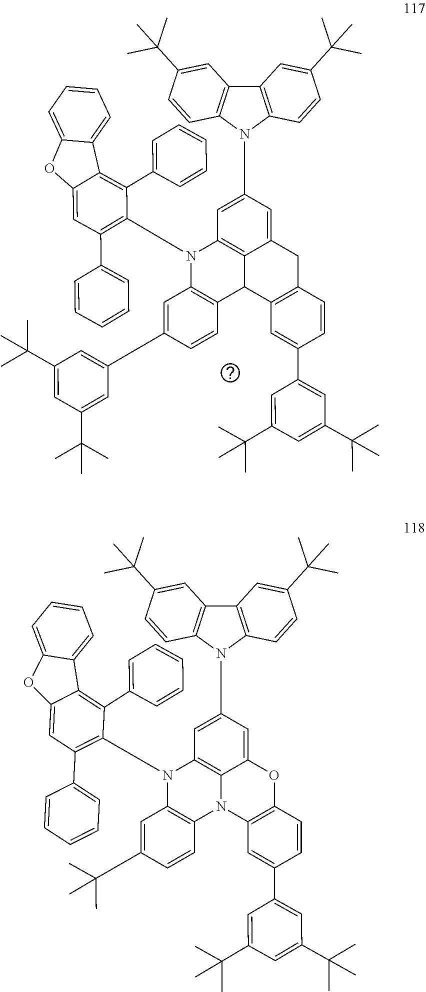 [SMILES: CC(C)(C)c1cc(-c2ccc3c(c2)C2c4ccc(-c5cc(C(C)(C)C)cc(C(C)(C)C)c5)cc4N(c4c(-c5ccccc5)cc5oc6ccccc6c5c4-c4ccccc4)c4cc(-n5c6ccc(C(C)(C)C)cc6c6cc(C(C)(C)C)ccc65)cc(c42)C3)cc(C(C)(C)C)c1 ; CC(C)(C)c1cc(-c2ccc3c(c2)N2c4ccc(C(C)(C)C)cc4N(c4c(-c5ccccc5)cc5oc6ccccc6c5c4-c4ccccc4)c4cc(-n5c6ccc(C(C)(C)C)cc6c6cc(C(C)(C)C)ccc65)cc(c42)O3)cc(C(C)(C)C)c1] 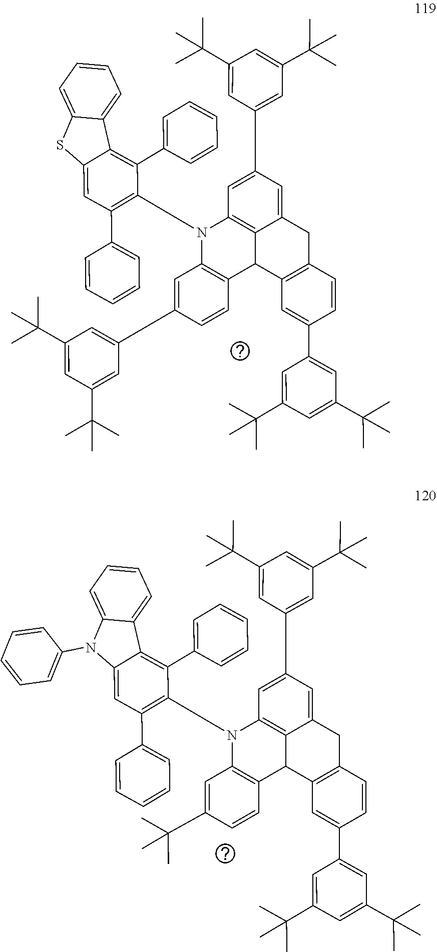 [SMILES: CC(C)(C)c1cc(-c2ccc3c(c2)C2c4ccc(-c5cc(C(C)(C)C)cc(C(C)(C)C)c5)cc4N(c4c(-c5ccccc5)cc5sc6ccccc6c5c4-c4ccccc4)c4cc(-c5cc(C(C)(C)C)cc(C(C)(C)C)c5)cc(c42)C3)cc(C(C)(C)C)c1 ; CC(C)(C)c1cc(-c2ccc3c(c2)C2c4ccc(C(C)(C)C)cc4N(c4c(-c5ccccc5)cc5c(c4-c4ccccc4)c4ccccc4n5-c4ccccc4)c4cc(-c5cc(C(C)(C)C)cc(C(C)(C)C)c5)cc(c42)C3)cc(C(C)(C)C)c1] 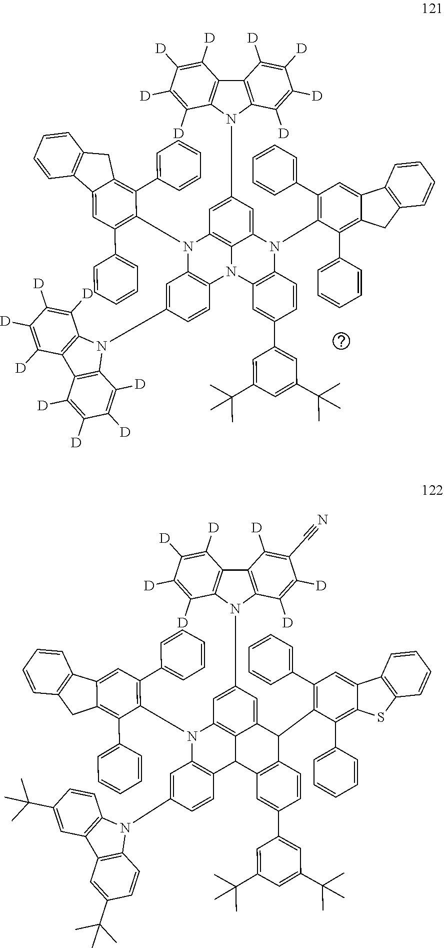 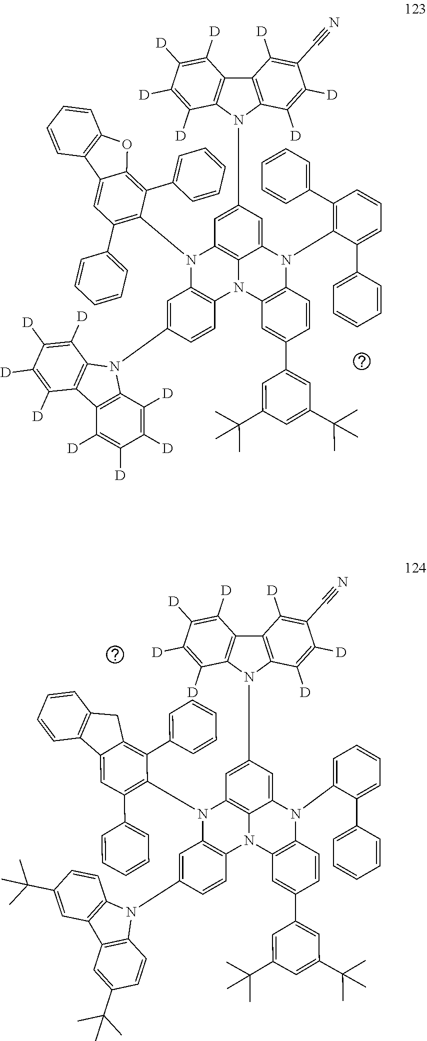 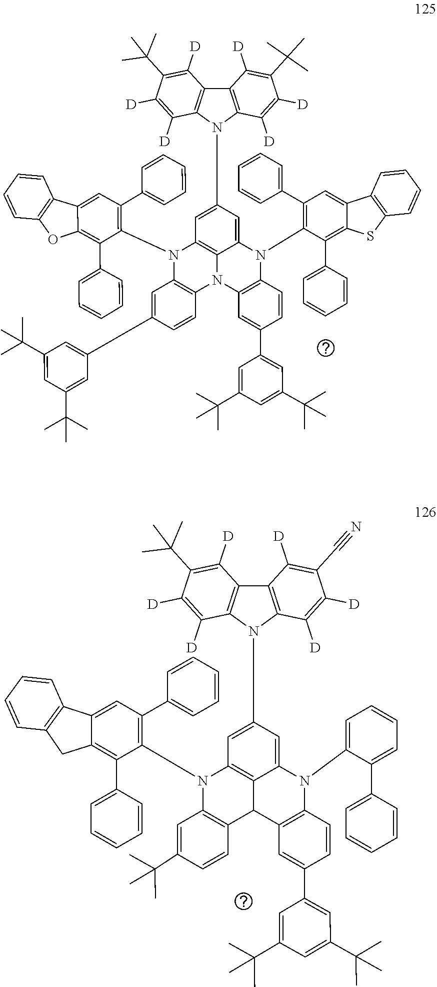 [SMILES: [2H]c1c(C(C)(C)C)c([2H])c2c3c([2H])c(C(C)(C)C)c([2H])c([2H])c3n(-c3cc4c5c(c3)N(c3c(-c6ccccc6)cc6c(sc7ccccc76)c3-c3ccccc3)c3ccc(-c6cc(C(C)(C)C)cc(C(C)(C)C)c6)cc3N5c3ccc(-c5cc(C(C)(C)C)cc(C(C)(C)C)c5)cc3N4c3c(-c4ccccc4)cc4c(oc5ccccc54)c3-c3ccccc3)c2c1[2H] ; [2H]c1c(C#N)c([2H])c2c3c([2H])c(C(C)(C)C)c([2H])c([2H])c3n(-c3cc4c5c(c3)N(c3c(-c6ccccc6)cc6c(c3-c3ccccc3)Cc3ccccc3-6)c3cc(C(C)(C)C)ccc3C5c3cc(-c5cc(C(C)(C)C)cc(C(C)(C)C)c5)ccc3N4c3ccccc3-c3ccccc3)c2c1[2H]] 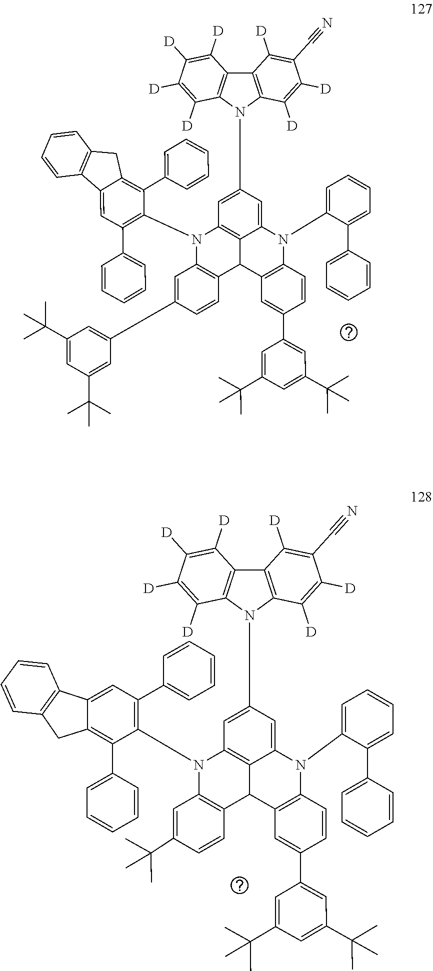 [SMILES: [2H]c1c([2H])c([2H])c2c(c1[2H])c1c([2H])c(C#N)c([2H])c([2H])c1n2-c1cc2c3c(c1)N(c1c(-c4ccccc4)cc4c(c1-c1ccccc1)Cc1ccccc1-4)c1cc(-c4cc(C(C)(C)C)cc(C(C)(C)C)c4)ccc1C3c1cc(-c3cc(C(C)(C)C)cc(C(C)(C)C)c3)ccc1N2c1ccccc1-c1ccccc1 ; [2H]c1c([2H])c([2H])c2c(c1[2H])c1c([2H])c(C#N)c([2H])c([2H])c1n2-c1cc2c3c(c1)N(c1c(-c4ccccc4)cc4c(c1-c1ccccc1)Cc1ccccc1-4)c1cc(C(C)(C)C)ccc1C3c1cc(-c3cc(C(C)(C)C)cc(C(C)(C)C)c3)ccc1N2c1ccccc1-c1ccccc1] 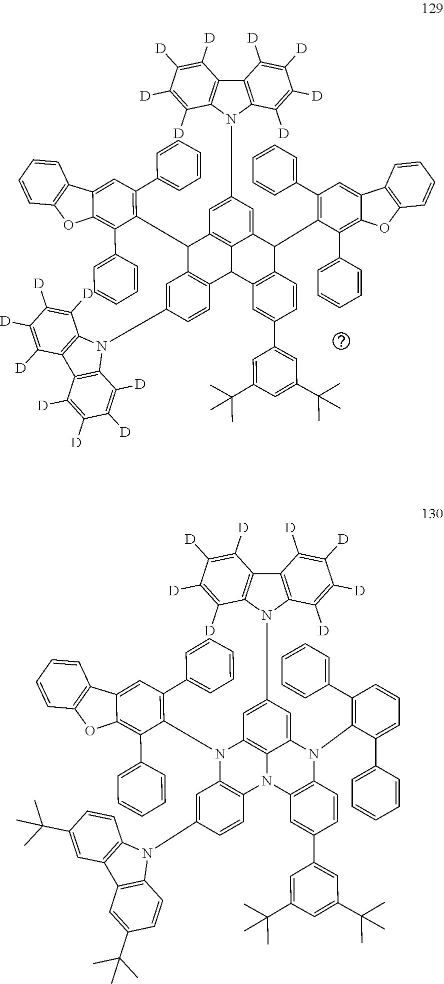 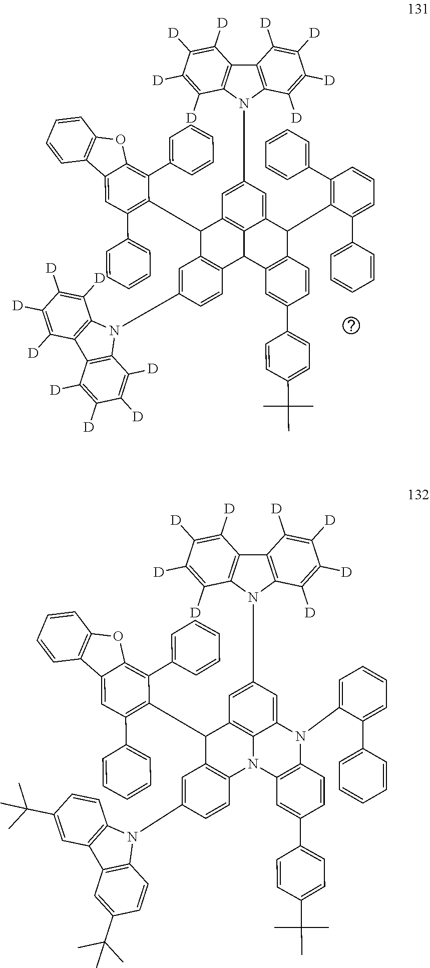 [SMILES: [2H]c1c([2H])c([2H])c2c(c1[2H])c1c([2H])c([2H])c([2H])c([2H])c1n2-c1ccc2c(c1)C(c1c(-c3ccccc3)cc3c(oc4ccccc43)c1-c1ccccc1)c1cc(-n3c4c([2H])c([2H])c([2H])c([2H])c4c4c([2H])c([2H])c([2H])c([2H])c43)cc3c1C2c1cc(-c2ccc(C(C)(C)C)cc2)ccc1C3c1c(-c2ccccc2)cccc1-c1ccccc1 ; [2H]c1c([2H])c([2H])c2c(c1[2H])c1c([2H])c([2H])c([2H])c([2H])c1n2-c1cc2c3c(c1)N(c1ccccc1-c1ccccc1)c1ccc(-c4ccc(C(C)(C)C)cc4)cc1N3c1ccc(-n3c4ccc(C(C)(C)C)cc4c4cc(C(C)(C)C)ccc43)cc1C2c1c(-c2ccccc2)cc2c(oc3ccccc32)c1-c1ccccc1] 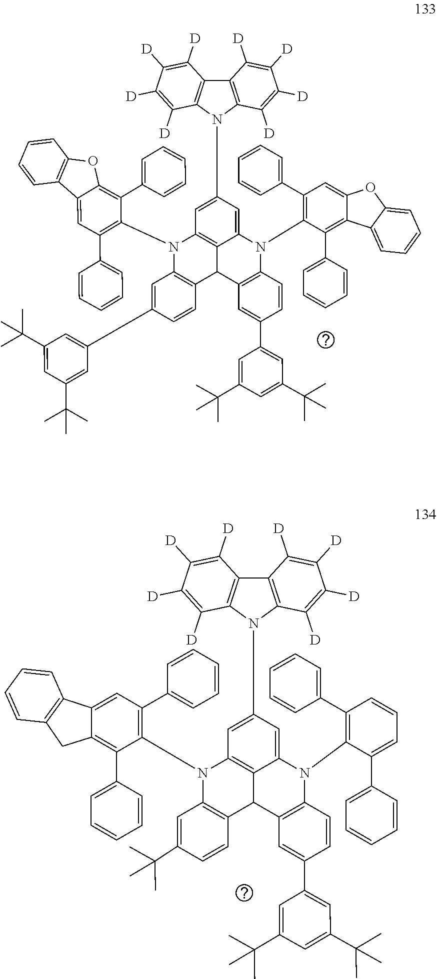 [SMILES: [2H]c1c([2H])c([2H])c2c(c1[2H])c1c([2H])c([2H])c([2H])c([2H])c1n2-c1cc2c3c(c1)N(c1c(-c4ccccc4)cc4oc5ccccc5c4c1-c1ccccc1)c1ccc(-c4cc(C(C)(C)C)cc(C(C)(C)C)c4)cc1C3c1ccc(-c3cc(C(C)(C)C)cc(C(C)(C)C)c3)cc1N2c1c(-c2ccccc2)cc2c(oc3ccccc32)c1-c1ccccc1 ; [2H]c1c([2H])c([2H])c2c(c1[2H])c1c([2H])c([2H])c([2H])c([2H])c1n2-c1cc2c3c(c1)N(c1c(-c4ccccc4)cc4c(c1-c1ccccc1)Cc1ccccc1-4)c1cc(C(C)(C)C)ccc1C3c1cc(-c3cc(C(C)(C)C)cc(C(C)(C)C)c3)ccc1N2c1c(-c2ccccc2)cccc1-c1ccccc1] 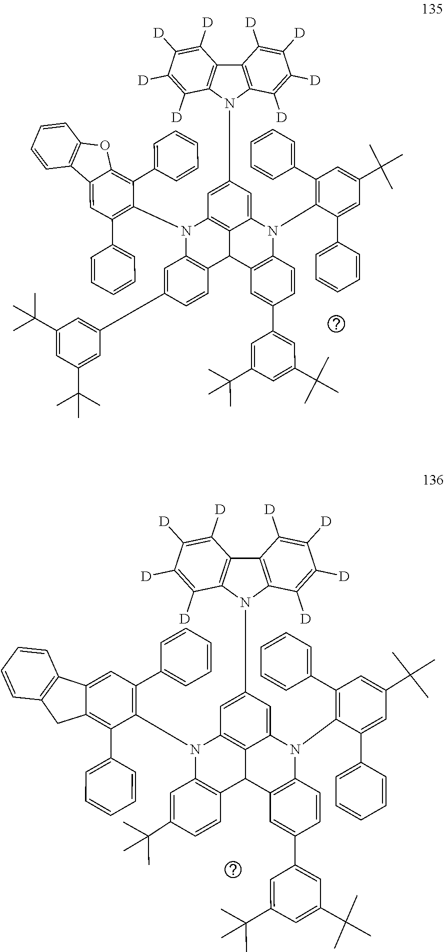 [SMILES: [2H]c1c([2H])c([2H])c2c(c1[2H])c1c([2H])c([2H])c([2H])c([2H])c1n2-c1cc2c3c(c1)N(c1c(-c4ccccc4)cc4c(oc5ccccc54)c1-c1ccccc1)c1cc(-c4cc(C(C)(C)C)cc(C(C)(C)C)c4)ccc1C3c1cc(-c3cc(C(C)(C)C)cc(C(C)(C)C)c3)ccc1N2c1c(-c2ccccc2)cc(C(C)(C)C)cc1-c1ccccc1 ; [2H]c1c([2H])c([2H])c2c(c1[2H])c1c([2H])c([2H])c([2H])c([2H])c1n2-c1cc2c3c(c1)N(c1c(-c4ccccc4)cc4c(c1-c1ccccc1)Cc1ccccc1-4)c1cc(C(C)(C)C)ccc1C3c1cc(-c3cc(C(C)(C)C)cc(C(C)(C)C)c3)ccc1N2c1c(-c2ccccc2)cc(C(C)(C)C)cc1-c1ccccc1] 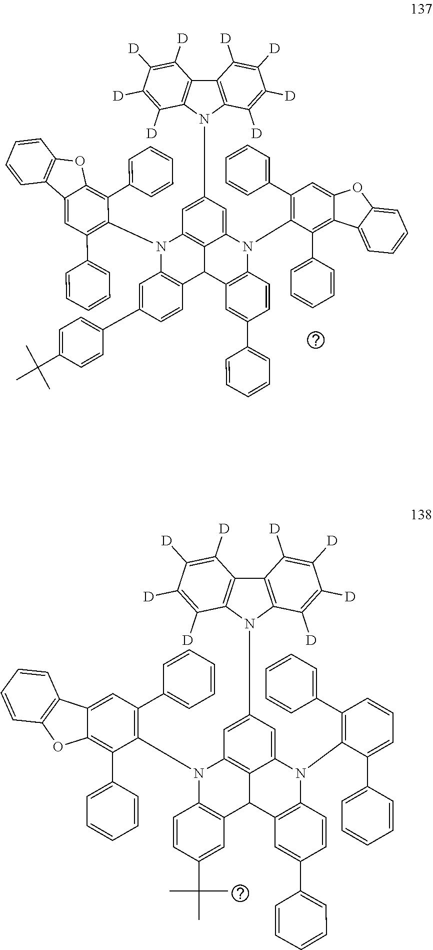 [SMILES: [2H]c1c([2H])c([2H])c2c(c1[2H])c1c([2H])c([2H])c([2H])c([2H])c1n2-c1cc2c3c(c1)N(c1c(-c4ccccc4)cc4oc5ccccc5c4c1-c1ccccc1)c1ccc(-c4ccccc4)cc1C3c1ccc(-c3ccc(C(C)(C)C)cc3)cc1N2c1c(-c2ccccc2)cc2c(oc3ccccc32)c1-c1ccccc1 ; [2H]c1c([2H])c([2H])c2c(c1[2H])c1c([2H])c([2H])c([2H])c([2H])c1n2-c1cc2c3c(c1)N(c1c(-c4ccccc4)cc4c(oc5ccccc54)c1-c1ccccc1)c1ccc(C(C)(C)C)cc1C3c1cc(-c3ccccc3)ccc1N2c1c(-c2ccccc2)cccc1-c1ccccc1] 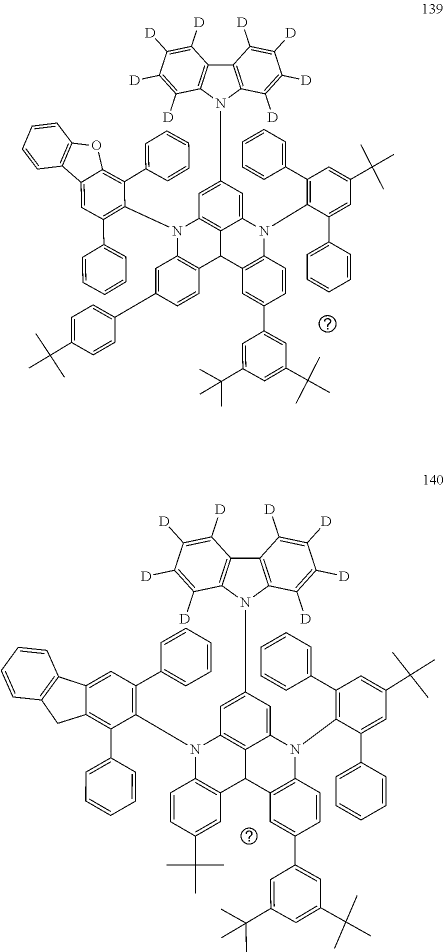 [SMILES: [2H]c1c([2H])c([2H])c2c(c1[2H])c1c([2H])c([2H])c([2H])c([2H])c1n2-c1cc2c3c(c1)N(c1c(-c4ccccc4)cc4c(oc5ccccc54)c1-c1ccccc1)c1cc(-c4ccc(C(C)(C)C)cc4)ccc1C3c1cc(-c3cc(C(C)(C)C)cc(C(C)(C)C)c3)ccc1N2c1c(-c2ccccc2)cc(C(C)(C)C)cc1-c1ccccc1 ; [2H]c1c([2H])c([2H])c2c(c1[2H])c1c([2H])c([2H])c([2H])c([2H])c1n2-c1cc2c3c(c1)N(c1c(-c4ccccc4)cc4c(c1-c1ccccc1)Cc1ccccc1-4)c1ccc(C(C)(C)C)cc1C3c1cc(-c3cc(C(C)(C)C)cc(C(C)(C)C)c3)ccc1N2c1c(-c2ccccc2)cc(C(C)(C)C)cc1-c1ccccc1] 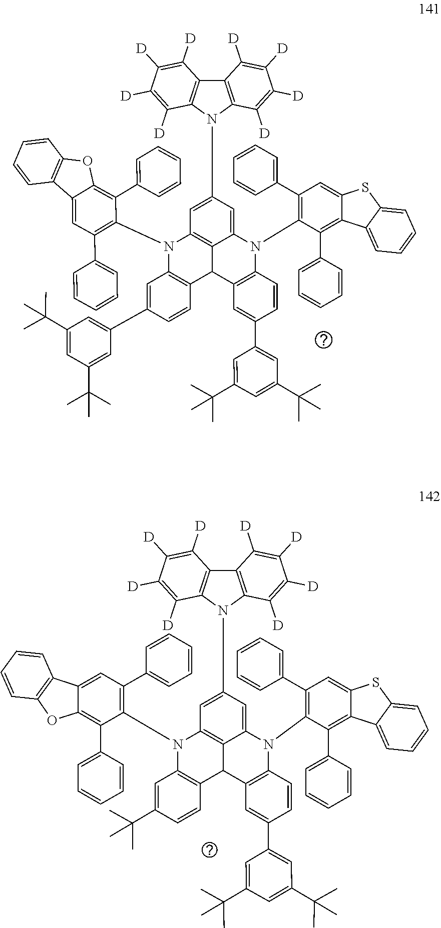 [SMILES: [2H]c1c([2H])c([2H])c2c(c1[2H])c1c([2H])c([2H])c([2H])c([2H])c1n2-c1cc2c3c(c1)N(c1c(-c4ccccc4)cc4sc5ccccc5c4c1-c1ccccc1)c1ccc(-c4cc(C(C)(C)C)cc(C(C)(C)C)c4)cc1C3c1ccc(-c3cc(C(C)(C)C)cc(C(C)(C)C)c3)cc1N2c1c(-c2ccccc2)cc2c(oc3ccccc32)c1-c1ccccc1 ; [2H]c1c([2H])c([2H])c2c(c1[2H])c1c([2H])c([2H])c([2H])c([2H])c1n2-c1cc2c3c(c1)N(c1c(-c4ccccc4)cc4sc5ccccc5c4c1-c1ccccc1)c1ccc(-c4cc(C(C)(C)C)cc(C(C)(C)C)c4)cc1C3c1ccc(C(C)(C)C)cc1N2c1c(-c2ccccc2)cc2c(oc3ccccc32)c1-c1ccccc1] 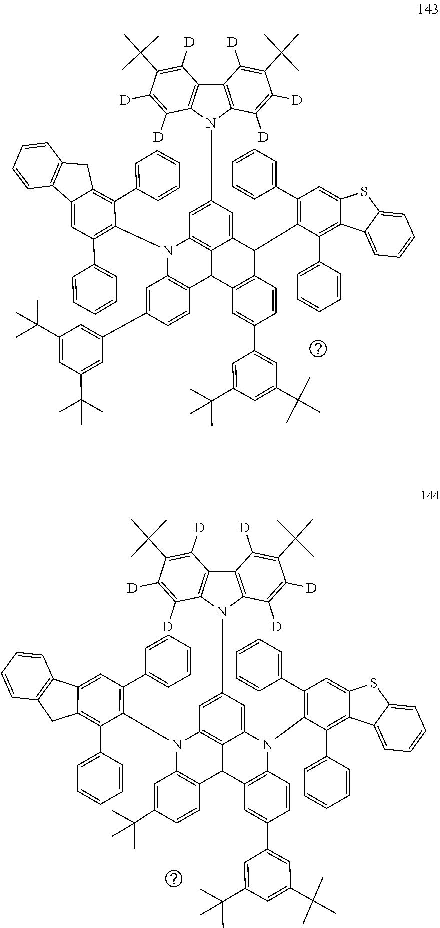 [SMILES: [2H]c1c(C(C)(C)C)c([2H])c2c3c([2H])c(C(C)(C)C)c([2H])c([2H])c3n(-c3cc4c5c(c3)N(c3c(-c6ccccc6)cc6c(c3-c3ccccc3)Cc3ccccc3-6)c3cc(-c6cc(C(C)(C)C)cc(C(C)(C)C)c6)ccc3C5c3cc(-c5cc(C(C)(C)C)cc(C(C)(C)C)c5)ccc3C4c3c(-c4ccccc4)cc4sc5ccccc5c4c3-c3ccccc3)c2c1[2H] ; [2H]c1c(C(C)(C)C)c([2H])c2c3c([2H])c(C(C)(C)C)c([2H])c([2H])c3n(-c3cc4c5c(c3)N(c3c(-c6ccccc6)cc6sc7ccccc7c6c3-c3ccccc3)c3ccc(-c6cc(C(C)(C)C)cc(C(C)(C)C)c6)cc3C5c3ccc(C(C)(C)C)cc3N4c3c(-c4ccccc4)cc4c(c3-c3ccccc3)Cc3ccccc3-4)c2c1[2H]] 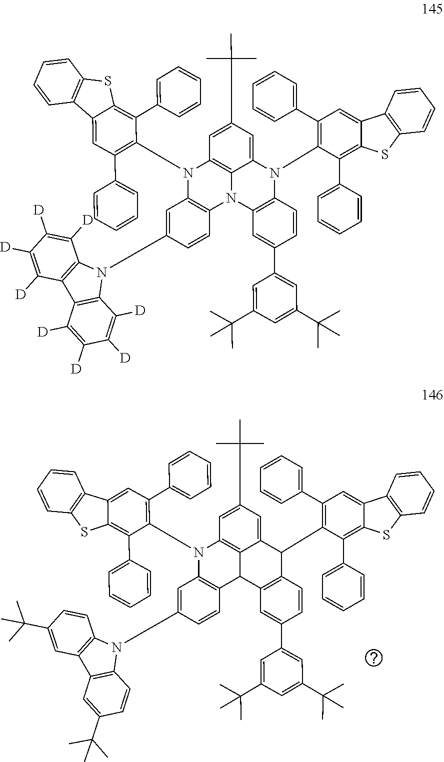 [SMILES: [2H]c1c([2H])c([2H])c2c(c1[2H])c1c([2H])c([2H])c([2H])c([2H])c1n2-c1ccc2c(c1)N(c1c(-c3ccccc3)cc3c(sc4ccccc43)c1-c1ccccc1)c1cc(C(C)(C)C)cc3c1N2c1cc(-c2cc(C(C)(C)C)cc(C(C)(C)C)c2)ccc1N3c1c(-c2ccccc2)cc2c(sc3ccccc32)c1-c1ccccc1 ; CC(C)(C)c1cc(-c2ccc3c(c2)C2c4ccc(-n5c6ccc(C(C)(C)C)cc6c6cc(C(C)(C)C)ccc65)cc4N(c4c(-c5ccccc5)cc5c(sc6ccccc65)c4-c4ccccc4)c4cc(C(C)(C)C)cc(c42)C3c2c(-c3ccccc3)cc3c(sc4ccccc43)c2-c2ccccc2)cc(C(C)(C)C)c1] 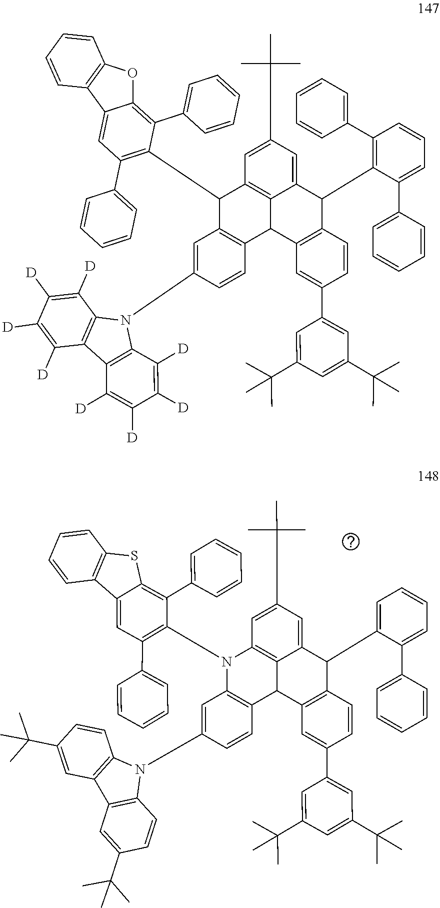 [SMILES: [2H]c1c([2H])c([2H])c2c(c1[2H])c1c([2H])c([2H])c([2H])c([2H])c1n2-c1ccc2c(c1)C(c1c(-c3ccccc3)cc3c(oc4ccccc43)c1-c1ccccc1)c1cc(C(C)(C)C)cc3c1C2c1cc(-c2cc(C(C)(C)C)cc(C(C)(C)C)c2)ccc1C3c1c(-c2ccccc2)cccc1-c1ccccc1 ; CC(C)(C)c1cc(-c2ccc3c(c2)C2c4ccc(-n5c6ccc(C(C)(C)C)cc6c6cc(C(C)(C)C)ccc65)cc4N(c4c(-c5ccccc5)cc5c(sc6ccccc65)c4-c4ccccc4)c4cc(C(C)(C)C)cc(c42)C3c2ccccc2-c2ccccc2)cc(C(C)(C)C)c1] 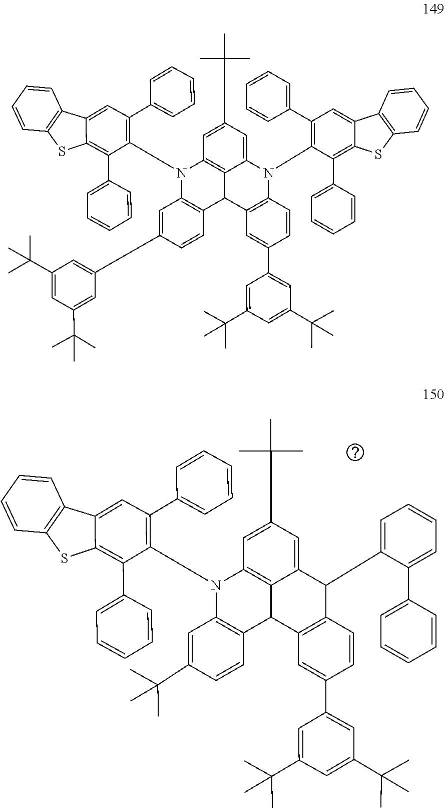 [SMILES: CC(C)(C)c1cc(-c2ccc3c(c2)C2c4ccc(-c5cc(C(C)(C)C)cc(C(C)(C)C)c5)cc4N(c4c(-c5ccccc5)cc5c(sc6ccccc65)c4-c4ccccc4)c4cc(C(C)(C)C)cc(c42)N3c2c(-c3ccccc3)cc3c(sc4ccccc43)c2-c2ccccc2)cc(C(C)(C)C)c1 ; CC(C)(C)c1cc(-c2ccc3c(c2)C2c4ccc(C(C)(C)C)cc4N(c4c(-c5ccccc5)cc5c(sc6ccccc65)c4-c4ccccc4)c4cc(C(C)(C)C)cc(c42)C3c2ccccc2-c2ccccc2)cc(C(C)(C)C)c1] 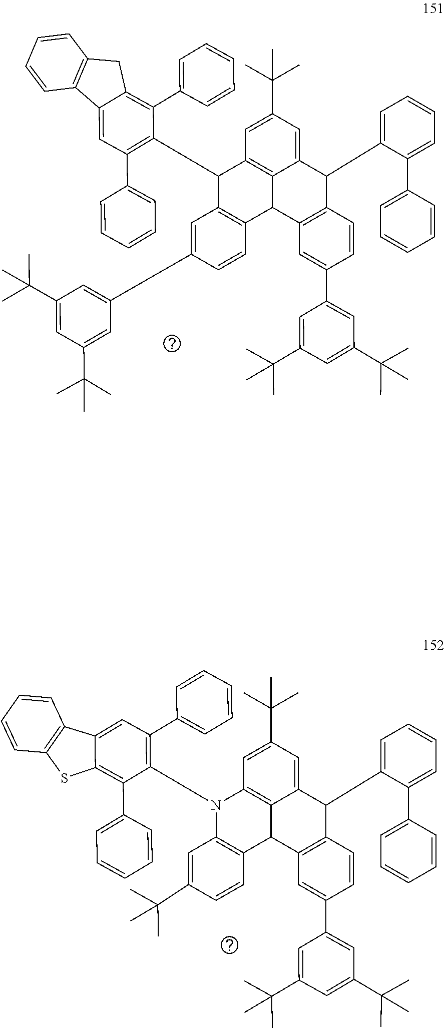 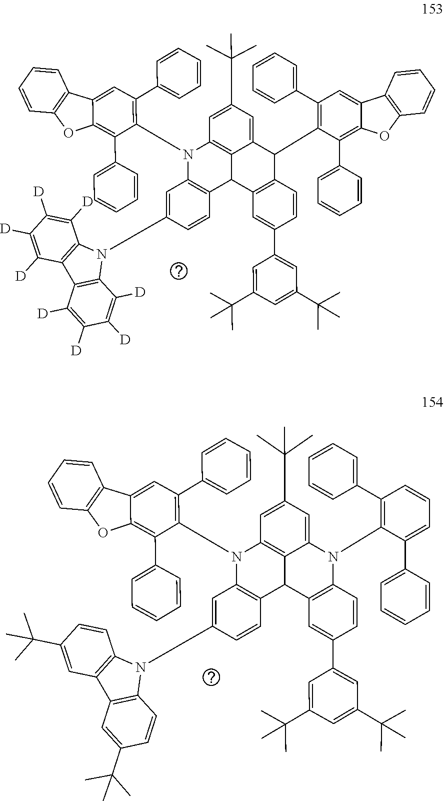 [SMILES: [2H]c1c([2H])c([2H])c2c(c1[2H])c1c([2H])c([2H])c([2H])c([2H])c1n2-c1ccc2c(c1)N(c1c(-c3ccccc3)cc3c(oc4ccccc43)c1-c1ccccc1)c1cc(C(C)(C)C)cc3c1C2c1cc(-c2cc(C(C)(C)C)cc(C(C)(C)C)c2)ccc1C3c1c(-c2ccccc2)cc2c(oc3ccccc32)c1-c1ccccc1 ; CC(C)(C)c1cc(-c2ccc3c(c2)C2c4ccc(-n5c6ccc(C(C)(C)C)cc6c6cc(C(C)(C)C)ccc65)cc4N(c4c(-c5ccccc5)cc5c(oc6ccccc65)c4-c4ccccc4)c4cc(C(C)(C)C)cc(c42)N3c2c(-c3ccccc3)cccc2-c2ccccc2)cc(C(C)(C)C)c1] 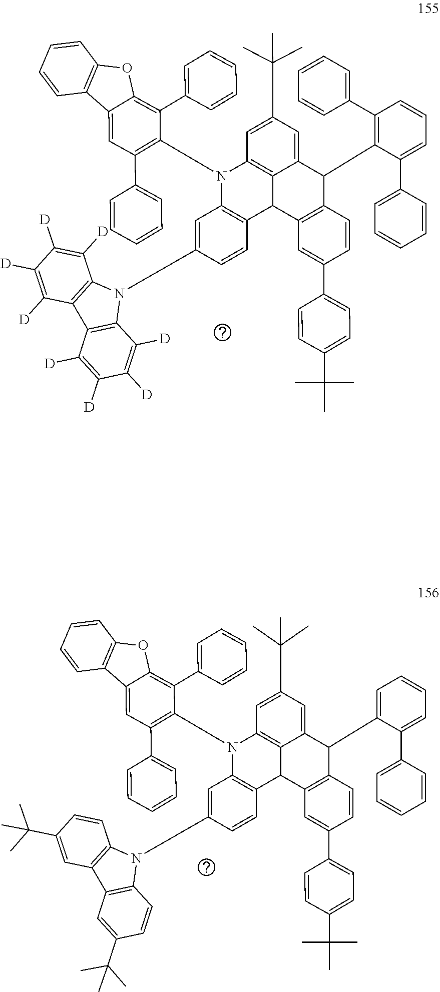 [SMILES: [2H]c1c([2H])c([2H])c2c(c1[2H])c1c([2H])c([2H])c([2H])c([2H])c1n2-c1ccc2c(c1)N(c1c(-c3ccccc3)cc3c(oc4ccccc43)c1-c1ccccc1)c1cc(C(C)(C)C)cc3c1C2c1cc(-c2ccc(C(C)(C)C)cc2)ccc1C3c1c(-c2ccccc2)cccc1-c1ccccc1 ; CC(C)(C)c1ccc(-c2ccc3c(c2)C2c4ccc(-n5c6ccc(C(C)(C)C)cc6c6cc(C(C)(C)C)ccc65)cc4N(c4c(-c5ccccc5)cc5c(oc6ccccc65)c4-c4ccccc4)c4cc(C(C)(C)C)cc(c42)C3c2ccccc2-c2ccccc2)cc1] 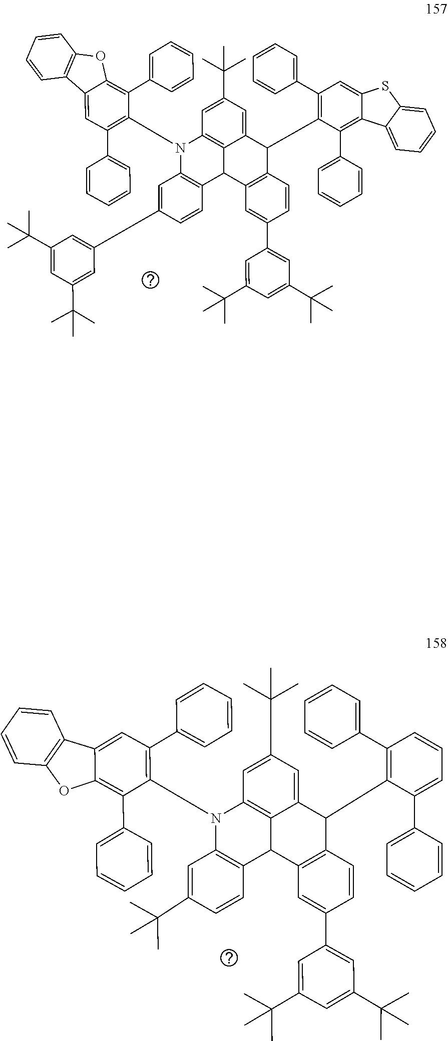 [SMILES: CC(C)(C)c1cc(-c2ccc3c(c2)C2c4ccc(-c5cc(C(C)(C)C)cc(C(C)(C)C)c5)cc4N(c4c(-c5ccccc5)cc5c(oc6ccccc65)c4-c4ccccc4)c4cc(C(C)(C)C)cc(c42)C3c2c(-c3ccccc3)cc3sc4ccccc4c3c2-c2ccccc2)cc(C(C)(C)C)c1 ; CC(C)(C)c1cc(-c2ccc3c(c2)C2c4ccc(C(C)(C)C)cc4N(c4c(-c5ccccc5)cc5c(oc6ccccc65)c4-c4ccccc4)c4cc(C(C)(C)C)cc(c42)C3c2c(-c3ccccc3)cccc2-c2ccccc2)cc(C(C)(C)C)c1] 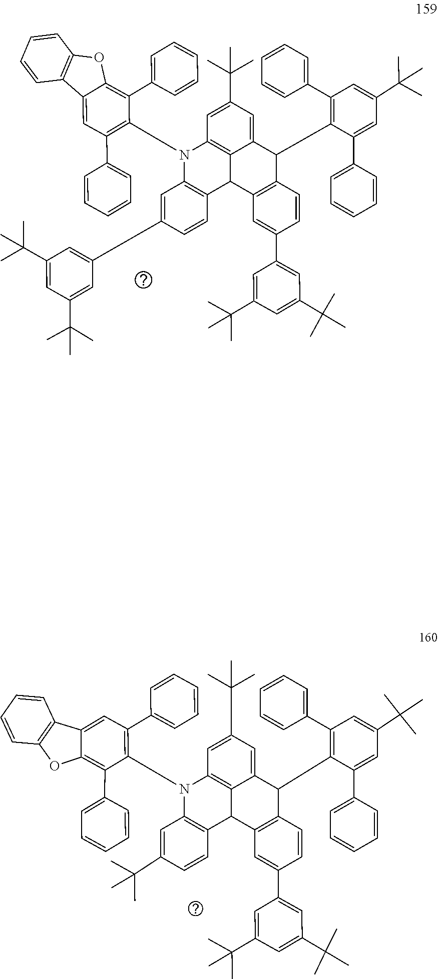 [SMILES: CC(C)(C)c1cc(-c2ccc3c(c2)C2c4ccc(-c5cc(C(C)(C)C)cc(C(C)(C)C)c5)cc4N(c4c(-c5ccccc5)cc5c(oc6ccccc65)c4-c4ccccc4)c4cc(C(C)(C)C)cc(c42)C3c2c(-c3ccccc3)cc(C(C)(C)C)cc2-c2ccccc2)cc(C(C)(C)C)c1 ; CC(C)(C)c1cc(-c2ccc3c(c2)C2c4ccc(C(C)(C)C)cc4N(c4c(-c5ccccc5)cc5c(oc6ccccc65)c4-c4ccccc4)c4cc(C(C)(C)C)cc(c42)C3c2c(-c3ccccc3)cc(C(C)(C)C)cc2-c2ccccc2)cc(C(C)(C)C)c1] 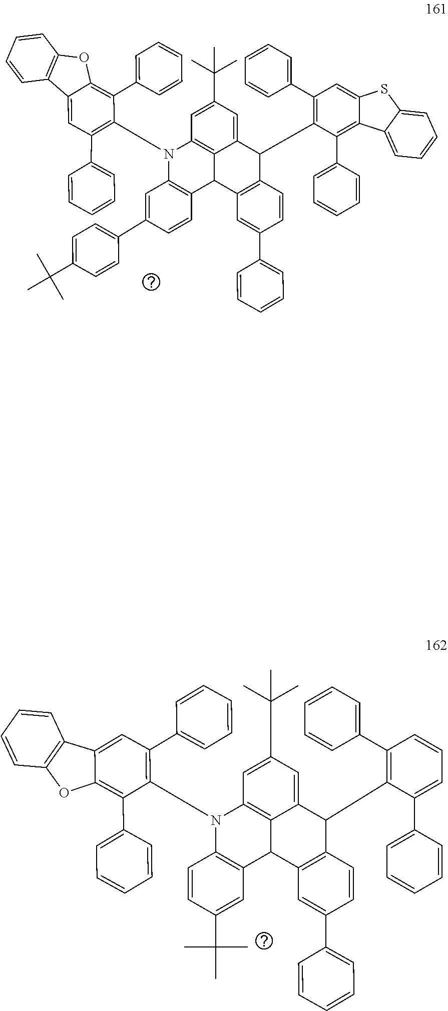 [SMILES: CC(C)(C)c1ccc(-c2ccc3c(c2)N(c2c(-c4ccccc4)cc4c(oc5ccccc54)c2-c2ccccc2)c2cc(C(C)(C)C)cc4c2C3c2cc(-c3ccccc3)ccc2C4c2c(-c3ccccc3)cc3sc4ccccc4c3c2-c2ccccc2)cc1 ; CC(C)(C)c1ccc2c(c1)C1c3cc(-c4ccccc4)ccc3C(c3c(-c4ccccc4)cccc3-c3ccccc3)c3cc(C(C)(C)C)cc(c31)N2c1c(-c2ccccc2)cc2c(oc3ccccc32)c1-c1ccccc1] 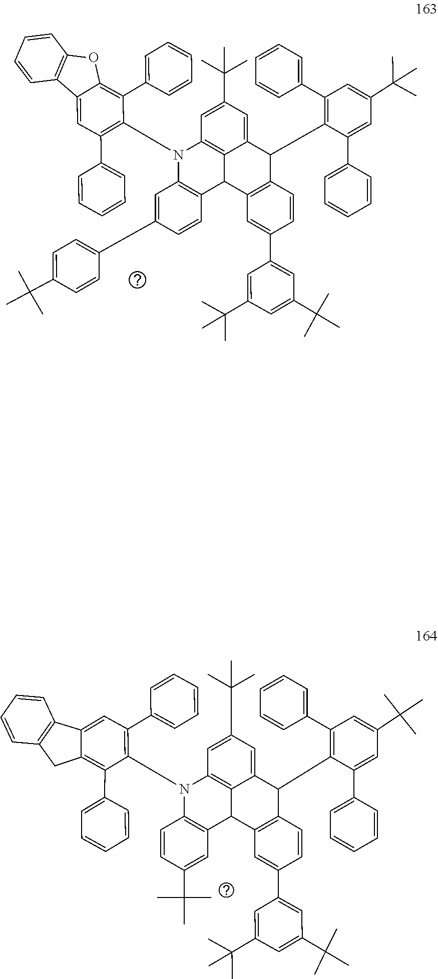 [SMILES: CC(C)(C)c1ccc(-c2ccc3c(c2)N(c2c(-c4ccccc4)cc4c(oc5ccccc54)c2-c2ccccc2)c2cc(C(C)(C)C)cc4c2C3c2cc(-c3cc(C(C)(C)C)cc(C(C)(C)C)c3)ccc2C4c2c(-c3ccccc3)cc(C(C)(C)C)cc2-c2ccccc2)cc1 ; CC(C)(C)c1cc(-c2ccc3c(c2)C2c4cc(C(C)(C)C)ccc4N(c4c(-c5ccccc5)cc5c(c4-c4ccccc4)Cc4ccccc4-5)c4cc(C(C)(C)C)cc(c42)C3c2c(-c3ccccc3)cc(C(C)(C)C)cc2-c2ccccc2)cc(C(C)(C)C)c1] 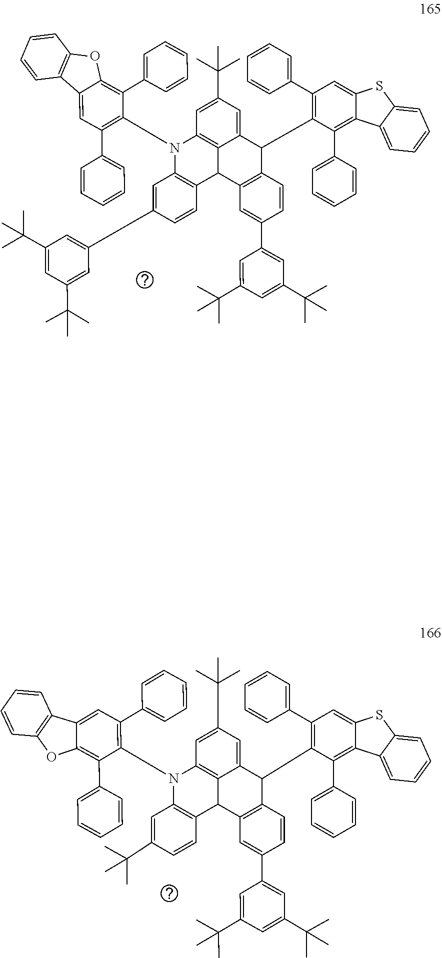 [SMILES: CC(C)(C)c1cc(-c2ccc3c(c2)C2c4ccc(-c5cc(C(C)(C)C)cc(C(C)(C)C)c5)cc4N(c4c(-c5ccccc5)cc5c(oc6ccccc65)c4-c4ccccc4)c4cc(C(C)(C)C)cc(c42)C3c2c(-c3ccccc3)cc3sc4ccccc4c3c2-c2ccccc2)cc(C(C)(C)C)c1 ; CC(C)(C)c1cc(-c2ccc3c(c2)C2c4ccc(C(C)(C)C)cc4N(c4c(-c5ccccc5)cc5c(oc6ccccc65)c4-c4ccccc4)c4cc(C(C)(C)C)cc(c42)C3c2c(-c3ccccc3)cc3sc4ccccc4c3c2-c2ccccc2)cc(C(C)(C)C)c1] 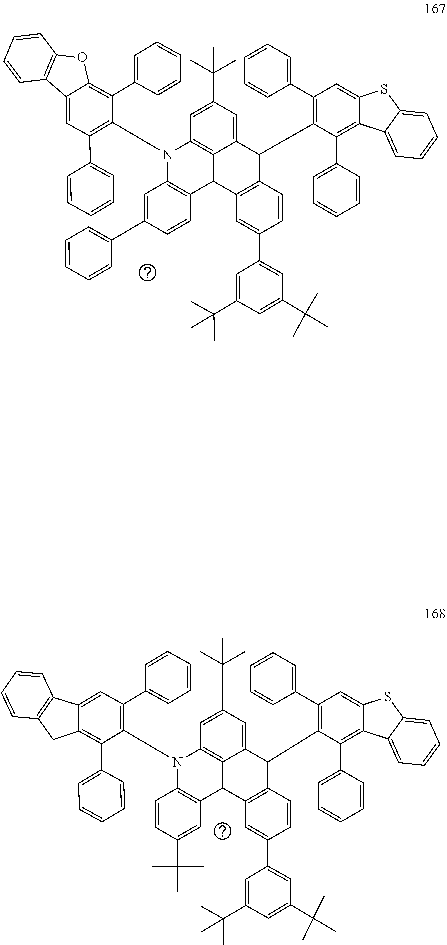 [SMILES: CC(C)(C)c1cc(-c2ccc3c(c2)C2c4ccc(-c5ccccc5)cc4N(c4c(-c5ccccc5)cc5c(oc6ccccc65)c4-c4ccccc4)c4cc(C(C)(C)C)cc(c42)C3c2c(-c3ccccc3)cc3sc4ccccc4c3c2-c2ccccc2)cc(C(C)(C)C)c1 ; CC(C)(C)c1cc(-c2ccc3c(c2)C2c4cc(C(C)(C)C)ccc4N(c4c(-c5ccccc5)cc5c(c4-c4ccccc4)Cc4ccccc4-5)c4cc(C(C)(C)C)cc(c42)C3c2c(-c3ccccc3)cc3sc4ccccc4c3c2-c2ccccc2)cc(C(C)(C)C)c1] 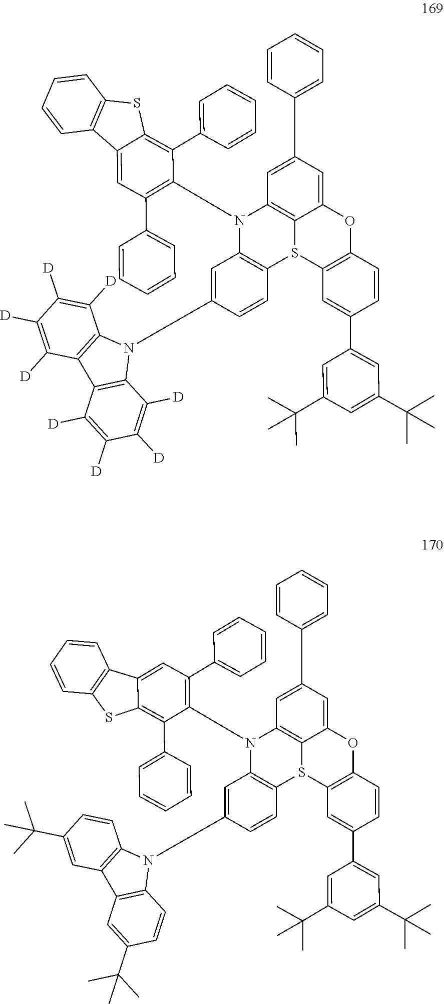 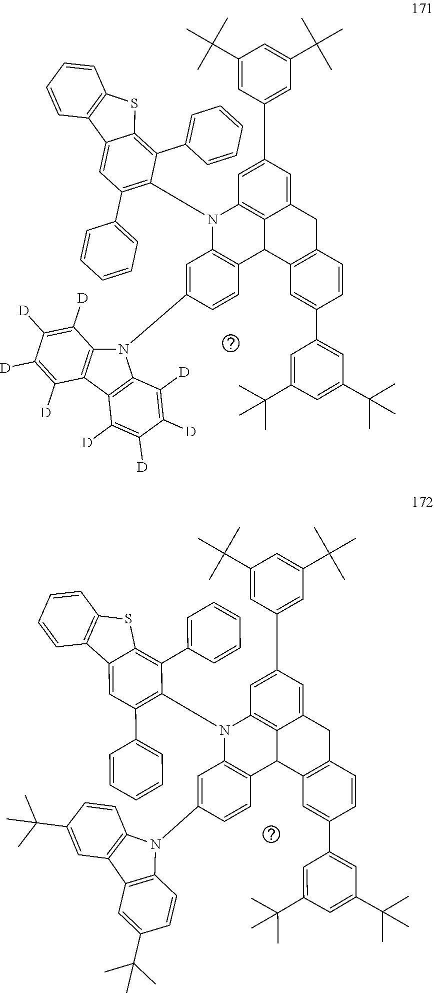 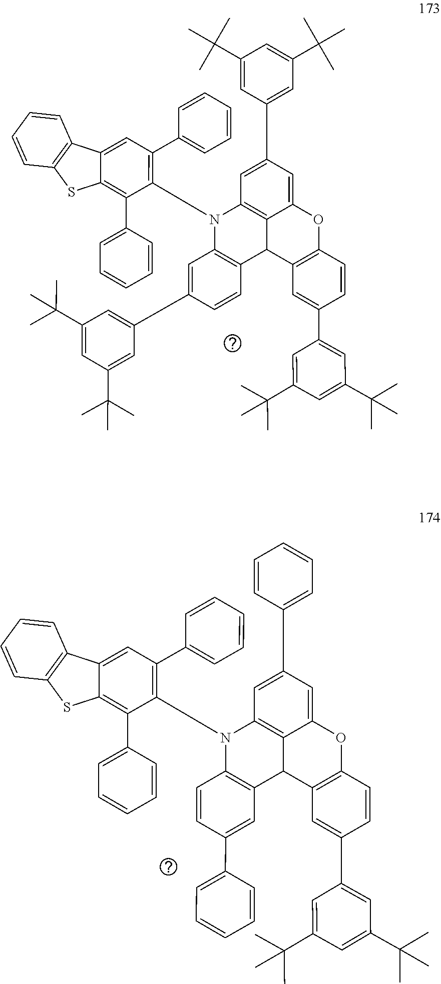 [SMILES: CC(C)(C)c1cc(-c2ccc3c(c2)C2c4ccc(-c5cc(C(C)(C)C)cc(C(C)(C)C)c5)cc4N(c4c(-c5ccccc5)cc5c(sc6ccccc65)c4-c4ccccc4)c4cc(-c5cc(C(C)(C)C)cc(C(C)(C)C)c5)cc(c42)O3)cc(C(C)(C)C)c1 ; CC(C)(C)c1cc(-c2ccc3c(c2)C2c4cc(-c5ccccc5)ccc4N(c4c(-c5ccccc5)cc5c(sc6ccccc65)c4-c4ccccc4)c4cc(-c5ccccc5)cc(c42)O3)cc(C(C)(C)C)c1] 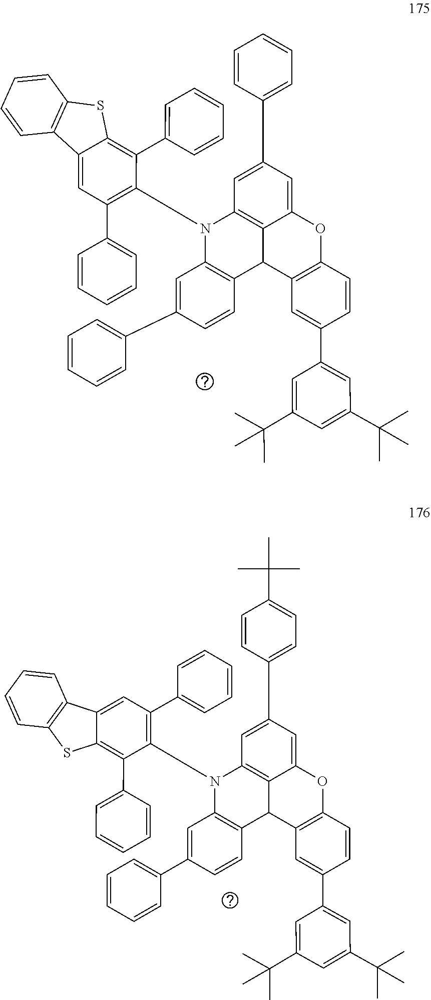 [SMILES: CC(C)(C)c1cc(-c2ccc3c(c2)C2c4ccc(-c5ccccc5)cc4N(c4c(-c5ccccc5)cc5c(sc6ccccc65)c4-c4ccccc4)c4cc(-c5ccccc5)cc(c42)O3)cc(C(C)(C)C)c1 ; CC(C)(C)c1ccc(-c2cc3c4c(c2)N(c2c(-c5ccccc5)cc5c(sc6ccccc65)c2-c2ccccc2)c2cc(-c5ccccc5)ccc2C4c2cc(-c4cc(C(C)(C)C)cc(C(C)(C)C)c4)ccc2O3)cc1] 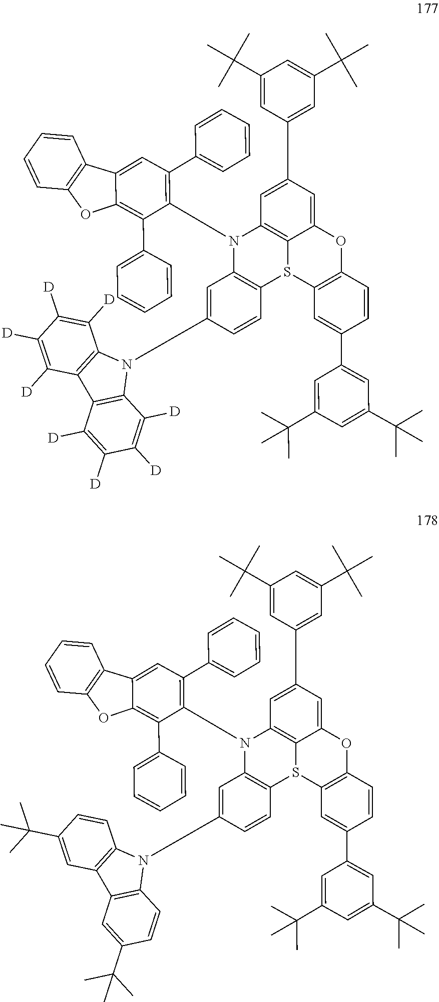 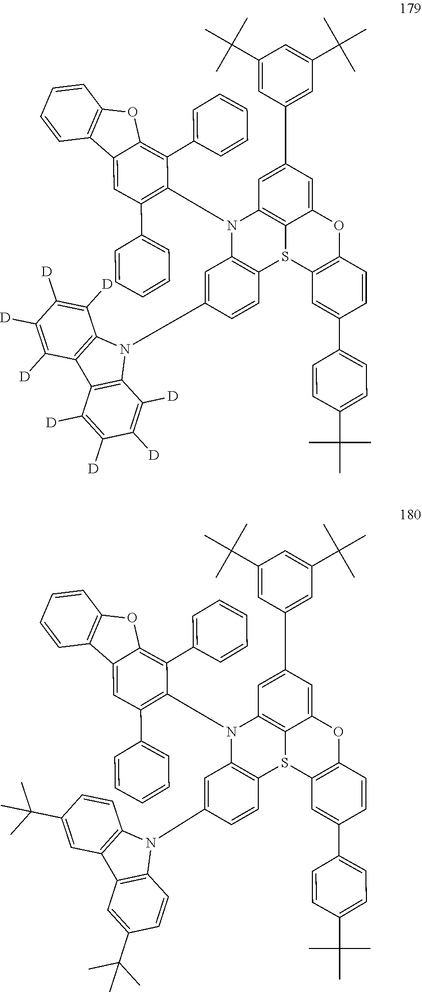 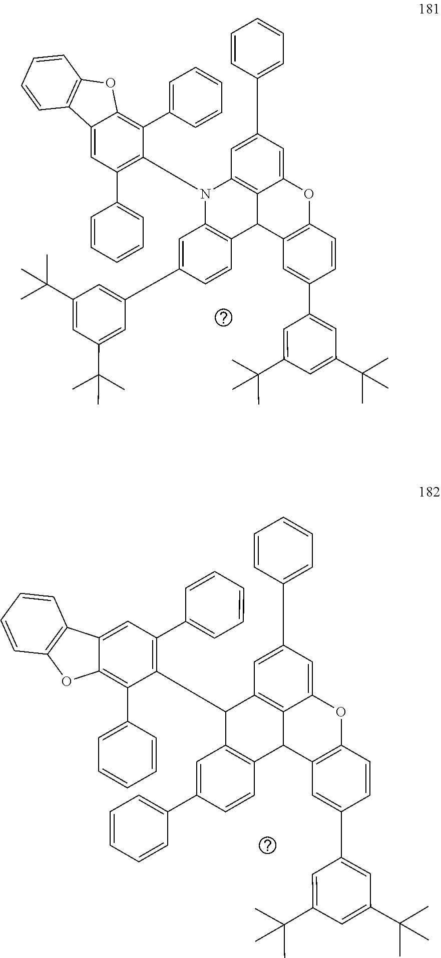 [SMILES: CC(C)(C)c1cc(-c2ccc3c(c2)C2c4ccc(-c5cc(C(C)(C)C)cc(C(C)(C)C)c5)cc4N(c4c(-c5ccccc5)cc5c(oc6ccccc65)c4-c4ccccc4)c4cc(-c5ccccc5)cc(c42)O3)cc(C(C)(C)C)c1 ; CC(C)(C)c1cc(-c2ccc3c(c2)C2c4ccc(-c5ccccc5)cc4C(c4c(-c5ccccc5)cc5c(oc6ccccc65)c4-c4ccccc4)c4cc(-c5ccccc5)cc(c42)O3)cc(C(C)(C)C)c1] 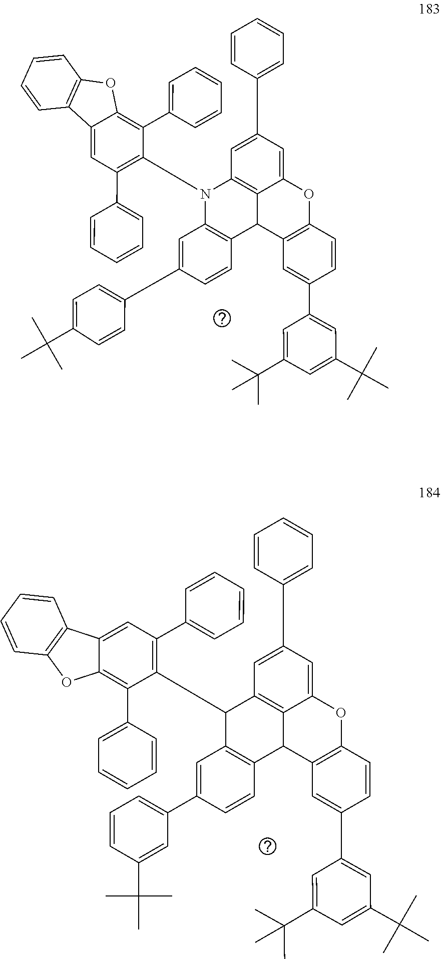 [SMILES: CC(C)(C)c1ccc(-c2ccc3c(c2)N(c2c(-c4ccccc4)cc4c(oc5ccccc54)c2-c2ccccc2)c2cc(-c4ccccc4)cc4c2C3c2cc(-c3cc(C(C)(C)C)cc(C(C)(C)C)c3)ccc2O4)cc1 ; CC(C)(C)c1cccc(-c2ccc3c(c2)C(c2c(-c4ccccc4)cc4c(oc5ccccc54)c2-c2ccccc2)c2cc(-c4ccccc4)cc4c2C3c2cc(-c3cc(C(C)(C)C)cc(C(C)(C)C)c3)ccc2O4)c1] 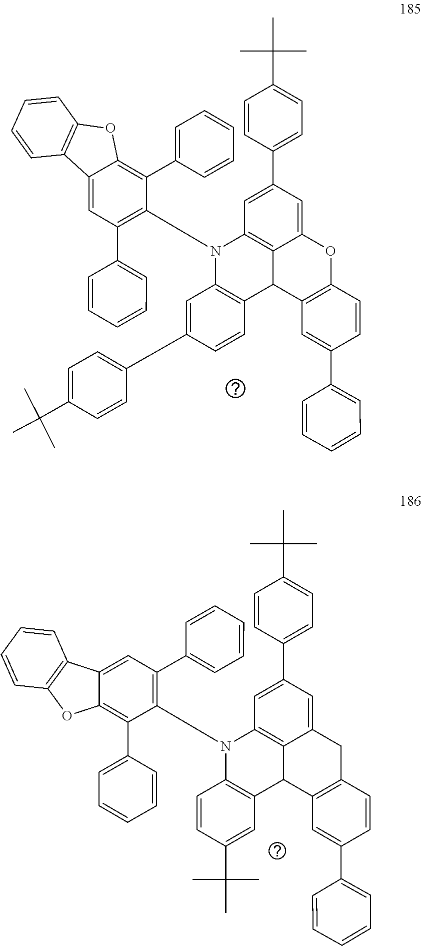 [SMILES: CC(C)(C)c1ccc(-c2ccc3c(c2)N(c2c(-c4ccccc4)cc4c(oc5ccccc54)c2-c2ccccc2)c2cc(-c4ccc(C(C)(C)C)cc4)cc4c2C3c2cc(-c3ccccc3)ccc2O4)cc1 ; CC(C)(C)c1ccc(-c2cc3c4c(c2)N(c2c(-c5ccccc5)cc5c(oc6ccccc65)c2-c2ccccc2)c2ccc(C(C)(C)C)cc2C4c2cc(-c4ccccc4)ccc2C3)cc1] 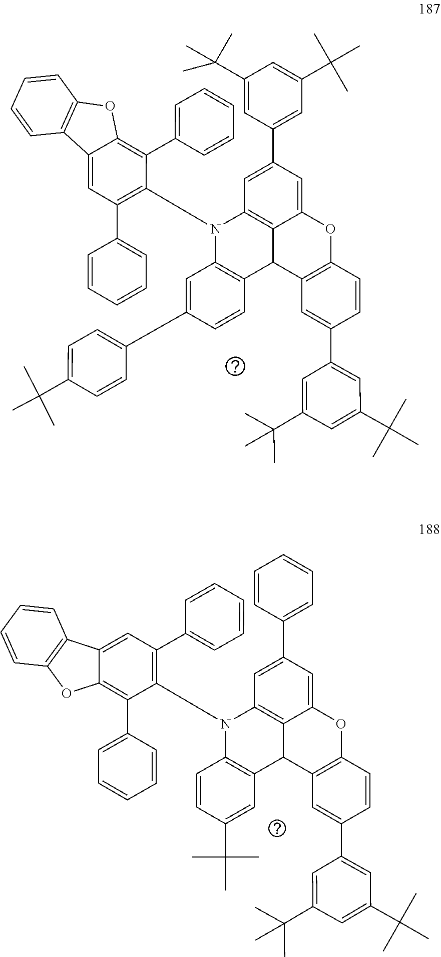 [SMILES: CC(C)(C)c1ccc(-c2ccc3c(c2)N(c2c(-c4ccccc4)cc4c(oc5ccccc54)c2-c2ccccc2)c2cc(-c4cc(C(C)(C)C)cc(C(C)(C)C)c4)cc4c2C3c2cc(-c3cc(C(C)(C)C)cc(C(C)(C)C)c3)ccc2O4)cc1 ; CC(C)(C)c1cc(-c2ccc3c(c2)C2c4cc(C(C)(C)C)ccc4N(c4c(-c5ccccc5)cc5c(oc6ccccc65)c4-c4ccccc4)c4cc(-c5ccccc5)cc(c42)O3)cc(C(C)(C)C)c1] 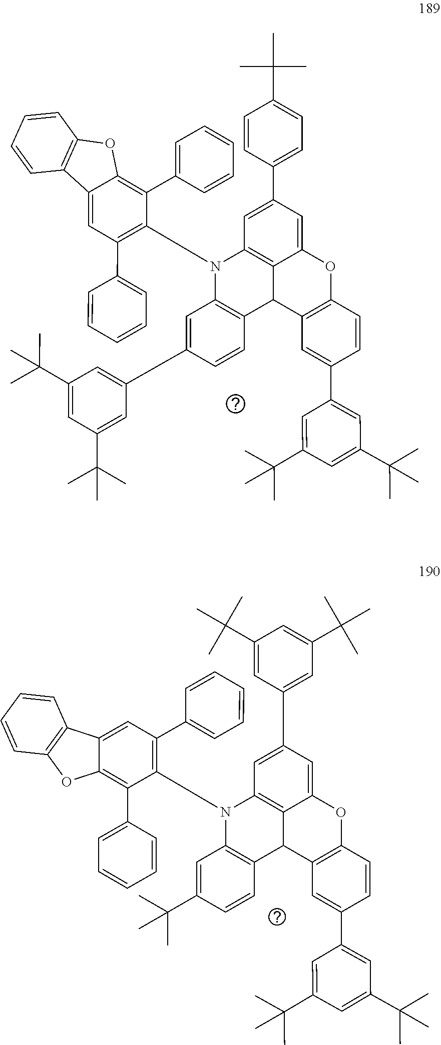 [SMILES: CC(C)(C)c1ccc(-c2cc3c4c(c2)N(c2c(-c5ccccc5)cc5c(oc6ccccc65)c2-c2ccccc2)c2cc(-c5cc(C(C)(C)C)cc(C(C)(C)C)c5)ccc2C4c2cc(-c4cc(C(C)(C)C)cc(C(C)(C)C)c4)ccc2O3)cc1 ; CC(C)(C)c1cc(-c2ccc3c(c2)C2c4ccc(C(C)(C)C)cc4N(c4c(-c5ccccc5)cc5c(oc6ccccc65)c4-c4ccccc4)c4cc(-c5cc(C(C)(C)C)cc(C(C)(C)C)c5)cc(c42)O3)cc(C(C)(C)C)c1] 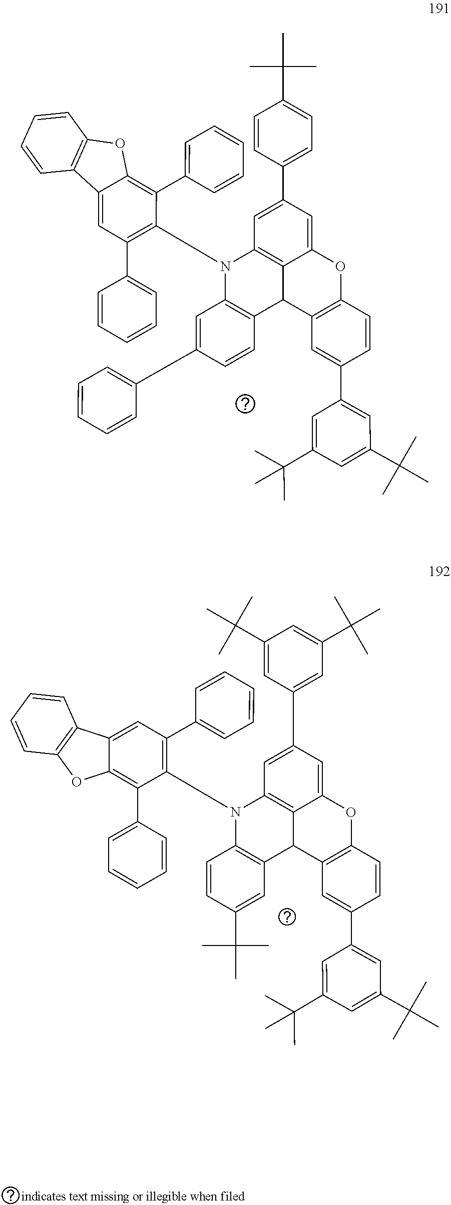 [SMILES: CC(C)(C)c1ccc(-c2cc3c4c(c2)N(c2c(-c5ccccc5)cc5c(oc6ccccc65)c2-c2ccccc2)c2cc(-c5ccccc5)ccc2C4c2cc(-c4cc(C(C)(C)C)cc(C(C)(C)C)c4)ccc2O3)cc1 ; CC(C)(C)c1cc(-c2ccc3c(c2)C2c4cc(C(C)(C)C)ccc4N(c4c(-c5ccccc5)cc5c(oc6ccccc65)c4-c4ccccc4)c4cc(-c5cc(C(C)(C)C)cc(C(C)(C)C)c5)cc(c42)O3)cc(C(C)(C)C)c1]
